(19) United States
(12) Patent Application Publication
(10) Pub. No.: US 2024/0325162 A1
(43) Pub. Date: Oct. 3, 2024

(54) INTERVERTEBRAL DEVICES, AND ASSOCIATED SYSTEMS AND METHODS

(71) Applicant: BLOOM BIOMEDICAL, INC., Irvine, CA (US)

(72) Inventors: Jason Liauw, Laguna Beach, CA (US); Nicholas Hu, Irvine, CA (US); Philippe Marchand, Montreal (CA); Lilibeth Martinez, Irvine, CA (US); Jack Izen, Irvine, CA (US); Jacob Taylor, Irvine, CA (US)

(21) Appl. No.: 18/619,125

(22) Filed: Mar. 27, 2024

Related U.S. Application Data
(60) Provisional application No. 63/492,731, filed on Mar. 28, 2023.
(60) Provisional application No. 63/617,743, filed on Jan. 4, 2024.
(60) Provisional application No. 63/562,033, filed on Mar. 6, 2024.

(51) Int. Cl. A61F 2/44; A61B 17/70; A61F 2/46

(52) U.S. Cl. CPC A61F 2/441; A61B 17/7043; A61B 17/7097; A61F 2/4611; A61F 2002/4635

(57) ABSTRACT

Spinal surgical procedures for implanting intervertebral devices, and associated devices and systems are described herein. A representative spinal surgical procedure can include gaining access to a diseased disc of a spine of a patient via a minimally-invasive a lateral, transpedicular, transfacet, or transforaminal access pathway. Various instruments can be inserted through the trocar to (i) remove some or all of the diseased disc, (ii) expand a disc space around the diseased disc, (iii) insert an intervertebral device into the disc space, and/or (iv) fill the intervertebral device with a fill material. Each of the aforementioned steps can be performed through the minimally-invasive access pathway provided by the trocar. This can minimize disruption to the flesh of the patient—minimizing patient pain and recovery time.

### CROSS-REFERENCE TO RELATED APPLICATIONS

This application claims the benefit of (i) U.S. Provisional Patent Application No. 63/492,731, filed Mar. 28, 2023, and titled “INTERVERTEBRAL DEVICES, AND ASSOCIATED SYSTEMS AND METHODS,” (ii) U.S. Provisional Patent Application No. 63/617,743, filed Jan. 4, 2024, and titled “INTERVERTEBRAL DEVICES, AND ASSOCIATED SYSTEMS AND METHODS,” and (iii) U.S. Provisional Patent Application No. 63/562,033, filed Mar. 6, 2024, and titled “INTERVERTEBRAL DEVICES, AND ASSOCIATED SYSTEMS AND METHODS,” each of which is incorporated herein by reference in its entirety.

### TECHNICAL FIELD

The present technology is directed to intervertebral devices, such as intervertebral fusion devices, that can be deployed minimally invasively, and associated system and methods.

### BACKGROUND

Degenerative joint disease in the spine usually involves the tandem degeneration of the intervertebral disc and the two posterior facet joints which act as a tripod to provide stabilization between the vertebrae of the spine. Degeneration of these joints are respectively termed disc degenerative disease (DDD) or disc arthropathy, and facet degenerative disease or facet arthropathy. The result of DDD is often thinning of the disc and a collapse of the disc height. The neural foramen are the openings through which the spinal nerve roots go through when leaving the spinal column, and the height of these foramina directly correspond to the height of the intervertebral disc. When the intervertebral disc height collapses, the natural height of the neural foramina also collapses and, accordingly, the exiting nerve root is compressed which can elicit nerve pain or radicular pain down the legs. Intervertebral disc height collapse can further cause ligamentous laxity and bulging in the spine. These ligaments, namely the posterior longitudinal ligament (PLL) and ligamentum flavum, surround the spinal cord and can bulge into the spinal canal as the disc height collapses. The result is compression of the whole spinal cord and/or the thecal sac positioned centrally in the spinal canal, which can cause radicular pain down the legs in addition to weakness and fatigue in the legs, (e.g., neurogenic claudication).

The surgical treatment of back pain and sciatic pain include etiologies of pain which all stem from disc degenerative joint disease in the spine. Surgical treatments include treating mechanical instability of the spine, treating nerve root compression, and restoring natural alignment of the spine, among others.

Mechanical instability is a result of degeneration of both the intervertebral disc and/or the posterior facet joints. The mechanical instability causes painful arthritic pain or arthropathy. Treating mechanical instability of the spine can include spinal fixation. Much of spinal fixation is geared toward reducing mechanical instability and thereby reducing the movement of arthritic joints that get inflamed with movement. There are numerous methods of spinal fixation including, for example, surgically implanting anterior column and/or posterior column fixation devices. The most pervasive spinal fixation method to this day involves posterior screws used in combination with interbody cages for anterior column support.

As described above, nerve root compression can occur centrally around the spinal cord or thecal sac, or laterally around the exiting nerve root at the neural foramen. Pain from nerve root compression often travels according to a nerve root dermatomal distribution, causing radicular or sciatic pain. Spinal surgical decompression is one method of treating nerve root compression, and aims to remove nerve pain by taking the compression off the nerve roots. This can be accomplished directly through removal of bone/ligament/disc compressing the nerve. It can also be accomplished indirectly by mechanically increasing the intervertebral height with an intervertebral interbody spacer or interlaminar spacer, thereby restoring neural foraminal height and reducing the bulging of the ligaments/disc that bulge into the spinal canal centrally.

One of the newer paradigms in spinal fixation and fusion is restoring natural alignment of the spine. For example, sagittal balance can be restored with intervertebral spacers that restore natural lordotic curvature of the spine. The restoration of neutral spinal alignment enables the patient to walk or stand with good posture rather than leaning forward. This reduces the strain on the paraspinal musculature in the spine. Not restoring natural spinal alignment when performing spinal fixation often results in “flat back syndrome,” in which patients have chronic lower back pain due to muscular fatigue. Additionally, inducing natural sagittal balance with the use of lordotic spacers has become a mainstay in preventing degeneration at the adjacent intervertebral spaces above and below a spinal fusion.

Vertebral interbody spacers have become an important part of spinal fixation for reasons which relate to the fundamentals of treating back pain and nerve pain—for example, treating mechanical instability, treating nerve root compression, and restoring the natural alignment of the spine. Intervertebral spacers can improve the treatment of mechanical instability. For example, the insertion of an intervertebral spacer provides for more anterior support in fixation of the spine. This helps stabilize mechanical instability. Some intervertebral spacers with large footprints can be used as standalone fixation devices. They can also be used in conjunction with posterior spinal fixation, as they add anterior column support allowing for more rigid stabilization which prevents hardware loosening and failure of fusion.

Intervertebral spacers can also improve treatment of nerve root compression. For example, intervertebral spacers can be used to increase the height of a collapsed and degenerated intervertebral disc space. This allows for indirect restoration of the height of the associated neural foramina which can relieve compression of the spinal nerve root exiting at that vertebral level. Additionally, increasing the height of the intervertebral disc space can restore tension to collapsed and bulging ligaments in the spinal canal, namely the PLL and ligamentum flavum. Restoring tension to these ligaments through distraction, termed ligamentum taxis, can reduce the bulging of these ligaments into the spinal canal. The combination of decompression of the neural foramina and spinal canal can reduce radicular sciatic pain and improves neurogenic claudication.

Intervertebral spacers can also help restore the natural alignment of the spine. For example, when the spine falls out of neutral global alignment-which predominantly refers to being hunched forward or out of sagittal alignment—the patient will experience muscular pain as the back strains throughout the day in an attempt to force the patient into a more neutral posture. The lumbar spine has built in natural lordosis which allows us to stand in an upright neutral position. As discs degenerate, thin, and collapse in height, the lumbar spine often loses that lordosis, which is why older patients with degenerated spines are often hunched forward. The popularity of intervertebral spacers has been driven by the fact that restoration of disc height or anterior column height can help bring a patient's spine into a more neutral or lordotic position. In this manner, a harmony of spinal balance can be achieved with a fusion. Indeed, there has been a rise in use of lordotic and hyperlordotic intervertebral spacers which further induce lordosis in the lumbar spine to help compensate for the kyphosis at other degenerated levels. Fusing a spine without the use of intervertebral spacers in the past often resulted in spinal fixation in a flat or kyphotic position which can leave a patient in chronic pain, termed “flat back” syndrome. Fusing without the use of intervertebral spacing is slowly becoming antiquated. There are currently no existing percutaneous interbody systems that allow for specified lordotic correction in the spine.

Interbody fusion implants can be placed into the disc space through either posterior, lateral, or anterior approach trajectories. The two posterior approaches are a posterior lumbar interbody fusion (PLIF) in which an interbody fusion implant is placed through a laminectomy, and transforaminal lumbar interbody fusion (TLIF) in which the facet joint is resected and the interbody fusion implant is placed through a postero-lateral trajectory. A lateral approach to the spine for placement of an interbody fusion implant is termed lateral lumbar or extreme lateral lumbar interbody fusion (LLIF/XLIF). The two anterior approaches to the lumbar spine are a directly anterior open approach, termed anterior lumbar interbody fusion (ALIF) or an antero-lateral approach, termed oblique lumbar interbody fusion (OLIF). Each of these approaches, with the exception of ALIF, can be performed through either an open or minimally invasive approach using retractors. Current minimally-invasive interbody fusion implants utilize an oblique postero-lateral approach with similar trajectory to a TLIF, but necessitate a slightly more lateral trajectory to get under the facet joint, targeting the Kambin's triangle. Typically, the facet is not removed with these approaches but rather the disc space is dilated in the Kambin's triangle.

One drawback with insertion of interbody cages is that there is still a significant amount of dissection and tissue trauma with open or minimally invasive open approaches to gain access to the disc space which equates to more post-operative pain and longer recovery. The use of more minimally invasive retractors allows for less tissue trauma, but still involves tissue retraction and retraction of the nerve root potentially-which can result in non-trivial post-operative pain. Further, the current approach with more minimally invasive retractors is to use smaller interbody implants that fit through the access port. However, a drawback of using smaller implants is that there is less contact surface area with the vertebrae above and below, leading to poor support which increases the risk of subsidence of the implant itself into the adjacent vertebrae. Percutaneous approaches to the spine with tubular dilators are an approach used to even further limit tissue dissection and retraction, but adoption has been limited due to poor visualization of the exiting nerve at the Kambin's triangle, and the risk of nerve injury when trying to dilate within a collapsed foramen where the safe zone of the Kambin's triangle is even narrower. Additionally, current percutaneous techniques are unfamiliar to many surgeons requiring additional training, and the unfamiliarity can often increase procedure time and risk.

Current development in interbody fusion devices and techniques, in addition to minimizing the approach, have also focused on the restoration of disc height and lordosis to restore spinal alignment. Existing open and minimally invasive techniques employ the use of instrumentation such as rasps, curettes, shavers, and dilators to clear the disc space and release the vertebral body ligamentous attachments to enable distraction of the intervertebral space. A lot of these instruments when used in an endoscopic or minimally invasive approach are hindered by the inability of the system instrumentation to enable the surgeon to prepare the disc space adequately for the deployed geometry of the implant since access is constrained during the procedure.

There are a number of existing choices of interbody fusion devices. Original interbody fusion devices were static polyetheretherketone (PEEK) or metal cages. To enable improved lordotic correction, these static cages were either shaped with built in lordotic angulation or were inserted and packed on the anterior most portion of the vertebral body and screws were compressed posteriorly to induce lordosis. The drawback of both static and expandable posteriorly inserted cages, however, remains that they often subside due to their small footprint and/or contact surface area on the vertebral endplate along with the fact that they do not conform to the vertebral body well which leads to point loading and endplate fracturing. Expandable cages especially are more susceptible to causing endplate fractures as expansion causes higher forces at the endplate/implant interface. Expandable cages that focus on inducing lordosis expand along the anterior wall but also cause point loading along the implant. These expandable cages can also reduce the overall contact surface area by lifting the vertebrae away from the more posterior portions of the rigid implant structure.

While lateral and anteriorly placed cages do possess more surface area and thereby have improved endplate coverage, they still engender a separate incision and dissection for the approach. Currently, endoscopically inserted interbody fusion devices can only expand in height.

Lumbar intervertebral fusion devices are indicated for use in skeletally mature patients with DDD at one, two, or more than two contiguous levels from L2-S1. DDD is defined as back pain of discogenic origin with degeneration of the disc confirmed via history and radiographic studies. Patients with DDD can also have spondylolisthesis at the involved level(s). Intervertebral devices are indicated to be used with a supplemental fixation system and autograft bone. Intervertebral fusion devices aim to restore disc height and lumbar lordosis. There are several methods of insertion for these devices, with limitations varying across the different approaches to insertion. Many insertion methods adopt either an anterior or posterior approach, where anterior insertion entails a greater risk for complications, but accomplishes superior restoration of height and lumbar lordosis.

Due to the nature of the incisions and complications involved with anterior methods of inserting intervertebral devices, surgeons are gravitating towards posterior methods of insertion. Specifically, the Transforaminal Lumbar Interbody Fusion (TLIF) approach has come to dominate the field of interbody fusion. However, insertion at a posterior-lateral angle comes with the limitation to the footprint size, which can result in higher incidence of endplate fractures and subsidence, given the small window of insertion limits the size of an interbody.

### BRIEF DESCRIPTION OF THE DRAWINGS

Many aspects of the present technology can be better understood with reference to the following drawings. The components in the drawings are not necessarily to scale. Instead, emphasis is placed on clearly illustrating the principles of the present technology.

FIGS. 1A and 1B are a side view and a top view, respectively, of a portion of a spine illustrating an access step of a spinal surgical procedure in accordance with embodiments of the present technology.

FIGS. 1C and 1D are a side view and a top view, respectively, of a portion of the spine illustrating a discectomy step of the spinal surgical procedure in accordance with embodiments of the present technology.

FIGS. 1E and 1F are side views of a portion of the spine illustrating a first stage and a second stage of a balloon deployment step of the spinal surgical procedure, respectively, in accordance with embodiments of the present technology.

FIGS. 1G and 1H are corresponding top views of the portion of the spine shown in FIGS. 1E and 1F, respectively, illustrating the first stage and the second stage of the balloon deployment step in accordance with embodiments of the present technology.

FIGS. 1I-1K are side view, another side view, and a top view, respectively, of a portion of the spine illustrating an intervertebral device deployment step of the spinal surgical procedure in accordance with embodiments of the present technology.

FIGS. 1L-IN are side view, another side view, and a top view, respectively, of a portion of the spine illustrating a first stage of an intervertebral device fill step of the spinal surgical procedure in accordance with embodiments of the present technology.

FIGS. 1O-1Q are a corresponding side view, another side view, and a top view of the portion of the spine shown in FIGS. 1L-IN, respectively, illustrating a second stage of the intervertebral device fill step in accordance with embodiments of the present technology.

FIG. 1R is an enlarged side view of a portion of the spine illustrating an intervertebral device closure step of the spinal surgical procedure in accordance with embodiments of the present technology.

FIG. 1S is a side view of a portion of the spine of the patient illustrating a posterior fixation step of the spinal surgical procedure in accordance with embodiments of the present technology.

FIG. 2 is a flow diagram of a process or method for performing a spinal surgical procedure in accordance with embodiments of the present technology.

FIG. 3A is a side view of a portion of a spine illustrating a first posterior fixation step of a spinal surgical procedure in accordance with embodiments of the present technology.

FIG. 3B is a side view of a portion of the spine illustrating a second posterior fixation step of the spinal surgical procedure in accordance with embodiments of the present technology.

FIG. 3C is a side view of a portion of the spine illustrating a third posterior fixation step of the spinal surgical procedure in accordance with embodiments of the present technology.

FIG. 3D is a side view, including an enlarged portion, of a portion of the spine illustrating an access step of the spinal surgical procedure in accordance with embodiments of the present technology.

FIGS. 3E and 3F are side views, including enlarged portions, of a portion of the spine illustrating distraction steps of the spinal surgical procedure in accordance with embodiments of the present technology.

FIGS. 3G-3I are side views, including enlarged portions, of a portion of the spine illustrating distraction steps of the spinal surgical procedure in accordance with additional embodiments of the present technology.

FIGS. 3J and 3K are side views of a portion of the spine illustrating posterior fixation locking steps of the spinal surgical procedure in accordance with embodiments of the present technology.

FIG. 3L is a side view, including an enlarged portion, of a portion of the spine illustrating an intervertebral device deployment step of the spinal surgical procedure in accordance with embodiments of the present technology.

FIG. 3M is a side view of a portion of the spine illustrating finally-implanted first and second intervertebral devices and a posterior fixation assembly in accordance with embodiments of the present technology.

FIGS. 4A and 4B are side views of a portion of a spine before and after inflation of a balloon in accordance with embodiments of the present technology.

FIG. 5 is a flow diagram of a process or method for performing a spinal surgical procedure in accordance with embodiments of the present technology.

FIG. 6A is a perspective view of an access alignment assembly positioned on a patient in accordance with embodiments of the present technology.

FIG. 6B is a schematic perspective view of a marker grid of the access alignment assembly of FIG. 6A in accordance with embodiments of the present technology.

FIGS. 7A and 7B are a top view and a side view, respectively, of a trocar for providing access to a vertebra or disc of a spine in accordance with embodiments of the present technology.

FIG. 8A is a perspective view of a trocar and a pair of stylets for use with the trocar in accordance with embodiments of the present technology.

FIG. 8B is a side view of a spine illustrating access via the trocar and the stylets of FIG. 8A in accordance with embodiments of the present technology.

FIG. 8C is a side view of the stylets of FIGS. 8A and 8B in accordance with additional embodiments of the present technology.

FIG. 9 is a side view of a trocar in accordance with embodiments of the present technology.

FIG. 10 is a side view of a trocar in accordance with embodiments of the present technology.

FIGS. 11A and 11B are side views of a trocar in a first position and a second position, respectively, in accordance with embodiments of the present technology.

FIGS. 11C and 11D are side views of an expandable anchoring section of the trocar of FIGS. 11A and 11B in the second position in accordance with embodiments of the present technology.

FIG. 12A is a perspective view of a trocar in accordance with embodiments of the present technology.

FIGS. 12B and 12C are side views of the trocar of FIG. 12A inserted through an introducer in a first position and a second position, respectively, in accordance with embodiments of the present technology.

FIGS. 13A-13C are a coronal view, a top view, and another top view, respectively, of an intervertebral device deployment step of a spinal surgical procedure utilizing the trocar of FIGS. 12A-12C and the introducer of FIGS. 12B and 12C in accordance with embodiments of the present technology.

FIG. 14 is a side view of a posterior fixation assembly in accordance with embodiments of the present technology.

FIG. 15A is an exploded side view of a fixation member of a posterior fixation assembly in accordance with embodiments of the present technology.

FIG. 15B is a side cross-sectional view of a screw of the fixation member of FIG. 15A in accordance with embodiments of the present technology.

FIG. 15C is a side view of a posterior fixation assembly including a plurality of the fixation members of FIG. 15A in accordance with embodiments of the present technology.

FIGS. 15D and 15E are side views of a fixation member and a tower member of FIG. 15C secured to a vertebra of a spine in accordance with embodiments of the present technology.

FIG. 16 is a side view of a fixation and access assembly in accordance with embodiments of the present technology.

FIGS. 17A and 17B are side views of a trocar access system including an access trocar and a steerable trocar in accordance with embodiments of the present technology.

FIG. 18A is a side view of a discectomy device in accordance with embodiments of the present technology.

FIG. 18B is a side of the discectomy device of FIG. 18A in accordance with additional embodiments of the present technology.

FIG. 18C is a side of the discectomy device of FIG. 18A in accordance with additional embodiments of the present technology.

FIG. 19 is a side view of a discectomy device in accordance with embodiments of the present technology.

FIG. 20 is a perspective view of an elongate member of a discectomy device in accordance with embodiments of the present technology.

FIG. 21 is a perspective view of an elongate member of a discectomy device in accordance with embodiments of the present technology.

FIG. 22A is a side view of a discectomy device in accordance with embodiments of the present technology.

FIG. 22B is an enlarged perspective view of a portion of an elongate member of the discectomy device of FIG. 22A in accordance with embodiments of the present technology.

FIG. 23 is a perspective side view of a discectomy device in accordance with embodiments of the present technology.

FIGS. 24A-24D are side views of various portions of a discectomy device in accordance with embodiments of the present technology.

FIG. 25 is a side view of a discectomy device inserted through an introducer for accessing a spine of a patient in accordance with embodiments of the present technology.

FIG. 26 is a side view of a discectomy device in accordance with embodiments of the present technology.

FIGS. 27A and 27B are enlarged side views of a distal portion of a discectomy device in accordance with embodiments of the present technology.

FIG. 28 includes multiple side views of distal portions of curette-like or rasp-like discectomy devices in accordance with embodiments of the present technology.

FIGS. 29A-29D are perspective side views of distal portions of discectomy devices in accordance with embodiments of the present technology.

FIG. 30 is a side view of a distal portion of a balloon device positioned through a trocar in accordance with embodiments of the present technology.

FIG. 31 is a side view of a balloon of a balloon device deployed and expanded within a disc space of a spine of a patient in accordance with embodiments of the present technology.

FIG. 32A is a side view of a balloon of a balloon device in accordance with embodiments of the present technology.

FIG. 32B is a side view of the balloon of FIG. 32A deployed and expanded within a disc space of a spine of a patient in accordance with embodiments of the present technology.

FIG. 33A is a side view of a balloon of a balloon device in accordance with embodiments of the present technology.

FIG. 33B is a side view of the balloon of FIG. 33A deployed and expanded within a disc space of a spine of a patient in accordance with embodiments of the present technology.

FIG. 34 is a side view of a balloon device deployed and expanded within a disc space of a spine of a patient in accordance with embodiments of the present technology.

FIG. 35 is a graph illustrating a representative pressure-volume curve sensed by a pressure sensing assembly during expansion of a balloon in accordance with embodiments of the present technology.

FIG. 36 is a side view of a balloon of a balloon device in accordance with embodiments of the present technology.

FIG. 37 is a side view of a balloon of a balloon device in accordance with embodiments of the present technology.

FIG. 38 is a side view of a balloon of a balloon device in accordance with embodiments of the present technology.

FIG. 39 is a side view of a balloon of a balloon device deployed and expanded within a disc space of a spine of a patient in accordance with embodiments of the present technology.

FIG. 40A is as perspective side view of an intervertebral device in accordance with embodiments of the present technology.

FIG. 40B illustrates various patterns in which filaments of the intervertebral device of FIG. 40A can be braided together in accordance with embodiments of the present technology.

FIG. 40C illustrates various patterns in which the filaments of the intervertebral device of FIG. 40A can be woven together and/or can include axial reinforcement filaments in accordance with embodiments of the present technology.

FIG. 41 is side view of an intervertebral device deployed and expanded within a disc space of a spine of a patient in accordance with embodiments of the present technology.

FIG. 42 is a perspective view of an intervertebral device in accordance with embodiments of the present technology.

FIG. 43 is a perspective view of an intervertebral device in accordance with embodiments of the present technology.

FIG. 44 is a perspective of a filament of an intervertebral device in accordance with embodiments of the present technology.

FIGS. 45A-45D are side views of different steps of a method for securing multiple filaments of an intervertebral device together to a hub in accordance with embodiments of the present technology.

FIG. 45E is an enlarged view of a portion of FIG. 45D in accordance with embodiments of the present technology.

FIGS. 46A-46D are side views of different steps of a method for securing multiple filaments of an intervertebral device together to a hub in accordance with additional embodiments of the present technology.

FIGS. 47A and 47B are side views of different steps of a method for securing multiple filaments of an intervertebral device together to a hub in accordance with embodiments of the present technology.

FIGS. 48A and 48B are a perspective top view and a perspective side view, respectively, of an intervertebral device in accordance with embodiments of the present technology.

FIG. 49 is an enlarged perspective view of a fill material in accordance with embodiments of the present technology.

FIG. 50 is an enlarged side view of a fill material in accordance with embodiments of the present technology.

FIG. 51 is an enlarged perspective view of a fill material extending from an introducer in accordance with embodiments of the present technology.

FIG. 52 is a graph of packing density (y-axis) versus percentage of large fill particles relative to small fill particles (x-axis) in accordance with embodiments of the present technology.

FIG. 53 is a table of different moduli of elasticity of various materials that can be used for the particles of a fill material in accordance with embodiments of the present technology.

FIG. 54 is an enlarged side view of a fill material in accordance with embodiments of the present technology.

FIG. 55 is an enlarged side view of a fill material in accordance with embodiments of the present technology.

FIG. 56 is a perspective view of a fill material comprising a plurality of particles in accordance with embodiments of the present technology.

FIG. 57A is a perspective view of a fill material comprising a plurality of particles subject to an axial force via a loading machine in accordance with embodiments of the present technology.

FIG. 57B is a perspective view of one of the particles of the fill material of FIG. 57A in accordance with embodiments of the present technology.

FIG. 58A is a perspective view of a fill material comprising a plurality of particles subject to an axial force via a loading machine in accordance with embodiments of the present technology.

FIG. 58B is a perspective view of one of the particles of the fill material of FIG. 58A in accordance with embodiments of the present technology.

FIG. 59A is a perspective view of a fill material comprising a plurality of particles subject to an axial force via a loading machine in accordance with embodiments of the present technology.

FIG. 59B is a perspective view of one of the particles of the fill material of FIG. 59A in accordance with embodiments of the present technology.

FIG. 60A is a perspective view of a fill material comprising a plurality of particles subject to an axial force via a loading machine in accordance with embodiments of the present technology.

FIG. 60B is a perspective view of one of the particles of the fill material of FIG. 60A in accordance with embodiments of the present technology.

FIG. 61 is a perspective side view of a proximal portion of a filling device inserted through an introducer in accordance with embodiments of the present technology.

FIG. 62 is a perspective side view of a distal portion of a filling device in accordance with embodiments of the present technology.

FIG. 63 is a side view of a distal portion of a filling device and an intervertebral device deployed and expanded within a disc space of a spine of a patient in accordance with embodiments of the present technology.

FIG. 64 is a side view of a distal portion of a filling device and an intervertebral device deployed and expanded within a disc space of a spine of a patient in accordance with embodiments of the present technology.

FIG. 65 is a side view of a closure mechanism in accordance with embodiments of the present technology.

FIG. 66A is a front view of a tensioning and/or closure mechanism in accordance with embodiments of the present technology.

FIG. 66B is a side view of the tensioning and/or closure mechanism of FIG. 66A installed on an intervertebral device in accordance with embodiments of the present technology.

FIG. 67 is a side view of a tensioning and/or closure mechanism in accordance with embodiments of the present technology.

FIG. 68 is a top view of an intervertebral device deployed within a disc space of a spine and including a tensioning mechanism in accordance with embodiments of the present technology.

FIG. 69A is a side view of an intervertebral device coupled to a deployment shaft in accordance with embodiments of the present technology.

FIGS. 69B and 69C are an enlarged view of a coupling between the intervertebral device of FIG. 69A and the deployment shaft of FIG. 69B and a side view of the deployment shaft of FIG. 69A, respectively, in accordance with embodiments of the present technology.

FIG. 70A is a perspective view of an intervertebral device and a deployment shaft in accordance with embodiments of the present technology.

FIG. 70B is a perspective view of a fill cartridge in accordance with embodiments of the present technology.

FIG. 70C is an enlarged view of a portion of the fill cartridge of FIG. 70B in accordance with embodiments of the present technology.

FIG. 70D is a perspective view of a fill member of FIGS. 70B and 70C in accordance with embodiments of the present technology.

FIG. 71A is a side view of a spine during a corpectomy procedure in accordance with embodiments of the present technology.

FIG. 71B is a side view of the spine of FIG. 71A during another stage of the corpectomy procedure in accordance with embodiments of the present technology.

FIG. 72A is a side view of a portion of a spinal fixation system attached to a spine of a patient including in accordance with embodiments of the present technology.

FIG. 72B is an identical side view of the portion of the spinal fixation system attached to the spine of FIG. 72A illustrating various distances, angles, and/or points of rotation that can be manipulated to drive other target distances, angles, and/or points of rotation in accordance with embodiments of the present technology.

FIG. 73A is a partially schematic side view of the portion of the spinal fixation system of FIGS. 72A and 72B in accordance with embodiments of the present technology.

FIG. 73B is a side view of a portion of the spine of FIGS. 72A-73A further illustrating an additional lower vertebra in accordance with embodiments of the present technology.

FIG. 74 is an identical side view of the portion of the spinal fixation system attached to the spine of FIG. 72A illustrating an additional driver inserted through a first tower member and engaging a first fixation member in accordance with embodiments of the present technology.

FIG. 75 is a side view of a posterior spinal fixation instrument/system in accordance with embodiments of the present technology.

FIG. 76 is a side view of a posterior spinal fixation instrument/system in accordance with embodiments of the present technology.

FIG. 77 is a side view of a portion of a spinal position sensing system configured to be attached to a spine of a patient in accordance with embodiments of the present technology.

FIG. 78A is an isometric view of a connector guide member in accordance with embodiments of the present technology.

FIG. 78B is an isometric view of the connector guide member of FIG. 78A coupling a trocar to a spinal fixation system attached to a portion of a spine of a patient in accordance with embodiments of the present technology.

FIG. 79A is a top view of a connector guide member in accordance with embodiments of the present technology.

FIGS. 79B and 79C are sides views of a clamp member of the connector guide member of FIG. 79A in a fully engaged position and a partially engaged position, respectively, in accordance with embodiments of the present technology.

FIG. 79D is an isometric view of the connector guide member of FIGS. 79A-79C coupling a trocar to a spinal fixation system attached to a portion of a spine of a patient in accordance with embodiments of the present technology.

FIG. 80 is an isometric view of a connector guide member coupling a trocar to a spinal fixation system attached to a portion of a spine of a patient in accordance with embodiments of the present technology.

FIGS. 81A and 81B are isometric views of a connector guide member coupling a trocar to a spinal fixation system attached to a portion of a spine of a patient in accordance with embodiments of the present technology.

FIG. 82 is an isometric view of a connector guide member coupling a trocar to a spinal fixation system attached to a portion of a spine of a patient in accordance with embodiments of the present technology.

FIG. 83A is an isometric view of a connector guide member coupling a trocar to a spinal fixation system attached to a portion of a spine of a patient in accordance with embodiments of the present technology.

FIG. 83B is a cross-sectional side view of the connector guide member of FIG. 83A in accordance with embodiments of the present technology.

FIGS. 84A-84C are cross-sectional isometric views of the connector guide member of FIGS. 83A and 83B in accordance additional embodiments of the present technology.

FIG. 85 is a flow diagram of a process or method for performing a spinal surgical procedure on a spine of a patient in which a connector guide member is used to fix a trocar to a spanning member of a posterior fixation system in accordance with embodiments of the present technology.

FIG. 86 is an isometric view of a connector guide member coupling a trocar to a spinal fixation system attached to a portion of a spine of a patient in accordance with embodiments of the present technology.

FIGS. 87-89 are flow diagrams of processes or methods for performing a spinal surgical procedure on a spine of a patient in which the connector guide member of FIG. 86 is used to fix a trocar to a fixation member of a posterior fixation system in accordance with embodiments of the present technology.

FIG. 90A is a side view of a portion of a spine of a patient illustrating a navigation and trajectory planning step of a spinal surgical procedure in accordance with embodiments of the present technology.

FIG. 90B is a side view of a portion of the spine illustrating an access step of the spinal surgical procedure in accordance with embodiments of the present technology.

FIG. 90C is a side view and an enlarged front view of a portion of the spine illustrating a first intervertebral device deployment step of the spinal surgical procedure in accordance with embodiments of the present technology.

FIG. 90D is a side view and an enlarged front view of a portion of the spine illustrating a second intervertebral device deployment step of the spinal surgical procedure in accordance with embodiments of the present technology.

### DETAILED DESCRIPTION

Aspects of the present technology are directed generally toward intervertebral devices, such as intervertebral fusion devices, that can be deployed minimally invasively or percutaneously, and associated system and methods. In several of the embodiments described below, a method for performing a spinal surgical procedure to implant an intervertebral device can include inserting a trocar into a patient to proximate to a diseased disc via a lateral, transpedicular, transfacet, transforaminal, and/or other approach. The trocar can provide an access pathway for subsequent instruments to be inserted thereto for treating the diseased disc. The method can further include inserting a discectomy device through the trocar and using the discectomy device to remove some or all of the diseased disc to form a disc space. Next, a balloon can be inserted through the trocar into the disc space and expanded to further disrupt and/or clear any remaining portion of the diseased disc and to lift an upper vertebra adjacent the diseased disc relative to a lower vertebra adjacent the diseased disc (e.g., to create lordosis). Then, the intervertebral device can be inserted through the trocar into the disc space and expanded within the disc space. The intervertebral device can comprise a braid, weave, mesh, and/or the like of filaments. Next, the intervertebral device can be filled with a fill material, such as a plurality of particles that form a gabion-like structure when loaded. In some embodiments, the intervertebral device is tensioned to better pack the fill material therein and/or to induce the gabion-like structure. The intervertebral device can then be closed to inhibit or even prevent egress of the fill material and released (e.g., from a delivery shaft) within the disc space. Finally, a posterior fixation assembly can be attached to the upper and lower vertebrae adjacent the disc space to stabilize the vertebrae and provide for bone ingrowth into the intervertebral device and the fill material therein.

Notably, each of the steps of the spinal surgical method can be performed through the open surgery, minimally-invasive, or percutaneous port/access pathway provided by the trocar. In some aspects of the present technology, this can minimize disruption to the tissue of the patient—minimizing patient pain and recovery time. Furthermore, the spinal surgical method can traverse safer pathways that require smaller size compared to conventional techniques. For example, the trocar and various instruments can be sized to fit through a transpedicular (or other) approach that encompasses a pathway (e.g., corridor) of less than 4.5 millimeters.

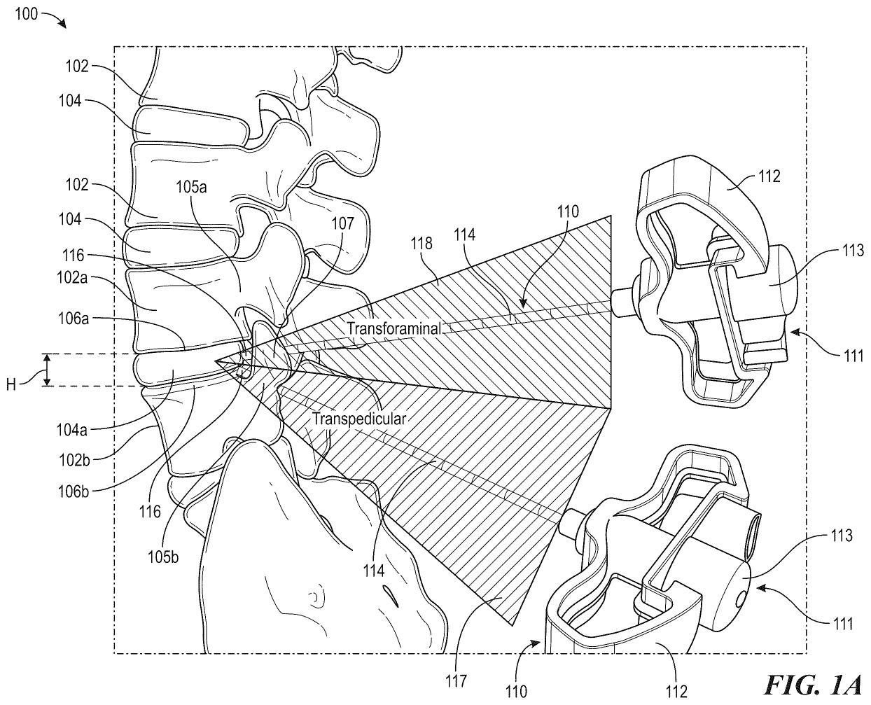

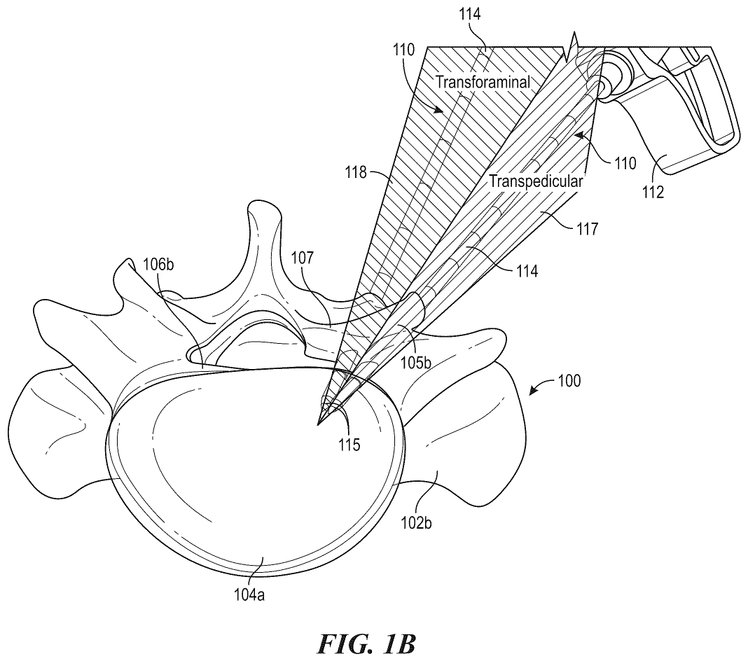

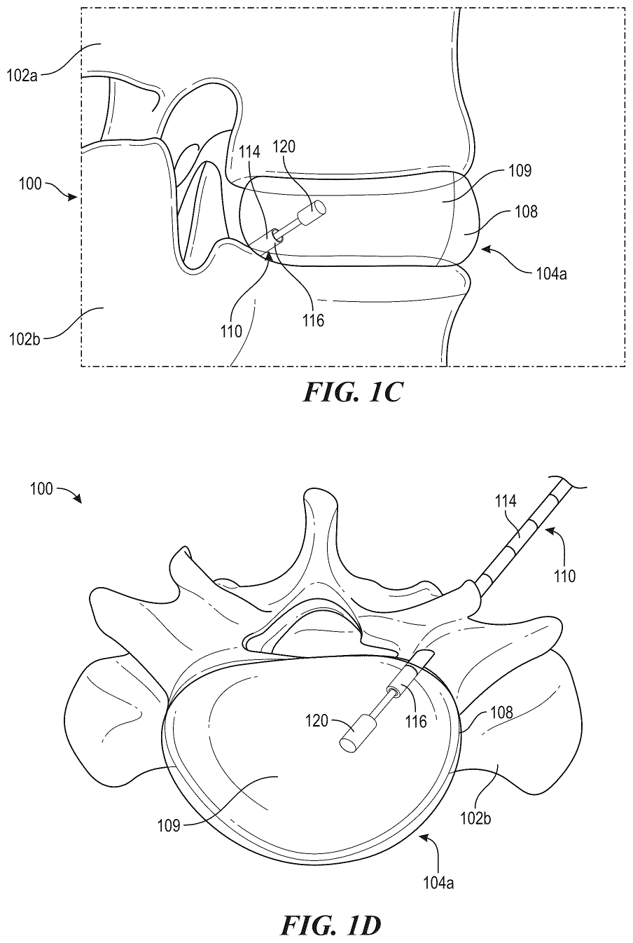

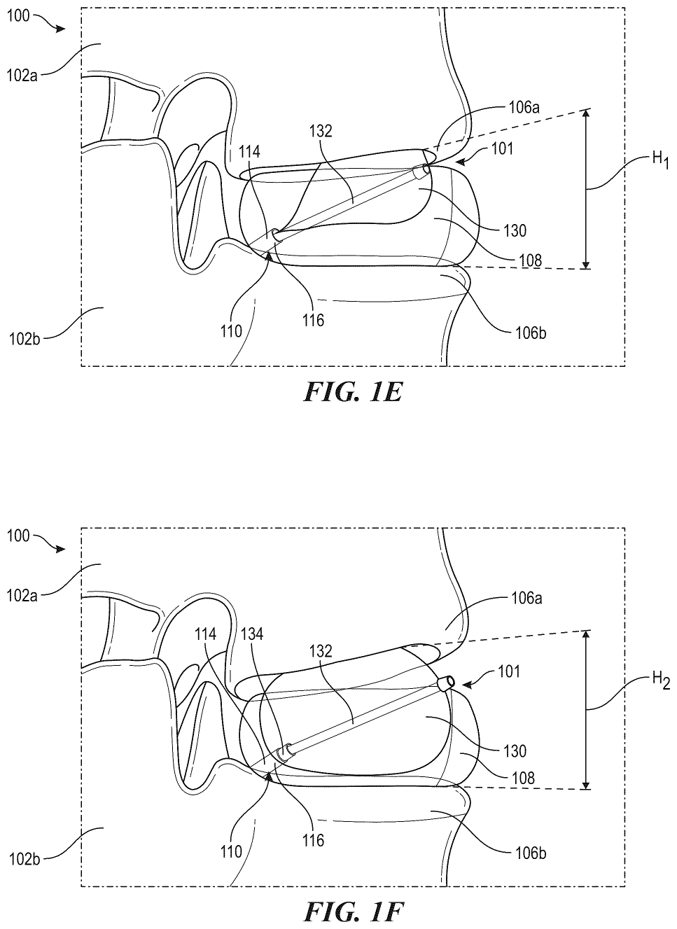

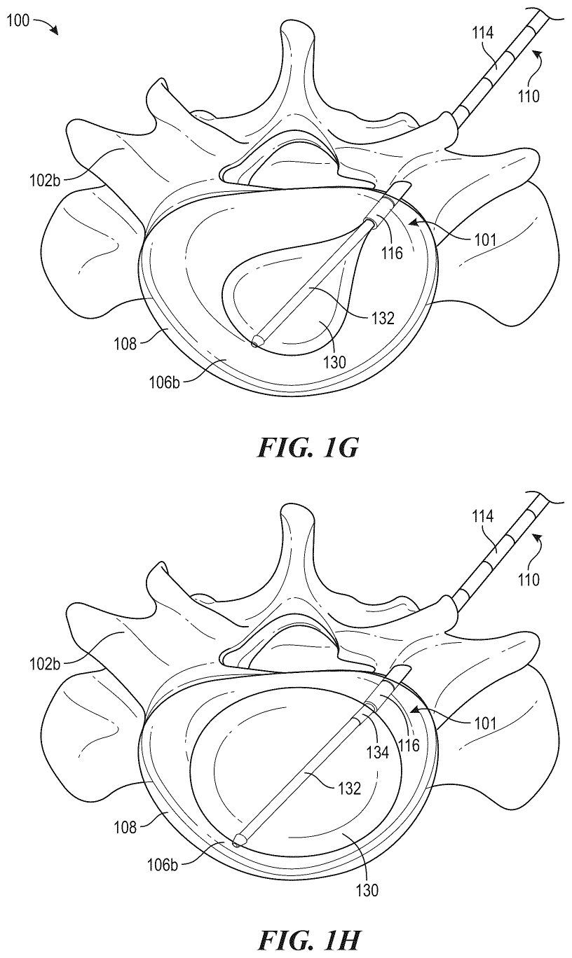

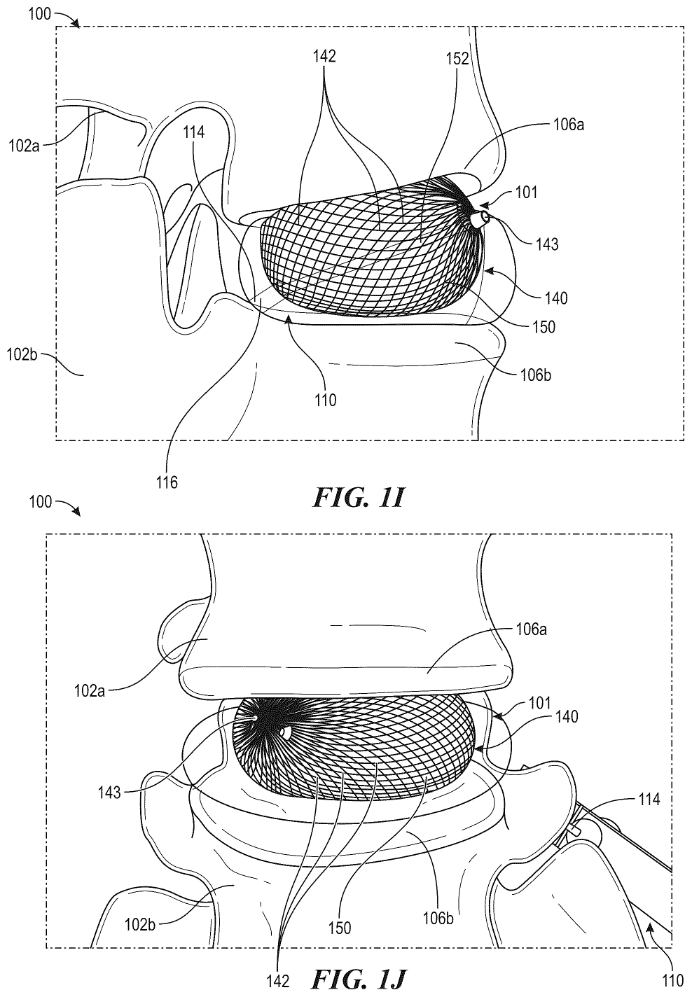

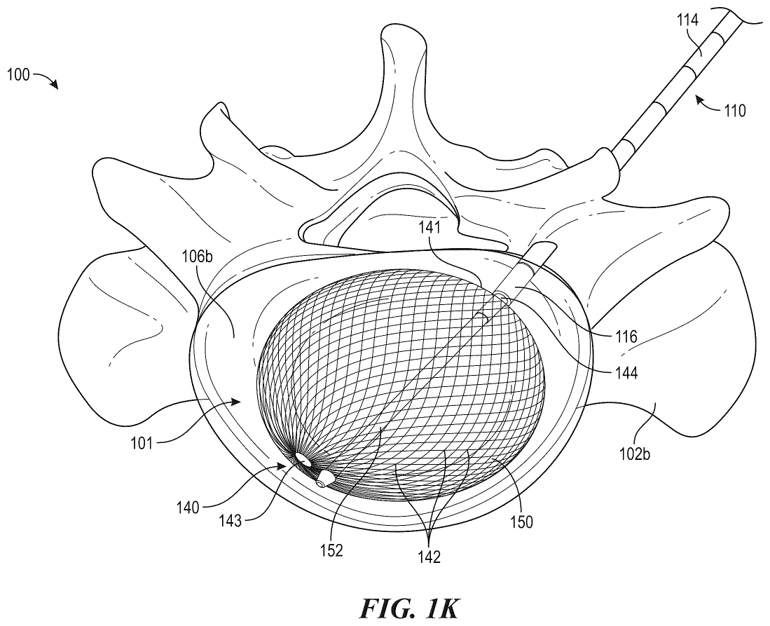

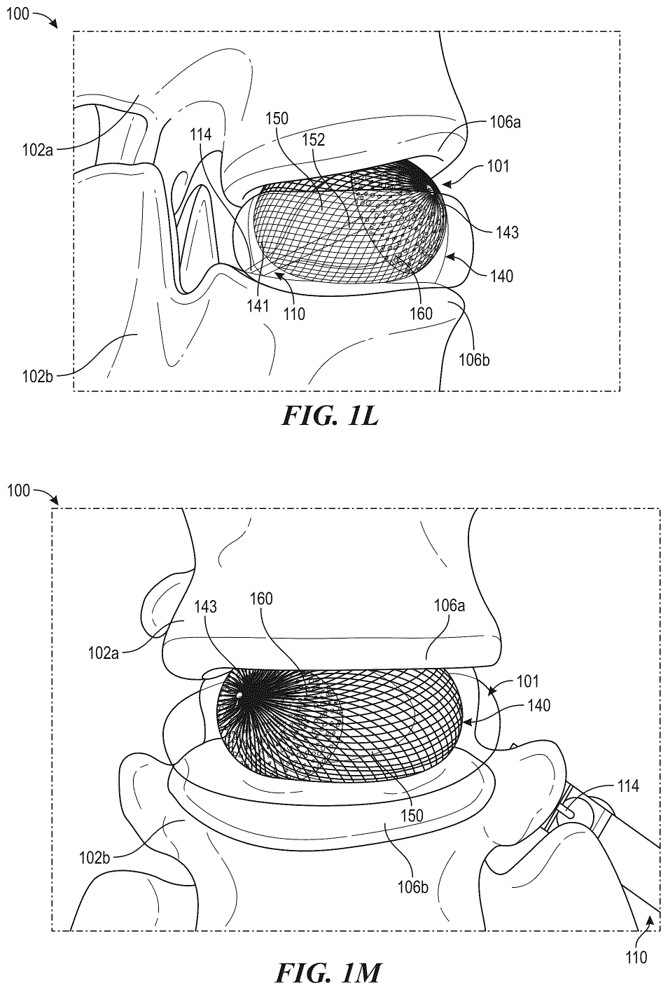

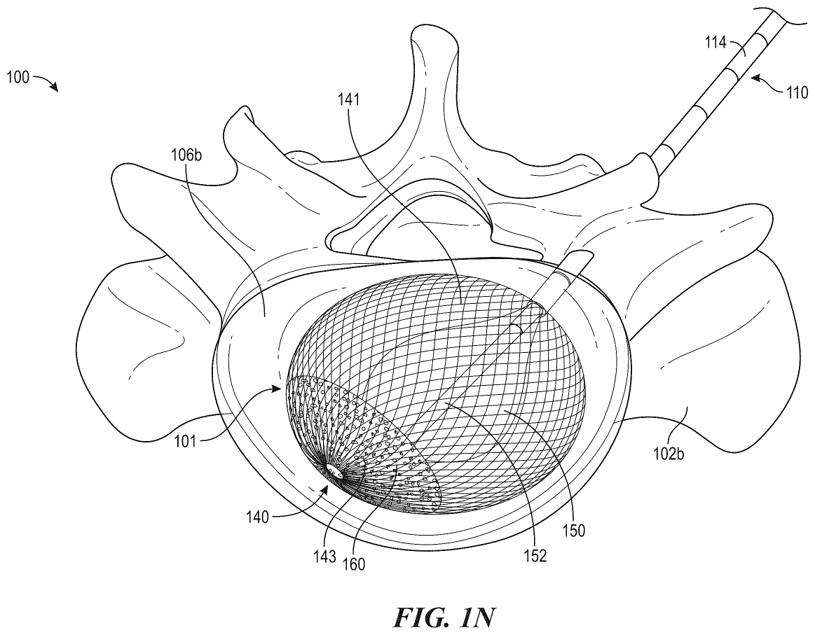

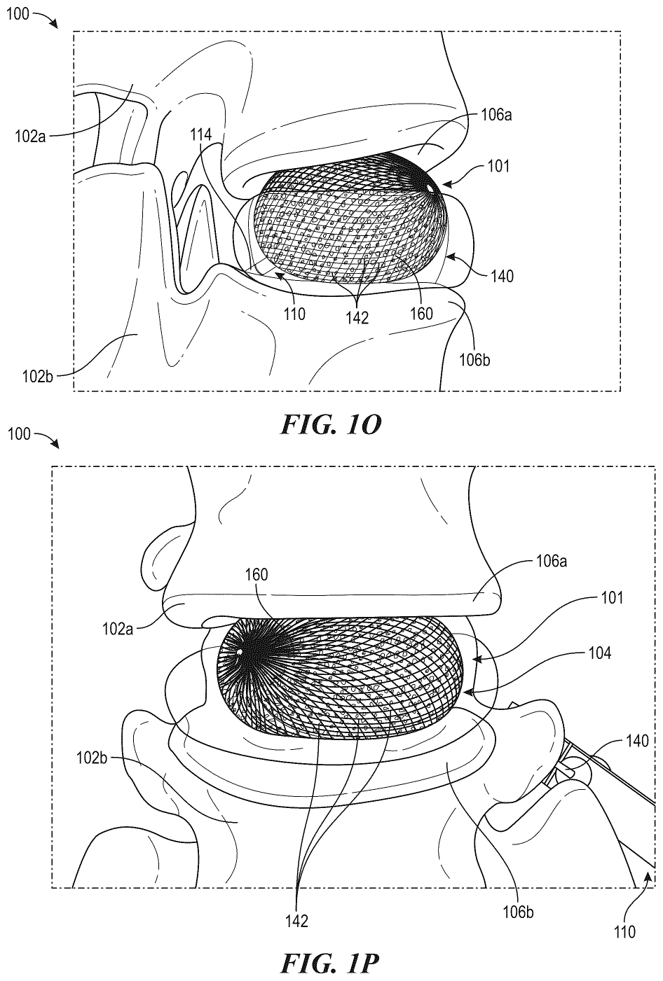

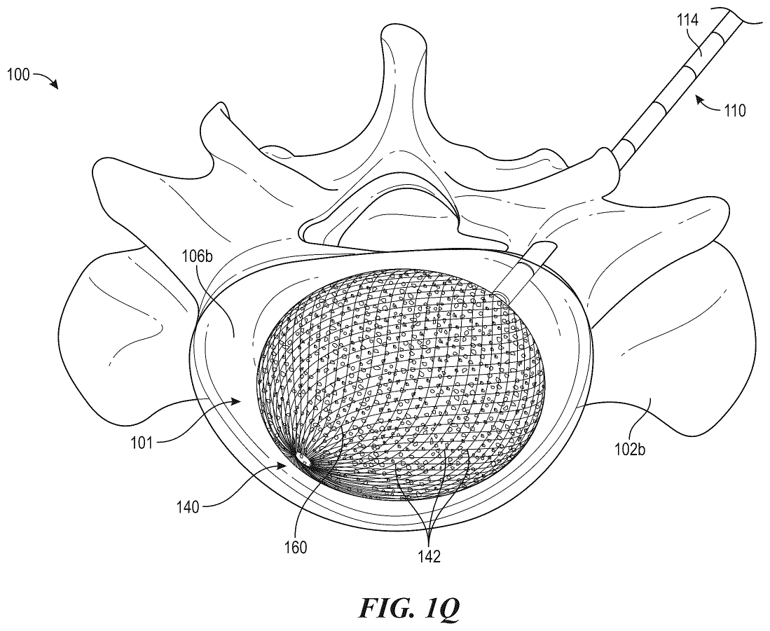

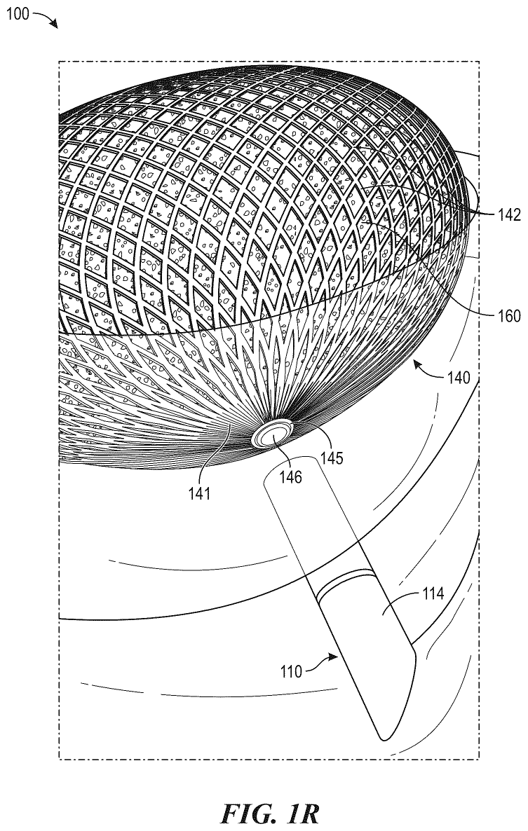

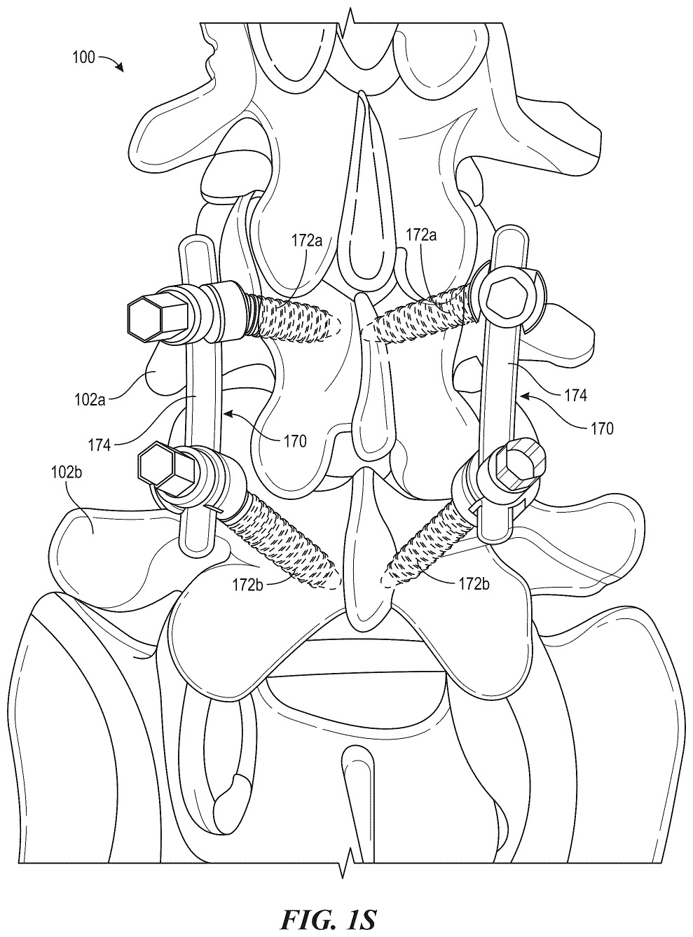

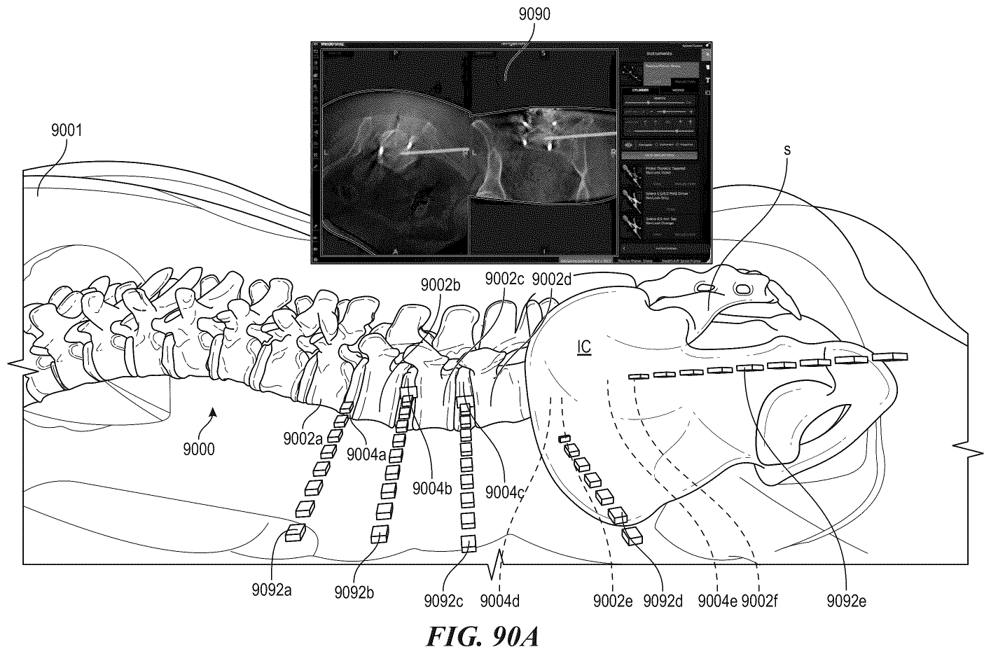

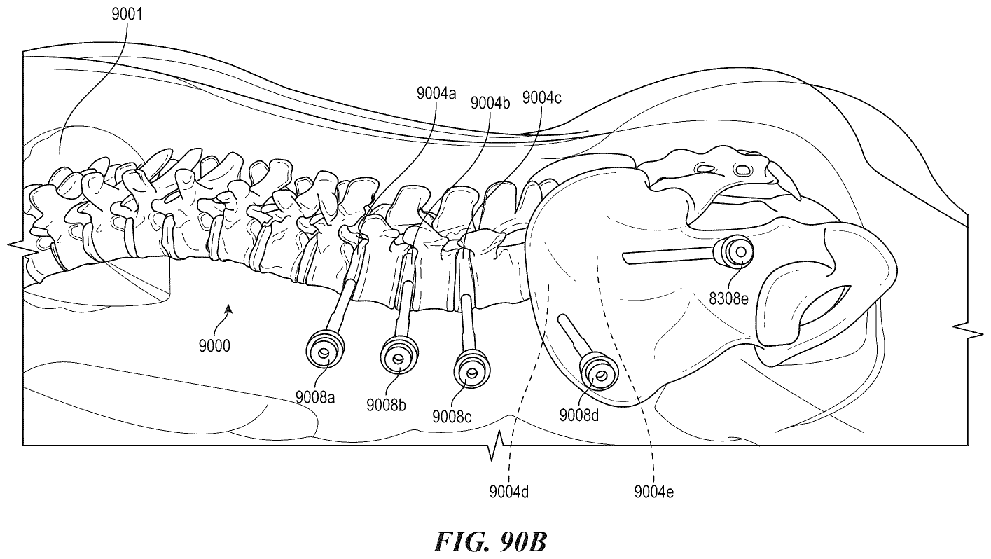

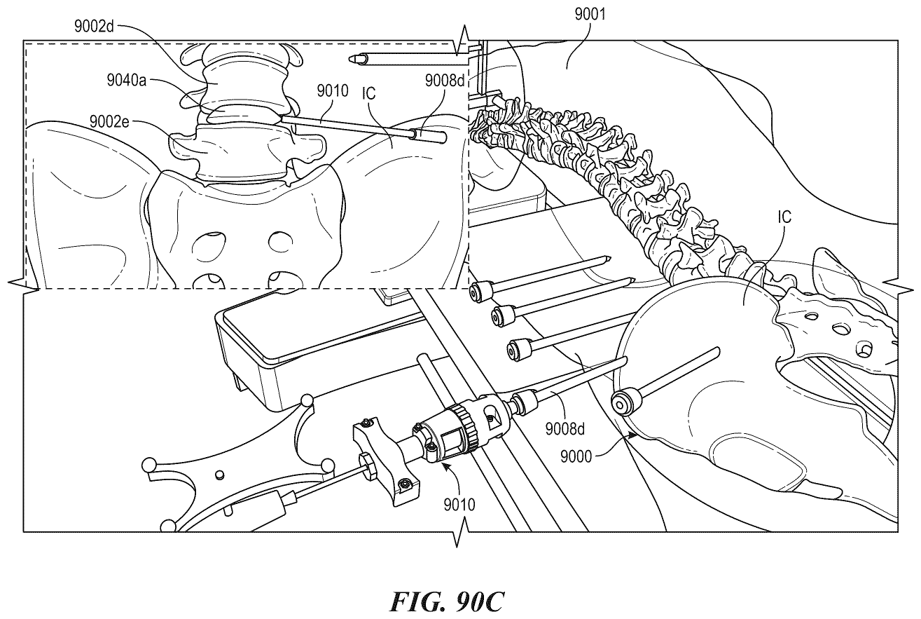

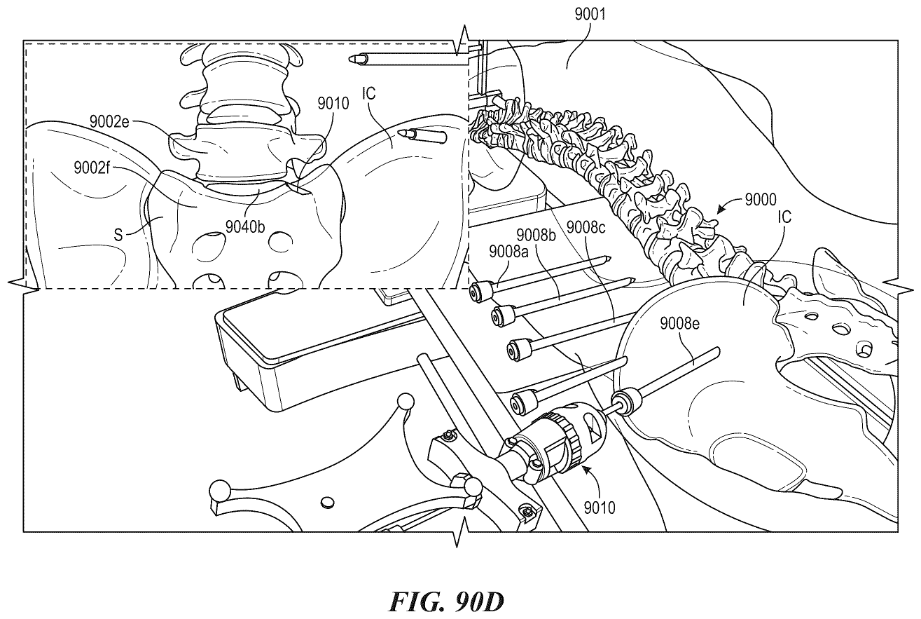

Certain details are set forth in the following description and in FIGS. 1A-90D to provide a thorough understanding of various embodiments of the present technology. In other instances, well-known structures, materials, operations, and/or systems often associated with spinal surgical procedures, intervertebral devices, spinal fusion procedures, posterior fixation assemblies, and the like, are not shown or described in detail in the following disclosure to avoid unnecessarily obscuring the description of the various embodiments of the technology. Those of ordinary skill in the art will recognize, however, that the present technology can be practiced without one or more of the details set forth herein, and/or with other structures, methods, components, and so forth. The terminology used below is to be interpreted in its broadest reasonable manner, even though it is being used in conjunction with a detailed description of certain examples of embodiments of the technology.

With regard to the terms “distal” and “proximal” within this description, unless otherwise specified, the terms can reference a relative position of the portions of a spinal access system with reference to an operator and/or a location in the spinal anatomy. Also, as used herein, the designations “rearward,” “forward,” “upward,” “downward,” and the like are not meant to limit the referenced component to a specific orientation. It will be appreciated that such designations refer to the orientation of the referenced component as illustrated in the Figures; the systems of the present technology can be used in any orientation suitable to the user. Moreover, the terms “distal” and “proximal” can additionally be referred to as “leading” and “trailing,” respectively, and/or the like.

The accompanying Figures depict embodiments of the present technology and are not intended to limit its scope unless expressly indicated. The sizes of various depicted elements are not necessarily drawn to scale, and these various elements may be enlarged to improve legibility. Component details may be abstracted in the Figures to exclude details such as position of components and certain precise connections between such components when such details are unnecessary for a complete understanding of how to make and use the present technology. Many of the details, dimensions, angles and other features shown in the Figures are merely illustrative of particular embodiments of the disclosure. Accordingly, other embodiments can have other details, dimensions, angles and features without departing from the present technology. In addition, those of ordinary skill in the art will appreciate that further embodiments of the present technology can be practiced without several of the details described below.

To the extent any materials incorporated herein by reference conflict with the present disclosure, the present disclosure controls. The headings provided herein are for convenience only and should not be construed as limiting the subject matter disclosed.

### I. SELECTED EMBODIMENTS OF INTERVERTEBRAL DEVICES, SYSTEMS, AND METHODS

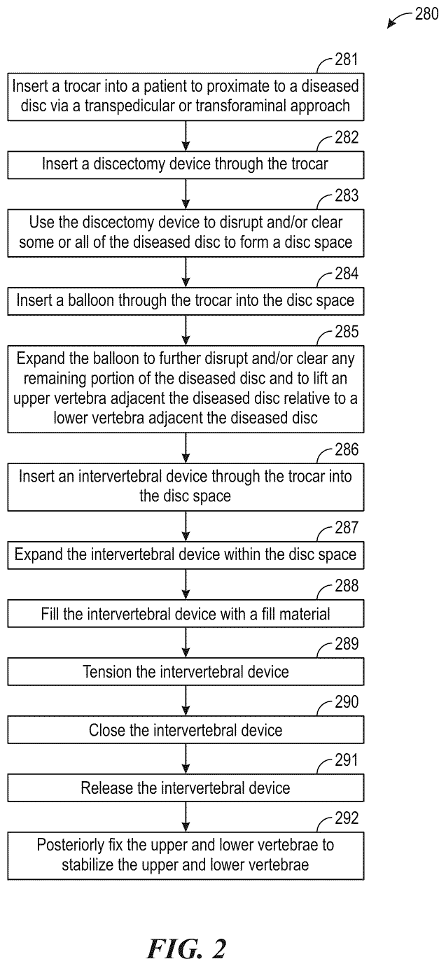

FIGS. 1A-1S are different views of a spinal surgical procedure (e.g., a spinal surgical method) on a spine 100 of a patient in accordance with embodiments of the present technology. The spinal surgical procedure can be a spinal fusion procedure (e.g., a single-level fusion) in which an existing diseased disc of the spine is fully or partially removed, and an intervertebral device is inserted into the disc space to support the adjacent vertebrae and provide for bone ingrowth therein. FIGS. 1A-IS provide an overview of some general aspects/steps of the spinal surgical procedure, and FIGS. 2-90D illustrate additional embodiments and/or aspects of the various steps, devices, and/or systems that can be used therein. In some embodiments, some of the steps of the spinal surgical procedure and/or the devices and systems used therein illustrated in FIGS. 1A-IS can include some features that are at least generally similar in structure and function, or identical in structure and function, to the corresponding features of the devices, systems, and/or methods described in International Patent Application No. PCT/US2021/051829, titled “INTERVERTEBRAL FUSION DEVICE WITH BONE GRAFT LUMBAR,” and filed Sep. 23, 2021, which is incorporated herein by reference in its entirety.

FIGS. 1A and 1B are a side view (e.g., a lateral view) and a top view (e.g., an axial view), respectively, of a portion of the spine 100 illustrating an access step of the spinal surgical procedure in accordance with embodiments of the present technology. Referring to FIGS. 1A and 1B, the spine includes a plurality of vertebrae 102 (including an individually identified first or upper vertebra 102a and a second or lower vertebra 102b) separated by discs 104 (e.g., intervertebral discs; including an individually identified diseased disc 104a). The diseased disc 104a can result from degenerative joint disease and/or disc arthropathy. The result of the diseased disc 104a often is a thinning of the diseased disc 104a and a collapse of a disc height H (FIG. 1A) of the diseased disc 104a. Such collapse of the disc height H can lead to collapse of neural foramina and, accordingly, compression of the exiting nerve root which can elicit nerve pain and/or radicular pain down the legs of the patient. Likewise, collapse of the disc height H can further cause ligamentous laxity and bulging in the spine. The result is compression of the whole spinal cord and/or the thecal sac positioned centrally in the spinal canal, which can cause radicular pain down the legs in addition to weakness and fatigue in the legs (e.g., neurogenic claudication).

In the illustrated embodiment, a trocar 110 is used to access the diseased disc 104a via either a transpedicular or transforaminal (e.g., transfacet) approach. Referring to FIG. 1A, the trocar 110 can include a handle 112 coupled to a hollow cannula 114 defining a lumen. The trocar 110 and/or the cannula 114 can be referred to as a sheath, a shaft, a stylet, an access port, an introducer, a tube, and/or the like. In some embodiments, the handle 112 includes a seal that selectively provides access to the lumen of the cannula 114. In some embodiments during the illustrated access step of the spinal surgical procedure, an introducer 111 is positioned within the cannula 114. For example, the introducer 111 can include a handle 113 (e.g., a proximal portion) that can be selectively locked to the handle 112 of the trocar 110 and an elongate member (obscured by the cannula 114 in FIGS. 1A and 1B; e.g., a needle, an awl) having a tip 115 (FIG. 1B) configured to extend distally out of the cannula 114 past a distal end portion 116 of the cannula 114 when the handles 112, 113 are locked together. The tip 115 can be sharpened, pointed, angled, and/or the like to facilitate insertion of the trocar 110 through the flesh and bone (e.g., the upper vertebra 102a or the lower vertebra 102b) of the patient to proximate to the diseased disc 104a (e.g., near the diseased disc 104a, within the diseased disc 104a). The trocar 110 and the introducer 111 can be pushed, rotated, and/or otherwise advanced through the flesh and bone to proximate the diseased disc 104a.

Referring to FIGS. 1A and 1B, in the illustrated embodiment the transpedicular approach can include advancing the trocar 110 and the introducer 111 through a pedicle 105b and an upper endplate 106b of the lower vertebra 102b. In some embodiments, such an approach can traverse the same insertion trajectory as a subsequent pedicle screw used for posterior fixation (e.g., as described in detail below with reference to FIG. 1S). The transforaminal approach can include advancing the trocar 110 and the introducer 111 through a facet joint 107 between the upper and lower vertebrae 102a-b. Referring to FIG. 1A, in other embodiments the transpedicular approach can include advancing the trocar 110 and the introducer 111 through a pedicle 105a and a lower endplate 106a of the upper vertebra 102a. Both the transpedicular approach, transfacet approach and the transforaminal approach access the diseased disc 104a through bone of the upper and/or lower vertebrae 102a-b along a trajectory that avoids the spinal nerve roots, and therefore avoids retraction of the spinal nerve roots during the spinal surgical procedure which could pose a risk to the patient. Accordingly, in some aspects of the present technology both trajectories can inhibit or even prevent the trocar 110 from contacting and potentially damaging the nerve roots as they can go through bone and circumvent traditional access corridors that go through spaces that the nerves can also be found in. In contrast, many conventional access techniques access the disc space by squeezing around the bony elements of the vertebrae—for example, through openings in which the spinal nerve roots exit the spinal column—increasing the likelihood of nerve damage during access.

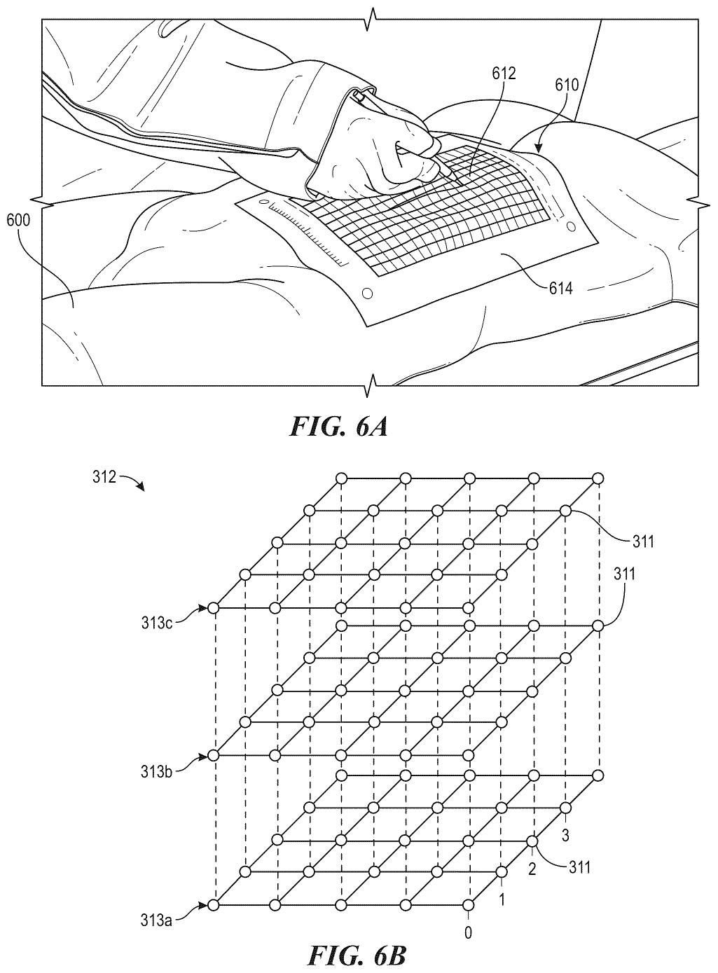

Referring to FIGS. 1A and 1B, the transpedicular approach can comprise a variety of different access angles shown as a shaded range 117 and the transforaminal and transfacet approaches can likewise comprise a variety of different access angles shown as a shaded range 118. The specific access angle(s) and trajectory for the trocar 110 can be determined via radiographic (e.g., X-Ray) imaging and/or other medical imaging procedures performed before (e.g., preoperatively) and/or during the spinal surgical procedure (e.g., intraoperatively) as described, for example, in detail below with reference to FIGS. 6A-17B.

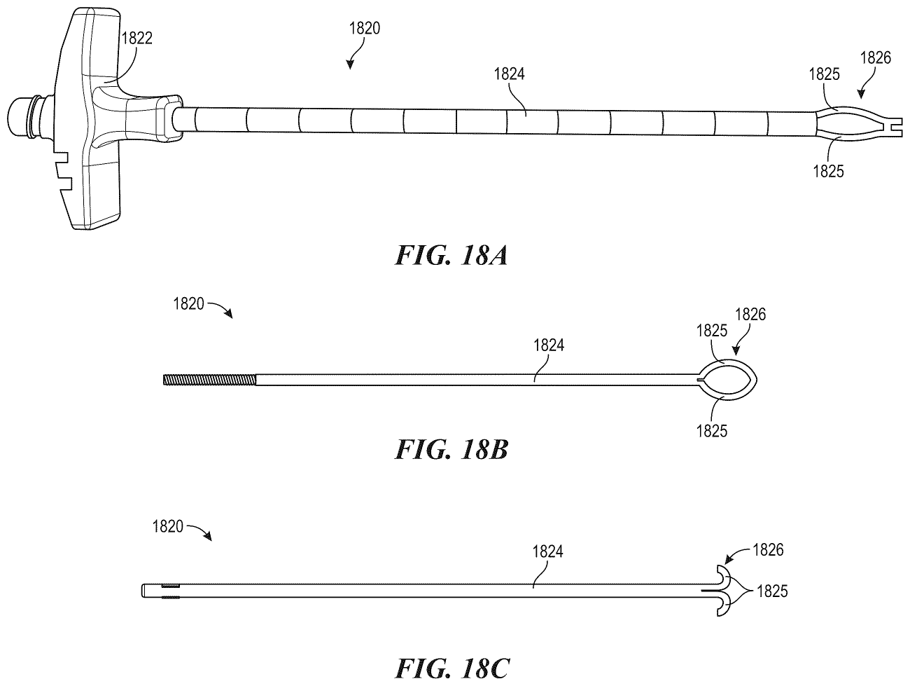

FIGS. 1C and 1D are a side view (e.g., a lateral view) and a top view (e.g., an axial view), respectively, of a portion of the spine 100 illustrating a discectomy step of the spinal surgical procedure in accordance with embodiments of the present technology. Referring to FIGS. 1C and 1D, after accessing the diseased disc 104a, the introducer 111 (FIGS. 1A and 1B) can be removed from within the cannula 114 of the trocar 110 and a discectomy device 120 can be inserted through the cannula 114 past the distal end portion 116 thereof and into the diseased disc 104a. In some embodiments, the discectomy device 120 is self-expanding, bladed, sharpened, rotatable, translatable, and/or otherwise configured to engage, disrupt, and/or dislodge the diseased disc 104a. Further embodiments of discectomy devices are described in detail below with reference to FIGS. 18A-29D.

The diseased disc 104a can include a ligamentous ring 108 (e.g., comprising an annulus fibrosus, a posterior longitudinal ligament, and/or an anterior longitudinal ligament) surrounding a nucleus 109 (e.g., a nucleus pulposus). The ligamentous ring 108 is shown as partially transparent and the nucleus 109 is shown as transparent in FIGS. 1C and 1D for clarity. The ligamentous ring 108 can connect the upper and lower vertebrae 102a-b and keep the nucleus 109 intact when forces are applied to the spine 100, while the nucleus 109 can provide cushioning between the upper and lower vertebrae 102a-b. Accordingly, the nucleus 109 can be softer and easier to disrupt and remove than the ligamentous ring 108. The discectomy device 120 can be manipulated to engage either or both of the ligamentous ring 108 and the nucleus 109 to clear and remove such material.

In some embodiments, the discectomy device 120 can be inserted through a separate inner trocar (not shown) inserted through the trocar 110, which functions as an outer trocar. The inner trocar can be curved or otherwise shaped to facilitate deployment of the discectomy device 120 to different portions of the diseased disc 104a.

After a sufficient amount of the diseased disc 104a has been removed and/or disrupted, the spinal surgical procedure can include deploying a balloon into the disc space. For example, FIGS. 1E and 1F are side views (e.g., lateral views) of a portion of the spine 100 illustrating a first stage and a second stage of a balloon deployment step of the spinal surgical procedure, respectively, in accordance with embodiments of the present technology. Likewise, FIGS. 1G and 1H are corresponding top views (e.g., axial views) of the portion of the spine 100 shown in FIGS. 1E and 1F, respectively, illustrating the first stage and the second stage of the balloon deployment step in accordance with embodiments of the present technology. Referring to FIGS. 1E-1H together, after a sufficient amount of the diseased disc 104a (FIGS. 1A-1D) has been removed by the discectomy device 120 (FIGS. 1C and 1D), the discectomy device 120 can be removed from the cannula 114 of the trocar 110 and a first balloon 130 can be inserted through the cannula 114 past the distal end portion 116 into a disc space 101 (e.g., within any remaining portion of the ligamentous ring 108) where the diseased disc 104a (FIGS. 1A-1D) has been fully or partially removed in the discectomy step. The first balloon 130 can have a distal portion coupled to an inner balloon shaft 132 and a proximal portion coupled to an outer balloon shaft 134 (obscured in FIGS. 1E and 1G) that are advanceable through the cannula 114. The first balloon 130, the inner balloon shaft 132, and the outer balloon shaft 134 can be collectively referred to as a balloon device or a balloon expansion device.

In the first stage shown in FIGS. 1E and 1G, the first balloon 130 is partially inserted into the disc space 101 and partially inflated within the disc space 101. The first balloon 130 can be inflated via an external pressure source coupled to a lumen of the outer balloon shaft 134. In the second stage shown in FIGS. 1F and 1H, the first balloon 130 is fully inserted into the disc space 101 and fully inflated within the disc space 101.

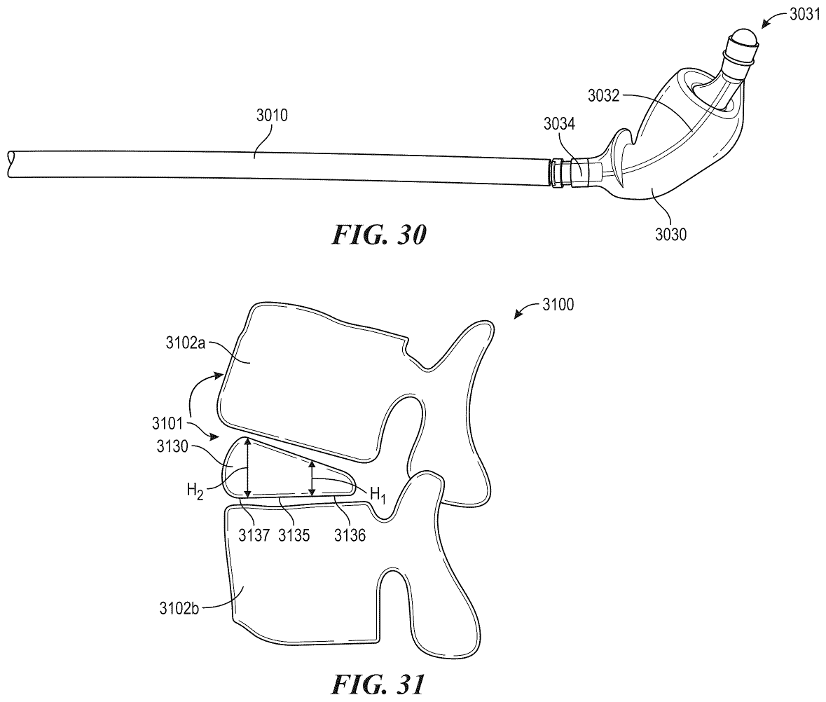

Expansion of the first balloon 130 within the disc space 101 can act to break, distract, and/or otherwise disrupt any remaining portion of the diseased disc 104a, such as some or all of the ligamentous ring 108. More specifically, the first balloon 130 can expand horizontally (e.g., along a plane extending between the lower and upper endplates 106a-b) to directly disrupt and break the ligamentous ring 108 and/or can expand vertically to indirectly disrupt and break the ligamentous ring 108 by moving the upper and lower vertebrae 102a-b away from one another. Likewise, as best seen in FIGS. 1E and 1F, inflating the first balloon 130 can enlarge the disc space 101 by increasing a height of the disc space 101 (e.g., from a first value H₁ shown in FIG. 1E to a second value H₂ shown in FIG. 1F greater than the first value). That is, the first balloon 130 can lift the upper vertebra 102a away from the lower vertebra 102b. As described in greater detail below with reference to FIGS. 30-39, the first balloon 130 can be configured (shaped, sized, constructed) to expand to a selected shape to, for example, provide differential lifting of the upper and lower vertebrae 102a-b. In some embodiments, the first balloon 130 expands to contact a substantial portion of the upper endplate 106b of the lower vertebra 102b and/or a substantial portion of the lower endplate 106a of the upper vertebra 102a. In some aspects of the present technology, this can avoid point loading on the lower and upper endplates 106a-b to inhibit or even prevent fracture of the lower and upper endplates 106a-b. In contrast, many conventional techniques expand a disc space via an interbody with significant risk of endplate fracture.

In some embodiments, the first balloon 130 can be inserted through a separate inner trocar (not shown) inserted through the trocar 110, which functions as an outer trocar. The inner trocar can be curved or otherwise shaped to facilitate deployment of the first balloon 130 to a specified portion of the disc space 101. Additionally, a pressure within and/or volume of the first balloon 130 can be monitored to provide feedback to a user (e.g., a surgeon) about a state of breakage of the ligamentous ring 108 and/or a state of lifting of the upper and lower vertebrae 102a-b.

After expanding the first balloon 130 within the disc space 101 to disrupt any remaining portion of the diseased disc 104a and to lift the upper vertebra 102a relative to the lower vertebra 102b, the spinal surgical procedure can include deploying an intervertebral device into the disc space 101. For example, FIGS. 1I-1K are side view (e.g., a lateral view), another side view (e.g., an anteriorly-facing view), and a top view (e.g., an axial view), respectively, of a portion of the spine 100 illustrating an intervertebral device deployment step of the spinal surgical procedure in accordance with embodiments of the present technology. Referring to FIGS. 1I-1K together, after expanding the first balloon 130 (FIGS. 1G and 1H) within the disc space 101, the first balloon 130 can be removed from the cannula 114 of the trocar 110 and an intervertebral device 140 can be inserted through the cannula 114 past the distal end portion 116 into the disc space 101 and expanded within the disc space 101.

The intervertebral device 140 can be referred to as an interbody, an implant, a disc-replacement device, a cage, and/or the like. The intervertebral device 140 can be a braid, mesh, or knit of filaments 142 that terminate and/or are joined together at a proximal portion 141 (e.g., a proximal hub; obscured in FIGS. 1I and 1L) and a distal portion 143 (e.g., a distal hub). The intervertebral device 140 can be coupled to a deployment shaft 144 (obscured in FIGS. 1I and 1J) for advancing the intervertebral device 140 through the cannula 114 and into the disc space 101. For example, the proximal portion 141 of the intervertebral device 140 can be releasably coupled to a distal portion of the deployment shaft 144 such that the intervertebral device 140 can be detached from the deployment shaft 144 after it is suitably positioned within the disc space 101.

In the illustrated embodiment, the intervertebral device 140 has been deployed into the disc space 101 and subsequently expanded within the disc space 101 by a second balloon 150 positioned within the intervertebral device 140. The second balloon 150 can be similar to the first balloon described in detail with reference to FIGS. 1E-1H. For example, the second balloon 150 can be coupled to one or more balloon shafts 152 that are insertable through the cannula 114 (e.g., via a lumen in the deployment shaft 144 coupled to the intervertebral device 140) and that have one or more inflation lumens for inflating the second balloon 150 via an external pressure source. Inflating the second balloon 150 expands the intervertebral device 140 into contact with the lower and upper endplates 106a-b of the upper and lower vertebrae 102a-b, respectively, and can also act to re-enlarge (e.g., re-lift) the disc space 101 by increasing the height of the disc space 101 (e.g., from the first value H₁ shown in FIG. 1E to the second value H₂ shown in FIG. 1F greater than the first value). In some aspects of the present technology, re-enlarging the disc space 101 can be easier—requiring less force—than initially enlarging the disc space 101 with the first balloon 130.

In some embodiments, the second balloon 150 can be made thinner than the first balloon 130 because (i) it is configured to be expanded within the intervertebral device 140 such that it does not contact the lower or upper endplates 106a-b and/or (ii) it does not need to exert as much force as the first balloon 130 to lift the upper vertebra 102a relative to the lower vertebra 102b after previously lifting the upper vertebra 102a with the first balloon 130 and disrupting the ligamentous ring 108 (FIGS. 1G and 1H). Moreover, the second balloon 150 can be thinner and/or smaller than the first balloon 130 while also imparting more force against the upper and lower vertebrae 102a-b when expanded because the intervertebral device 140 acts as a reinforcement for the second balloon 150 that imparts a greater burst resistance to the second balloon 150. In some embodiments, the first balloon 130 and the second balloon 150 can be the same balloon. For example, the first balloon 130 can be initially inserted through the trocar 110 and inflated as shown in the balloon deployment step illustrated in FIGS. 1E-1H, before being deflated and removed from the trocar 110 and subsequently inserted through the deployment shaft 144 of the intervertebral device 140 for expanding the intervertebral device 140.

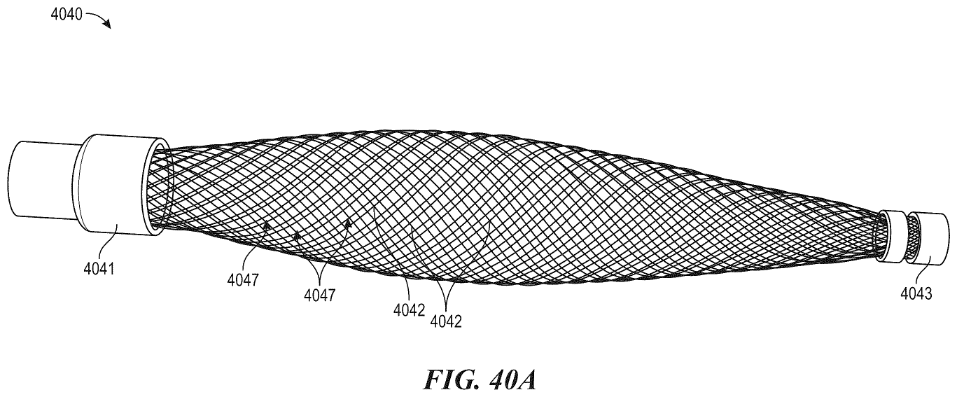

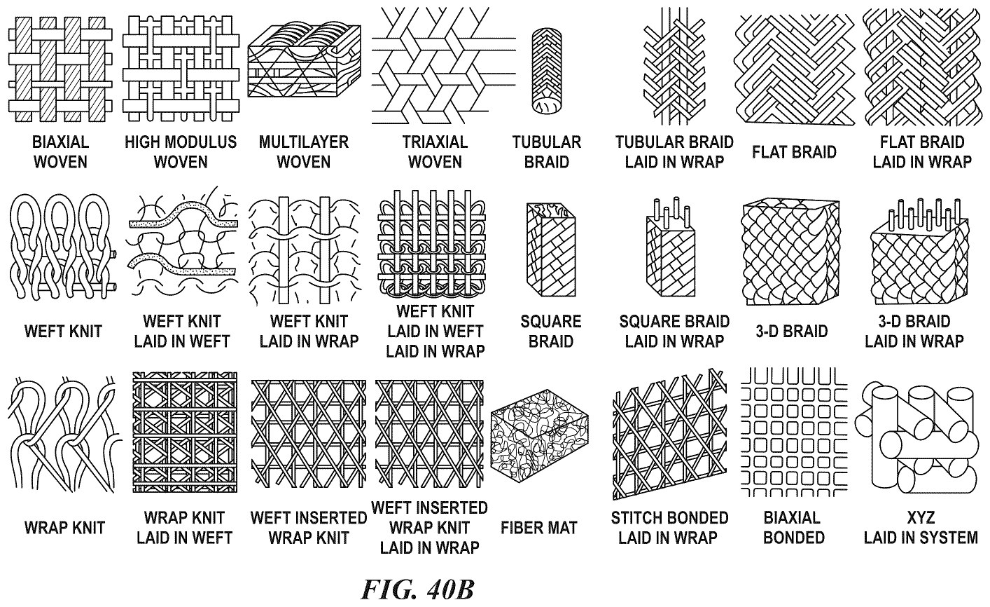

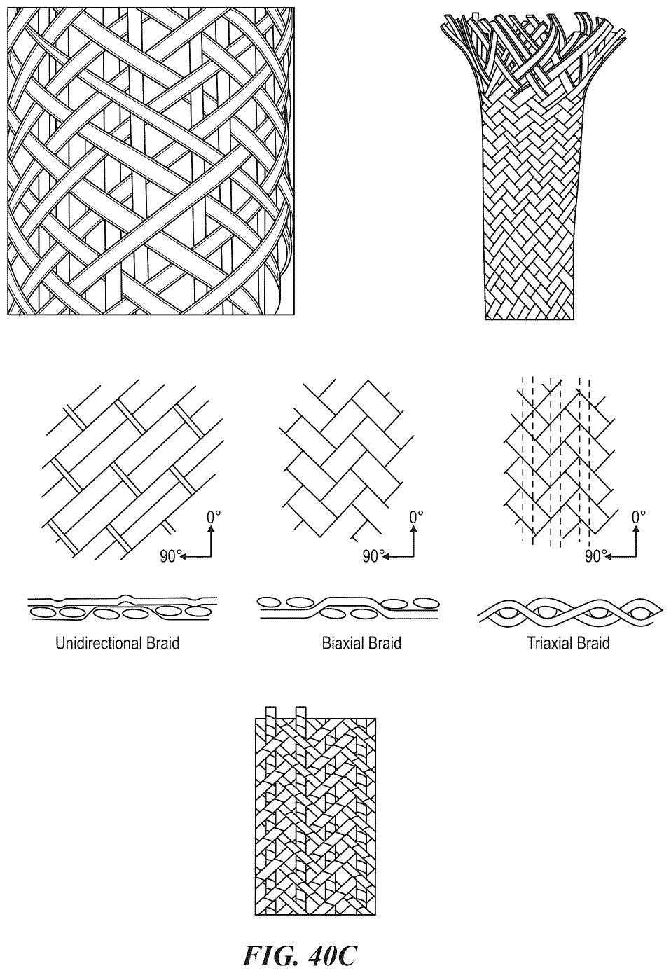

In some embodiments, the intervertebral device 140 can be configured (e.g., shaped, sized) to maximize a contact surface area between and promote conformance between the upper and/or lower endplates 106a-b and the intervertebral device 140. That is, the intervertebral device 140 can conform to the upper and/or lower endplates 106a-b. Likewise, the intervertebral device 140 can be configured to expand selectively or differentially to lift a certain portion of the upper vertebra 102a more than another portion to restore a natural alignment of the spine 100 as, for example, described in detail below with reference to FIGS. 40A-48B.

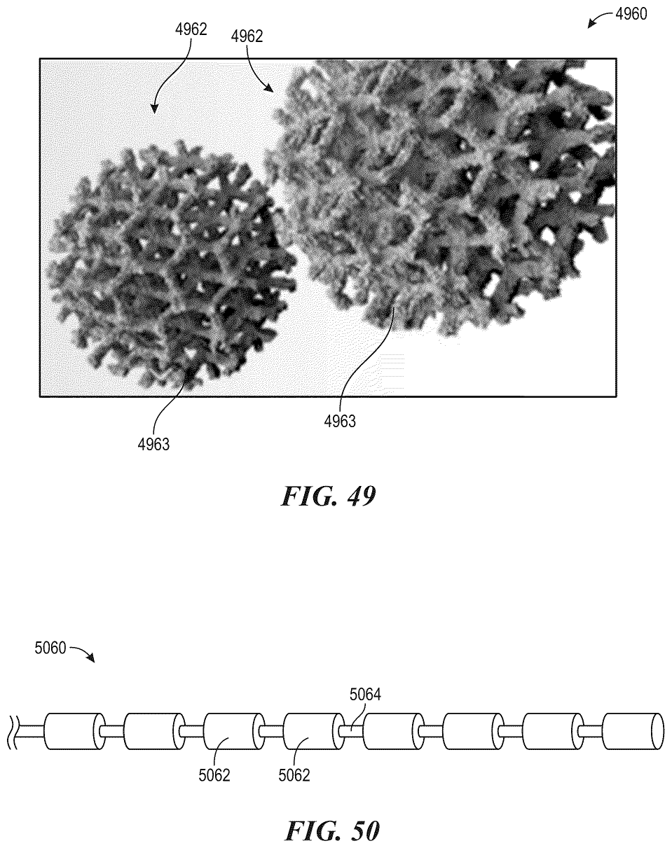

After deploying the intervertebral device 140 in the disc space 101, the spinal surgical procedure can include filling the intervertebral device 140 with a fill material. For example, FIGS. 1L-1N are side view (e.g., a lateral view), another side view (e.g., an anteriorly-facing view), and a top view (e.g., an axial view), respectively, of a portion of the spine 100 illustrating a first stage of an intervertebral device fill step of the spinal surgical procedure in accordance with embodiments of the present technology. Likewise, FIGS. 1O-1Q are a corresponding side view, other side view, and a top view of the portion of the spine 100 shown in FIGS. 1L-1N, respectively, illustrating a second stage of the intervertebral device fill step in accordance with embodiments of the present technology. Referring to FIGS. 1L-1Q, after deploying the intervertebral device 140 and expanding the second balloon 150, the intervertebral device 140 can be filled with a fill material 160. The fill material 160 can comprise a load-bearing material configured to support the upper and lower vertebrae 102a-b in a desired (e.g., lifted) position that restores the height of the disc space 101, and can be configured to promote bone ingrowth therein. As described in greater detail below with reference to FIGS. 49-60B, the fill material 160 can comprise one or more of a liquid, a gas, and a solid, such as one or more of cement, demineralized bone putty, epoxy, rigid particles, small metal particles, demineralized bone, sand-like particles such as biomaterials, silica particles, ceramic particles, metal particles, bone particles, beads, gabion structures, bone fragments, and/or the like.

Referring to FIGS. 1L-1N, the fill material 160 can be injected into the interior of the intervertebral device 140 through the balloon shaft 152. Accordingly, the second balloon 150 can be partially deflated at the same time the fill material 160 is injected to provide space for the fill material 160. In some aspects of the present technology, the second balloon 150 can at least partially hold the intervertebral device 140 open during filling with the fill material 160 to reduce a resistance of the intervertebral device 140 to filling and/or to guide the fill material 160 to assume a particular geometry within the intervertebral device 140 (e.g., a geometry selected to induce lordosis, kyphosis, and/or the like of the spine 100). In the illustrated embodiment, the distal portion 143 of the intervertebral device 140 is filled with the fill material 160 before the proximal portion 141 (e.g., the intervertebral device 140 is filled in a direction from the distal portion 143 toward the proximal portion 141). In other embodiments, the fill material 160 can be filled in a direction from the proximal portion 141 toward the distal portion and/or in another direction.

In other embodiments, the second balloon 150 and balloon shaft 152 can be removed from the trocar 110 prior to filling the intervertebral device 140 with the fill material 160, and the fill material 160 can be injected directly through the deployment shaft 144 (FIG. 1K) and/or another shaft inserted therethrough. In yet other embodiments, the fill material 160 can be injected into the second balloon 150 such that the fill material 160 fills the second balloon 150. In such embodiments, the second balloon 150 can remain within the intervertebral device 140 after implantation, can be removed (e.g., popped) after filling, or can be made of dissolvable or bioresorbable material.

Referring to FIGS. 1O-1Q, the fill material 160 can entirely or substantially fill the intervertebral device 140 in the second stage of the intervertebral device fill step. Filling the intervertebral device 140 with the fill material 160 can expand (e.g., again expand or partially expand) the intervertebral device 140 into contact with the lower and upper endplates 106a-b of the upper and lower vertebrae 102a-b, respectively, and can also act to re-enlarge (e.g., re-lift) the disc space 101 by increasing the height of the disc space 101 (e.g., from the first value H₁ shown in FIG. 1E to the second value H₂ shown in FIG. 1F greater than the first value). In some embodiments, the intervertebral device 140 can be configured (e.g., shaped, sized) to maximize a contact surface area between and promote conformance between the upper and/or lower endplates 106a-b and the intervertebral device 140 when the intervertebral device 140 is filled with the fill material 160. Likewise, the intervertebral device 140 can be configured to expand selectively or differentially when filled with the fill material 160 to selectively increase a distance between a portion of the upper and lower vertebrae 102a-b (e.g., to lift a certain portion of the upper vertebra 102a) more than another portion to restore a natural alignment of the spine 100. After filling the intervertebral device 140 with the fill material 160, the second balloon 150 can be removed from the cannula 114 of the trocar 110.

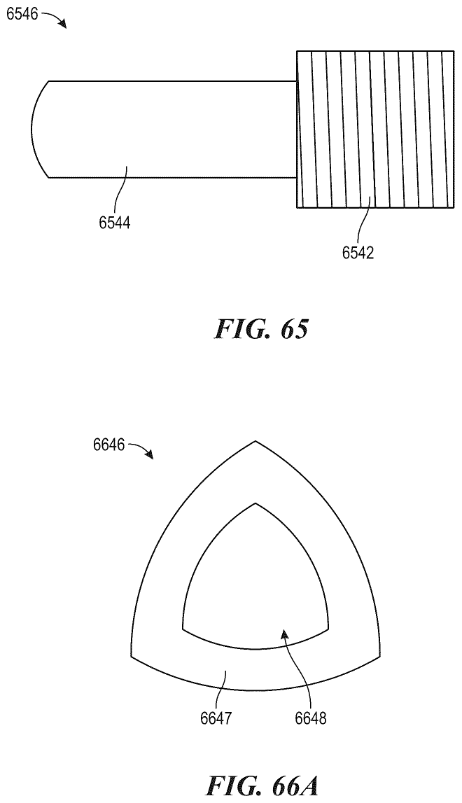

In some embodiments, after filling the intervertebral device 140 with the fill material 160, the intervertebral device 140 can be tensioned to, for example, reduce a volume and/or surface area of the intervertebral device 140 to pack the fill material 160 together and/or increase the rigidity of the fill material 160 or overall device. As described in greater detail below with reference to FIGS. 65-70D, tensioning the intervertebral device 140 can include exerting a force on the filaments 142 to draw the filaments closer together and/or more tightly together. In some embodiments, tensioning the intervertebral device 140 can happen before or concurrently with filling the intervertebral device 140 with the fill material 160 and/or closing the proximal portion 141 of the intervertebral device 140, as described in detail below with reference to FIG. 1R.

In some embodiments, the intervertebral device 140 is tensioned by filling the intervertebral device 140 with the fill material 160. For example, filling the intervertebral device 140 with the fill material 160 can expand the volume of the intervertebral device 140 against the constraint of the upper and lower vertebrae 102a-b (e.g., a constrained surface area which the intervertebral device 140 contacts)—thereby tensioning the intervertebral device 140. In some embodiments, the intervertebral device 140 is filled to have a generally spherical shape. Such a spherical shape can have the best efficiency of surface area to volume. After the source of pressure used to inject the fill material 160 is removed, or when any balloons (e.g., the second balloon 150) holding the space are removed, the anatomy may exert a compacting/deforming force on the filled intervertebral device 140, flattening it to a less ideal shape. This can create a larger surface area given a consistent volume and thus apply tension to the filaments 142.

After tensioning and filling the intervertebral device 140, the spinal surgical procedure can include detaching the intervertebral device 140 from the deployment shaft 144 (FIG. 1K) and closing the proximal portion 141 of the intervertebral device 140. For example, FIG. 1R is an enlarged side view (e.g., a posteriorly-facing view) of a portion of the spine 100 illustrating an intervertebral device closure step of the spinal surgical procedure in accordance with embodiments of the present technology. In the illustrated embodiment, the deployment shaft 144 has been detached from the proximal portion 141 of the intervertebral device 140 and a closure mechanism 146 has been secured to the proximal portion 141 of the intervertebral device 140 such that the filaments 142 and the closure mechanism 146 maintain the fill material 160 within the intervertebral device 140 and inhibit or even prevent egress of the fill material 160 out of the intervertebral device 140. In the illustrated embodiment, the closure mechanism 146 is a nut or screw that is secured within a corresponding opening 145 in the proximal portion 141 of the intervertebral device 140. The closure mechanism 146 can be advanced and actuated (e.g., rotated) by a separate shaft or instrument inserted through the cannula 114 of the trocar 110. In some embodiments, a counter-torque and/or anti-rotation feature is used to allow threading and unthreading of the closure mechanism 146. In other embodiments, the closure mechanism 146 can be a clip, slider, self-closing valve, and/or other mechanism as described in detail below with reference to FIGS. 65-70D.

After filling and closing the intervertebral device 140, the trocar 110 can be removed from the patient. Referring to FIGS. 1A-IR together, in some aspects of the present technology each of the steps of the spinal surgical procedure can be performed through the minimally-invasive port/access pathway provided by the cannula 114 of the trocar 110. That is, for example: (i) the diseased disc 104a can be accessed and at least partially removed through the cannula 114 of the trocar 110, (ii) the upper vertebra 102a can be lifted and the ligamentous ring 108 further disrupted via the first balloon 130 through the cannula 114 of the trocar 110, (iii) the intervertebral device 140 can be advanced through and deployed from the cannula 114 of the trocar 110 within the disc space 101, (iv) the intervertebral device 140 can be expanded within the disc space 101 via the second balloon 150 through the cannula 114 of the trocar 110, (v) the intervertebral device 140 can be filled with the fill material 160 through the cannula 114 of the trocar 110, and (vi) the intervertebral device 140 can be closed to secure the fill material 160 through the cannula 114 of the trocar 110. Accordingly, embodiments of the present technology can minimize disruption to the flesh of the patient undergoing the surgical procedure-minimizing patient pain and recovery time.

In some embodiments, after removing the trocar 110 from the patient, the spinal surgical procedure further includes attaching a posterior fixation assembly to the spine 100 of the patient. For example, FIG. 1S is a side view (e.g., an anteriorly-facing view) of a portion of the spine 100 of the patient illustrating a posterior fixation step of the spinal surgical procedure in accordance with embodiments of the present technology. In the illustrated embodiment, a posterior fixation assembly 170 is fixedly attached to the upper and lower vertebrae 102a-b to, for example, substantially stabilize the upper and lower vertebrae 102a-b relative to one another. The posterior fixation assembly 170 can include one or more first fixation members 172a secured within the upper vertebra 102a and one or more second fixation members 172b secured within the lower vertebra 102b. The upper and lower fixation members 172a-b can be pedicle screws, cortical screws, wires, bands, interspinous clamps, interlaminar clamps, plates, dowels, and/or the like. Pairs of the upper and lower fixation members 172a-b can be secured together via spanning members 174, such as rods, wires, bands, plates, clamps, and/or the like. The stabilization provided by the posterior fixation assembly 170 can promote bone ingrowth into the intervertebral device 140 (FIGS. 1I-1R).

Referring to FIGS. 1A-1R together, some steps of the spinal surgical procedure can be omitted and/or the various steps can be performed in a different order. For example, the posterior fixation step can be omitted, the balloon expansion step can occur before the mechanical discectomy step, the mechanical discectomy step can be omitted if sufficient disc material is removed via the balloon expansion step, and so on.

FIG. 2 is a flow diagram of a process or method 280 for performing a spinal surgical procedure, such as the spinal surgical procedure (e.g., a spinal fusion procedure) illustrated with reference to FIGS. 1A-1S, in accordance with embodiments of the present technology. At block 281, the method 280 can include inserting a trocar into a patient to proximate a diseased disc via a transpedicular or transforaminal approach, such as described in detail above with reference to FIGS. 1A and 1B. At blocks 282 and 283, the method 280 can include inserting a discectomy device through the trocar and using the mechanical discectomy device to disrupt and/or clear some or all of the diseased disc to form a disc space, respectively, such as described in detail above with reference to FIGS. 1C and 1D. At blocks 284 and 285, the method 280 can include inserting a balloon through the trocar into the disc space and expanding the balloon to further disrupt and/or clear any remaining portion of the diseased disc and to lift an upper vertebra adjacent the diseased disc relative to a lower vertebra adjacent the diseased disc, respectively, as described in detail with reference to FIGS. 1E-1H. In some embodiments, blocks 285 and 286 can be performed before blocks 283 and 284.

At blocks 286 and 287, the method 280 can include inserting an intervertebral device through the trocar into the disc space and expanding the intervertebral device within the disc space, respectively, as described in detail with reference to FIGS. 1I-1K. At block 288, the method 280 can include filling the intervertebral device with a fill material, as described in detail above with reference to FIGS. 1L-1Q. In some embodiments, the same or a different balloon is used to expand the intervertebral device within the disc space at block 287 while, in other embodiments, filling the intervertebral device with the fill material at block 288 can expand the intervertebral device.

At block 289, the method 280 can include tensioning the intervertebral device to, for example, pack the fill material within the intervertebral device. At block 290, the method 280 can include closing the intervertebral device such that the fill material remains therein, as described in detail above with reference to FIG. 1R. At block 291, the method 280 can include releasing the intervertebral device within the disc space by, for example, detaching the intervertebral device from a delivery shaft. Finally, at block 291 the method 280 can include posteriorly fixing the upper and lower vertebrae to stabilize the upper and lower vertebrae.

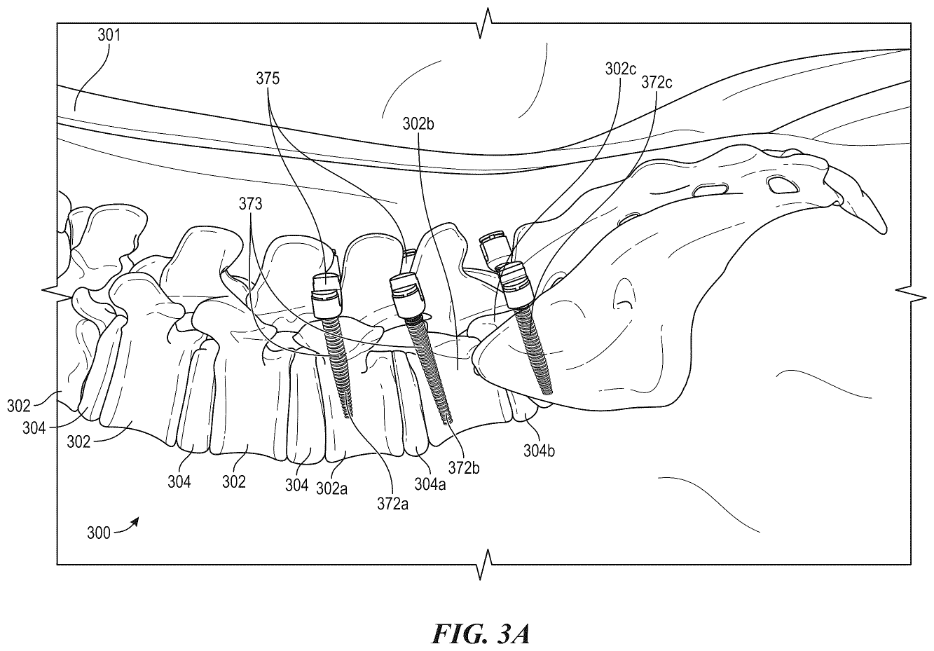

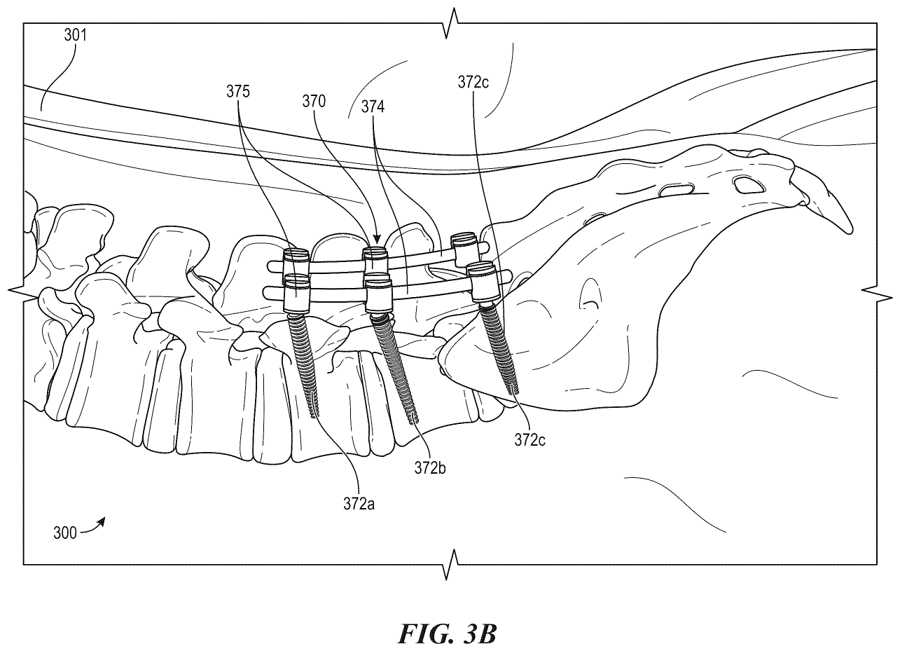

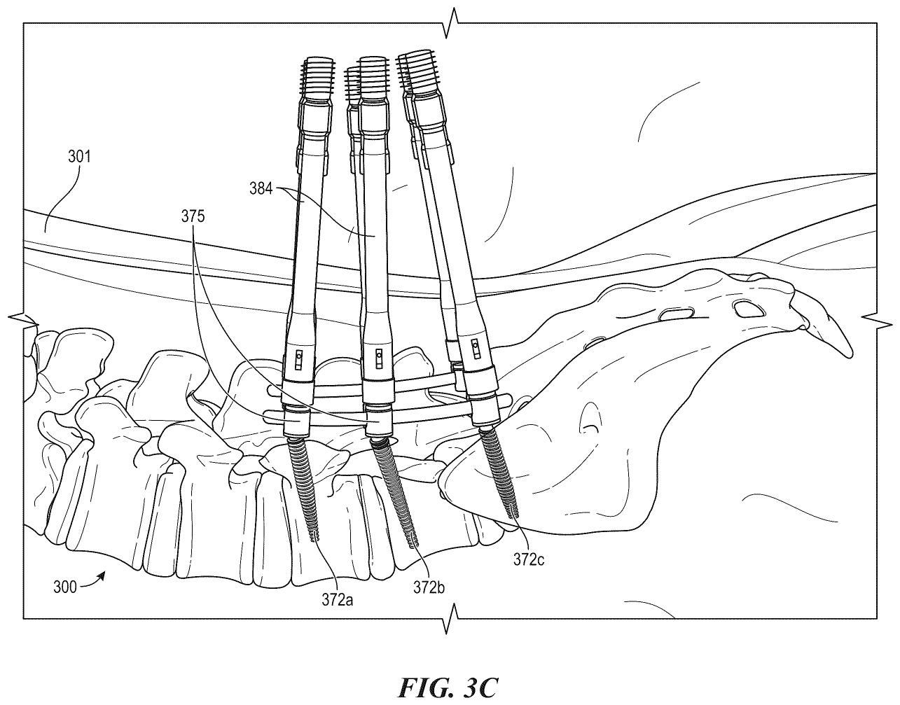

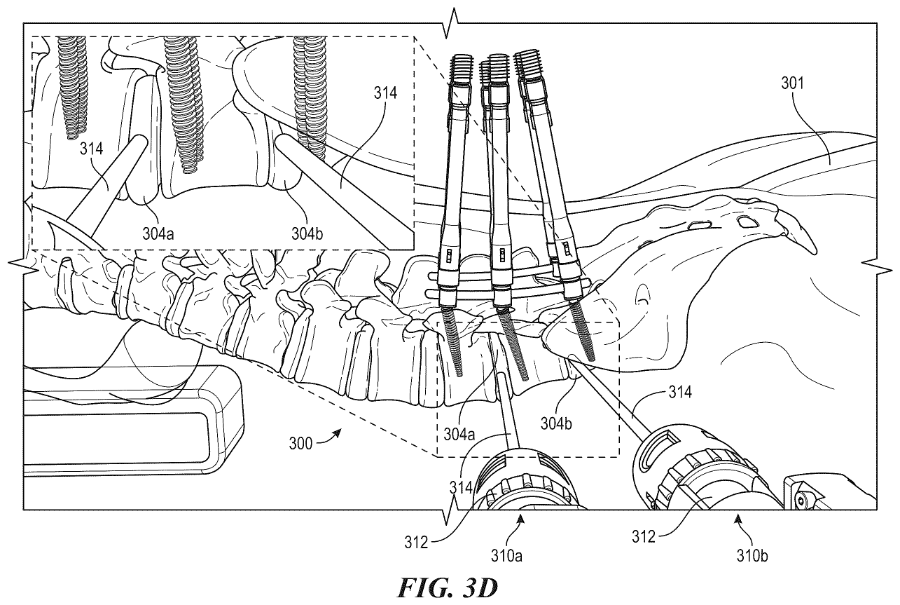

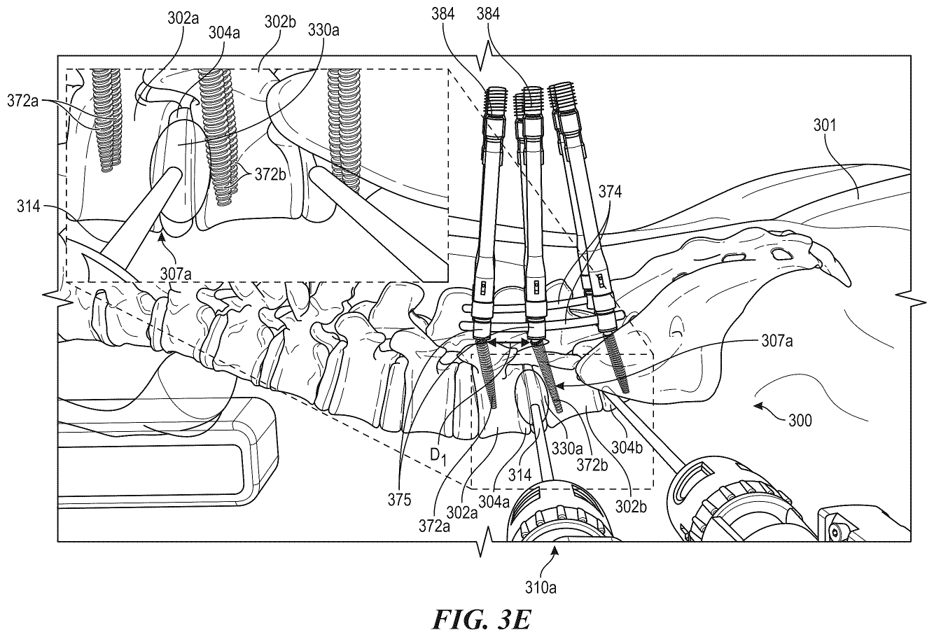

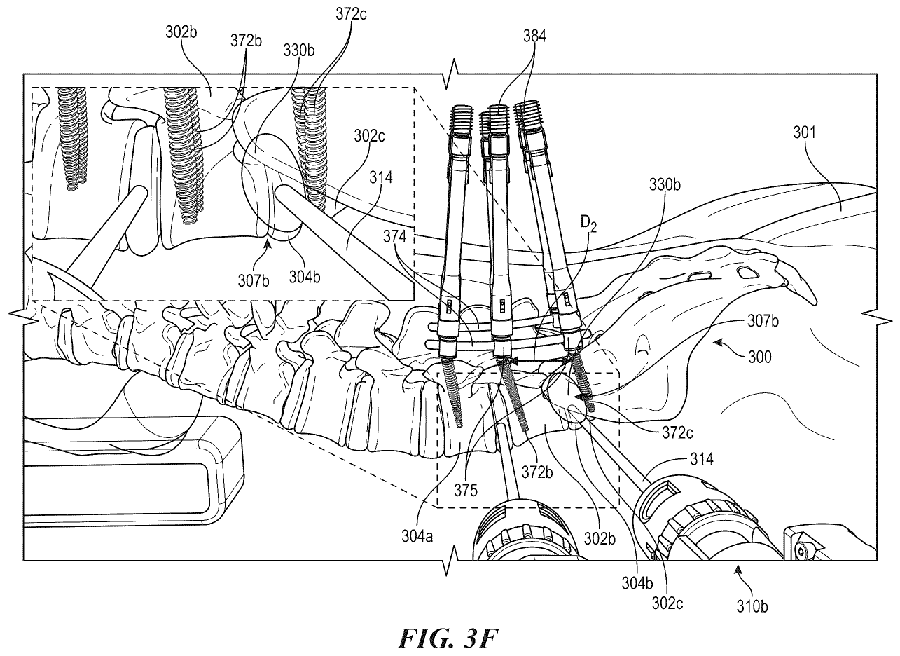

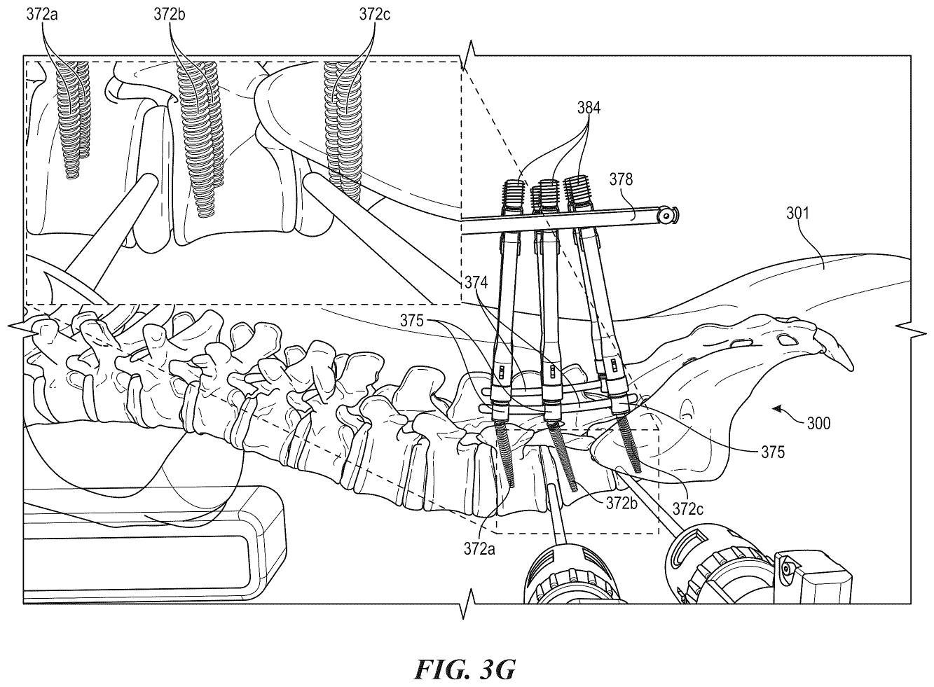

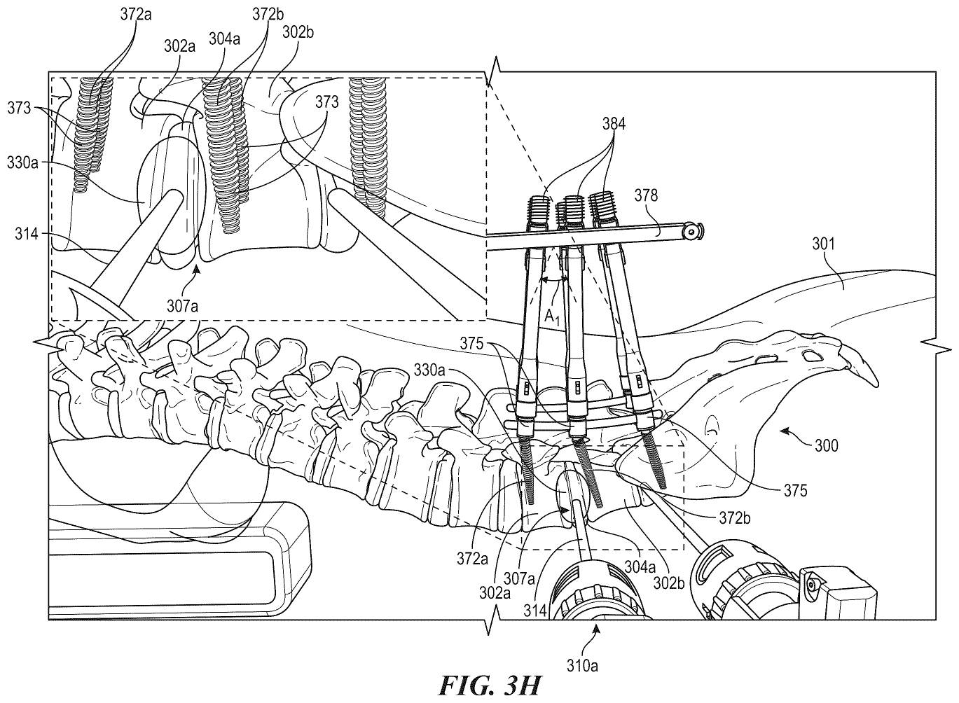

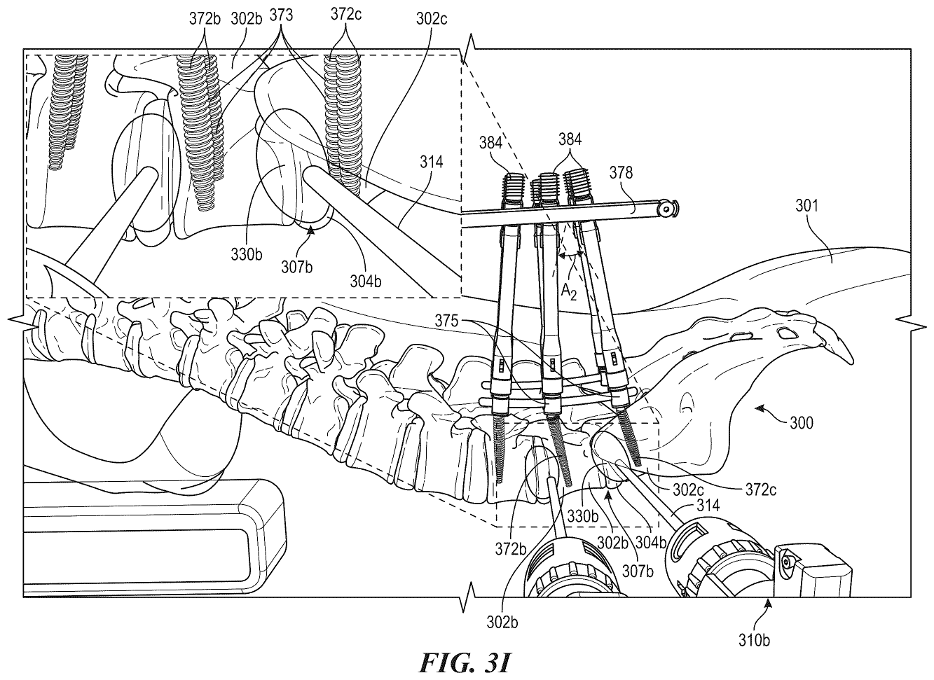

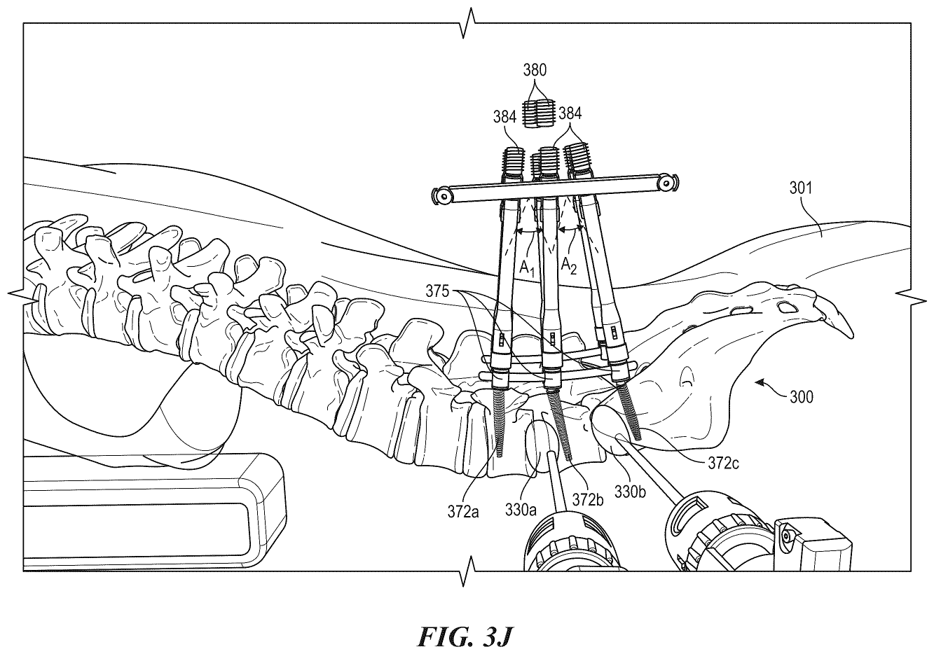

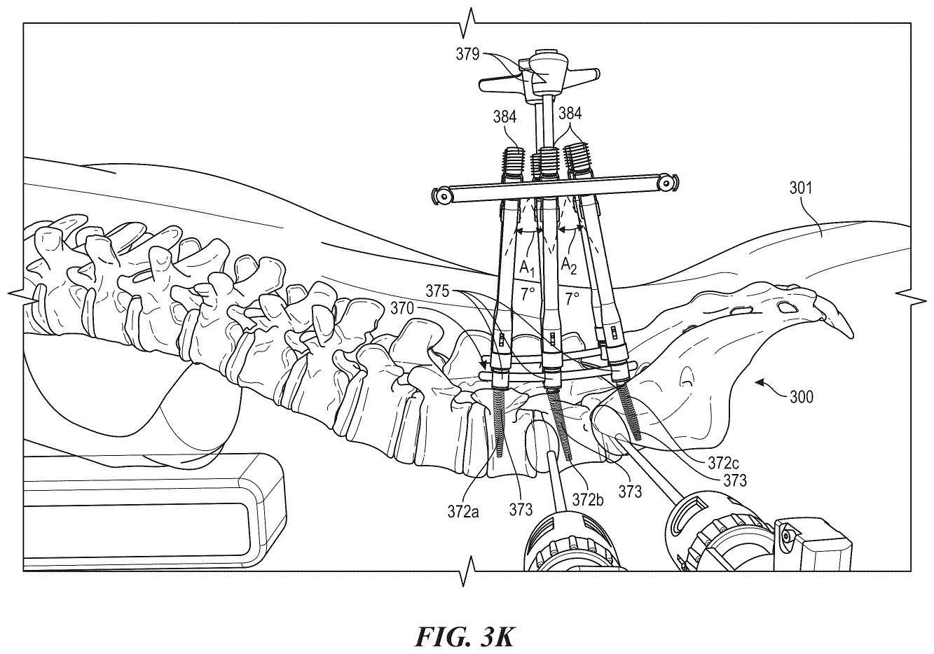

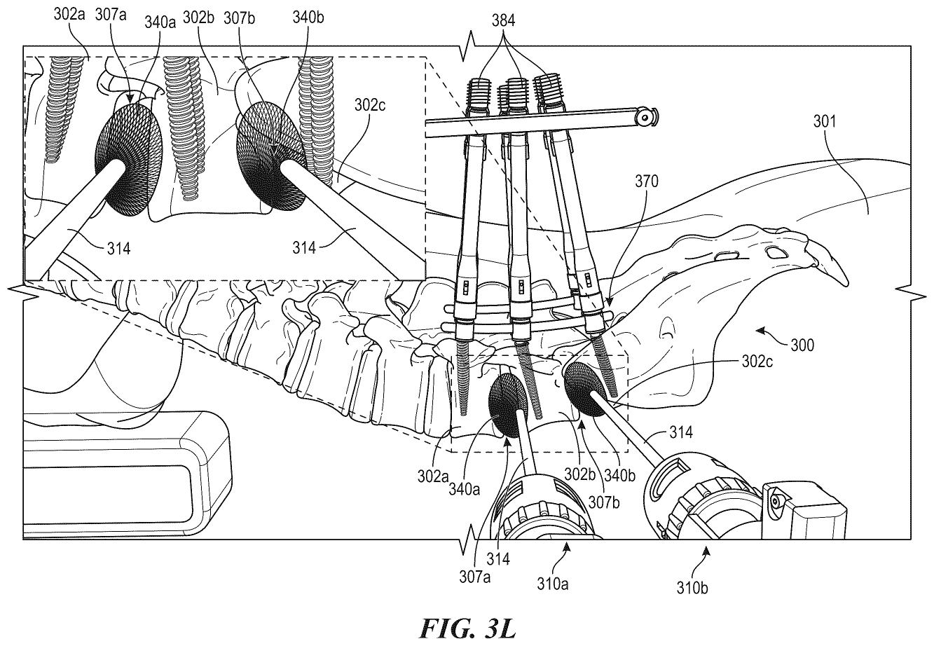

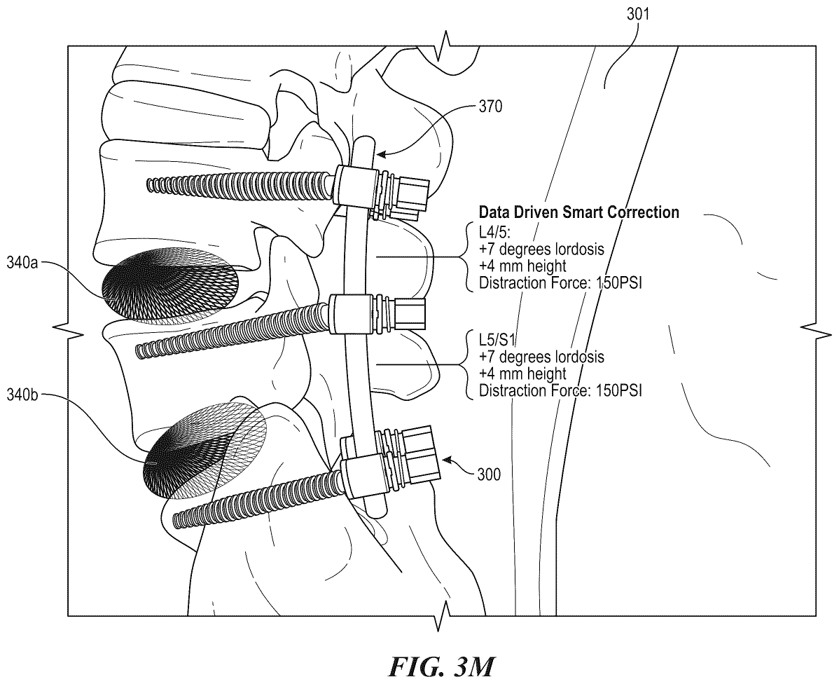

FIGS. 3A-3M are different views of a spinal surgical procedure (e.g., a spinal surgical method) on a spine 300 of a patient 301 (shown as partially transparent for clarity) in accordance with additional embodiments of the present technology. The spinal surgical procedure can be a two-level spinal fusion procedure in which two existing diseased discs of the spine are fully or partially removed, and in which two intervertebral devices are inserted into the disc space to support the adjacent vertebrae and provide for bone ingrowth therein. In other embodiments, the spinal surgical procedure can be a single-level spinal fusion procedure, or a multi-level (e.g., more than two-level) spinal fusion procedure. FIGS. 3A-3M provide an overview of some general aspects/steps of the spinal surgical procedure, and FIGS. 1A-2 and 4A-90D illustrate additional embodiments and/or aspects of the various steps, devices, and/or systems that can be used therein. In some embodiments, some of the steps of the spinal surgical procedure and/or the devices and systems used therein illustrated in FIGS. 3A-3M can include some features that are at least generally similar in structure and function, or identical in structure and function, to the corresponding features of the devices, systems, and/or methods described in detail above with reference to FIGS. 1A-2.

FIG. 3A is a side view (e.g., a lateral view) of a portion of the spine 300 illustrating a first posterior fixation step (e.g., screw insertion step) of the spinal surgical procedure in accordance with embodiments of the present technology. The spine 300 includes a plurality of vertebrae 302 (including an individually identified first or upper vertebra 302a, a second or middle vertebra 302b, and a third or lower vertebra 302c) separated by discs 304 (e.g., intervertebral discs; including an individually identified first diseased disc 304a and a second diseased disc 304b). The upper, middle, and lower vertebrae 302a-c are shown as partially transparent in FIGS. 3A-3M for clarity. The diseased discs 304a-b can result from degenerative joint disease and/or disc arthropathy. In the illustrated embodiment, the upper vertebra 302a is the L4 lumbar vertebra, the middle vertebra is the L5 lumbar vertebra, and the lower vertebra 302c is the S1 sacral vertebra.

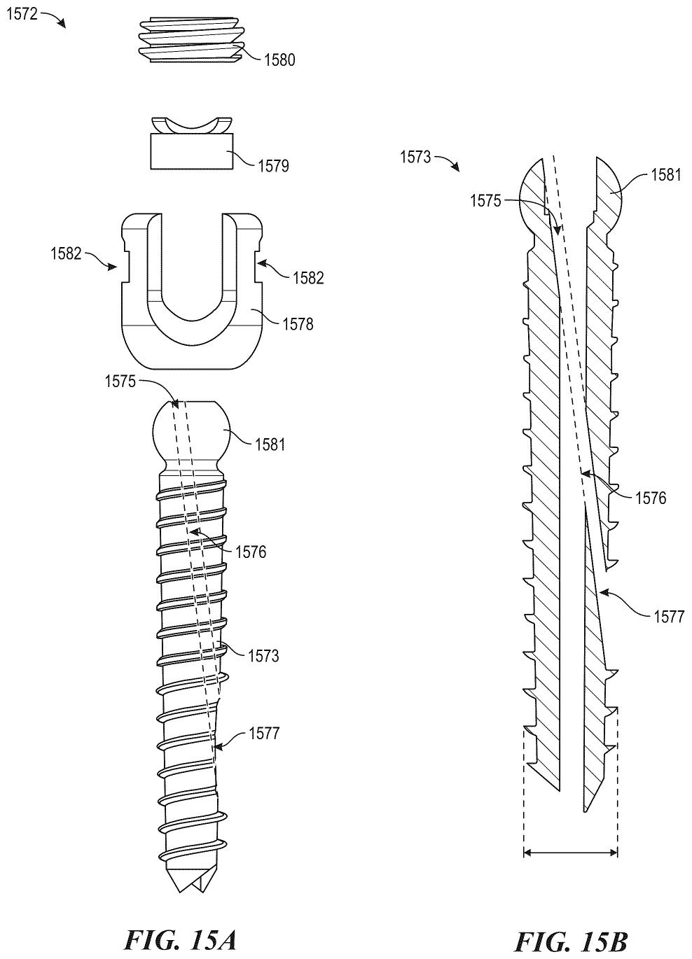

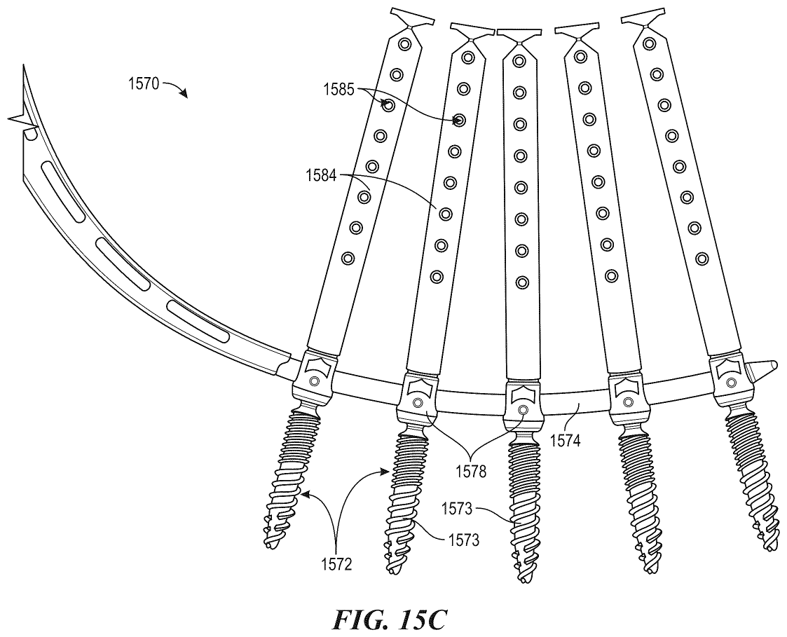

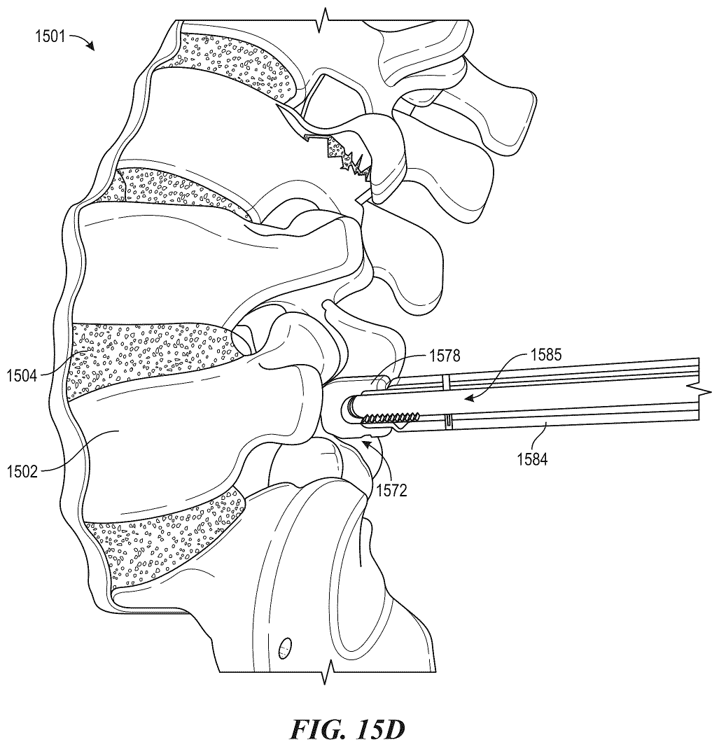

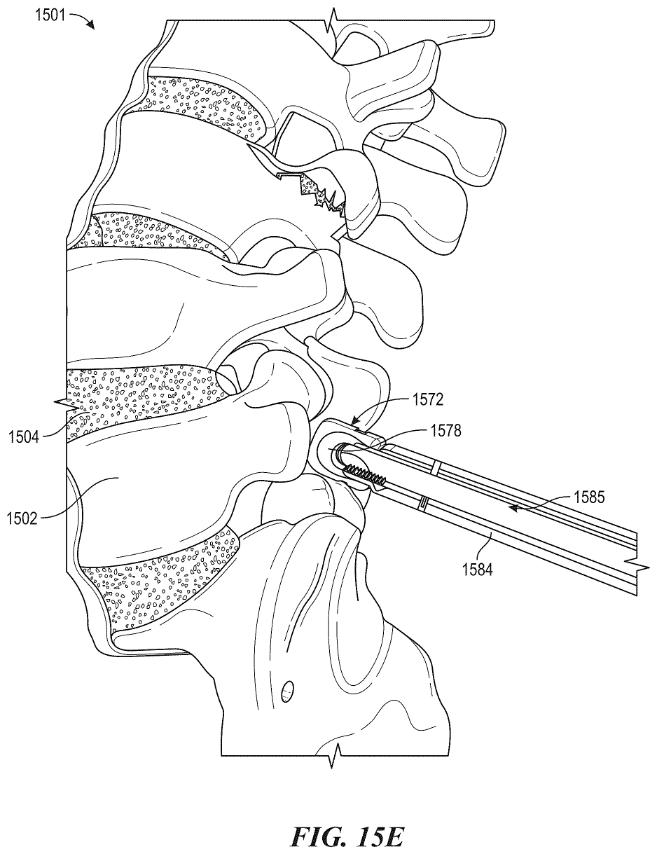

In the first posterior fixation step, one or more (e.g., two) first fixation members 372a are secured within the upper vertebra 302a, one or more (e.g., two) second fixation members 372b are secured within the middle vertebra 302b, and one or more (e.g., two) third fixation members 372c are secured within the lower vertebra 302c. The first, second, and third fixation members 372a-c (collectively “fixation members 372”) can be pedicle screws, cortical screws, wires, bands, interspinous clamps, interlaminar clamps, plates, dowels, and/or the like. For example, in the illustrated embodiment the fixation members 372 are pedicle screws each including a threaded screw body 373 configured to be screwed into and secured within the corresponding one of the vertebrae 302 and a polyaxial head or tulip 375 rotatably coupled to the screw body 373. In some embodiments, the fixation members 372 can include some features similar and/or identical in structure and/or function to the fixation member 1572 described in detail with reference to FIGS. 15A and 15B.

FIG. 3B is a side view (e.g., a lateral view) of a portion of the spine 300 illustrating a second posterior fixation step (e.g., a rod insertion step) of the spinal surgical procedure in accordance with embodiments of the present technology. In the second posterior fixation step, one or more spanning members 374 can be coupled to the fixation members 372 to secure the fixation members 372 together. The spanning members 374 can be rods, wires, bands, plates, clamps, and/or the like. In the illustrated embodiment, there are two of the spanning members 374 each comprising a rod, and individual ones of the spanning members 374 are coupled to a corresponding tulip 375 of one of the first fixation members 372a, one of the second fixation members 372b, and one of the third fixation members 372c. The fixation members 372 and the spanning members 374 can together define/comprise a posterior fixation assembly 370.

FIG. 3C is a side view (e.g., a lateral view) of a portion of the spine 300 illustrating a third posterior fixation step (e.g., a tower insertion step) of the spinal surgical procedure in accordance with embodiments of the present technology. In the third posterior fixation step, tower members 384 (e.g., towers, positioning tubes, access channels) can be releasably secured (e.g., rigidly) to corresponding ones of the tulips 375 of the fixation members 372. The tower members 384 can each provide an access channel for accessing a corresponding one of the fixation members 372 during subsequent steps of the spinal surgical procedure described in detail below.

FIG. 3D is a side view (e.g., a lateral view), including an enlarged portion, of a portion of the spine 300 illustrating an access step of the spinal surgical procedure in accordance with embodiments of the present technology. In the access step, a first trocar 310a can be used to access the first diseased disc 304a and a second trocar 310b can be used to access the second diseased disc 304b. In some embodiments, the first and second trocars 310a-b (collectively “trocars 310”) can be inserted via a minimally-invasive lateral approach. In other embodiments, one or both of the trocars 310 can be inserted via a transpedicular or transforaminal (e.g., transfacet) approach such as, for example, described in detail above with reference to FIGS. 1A and 1B. In yet other embodiments, one or both of the trocars 310 can be inserted via an anterior approach. The trocars 310 can include some features generally similar or identical in structure and/or function to the trocar 110 described in detail above with reference to FIGS. 1A-1S and/or elsewhere herein. For example, in the illustrated embodiment, the trocars 310 each include a handle 312 coupled to a hollow cannula 314 defining a lumen. In some embodiments, an introducer is positioned within the lumens of each of the trocars 310 during the access step as the trocars 310 are pushed, rotated, and/or otherwise advanced through the flesh of the patient 301 to proximate the diseased discs 304a-b.

FIGS. 3E and 3F are side views (e.g., a lateral views), including enlarged portions, of a portion of the spine 300 illustrating distraction steps (e.g., a parallel distraction steps) of the spinal surgical procedure in accordance with embodiments of the present technology. Referring to FIG. 3E, a first balloon 330a can be inserted through the cannula 314 of the first trocar 310a and inflated in a first disc space 307a between the upper vertebra 302a and the middle vertebra 302b to distract the first disc space 307a and create separation (e.g., height) between the upper and middle vertebrae 302a-b. For example, inflation of the first balloon 330a can force the upper and middle vertebrae 302a-b to move away from one another by a first distance D₁ of between about 1-15 millimeters, between about 1-10 millimeters, between about 1-8 millimeters, about 8 millimeters, etc. The first fixation members 372a and the second fixation members 372b (e.g., the screw bodies 373 thereof; FIG. 3A) are fixed within the upper and lower vertebrae 302a-b, respectively, such that they move therewith during expansion of the first balloon 330a. Accordingly, in some embodiments the tulips 375 (and coupled tower members 384) of the first fixation members 372a and/or the tulips 375 of the second fixation members 372b can slide along the spanning members 374 during expansion of the first balloon 330a.

Referring to FIG. 3F, a second balloon 330b can similarly be inserted through the cannula 314 of the second trocar 310b and inflated in a second disc space 307b between the middle vertebra 302b and the lower vertebra 302c to distract the second disc space 307b and create separation (e.g., height) between the middle and lower vertebrae 302b-c. For example, inflation of the second balloon 330b can force the middle and lower vertebrae 302b-c to move away from one another by a first distance D₂ of between about 1-15 millimeters, between about 1-10 millimeters, between about 1-8 millimeters, about 8 millimeters, etc. The second fixation members 372b and the third fixation members 372c (e.g., the screw bodies 373 thereof; FIG. 3A) are fixed within the middle and lower vertebrae 302b-c, respectively, such that they move therewith during expansion of the second balloon 330b. Accordingly, in some embodiments the tulips 375 (and coupled tower members 384) of the second fixation members 372b and/or the tulips 375 of the third fixation members 372c can slide along the spanning members 374 during expansion of the second balloon 330b.

Referring to FIGS. 3E and 3F, the first and second balloons 330a-b can include some features generally similar or identical in structure and/or function to those of the first balloon 130 described in detail above with reference to FIGS. 1E-1H and/or elsewhere herein. In some embodiments, a discectomy device is first inserted through the first trocar 310a to remove some or all of the first diseased disc 304a before distraction of the first disc space 307a with the first balloon 330a and/or the same or a different discectomy device is first inserted through the second trocar 310b to remove some or all of the second diseased disc 304b before distraction of the second disc space 307b with the second balloon 330b as, for example, described in detail above with reference to FIGS. 1C and 1D and/or elsewhere herein. The balloons 330a-b can be expanded sequentially (e.g., the first balloon 330a before the second balloon 330b, the second balloon 330b before the first balloon 330a) or simultaneously.

FIGS. 3G-3I are side views (e.g., a lateral views), including enlarged portions, of a portion of the spine 300 illustrating distraction steps (e.g., a lordosis and distraction steps) of the spinal surgical procedure in accordance with additional embodiments of the present technology. The distraction steps illustrated in FIGS. 3G-3I can be performed as an alternative to the distraction steps illustrated in FIGS. 3E and 3F—or can be performed after the distraction steps illustrated in FIGS. 3E and 3F.

Referring to FIG. 3G, a locking device 378 (e.g., a positioning tie) can be releasably secured to some or all of the tower members 385. The locking device 378 can be a clamp or similar structure configured to fixedly secure (e.g., lock) the position and orientation of the tower members 385 relative to one another. The tower members 385 are fixedly coupled to corresponding ones of the tulips 375 of the fixation members 372 such that the locking device 378 further acts to fixedly secure (e.g., lock) the position and orientation of the tulips 375 relative to another. That is, for example, the locking device 378 can inhibit or even prevent the tulips 375 from sliding (e.g., axially) along the spanning members 374 and/or rotating. In some embodiments, the locking device 378 can comprise one or more clips, clamps, and/or the like that are fixed to the spanning members 374 adjacent the tulips 375 to inhibit or even prevent the tulips 375 from sliding (e.g., axially) along the spanning members 374. More generally, the locking device 378 is configured to inhibit or even prevent (e.g., lock) axial movement of the tulips 375 along the spanning members 374 such that the fixation members 372 are constrained to pivot rather than move laterally relative to one another.

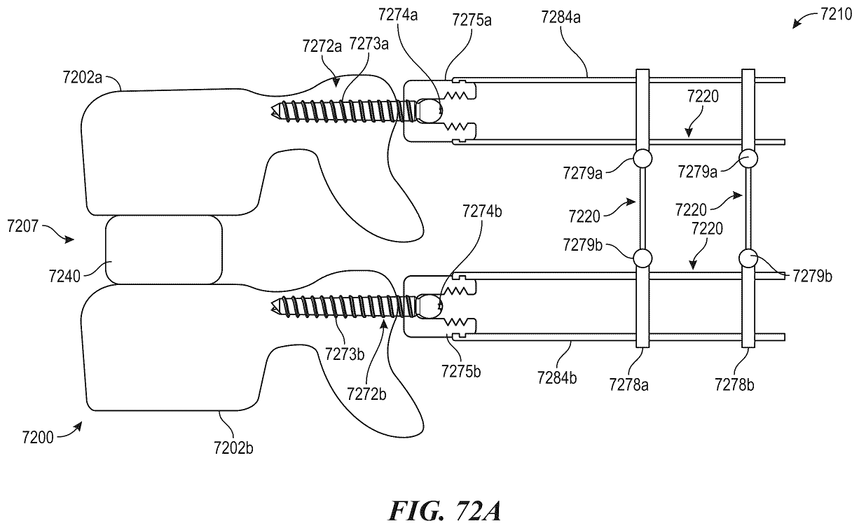

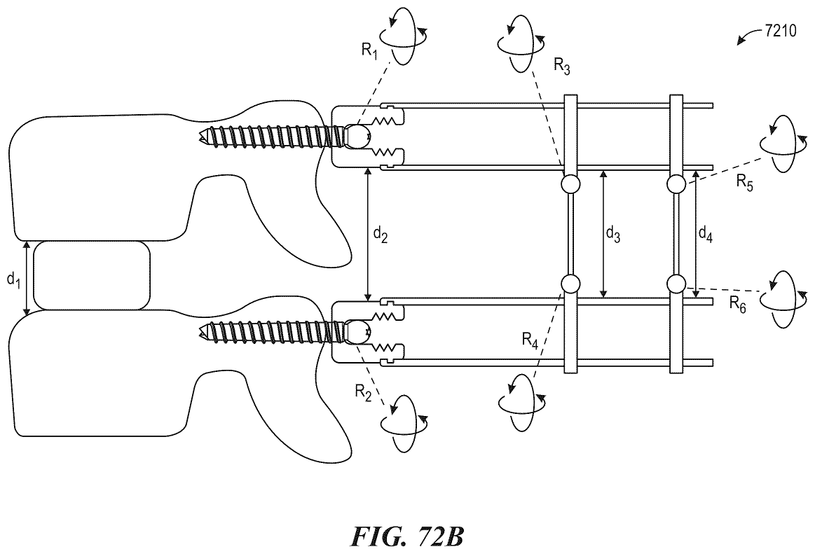

Referring to FIG. 3H, the first balloon 330a can be inserted through the cannula 314 of the first trocar 310a and inflated in the first disc space 307a between the upper vertebra 302a and the middle vertebra 302b to distract the first disc space 307a and create separation (e.g., height) and lordosis between the upper and middle vertebrae 302a-b. For example, inflation of the first balloon 330a can force the upper and middle vertebrae 302a-b to pivot away from one another by an angle A₁ of between about 1-15 degrees, between about 1-10 degrees, between about 2-8 degrees, about 7 degrees, etc. More specifically, the locking device 378 can fixedly secure the position and orientation of the tulips 375 relative to one another such that the threaded screw bodies 373 (labeled in the enlarged portion of the view) of the first and second fixation members 372a-b are constrained to pivot about/within the tulips 375. Such mechanical constraint of the first and second fixation members 372a-b constrains the upper and middle vertebrae 302a-b to pivot to create the angle A₁ as the first balloon 330a is expanded as opposed to simply moving laterally away from another as shown in, for example, FIG. 3E. In some embodiments, the tower members 384 and/or another component of the system can include one or more devices configured to measure the angle A₁ in real time or near real time (e.g., as described in detail below with reference to FIGS. 72A-77) to, for example, provide intelligent feedback to a surgeon or other operator of the effect of the expansion of the first balloon 330a on the curvature of the spine 300. Such devices for measuring the angle A₁ can be optical, electrical, mechanical, and/or the like.

Referring to FIG. 3I, the second balloon 330b can similarly be inserted through the cannula 314 of the second trocar 310b and inflated in the second disc space 307b between the middle vertebra 302b and the lower vertebra 302c to distract the second disc space 307b and create separation (e.g., height) and lordosis between the middle and lower vertebrae 302b-c. For example, inflation of the second balloon 330b can force the middle and lower vertebrae 302b-c to pivot away from one another by an angle A₂ of between about 1-15 degrees, between about 1-10 degrees, between about 2-8 degrees, about 7 degrees, etc. More specifically, the locking device 378 can fixedly secure the position and orientation of the tulips 375 relative to one another such that the threaded screw bodies 373 (labeled in the enlarged portion of the view) of the second and third fixation members 372b-c are constrained to pivot about/within the tulips 375. Such mechanical constraint of the second and third fixation members 372b-c constrains the middle and lower vertebrae 302b-c to pivot to create the angle A₂ as the second balloon 330b is expanded as opposed to simply moving laterally away from another as shown in, for example, FIG. 3F. In some embodiments, the tower members 384 and/or another component of the system can include one or more devices configured to measure the angle A₂ in real time or near real time (e.g., as described in detail below with reference to FIGS. 72A-77) to, for example, provide intelligent feedback to a surgeon or other operator of the effect of the expansion of the first balloon 330a on the curvature of the spine 300. Such devices for measuring the angle A₂ can be optical, electrical, mechanical, and/or the like. In some embodiments, the angle A₂ can be the same as or similar to the angle A₁ (FIG. 3H).

Referring to FIGS. 3H and 3I, the first and second balloons 330a-b can include some features generally similar or identical in structure and/or function to those of the first balloon 130 described in detail above with reference to FIGS. 1E-1H and/or elsewhere herein. In some embodiments, a discectomy device is first inserted through the first trocar 310a to remove some or all of the first diseased disc 304a before distraction of the first disc space 307a with the first balloon 330a and/or the same or a different discectomy device is first inserted through the second trocar 310b to remove some or all of the second diseased disc 304b before distraction of the second disc space 307b with the second balloon 330b as, for example, described in detail above with reference to FIGS. 1C and 1D and/or elsewhere herein. The balloons 330a-b can be expanded sequentially (e.g., the first balloon 330a before the second balloon 330b, the second balloon 330b before the first balloon 330a) or simultaneously.

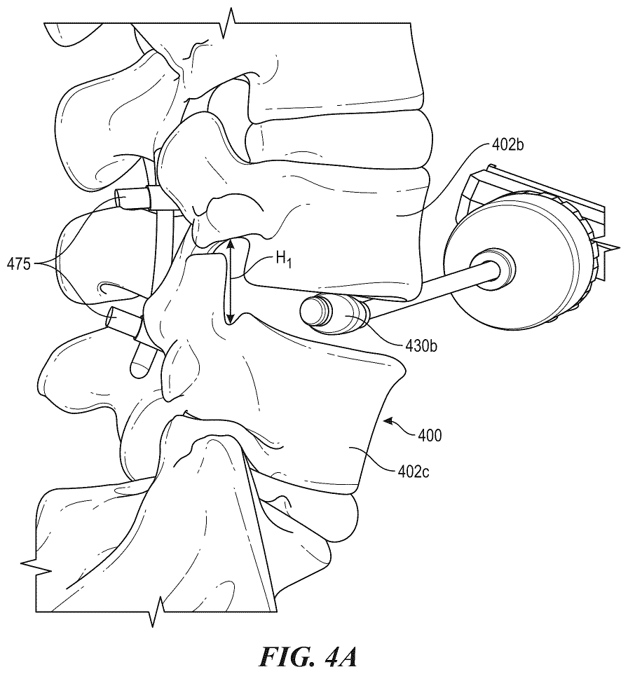

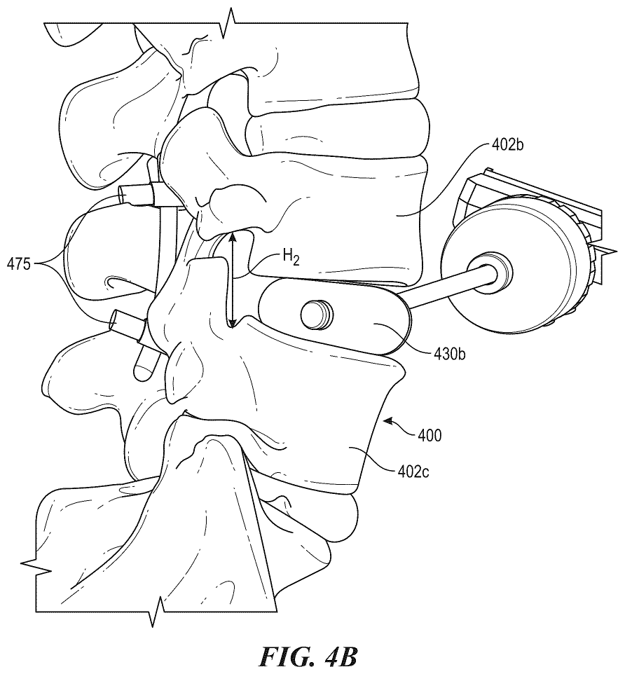

In some aspects of the present technology, inflation of the first and second balloons 330a-b can create lordosis of the spine 300 without compressing the foramen around the nerve root and, in some embodiments, can decompress the foramen around the nerve root. For example, the pivot points of the vertebrae 302 at the posterior fixation assembly 370 (e.g., at the tulips 375) are located behind (e.g., posterior to) the foramen and the nerve root such that inflation of the first and second balloons 330a-b increases the intervertebral foraminal height. More specifically, FIGS. 4A and 4B are side views (e.g., lateral views) of a portion of the spine 300 before and after inflation of the second balloon 330b in accordance with embodiments of the present technology. Referring to FIG. 4A, before inflation of the second balloon 330b the middle and lower vertebrae 302b-c can have/define a first foraminal height H₁. Referring to FIG. 4B, inflation of the second balloon 330b can create lordosis of the spine 300 and increase the height to a second foraminal height H₂ greater than the first foraminal height H₁. This can reduce compression around a nerve root extending from the foramen and is achieved because the pivot point for lordosis creation is at the tulips 375 positioned behind (e.g., posterior to) the foramen. In contrast, many conventional surgical techniques utilizing an interbody create lordosis by compressing the screws pivoting on the anterior edge. If the facet joint is not left intact and then the segments are semi-free floating during placement of the interbody and the posterior is reduced to create the angle, the foraminal height is reduced, potentially creating compression of the nerve root.

FIGS. 3J and 3K are side views (e.g., a lateral views) of a portion of the spine 300 illustrating posterior fixation locking steps of the spinal surgical procedure in accordance with embodiments of the present technology. The posterior fixation steps illustrated in FIGS. 3J and 3K can be performed after the distraction and lordosis steps illustrated in FIGS. 3G-3I. Referring to FIG. 3J, with the first balloon 330a and the second balloon 330b expanded to maintain the lordotic angles A₁ and A₂, a set screw 380 can be inserted through each of the tower members 384 and into the corresponding one of the tulips 375 of the fixation members 372. Referring to FIG. 3J, with the first balloon 330a and the second balloon 330b expanded to maintain the lordotic angles A₁ and A₂, the posterior fixation assembly 370 can be locked in position by inserting one or more drivers 379 through the tower members 384 and rotating the driver(s) 379 to tighten the set screws 380 (FIG. 3J) to, for example, lock (e.g., via friction) an orientation/position of the tulip 375 relative to (i) the screw body 373 of each of the fixation members 372 and (ii) the spanning members 374. In some embodiments, the fixation members 372 can be locked in position/orientation as described in detail below with reference to FIGS. 15A and 15B and/or elsewhere herein.

FIG. 3L is a side view (e.g., a lateral view), including an enlarged portion, of a portion of the spine 300 illustrating an intervertebral device deployment step of the spinal surgical procedure in accordance with embodiments of the present technology. In the illustrated embodiment, after locking the posterior fixation assembly 370 (FIG. 3K), the first and second balloons 330a-b can be deflated and removed through the cannulas 314 of the first and second trocars 310a-b, respectively, and (i) a first intervertebral device 340a can be inserted through the cannula 314 of the first trocar 310a and deployed within the first disc space 307a between the upper and middle vertebrae 302a-b and (ii) a second intervertebral device 340b can be inserted through the cannula 314 of the second trocar 310b and deployed within the second disc space 307b between the middle and lower vertebrae 302b-c. In some embodiments, the first and second intervertebral devices 340a-b can include some features generally similar or identical in structure and/or function to the intervertebral device 140 described in detail above with reference to FIGS. 1I-1R and/or elsewhere herein, and can be deployed in a generally similar or identical manner (e.g., including expanding, filling, tensioning, and closing). In some embodiments, a discectomy device is first inserted through the first trocar 310a to remove some or all of the first diseased disc 304a (FIG. 3A) before deployment of the first intervertebral device 340a and/or the same or a different discectomy device is first inserted through the second trocar 310b to remove some or all of the second diseased disc 304b (FIG. 3A) before deployment of the second intervertebral device 340b as, for example, described in detail above with reference to FIGS. 1C and 1D and/or elsewhere herein. Alternatively, the first and second intervertebral devices 340a-b can be deployed after the distraction steps illustrated in FIGS. 3E and 3F without locking of the posterior fixation assembly 370. The posterior fixation assembly 370 can then be locked after deployment of the first and second intervertebral devices 340a-b.

Finally, the tower members 384 and the first and second trocars 310a-b can be removed from the patient. FIG. 3M, for example, is a side view of a portion of the spine 300 illustrating the finally-implanted first and second intervertebral devices 340a-b and the posterior fixation assembly 370 in accordance with embodiments of the present technology.

Referring to FIGS. 3A-3M, in some aspects of the present technology the spinal surgical procedure can be performed without the use of retractors—as the first and second trocars 310a-b provide a minimally-invasive, retractor-less access port for the deployment of the of the first and second intervertebral devices 340a-b, distraction and lordosis of the first and second disc spaces 307a-b, etc. Not requiring the use of retractors can minimize trauma to muscle, viscera, nerves, and/or the like of the patient 301, which is a significant contributor to non-trivial post-operative pain in conventional spinal surgical procedures. Likewise, in additional aspects of the present technology the patient 301 can be positioned in a single position (e.g., a prone position) during the entirety of the spinal surgical procedure. In particular, the trocars 310a-b can provide an access port to the first and second disc spaces 307a-b that does not require direct visualization such that the patient 301 can be positioned in a single position during installation and manipulation of the posterior fixation assembly 370. Moreover, although a two-level spinal fusion procedure is illustrated in FIGS. 3A-3M, the spinal surgical procedure can be similarly carried out to treat only a single diseased one of the discs 304 and to fuse only two adjacent levels of the vertebrae 302, and/or to treat more than two diseased one of the discs 304 and to fuse more than three adjacent levels of the vertebrae 302.

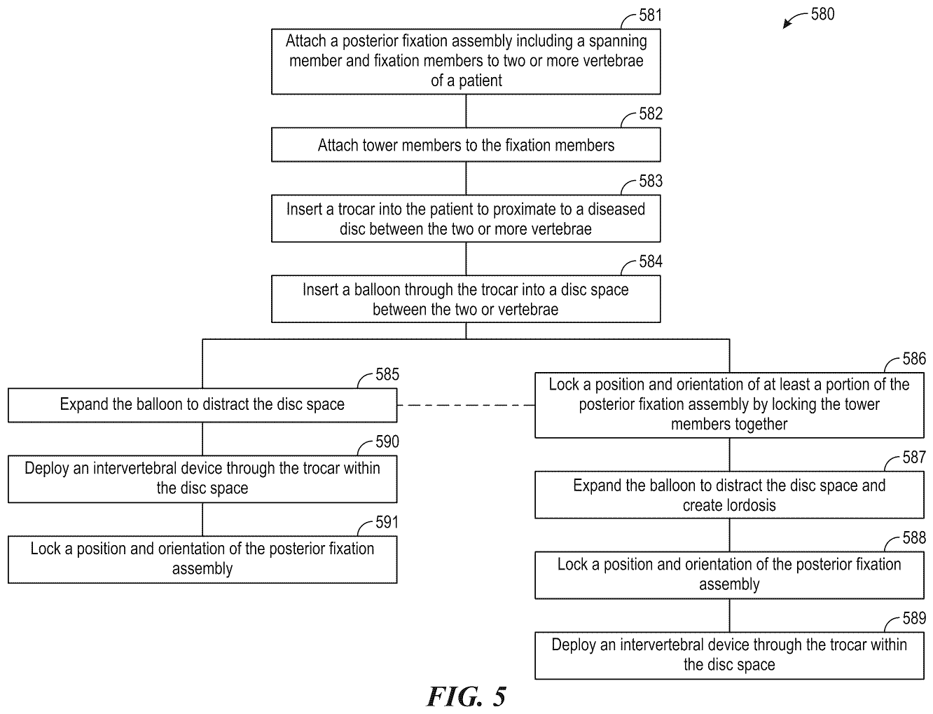

FIG. 5 is a flow diagram of a process or method 580 for performing a spinal surgical procedure, such as the spinal surgical procedure (e.g., a spinal fusion procedure) illustrated with reference to FIGS. 3A-3M, in accordance with embodiments of the present technology. At block 581, the method 580 can include attaching a posterior fixation assembly including at least one spanning member and fixation members to two or more vertebrae of a patient, such as described in detail above with reference to FIGS. 3A and 3B. At block 581, the method 580 can include attaching tower members to the fixation members, such as described in detail above with reference to FIG. 3C. At block 583, the method 580 can include inserting at least one trocar into the patient to proximate a diseased disc between the two or more vertebrae via, for example, a lateral approach, such as described in detail above with reference to FIG. 3D. In some embodiments, such as for a two-level fusion procedure, a first trocar is inserted to proximate a first diseased disc and a second trocar is inserted to proximate a second diseased disc. At block 584, the method can include inserting a balloon through the trocar into a disc space between the two or more vertebrae. In some embodiments, such as for a two-level fusion procedure, a first balloon is inserted into a first disc space via the first trocar and a second balloon is inserted into a second disc space via the second trocar.

After block 584, the method 580 can proceed to block 585 to include expanding the balloon(s) to distract the disc space(s), as described in detail above with reference to FIGS. 3E and 3F, or to block 586 to lock the position and orientation of at least a portion of the posterior fixation assembly (e.g., tulips of the fixation members) by locking the tower members together, as described in detail above with reference to FIG. 3G. In some embodiments, the method 580 can proceed from block 585 to block 586. At block 587, the method 580 can include expanding the balloon(s) to distract the disc space(s) and create lordosis of the two or more vertebrae. In some embodiments, the method 580 includes determining/measuring the created lordosis (e.g., lordotic angle) in real time or near real time.

At block 588, the method 580 can include locking the orientation and position of the posterior fixation assembly, as described in detail above with reference to FIGS. 3J and 3K. At block 589, the method 580 can include deploying an intervertebral device through the trocar within the disc space, as described in detail above with reference to FIG. 3L. In some embodiments, such as for a two-level fusion procedure, a first intervertebral device is deployed within the first disc space via the first trocar and a second intervertebral device is deployed within the second disc space via the second trocar.

If the method proceeds to block 585 and not to block 586, the method 580 can include deploying the intervertebral device(s) within the disc space(s) through the trocar(s) at block 590, and then locking a position and orientation of the posterior fixation assembly at block 591.

### II. SELECTED EMBODIMENTS OF INSTRUMENTS FOR PROVIDING SPINAL ACCESS, AND ASSOCIATED SYSTEMS AND METHODS

FIGS. 6A-17B illustrate embodiments of certain instruments and/or instrumentation that can be used to facilitate and/or aid access to a diseased disc, such as described in detail above with reference to FIGS. 1A and 1B and block 281 of the method 280 of FIG. 2 and FIG. 3D and block 583 of the method of FIG. 5. Accordingly, the embodiments described with reference to FIGS. 6A-17B can be utilized in the workflow of the spinal surgical procedures described in detail with reference to FIGS. 1A-5, and/or elsewhere herein.

FIG. 6A is a perspective view of an access alignment assembly 610 positioned on a patient 600 in accordance with embodiments of the present technology. In the illustrated embodiment, the access alignment assembly 610 includes a marker grid 612 coupled to a sterile adhesive or other layer 614. FIG. 6B is a schematic perspective view of the marker grid 612 in accordance with embodiments of the present technology. Referring to FIG. 6B, the marker grid 612 can comprise a plurality of radiopaque markers 611 positioned in a grid pattern along one of multiple grid layers 613 (e.g., an individually identified first grid layer 613a, a second grid layer 613b, and a third grid layer 613c). Referring to FIGS. 6A and 6B, when the access alignment assembly 610 is positioned on the patient 600, the first grid layer 613a can be positioned closest to the skin of the patient 600 (e.g., adjacent the skin of the patient), and the third grid layer 613c can be positioned farthest from the skin of the patient 600.

During a spinal surgical procedure, the patient 600 can be imaged using x-ray imaging, computed tomography (CT) imaging, magnetic resonance imaging (MRI), and/or the like while the access alignment assembly 610 is positioned on the patient 600. By taking images from at least two different perspectives (e.g., along two different orthogonal axes), an image processor can construct a three-dimensional (3D) model of a portion of the patient 600 including the imaged marker grid 612. The 3D model can be used to define an optimal entry point and trajectory for inserting a trocar through a transpedicular or transforaminal approach to a diseased disc of the patient 600. For example, the image processor can (i) determine an optimal entry point and output this information as a first coordinate (x, y) on the first grid layer 613a directly adjacent the skin of the patient and (ii) an optimal trajectory and output this information as a second coordinate in the second grid layer 613b and/or as a third coordinate in the third grid layer 613c. Then, a surgeon need only traverse the identified coordinates to ensure that they make the correct entry point and trajectory. In contrast, some conventional systems use imaging intraoperatively as a needle is advanced into the patient to locate the entry point and trajectory. However, the surgeon can be required to start and stop and rely on experience and anatomical references while taking x-rays along the way.

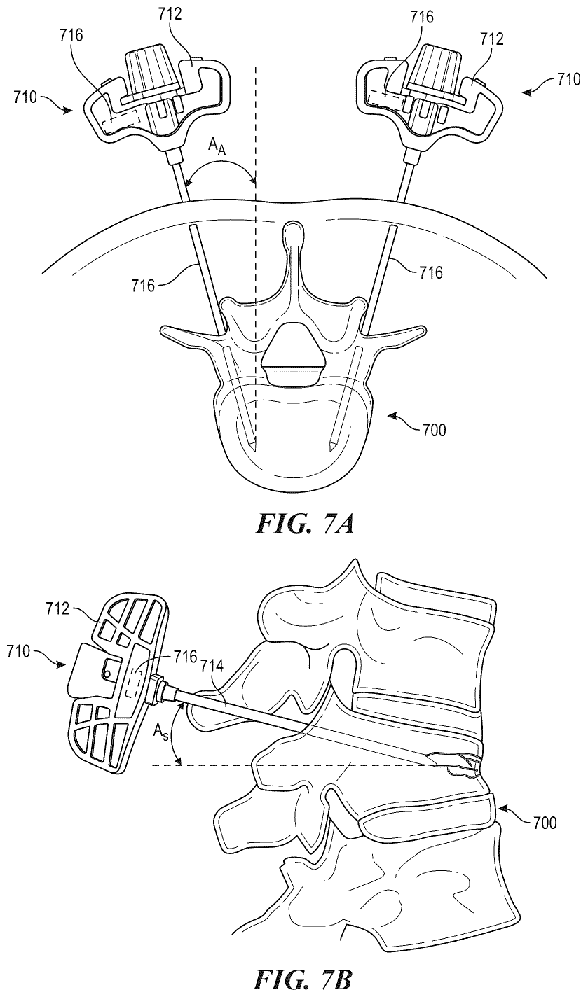

FIGS. 7A and 7B are a top (e.g., axial) view and a side (e.g., lateral) view, respectively, of a trocar 710 for providing access (e.g., lateral, transpedicular, transfacet, transforaminal, and/or other access) to a vertebra or disc of a spine 700 in accordance with embodiments of the present technology. Referring to FIGS. 7A and 7B, the trocar 710 includes a handle 712 coupled to a hollow cannula 114. In the illustrated embodiment, the trocar 710 can include an angle determination unit 716 built into the trocar 710 (e.g., into the handle 712) and configured to detect an angle of the cannula 114 relative to the spine 700, such as an axial angle A_{A} (FIG. 7A), a sagittal angle A_{S} (FIG. 7B), and/or another angle along a different axis. The angle determination unit 716 can comprise a gyroscope, protractor, and/or another device configured to determine an angle. During a spinal surgical procedure, a surgeon can position the cannula 714 at a predetermined entry point into the patient, and then adjust the angle of the cannula 714 based on a readout from the angle determination unit 716 to align the cannula 714 along a predetermined trajectory to provide transpedicular or transforaminal access to the spine 700.

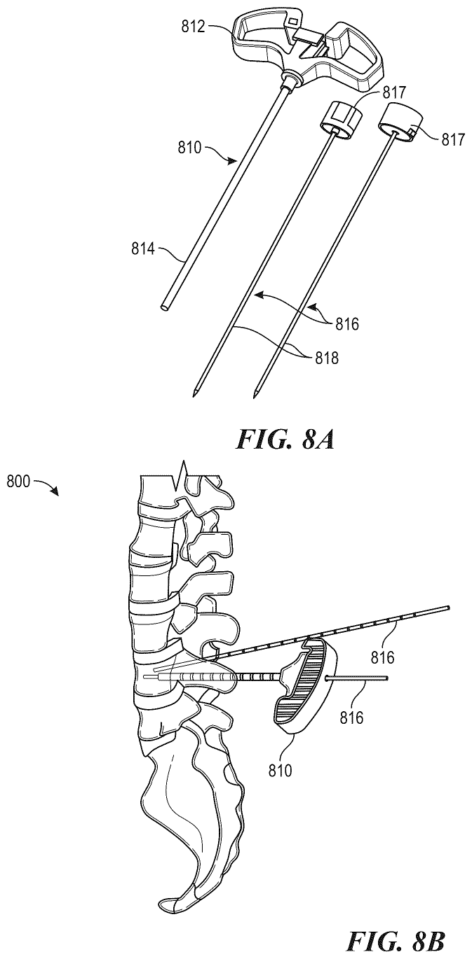

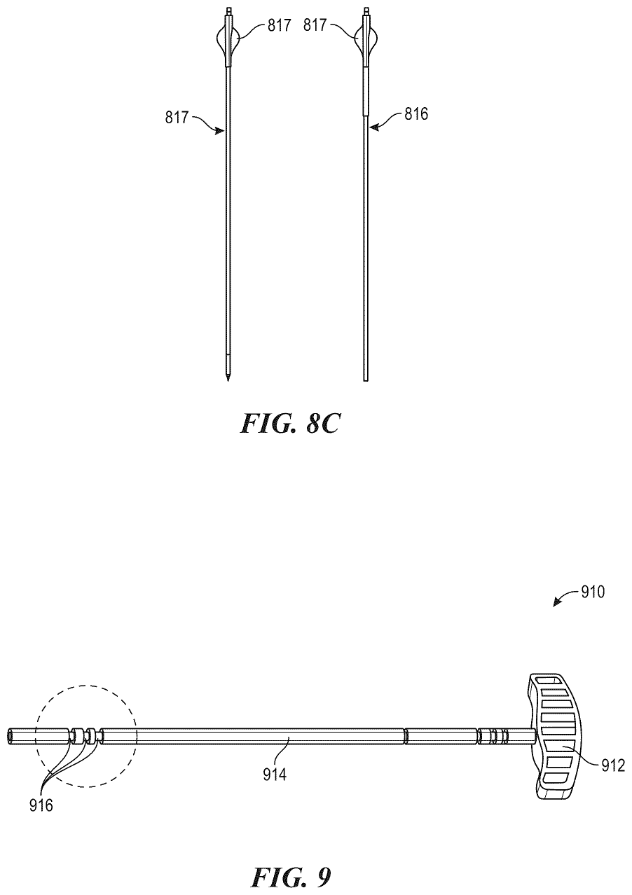

FIG. 8A is a perspective view of a trocar 810 and a pair of inner stylets 816 for use with the trocar 810 in accordance with embodiments of the present technology. In the illustrated embodiment, the trocar 810 includes a handle 812 coupled to a hollow cannula 814, and the stylets 816 each include a grip 817 (e.g., a handle, a hub) coupled to an elongate member 818 (e.g., a needle). The grip 817 of the stylets 816 can be detachable from the elongate member 818. During a spinal surgical procedure, a surgeon can first insert one of the stylets 816 into the patient along a determined trajectory through a determined entry point. Then, if the stylet 816 is correctly positioned, the surgeon can remove the grip 817 from the elongate member 818 and then advance the cannula 814 of the trocar 810 over the elongate member 818 to align the trocar along the determined trajectory. In some aspects of the present technology, first positioning the stylet 816 along the correct trajectory—before introducing the trocar 810—can reduce trauma to the patient in the case that the stylet 816 needs to be repositioned, as the elongate member 818 of the stylet 816 can have a smaller profile than the cannula 814 of the trocar 810 to minimize tissue trauma. FIG. 8B is a side (e.g., lateral) view illustrating the two stages of (i) first inserting the stylet 816 into a spine 800 of a patient and then (2) advancing the trocar 810 over the stylet 816 to access the spine 800 in accordance with embodiments of the present technology. After advancing the trocar 810 over the stylet 816, the stylet 816 can be removed from the cannula 814 such that additional devices (e.g., a balloon, an intervertebral device) can be advanced through the cannula 814.

FIG. 8C is a side view of the stylets 816 in accordance with additional embodiments of the present technology. In the illustrated embodiment, the grips 817 of the stylets 816 have a pen-like shape to facilitate manipulation by the surgeon. The grips 817 can be combined with additional features attachable to the stylets 816 such that the stylets 816 can be malleted into position.

FIG. 9 is a side view of a trocar 910 in accordance with embodiments of the present technology. In the illustrated embodiment, the trocar 910 includes a handle 912 coupled to a hollow cannula 914, and the cannula 914 has radiolucent notches 916 thereon to facilitate visualization of a depth of the cannula 914 within a patient via x-ray imaging. The cannula 914 can have any number of the notches 916 positioned at any longitudinal position along the cannula 914.

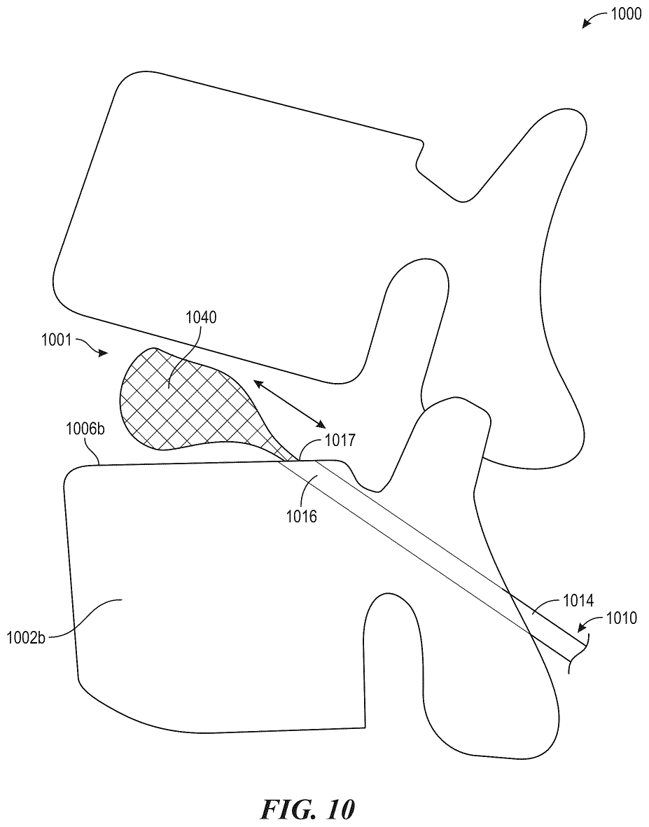

FIG. 10 is a side view of a trocar 1010 in accordance with embodiments of the present technology. In the illustrated embodiment, the trocar 1010 includes a handle (not shown) coupled to a hollow cannula 1014 which is positioned to access a spine 1000 of a patient. The cannula 1014 is inserted through a portion of a lower vertebra 1002b of the spine 1000 via, for example, a transpedicular approach. In the illustrated embodiment, the cannula 1014 includes a distal end portion 1016 having a beveled tip 1017 such that the distal end portion 1016 of the cannula 1014 can be substantially flush with an endplate 1006a (e.g., an upper endplate) of the lower vertebra 1002b when the trocar 1010 is inserted therethrough. In some aspects of the present technology, positioning the beveled tip 1017 flush with the endplate 1006a can facilitate introduction of instruments into a disc space 1001 above the lower vertebra 1002b without interference from the trocar 1010. For example, a balloon (not shown) or intervertebral device 1040 can be inserted into disc space 1001 through the trocar 1010 and expanded within the disc space 1001 without expanding into contact with the distal end portion 1016 of the cannula 1014, which could potentially damage the balloon or the intervertebral device 1040.

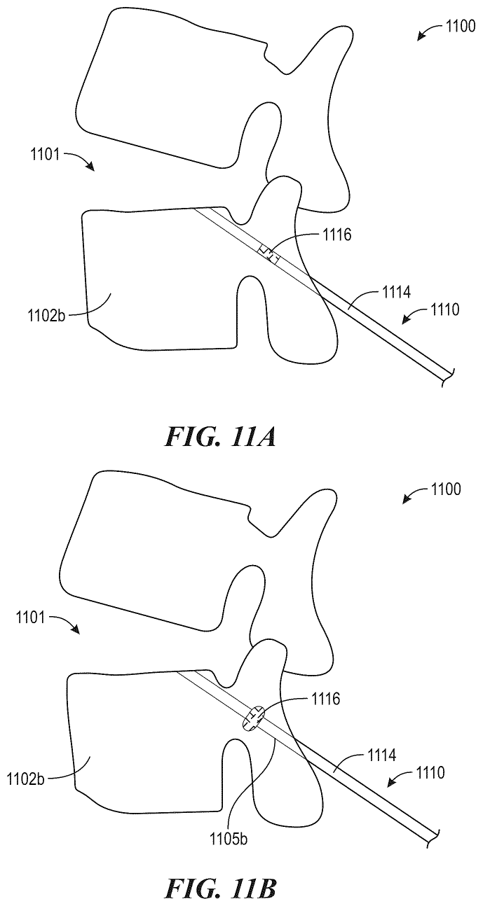

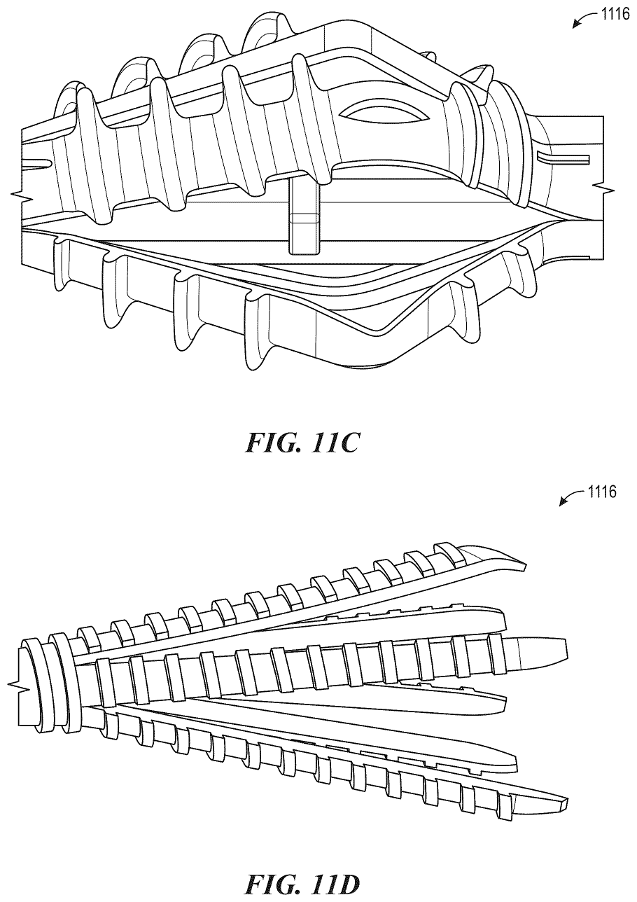

FIGS. 11A and 11B are side views of a trocar 1110 in a first position and a second position, respectively, in accordance with embodiments of the present technology. Referring to FIGS. 11A and 11B, in the illustrated embodiment the trocar 1110 includes a handle (not shown) coupled to a hollow cannula 1114 which is positioned to access a spine 1100 of a patient. The cannula 1114 is inserted through a portion of a lower vertebra 1102b of the spine 1100 via, for example, a transpedicular approach. The cannula 1114 can further include an expandable anchoring section 1116 positioned along a length thereof. The expandable anchoring section 1116 is compressed in the first position shown in FIG. 11A and is expanded in the second position shown in FIG. 11B. In some embodiments, the expandable anchoring section 1116 is similar to an expandable screw and can be actuated to flex outwardly from the first position to the second position via actuation (e.g., rotation) of the trocar 1110. For example, the expandable anchoring section 1116 can include a plurality of moveable arms that are configured to extend radially outward in the expanded second position. FIGS. 11C and 11D, for example, are side views of the expandable anchoring section 1116 in the expanded second position in accordance with embodiments of the present technology.

Referring to FIGS. 11A and 11B, the trocar 1110 can be inserted through the spine 1100 of the patient to gain access to a disc space 1101 thereof in the compressed first position, and then actuated to expand the expandable anchoring section 1116 to the expanded second position to anchor the position of the trocar 1110 relative to the disc space 1101. In the illustrated embodiment, the expandable anchoring section 1116 is positioned to expand within a pedicle 1105b of the lower vertebra 1102b to anchor the trocar 1110 therein. In some aspects of the present technology, the expandable anchoring section 1116 inhibits or even prevents axial and/or rotational shifting of the trocar 1110 during a spinal surgical procedure using the trocar 1110 (e.g., as additional devices are inserted through the cannula 1114).

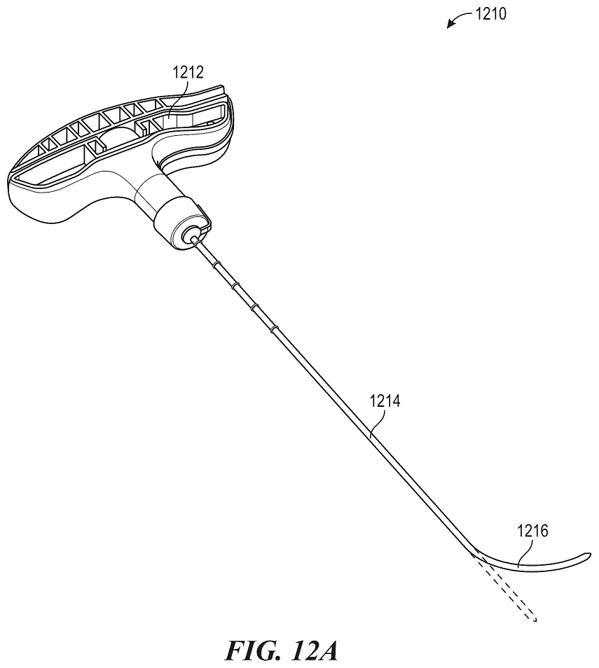

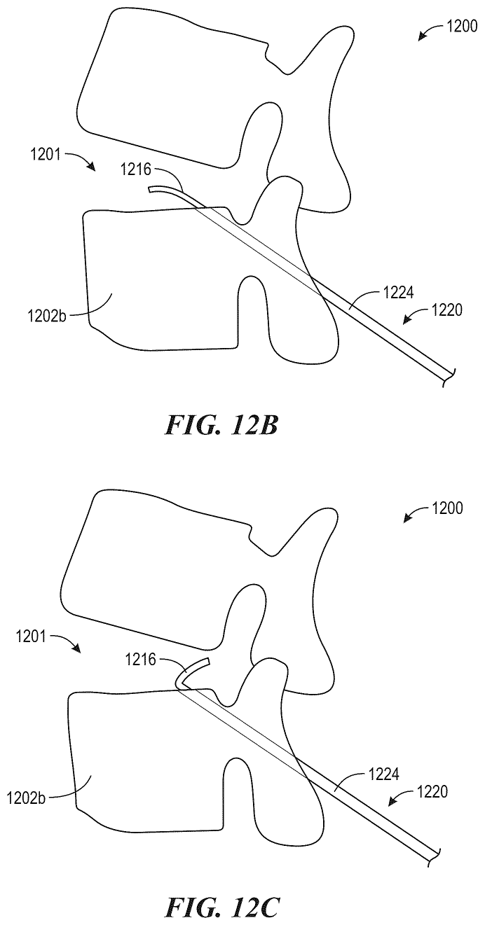

FIG. 12A is a perspective view of a trocar 1210 in accordance with embodiments of the present technology. In the illustrated embodiment, the trocar 1210 includes a handle 1212 coupled to a hollow cannula 1214 having a curved distal portion 1216. The distal portion 1216 can assume the curved shape when unconstrained, and can have a straight shape (e.g., as shown in dashed lines) if the distal portion 1216 is constrained within a straight lumen. The distal portion 1216 can be heat set or otherwise configured to assume the curved shape. In some embodiments, the cannula 1214 of the trocar 1210 is configured to be inserted through an introducer (e.g., a cannula of another trocar) with the distal portion 1216 having the straight shape, and the distal portion 1216 is configured to assume the curved shape when it extends out of the introducer. In other embodiments, a straight stylet or other elongate member can be inserted through the cannula 1214 to maintain the distal portion 1216 having the straight shape. The stylet or other elongate member can then be removed from (e.g., retracted proximally through) the distal portion 1216 to allow the distal portion 1216 to assume the curved shape.

The cannula 1214 can receive one or more instruments (e.g., balloons, intervertebral devices) during a spinal surgical procedure and guide the instruments into a disc space of a spine of a patient. In some aspects of the present technology, when the distal portion 1216 is positioned within the disc space, the trocar 1210 can be rotated and/or translated to provide access to different portions of the disc space. For example, FIGS. 12B and 12C are side views of the trocar 1210 inserted through an introducer 1220 (e.g., another trocar) in a first position and a second position, respectively, in accordance with embodiments of the present technology. Referring to FIGS. 12B and 12C, the introducer 1220 includes a handle (not shown) coupled to a hollow cannula 1224 which is positioned to access a spine 1200 of a patient. The cannula 1224 is inserted through a portion of a lower vertebra 1202b of the spine 1200 via, for example, a transpedicular approach.

In the illustrated embodiment, the distal portion 1216 of the trocar 1210 extends from the introducer 1220 into a disc space 1201 of the spine 1200. In FIG. 12A, the distal portion 1216 is curved toward the anterior portion of the disc space 1201 to, for example, facilitate introduction of an instrument (e.g., a discectomy device) toward the anterior portion. In FIG. 12B, the trocar 1210 is rotated such that distal portion 1216 is curved toward the posterior portion of the disc space 1201. Furthermore, advancing/retracting the distal portion relative to the cannula 1224 can further shift the position of the curved distal portion 1216 within the disc space 1201 to provide further steering and control. That is, rotation and translation of the curved distal portion 1216 relative to the cannula 1224 can sweep/steer the curved distal portion 1216 through the disc space 1201. In this manner, the curved distal portion 1216 facilitates access to different regions of the disc space 1201. That is, the curved distal portion 1216 can help guide an instrument received through the cannula 1214 to a desired positioned within the disc space 1201.

Referring to FIGS. 12A-12C, during a spinal surgical procedure, the trocar 1210 can be used to guide multiple instruments, or different trocars having different curved distal portions can be used at different times to impart different trajectories to instruments inserted therethrough. Likewise, multiple trocars having curved distal portions can be nested together to traverse multiple turns. In yet other embodiments, the cannula 1214 of the trocar 1210 can be shaped to curve in multiple directions/planes.

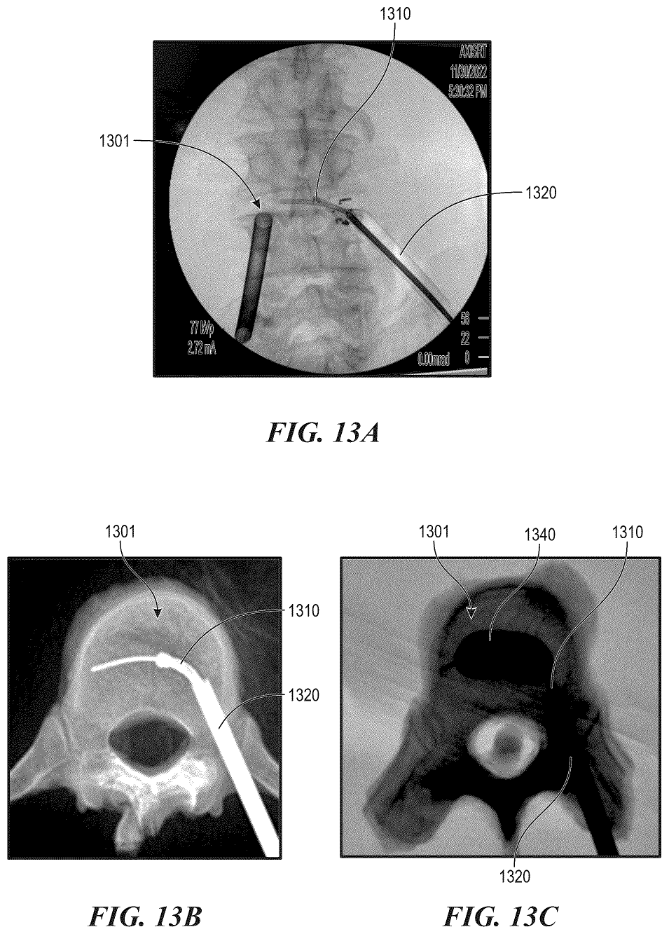

FIGS. 13A-13C are a coronal view, a top view, and another top view, respectively, of an intervertebral device deployment step of a spinal surgical procedure utilizing the trocar 1210 of FIG. 12A and the introducer 1220 of FIGS. 12B and 12C in accordance with embodiments of the present technology. Referring to FIGS. 13A and 13B, the curved trocar 1210 can be inserted through the straight introducer 1220 to provide access to a disc space 1301. Then, referring to FIG. 13C, an intervertebral device 1340 can be inserted over/through the curved trocar 1210 into the disc space 1301 for deployment therein. In some aspects of the present technology, the curved trocar 1210 can help guide the intervertebral device to a desired location within the disc space 1301, such as a centralized location therein. In other embodiments, the intervertebral device 1340 can be inserted through the straight introducer 1220 into the disc space 1301.

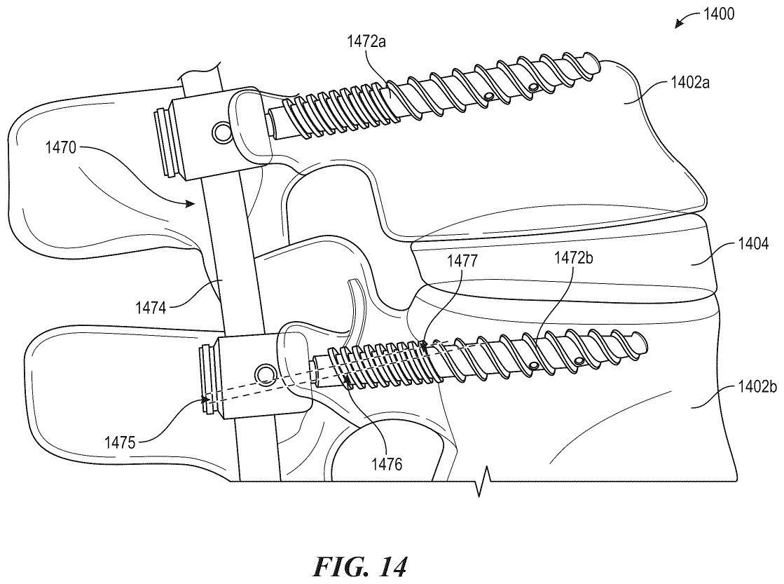

FIG. 14 is a side view of a posterior fixation assembly 1470 in accordance with embodiments of the present technology. In the illustrated embodiment, the posterior fixation assembly 1470 is fixedly attached to an upper vertebra 1402a and a lower vertebra 1402b of a spine 1400 of a patient to, for example, substantially stabilize the upper and lower vertebrae 1402a-b relative to one another. The posterior fixation assembly can include one or more first fixation members 1472a secured within the upper vertebrae 1402a and one or more second fixation members 1472b secured within the lower vertebra 1402b. The upper and lower fixation members 1472a-b can be pedicle screws and/or the like. Pairs of the upper and lower fixation members 1472a-b can be secured together via spanning members 1474, such as rods.

In the illustrated embodiment, the second fixation member 1472b includes a channel or cannulation 1476 extending partially therethrough. The cannulation 1476 can be a straight channel extending partially through the second fixation member 1472b from a posterior opening 1475 to an anterior opening 1477. The anterior opening 1477 can extend through a sidewall of the second fixation member 1472b and, accordingly, can be referred to as a side port or side fenestration. The cannulation 1476 can serve as a working channel to receive a trocar therethrough for accessing a diseased disc 1404 positioned between the upper and lower vertebrae 1402a-b. In some embodiments, for example, a curved trocar (e.g., the trocar 1210 described in detail with reference to FIGS. 12A-13C) can be inserted through the cannulation to access the diseased disc 1404 via a transpedicular approach. In other embodiments, the cannulation 1476 can be curved.

FIG. 15A is an exploded side view of a fixation member 1572 of a posterior fixation assembly in accordance with embodiments of the present technology. The fixation member 1572 can be a pedicle screw. In the illustrated embodiment, the fixation member 1572 includes a screw 1523 (e.g., a screw body), a tulip 1578, a grooved insert 1579 (e.g., saddle), and a set screw 1580. The screw 1573 can include a head portion 1581 configured to be coupled to/within the tulip 1578, and is configured to be inserted (e.g., screwed) into a vertebra to provide a screw-bone interface. The head portion 1581 can be spherical and the tulip 1578 can be rotatably coupled to the head portion 1581 such that the tulip 1578 can rotate along a sphere relative to the screw 1573. The tulip 1578 can include openings 1582 for receiving a spanning member (e.g., a rod) therethrough, and is configured (e.g., shaped and sized) to be releasably coupled to a tower member as, for example, described in further detail below with reference to FIGS. 15C-15E. Accordingly, the fixation member 1572 can be a poly-axial screw in which the tulip 1578 is spherically rotatable about the head portion 1581 of the screw 1573. The set screw 1580 can be rotated to compress the grooved insert 1579 against the tulip 1578 and/or a spanning member inserted therethrough to lock (e.g., via friction) an orientation/position of the tulip 1578 relative to the screw 1573.

FIG. 15B is a side cross-sectional view of the screw 1573 in accordance with embodiments of the present technology. Referring to FIGS. 15A and 15B, in the illustrated embodiment the screw 1573 includes a channel or cannulation 1576 extending partially therethrough. The cannulation 1576 can be a straight channel extending partially through the screw 1573 and/or the head portion 1581 from a posterior opening 1575 to an anterior opening 1577. The anterior opening 1577 can extend through a sidewall of the screw 1573 and, accordingly, can be referred to as a side port or side fenestration. The cannulation 1576 can serve as a working channel to receive a trocar therethrough for accessing a diseased disc.

FIG. 15C is a side view of a posterior fixation assembly 1570 including a plurality of the fixation members 1572 of FIG. 15A in accordance with embodiments of the present technology. The screws 1573 of each of the fixation members 1572 can be secured to a corresponding vertebra of a spine of patient. In the illustrated embodiment, a spanning member 1574 is coupled/secured to the tulips 1578 of the fixation members 1572 by, for example, being inserted through the openings 1582 (FIG. 15A) thereof. Each of the tulips 1578 is further releasably secured to a corresponding one of a plurality of tower members 1584 (e.g., towers). The tower members 1584 can provide an access channel 1585 for accessing the fixation members 1572, and can be rotated/pivoted to vary the orientation/position of the tulips 1528 relative to the screws 1573. For example, a driver can be inserted through the access channels 1585 and used to drive (e.g., rotate, screw) the screws into vertebral bone. Then, referring to FIGS. 15A-15C, after removing the driver, the tower members 1584 can be rotated to vary the orientation of the tulips 1578 and the corresponding openings 1582 to allow the spanning member 1574 to be slid therethrough to secure the fixation members 1572 together. Finally, the same or a different driver can be inserted through the access channels 1585 to tighten the set screws 1580 to secure the fixation members 1572 to the spanning member 1574 and lock the orientation of the tulips 1578 relative to the screws 1573. The tower members 1584 can be decoupled from the tulips 1578 at the end of a procedure.

FIGS. 15D and 15E are side views of a single one of the fixation members 1572 and a single one of the tower members 1584 of FIG. 15C secured to a vertebra 1502 of a spine 1501 in accordance with embodiments of the present technology. Referring to FIG. 15D, the tower member 1584 is positioned in a first position in which the tulip 1578 and the access channel 1585 are generally in line with the screw 1583 (FIGS. 15A-15C) of the fixation member 1572. In the first position, the screw 1583 can be driven (e.g., rotated via a driver inserted through the access channel 1585) into the vertebra 1502. Referring to FIG. 15E, the tower member 1584 can be rotated to rotate the tulip 1578 to a second position relative to the screw 1583 to generally align the tower member 1584 and the access channel 1585 with the cannulation 1576 (FIGS. 15A and 15B). Referring to FIGS. 15A-15E, in the second position (FIG. 15E), a trocar can be inserted through the access channel 1585 and the cannulation 1576 to access a diseased disc 1504 of the spine 1501. In some embodiments, the set screw 1580 can be rotated to lock the tulip 1578 in the second position to resist movement of the tower member 1584 during a spinal surgical procedure to implant an intervertebral device within the space of the disc 1504 (e.g., as described in detail above with reference to FIGS. 1A-2). For example, once locked, the tower member 1584 can resist movement from forces exerted by tissue and/or skin of the patient. Accordingly, the tower member 1584 can be rotated to an aligned position with the off-axis cannulation 1576 and locked in the aligned position. After implanting the intervertebral device, the tulip 1578 can be unlocked from the screw 1573 (e.g., via rotation of the set screw 1580) and the tulip 1578 can again be rotated to facilitate insertion of the spanning member 1574 (FIG. 15C). Finally, the tower member 1584 can be decoupled (released) from the fixation member 1572.

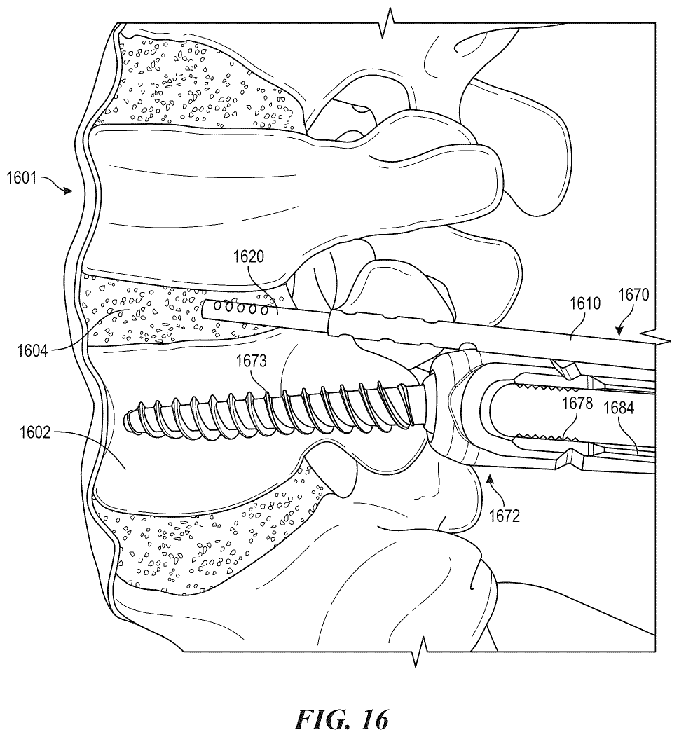

FIG. 16 is a side view of a fixation and access assembly 1670 in accordance with embodiments of the present technology. In the illustrated embodiment, the fixation and access assembly 1670 includes a fixation member 1672 coupled to a trocar 1610. The fixation member 1672 can include some features that are at least generally similar in structure and function, or identical in structure and function, to the corresponding features of the fixation member 1572 described in detail above with reference to FIGS. 15A-15E. For example, in the illustrated embodiment the fixation member 1672 includes a tulip 1678 rotatably coupled to a screw 1673. The tulip 1678 can be locked in orientation/position relative to the screw 1673 via rotation of a set screw (not shown) or other locking mechanism. The tulip 1678 can further be releasably coupled to a tower member 1684 for manipulating the orientation of the tulip 1678 relative to the screw 1673. The fixation member 1672 can be secured to (e.g., screwed into) a vertebra 1602 adjacent a diseased disc 1604 and used to provide posterior fixation.

In the illustrated embodiment, the trocar 1610 is coupled to the tulip 1678 (e.g., to a side portion of the tulip 1678). The trocar 1610 can be integral with (e.g., built into) the tulip 1678 or can be releasably coupled to the tulip 1678. In other embodiments, the trocar 1610 can be inserted through a channel or cannulation in the tulip 1678. The tulip 1678 can be manipulated by the tower member 1684 to position the trocar 1610 relative to the vertebra 1602, and/or the trocar 1610 can be manipulated to change the orientation of the tulip 1678. The trocar 1610 can be used to access the disc 1604 when the fixation member 1672 is secured to the vertebra 1602. For example, one or more instruments 1620 (e.g., discectomy devices, balloon devices, intervertebral devices, closure devices, tensioning devices, and/or the like described herein) can be inserted through the trocar 1610 to facilitate deployment of an intervertebral device in place of or in conjunction with the disc 1604.

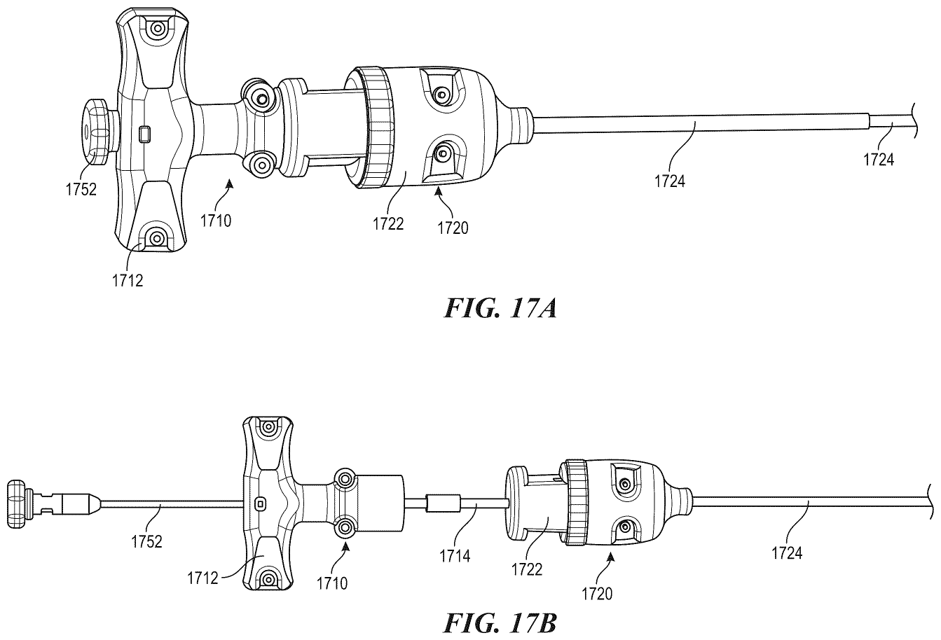

FIGS. 17A and 17B are side views of a trocar access system including an access trocar 1720 (e.g., a first trocar, an outer catheter, an access sheath, and/or the like) and a steerable trocar 1710 in accordance with embodiments of the present technology. The trocar access system is in a first (e.g., mated) position in FIG. 17A and a second (e.g., decoupled) position in FIG. 17B. Referring to FIGS. 17A and 17B, in the illustrated embodiment the access trocar 1720 includes a first handle 1722 coupled to a hollow first cannula 1724, and the steerable trocar 1710 includes a second handle 1712 coupled to a hollow second cannula 1714. Referring to FIG. 17A, the second cannula 1714 is configured to be inserted through the first cannula 1724 and at least partially out of the first cannula 1724. A distal portion of the second cannula 1714 can be configured to deflect or be actively steered (e.g., via the second handle 1712) within a disc space, such as described in detail above with reference to FIGS. 12B and 12C. In some embodiments, the second handle 1712 can mate with/couple to the first handle 1722 in the first position shown in FIG. 17A. Referring to FIGS. 17A and 17B, the second cannula 1714 can receive one or more instruments, such as an intervertebral device inserted 1752 during a spinal surgical procedure and guide the instruments into the disc space of a spine of a patient.

### III. SELECTED EMBODIMENTS OF DISCECTOMY DEVICES, AND ASSOCIATED SYSTEMS AND METHODS

FIGS. 18A-29D illustrate embodiments of discectomy devices that can be inserted through a trocar and used to remove some or all of a diseased disc, such as described in detail above with reference to FIGS. 1C and 1D and blocks 282 and 283 of the method 280 of FIG. 2. Accordingly, the embodiments described with reference to FIGS. 18A-29D can be utilized in the workflow of the spinal surgical procedures described in detail with reference to FIGS. 1A-2, FIGS. 3A-5, and/or elsewhere herein.

FIG. 18A is a side view of a discectomy device 1820 in accordance with embodiments of the present technology. In the illustrated embodiment, the discectomy device 1820 includes a handle 1822 coupled to an elongate shaft 1824. The elongate shaft 1824 includes a disc-cutting element 1826 (e.g., a disc-shaving element) at a distal end portion thereof. The disc-cutting element 1826 can include a plurality of (e.g., two) expandable members 1825 configured (e.g., shaped, sized) to expand radially outward relative to the elongate shaft 1824 for mechanically engaging and disrupting disc material. The expandable members 1825 can be formed from spring steel, nitinol, and/or another material such that the expandable members 1825 expand radially outward when deployed from an introducer (e.g., any of the trocars and/or introducers described in detail above). During a spinal surgical procedure, the disc-cutting element 1826 can be translated proximally and distally relative to the introducer and/or rotated relative to the disc space and/or the introducer to mechanically engage and disrupt the disc material. In some embodiments, the expandable members 1825 are sharpened to provide a cutting mechanism for cutting the disc material. After engaging the disc material, the expandable members 1825 can be compressed when drawn back into the introducer.

FIG. 18B is a side of the discectomy device 1800 of FIG. 18A in accordance with additional embodiments of the present technology. In the illustrated embodiment, the expandable members 1825 are shaped to have a more radially-expanded (e.g., semicircular) profile when expanded.

FIG. 18C is a side of the discectomy device 1800 of FIG. 18A in accordance with additional embodiments of the present technology. In the illustrated embodiment, the expandable members 1825 are shaped to extend distally and curve out radially and/or back proximally when expanded.

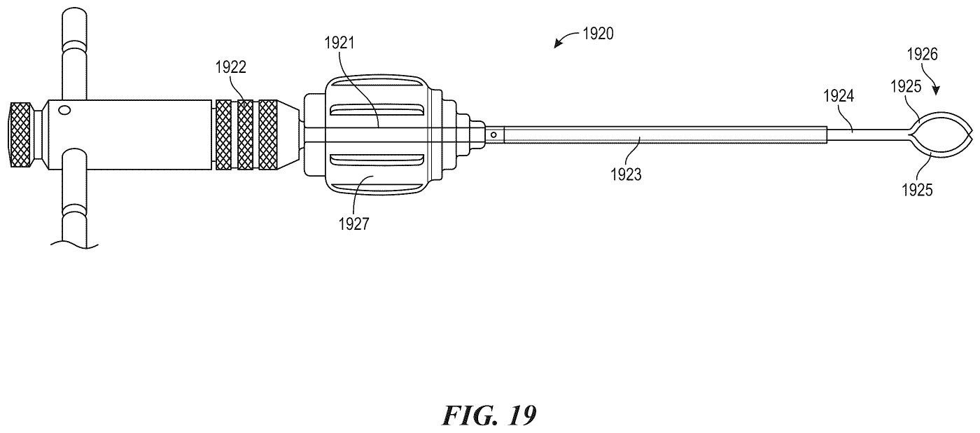

FIG. 19 is a side view of a discectomy device 1920 in accordance with embodiments of the present technology. In the illustrated embodiment, the discectomy device 1920 includes the elongate shaft 1824 and the disc-cutting element 1826 of FIG. 18B. The elongate shaft 1824 can further be coupled to a handle 1922 and include a proximal threaded portion 1921. The discectomy device 1920 can further include an outer shaft 1923 positioned at least partially over the elongate shaft 1824 and an actuator 1927 positioned over the threaded portion 1921. The actuator 1927 can include a threaded inner channel that mates with the threaded portion 1921 of the elongate shaft 1824. The actuator 1927 can be actuated (e.g., rotated) to retract the elongate shaft 1824 and the disc-cutting element 1826 into the outer shaft 1923 to collapse/compress the expandable members 1825 therein and/or to advance the outer shaft 1923 over the disc-cutting element 1826 to collapse the expandable members 1825 therein. Alternatively or additionally, the actuator 1927 can be actuated to retract the disc-cutting element 1826 relative to an introducer (e.g., a curved trocar) through which the discectomy device 1920 is inserted to collapse the disc-cutting element 1826 within the introducer. In some aspects of the present technology, the actuator 1927 provides a mechanical advantage that makes it easier to collapse the disc-cutting element 1826 when, for example, the expandable members 1825 are relatively rigid in the expanded configuration and therefore require a significant force to collapse.

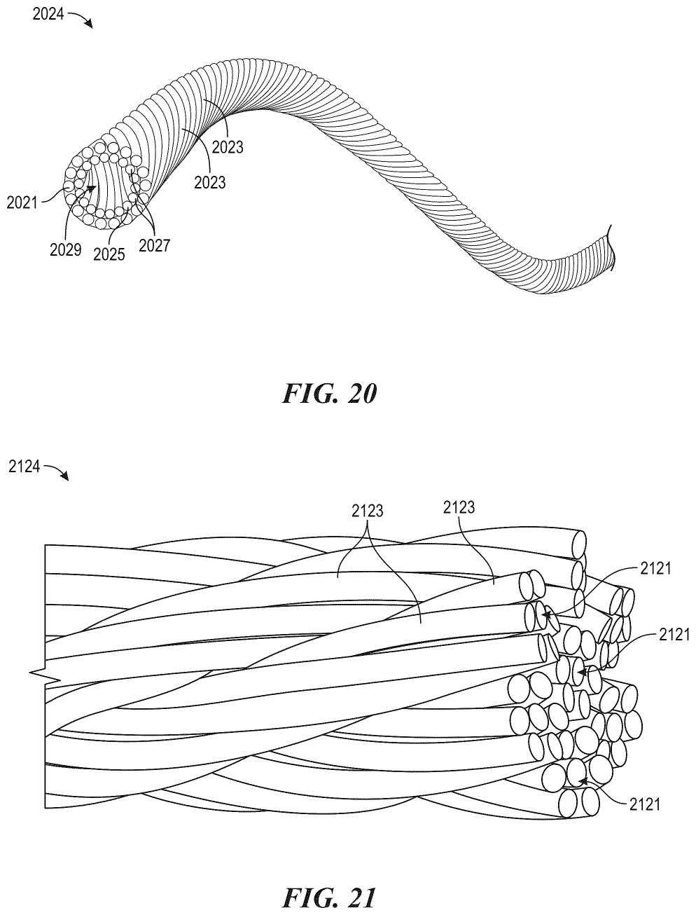

FIG. 20 is a perspective view of an elongate member 2024 of a discectomy device in accordance with embodiments of the present technology. In the illustrated embodiment, the elongate member 2024 is a helical hollow strand tube comprising a first (e.g., outer) layer 2021 comprising one or more helically wound filaments or strands 2023 and a second (e.g., inner) layer 2025 comprising one or more helically wound filaments or strands 2027. The second layer 2025 can define an inner channel 2029. The strands 2023 and 2027 can comprise stainless steel, cobalt-chrome, titanium, nitinol, tungsten, composite materials, other metal materials, and/or the like. In some aspects of the present technology, the construction of the elongate member 2024 allows the elongate member 2024 to transmit torque and pushing forces to a disc-cutting element coupled thereto-even when the elongate member 2024 traverses as a curved path, such as when it is introduced through a curved trocar. More specifically, the elongate member 2024 can have high whip free characteristics and high resistance to kinks that allows for the transmission of torque and pushing forces along a curved trajectory. In some embodiments, the elongate member 2024 includes only one of the first layer 2021 or the second layer 2025, or includes additional layers of helically wound filaments or strands.

FIG. 21 is a perspective view of an elongate member 2124 of a discectomy device in accordance with embodiments of the present technology. In the illustrated embodiment, the elongate member 2124 is a cable comprising a plurality of groups 2121 of individual filaments of strands 2123 that are braided/wound together. The groups 2121 are further braided/wound together to form the elongate member 2124. In the illustrated embodiment, each of the groups 2121 comprises seven of the strands 2123, and the elongate member 2124 includes seven of the groups 2121. In other embodiments, the groups 2121 can include more or fewer of the strands 2123, and/or the elongate member 2124 can include more or fewer of the groups 2121. The strands 2123 can comprise stainless steel, cobalt-chrome, titanium, nitinol, tungsten, composite materials, other metal materials, and/or the like. In some aspects of the present technology, the construction of the elongate member 2124 allows the elongate member 2124 to transmit torque and pushing forces to a disc-cutting element coupled thereto-even when the elongate member 2124 traverses as a curved path, such as when it is introduced through a curved trocar.

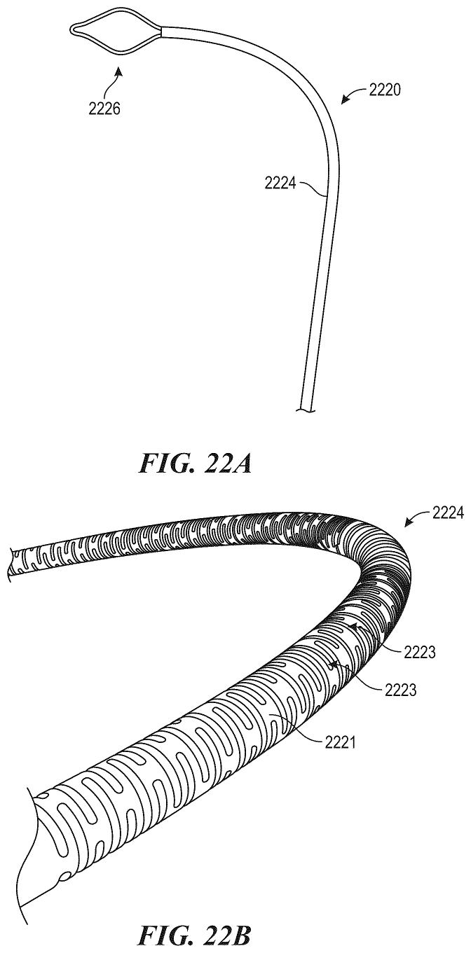

FIG. 22A is a side view of a discectomy device 2220 in accordance with embodiments of the present technology. In the illustrated embodiment, the discectomy device 2220 includes an elongate member 2224 coupled to a disc-cutting element 2226. The elongate member 2224 is shown in a curved position in FIG. 22A, such as a position in which the elongate member 2224 can navigate a curved introducer. FIG. 22B is an enlarged perspective view of a portion of the elongate member 2224 in accordance with embodiments of the present technology. In the illustrated embodiment, the elongate member 2224 comprises a flexible tube 2221 having openings or grooves 2223 formed therein. The grooves 2223 can be laser cut into the flexible tube 2221. In some embodiments, the elongate member is a flexible laser-cut hypotube.

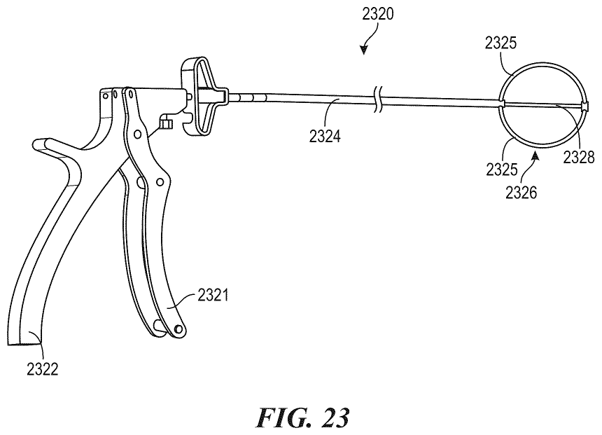

FIG. 23 is a perspective side view of a discectomy device 2320 in accordance with embodiments of the present technology. In the illustrated embodiment, the discectomy device 2320 includes (i) a handle 2322, (ii) an actuator 2321 (e.g., a trigger) operably coupled to the handle 2322, (iii) an outer elongate member 2324 fixedly coupled to the handle 2322, (iv) an inner elongate member 2328 extending through the outer elongate member 2324 and coupled to the actuator 2321, and (v) a disc-cutting element 2326 having a distal end portion coupled to the inner elongate member 2328 and a proximal end portion coupled to the outer elongate member 2324. The discectomy device 2320 can be inserted through an introducer to a disc space of a spine of a patient.

The disc-cutting element 2326 can include a plurality of (e.g., two) expandable members 2325 configured to expand radially outward relative for mechanically engaging and disrupting material of a disc within the disc space. More particularly, a user (e.g., a surgeon) can grip the handle 2322 and actuate (e.g., squeeze) the actuator 2321 to retract the inner elongate member 2328 relative to the outer elongate member 2324 to longitudinally compress the disc-cutting element 2326 and cause the expandable members 2325 to expand radially outward. In the illustrated embodiment, actuating the actuator 2321 radially expands both the expandable members 2325 (“bilateral expansion”). In other embodiments, actuating the actuator 2321 can radially expand only one of the expandable members 2325 (“unilateral expansion”). Likewise, the disc-cutting element 2326 can include only one, or more than two of the expandable members 2325.

In some aspects of the present technology, forcibly expanding the disc-cutting element 2326 within the disc space can allow for a greater area for removal of disc material within the disc space. For example, the introducer can have an inner lumen with a diameter of between about 1.5-4.5 millimeters, whereas the disc space can have a dimension of up to about 14 millimeters. Therefore, expanding the disc-cutting element 2326 to a dimension greater than the introducer can allow the disc-cutting element 2326 to better match the dimensions of the disc space to facilitate robust removal of the disc material. In some embodiments, the disc-cutting element 2326 is expandable to a dimension greater than a dimension (e.g., height) of the disc space. This can allow the disc-cutting element 2326 to flex against the adjacent vertebrae and scrape disc material therefrom.

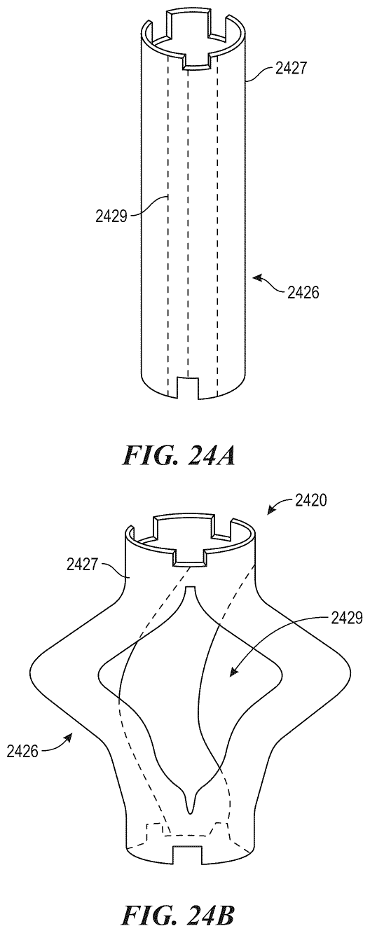

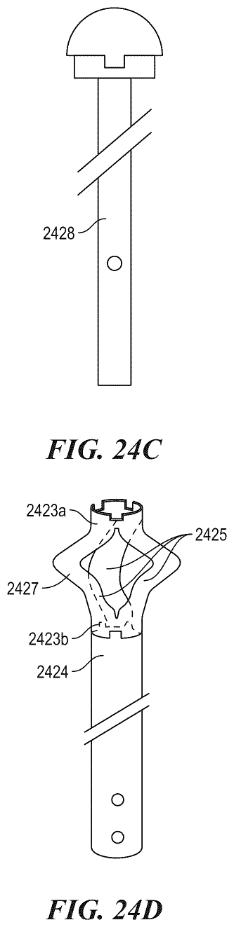

FIGS. 24A-24D are side views of various portions of a discectomy device 2420 in accordance with embodiments of the present technology. Referring to FIGS. 24A and 24B, the discectomy device 2420 can include a disc-cutting element 2426 comprising a hypotube 2427 having multiple (e.g., three) laser-cut slits 2429 extending longitudinally along the hypotube 2427 and spaced circumferentially about the hypotube 2427. The disc-cutting element 2426 is in a compressed position in FIG. 24A and an expanded position in FIG. 24B. Referring to FIGS. 24C and 24D, the discectomy device 2420 can further include an inner elongate member 2428 coupled to a distal end portion 2423a of the hypotube 2427 and an outer elongate member 2424 coupled to a proximal end portion 2423b of the hypotube 2427. Accordingly, the inner elongate member 2428 can be drawn proximally relative to the outer elongate member 2424 to longitudinally compress the hypotube 2427 via, for example, a handle (not shown; e.g., the handle 2322 of FIG. 23). Referring to FIGS. 24A-24D, longitudinal compression of the hypotube 2427 causes the hypotube 2427 to split along the slits 2429 and expand radially outward to form cutting members 2425 (FIG. 24D). The cutting members 2425 can engage and disrupt disc material in the expanded position.

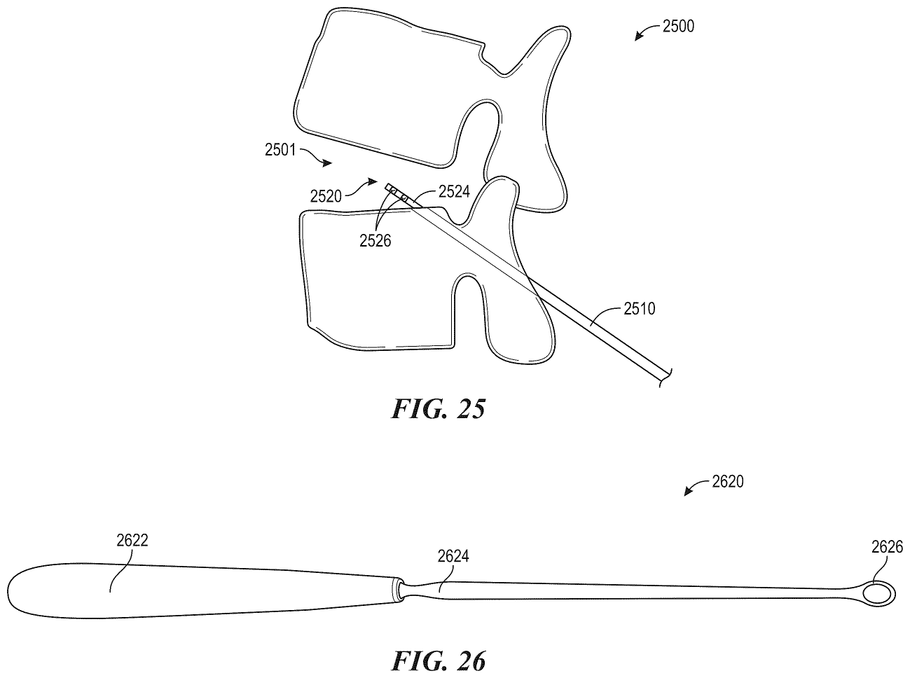

FIG. 25 is a side view of a discectomy device 2520 inserted through an introducer 2510 for accessing a spine 2500 of a patient in accordance with embodiments of the present technology. In the illustrated embodiment, the discectomy device 2520 includes an elongate member 2524 having one or more energy delivery elements 2526 positioned thereon. The energy delivery elements 2526 can be configured to deliver ablation energy, heat, and/or the like into a disc space 2501 to remove disc material therein. In some embodiments, the energy delivery elements 2526 are electrodes.

FIG. 26 is a side view of a discectomy device 2620 in accordance with embodiments of the present technology. In the illustrated embodiment, the discectomy device 2620 includes a grip or handle 2622 coupled to an elongate shaft 2624, and a disc-cutting element 2626 at a distal end portion of the elongate shaft 2624. The disc-cutting element 2626 can be a rigid ring for scraping disc material. Accordingly, the discectomy device 2620 can be similar to a curette device.

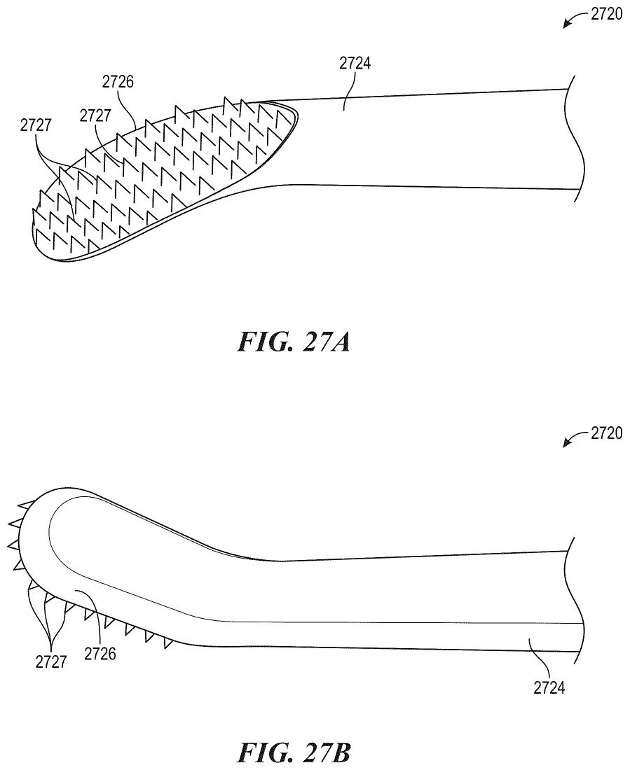

FIGS. 27A and 27B are enlarged side views of a distal portion of a discectomy device 2720 in accordance with embodiments of the present technology. Referring to FIGS. 27A and 27B, in the illustrated embodiment the discectomy device 2720 includes an elongate shaft 2724 having a disc-cutting element 2726 at a distal end portion thereof. The disc-cutting element 2726 can be angled relative to the elongate shaft 2724 and have a textured scraping surface 2727 (FIG. 27A). Accordingly, the discectomy device 2720 can be similar to a rasp device.

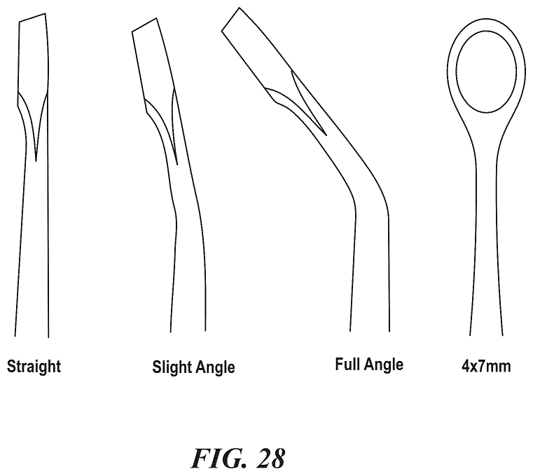

FIG. 28 includes multiple side views of distal portions of curette-like or rasp-like discectomy devices 2800 in accordance with embodiments of the present technology.

Referring to FIGS. 26-28, in some embodiments a rasp-like or curette-like disc-cutting element can be hingedly, pivotably, and/or otherwise movably coupled to an elongate shaft of a discectomy device such that the disc-cutting element can be pivoted within the disc space to access different portions of the disc space. In some embodiments, movement (e.g., pivoting) of the disc-cutting element can be controlled via a handle or other user control.

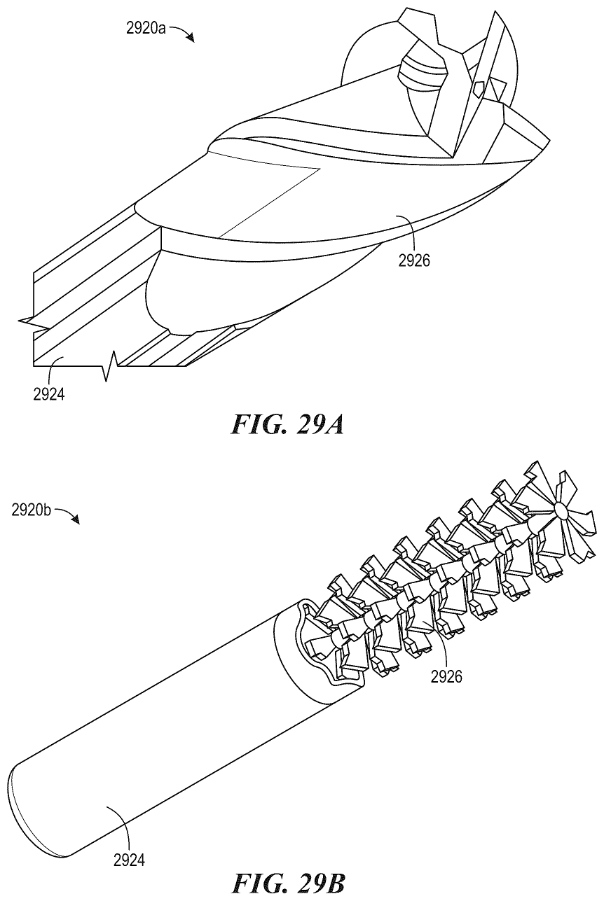

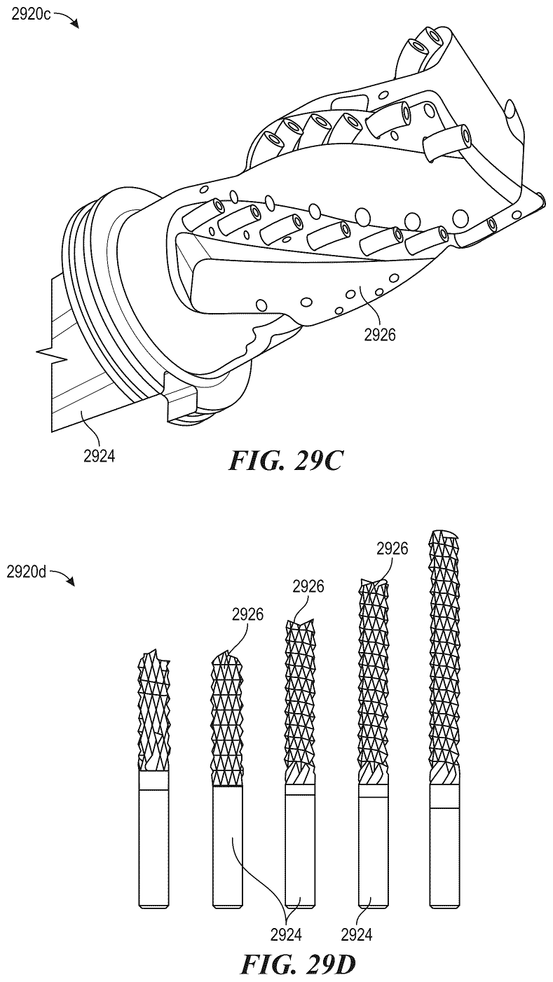

FIGS. 29A-29D are perspective side views of a distal portion of discectomy devices 2920a-2920d, respectively, in accordance with embodiments of the present technology. Referring to FIGS. 29A-29D together, each of discectomy devices 2920a-d includes a cutting portion 2926 coupled to an elongate member 2924 and is configured to be rotated (e.g., at a high rotation-per-minute (RPM)) such that the cutting portion 2926 engages and disrupts disc material similar to an end mill or burr. More specifically, the cutting portions 2926 can comprise end mills having grooves, teeth, flutes, channels, and/or the like that cut through the disc material when rotated and that can capture/trap the disc material therein for removal from the patient.

The discectomy devices 2920a-2920d devices can be inserted through a curved trocar, as described in detail above, and the curved trocar can be rotated and/or translated to sweep and steer the discectomy devices 2920a-d through the disc space. Accordingly, in some embodiments a portion of the elongate members 2934 is flexible to permit the discectomy devices 2920a-2920d to traverse through the curved introducer trocar. In other embodiments, the cutting portion 2926 is rigid such that is unable to traverse a curved trocar. In such embodiments, the curved trocar can be retracted into the straight trocar, and the discectomy devices 2920a-d can be inserted through the curved trocar and the straight trocar into the disc scape. The curved trocar can then be advanced to curve/steer the cutting portion 2926. Such embodiments can utilize any of the torque cables, laser cut hypotubes, and/or the like described herein (e.g., with reference to FIGS. 20-22B) such that the discectomy devices 2920a-d comprise a flexible portion that is conducive to steering.

In some embodiments, the cutting portions 2926 can be configured to expand when deployed from an introducer trocar and collapse when recaptured within the introducer trocar.

### IV. SELECTED EMBODIMENTS OF BALLOON DEVICES, AND ASSOCIATED SYSTEMS AND METHODS

FIGS. 30-39 illustrate embodiments of balloon devices that can be inserted through a trocar and expanded within a disc space to disrupt disc material within the disc space and/or to enlarge the disc space (e.g., by lifting a vertebra adjacent to the disc space), such as described in detail above with reference to FIGS. 1E-1H and blocks 284 and 285 of the method 280 of FIG. 2 and FIGS. 3E-3K and blocks 584, 585, and 587 of the method 580 of FIG. 5. Accordingly, the embodiments described with reference to FIGS. 30-39 can be utilized in the workflow of the spinal surgical procedures described in detail with reference to FIGS. 1A-2, FIGS. 3A-5, and/or elsewhere herein.

FIG. 30 is a side view of a distal portion of a balloon device 3031 positioned through an introducer or trocar 3010 in accordance with embodiments of the present technology. The balloon device 3031 includes an outer elongate member 3034, an inner elongate member 3032 extending at least partially through the outer elongate member 3034, and a balloon 3030 having a proximal portion coupled to the outer elongate member 3034 and a distal portion coupled to the inner elongate member 3032. In the illustrated embodiment, a distal portion of the inner elongate member 3032 that extends from the outer elongate member 3034 is curved. The inner elongate member 3032 can be heat set or otherwise configured to assume the curved shape. In some embodiments, the inner elongate member 3032 can assume a generally straight shape when advanced through the trocar 3010, and the inner elongate member 3032 is configured to assume the curved shape when it extends out of the trocar 3010. In other embodiments, the inner elongate member 3032 can be omitted and the balloon 3030 can be coupled freely to the distal end portion of the outer elongate member 3034.

In some aspects of the present technology, the curved shape of the inner elongate member 3032 can facilitate placement of the balloon 3030 at a desired position within a disc space. For example, as described in detail above with reference to FIGS. 12B and 12C, the curved shape of the inner elongate member 3032 can allow the balloon 3030 to be swept and steered through the disc space via rotation and/or translation of the balloon device 3031. In some embodiments, the balloon 3030 can be steered for placement along a midline and/or along an anterior edge of the disc space before the balloon 3030 is expanded.

FIG. 31 is a side (e.g., lateral) view of a balloon 3130 of a balloon device deployed and expanded within a disc space 3101 of a spine 3100 of a patient in accordance with embodiments of the present technology. In the illustrated embodiment, the balloon 3130 includes a wall 3135 having a first (e.g., posterior) portion 3136 and a second (e.g., anterior) portion 3137. The first portion 3136 of the wall 3135 can be thicker than the second portion 3137 of the wall 3135 such that the balloon 3130 differentially expands when inflated. For example, the first portion 3136 can expand less than the second portion 3137 such that the balloon 3130 has a height H₁ along the first portion 3136 that is less than a height H₂ along the second portion 3137. In some aspects of the present technology, such a differential shape of the balloon 3130 can allow the balloon to lift an anterior portion of an upper vertebra 3102a adjacent the disc space 3101 relative to a lower vertebra 3102b adjacent the disc space 3101 more than a posterior portion of the upper vertebra 3102a to help restore a natural lordosis of the spine 3100.

In other embodiments, different portions of the wall 3135 of the balloon 3130 can have different compliances/resistance to provide for different differential lifting of the upper vertebra 3102a. For example, different portions of the balloon 3130 can have different thicknesses, can comprise different materials, and/or can comprise fibers and/or other materials that affect the compliances of the different portions of the balloon 3130. For example, embedded fibers in the balloon 3130 can restrict expansion of the balloon 3130 past a certain dimension. Similarly, a greater wall thickness for a portion of the balloon 3130 can impart a greater resistance to expansion and vice versa.

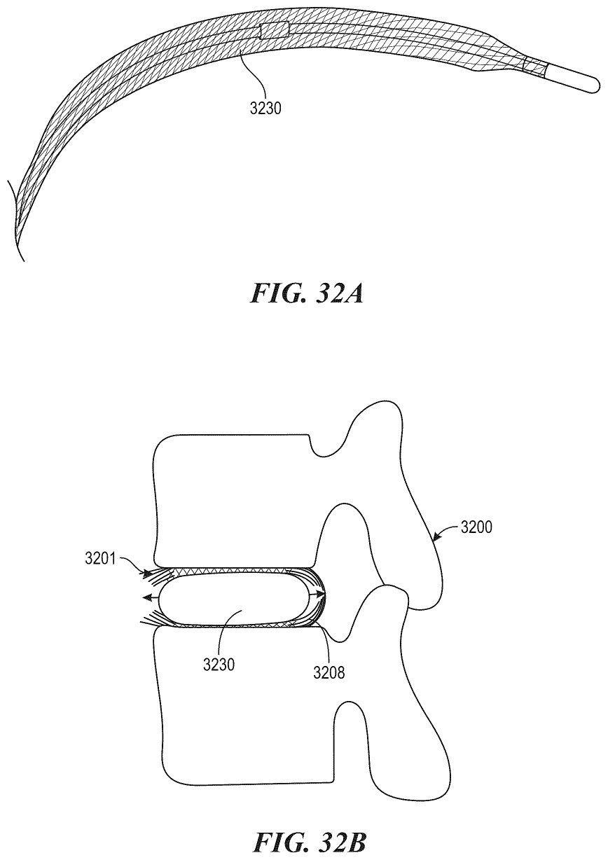

FIG. 32A is a side view of a balloon 3230 of a balloon device in accordance with embodiments of the present technology. FIG. 32B is a side (e.g., lateral) view of the balloon 3230 deployed and expanded within a disc space 3201 of a spine 3200 of a patient in accordance with embodiments of the present technology. Referring to FIGS. 32A and 32B, in the illustrated embodiment the balloon 3230 is compliant such that upon expansion in the disc space 3201, the balloon 3230 expands generally horizontally within the disc space 3201 to directly engage, disrupt, and/or break material of a disc therein, such as a ligamentous ring 3208 of the disc.

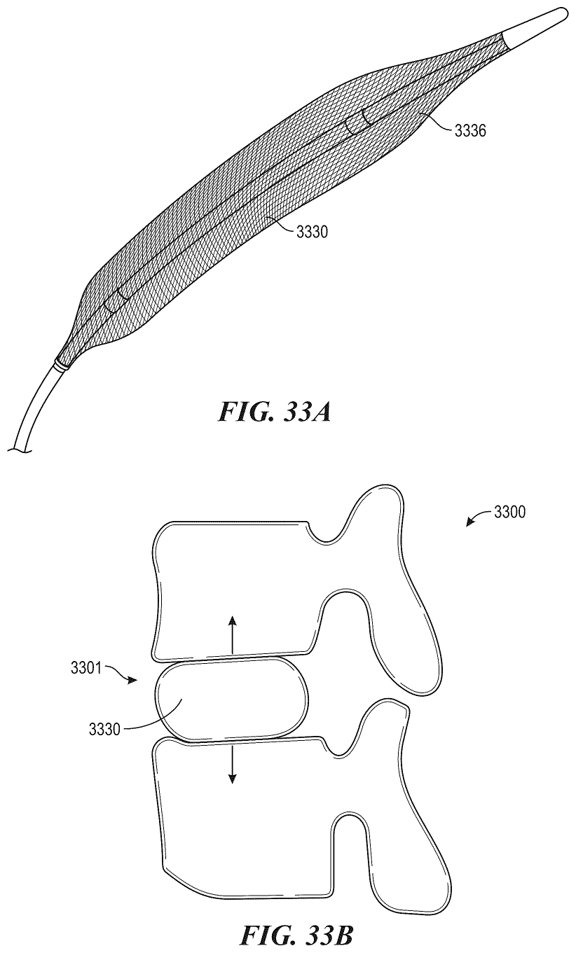

FIG. 33A is a side view of a balloon 3330 of a balloon device in accordance with embodiments of the present technology. FIG. 33B is a side (e.g., lateral) view of the balloon 3330 deployed and expanded within a disc space 3301 of a spine 3300 of a patient in accordance with embodiments of the present technology. Referring to FIGS. 33A and 33B, in the illustrated embodiment the balloon 3330 is non-compliant such that upon expansion in the disc space 3301, the balloon 3330 expands generally vertically within the disc space 3301 to force an upper vertebra 3302a of the spine 3300 adjacent the disc space 3301 away from a lower vertebra 3302b of the spine 3300 adjacent the disc space 3302 to indirectly engage, disrupt, and/or break material of a disc therein (e.g., a ligamentous ring of the disc). More specifically, the non-compliant balloon 3330 can expand vertically when it exceeds an intervertebral compliance. The balloon 3330 can be made non-compliant by incorporating a braided material 3336 into the wall of the balloon 3330.

More generally, the selected compliance and non-compliance of a balloon of the present technology can determine whether the balloon will expand in the directions of less resistance or whether it will expand into a predetermined shape. By expanding the balloon into a predetermined shape, the balloon ensures at least a minimum height will be achieved when the balloon is fully inflated. A non-compliant balloon will expand like a compliant balloon until the balloon walls are no longer in slack.

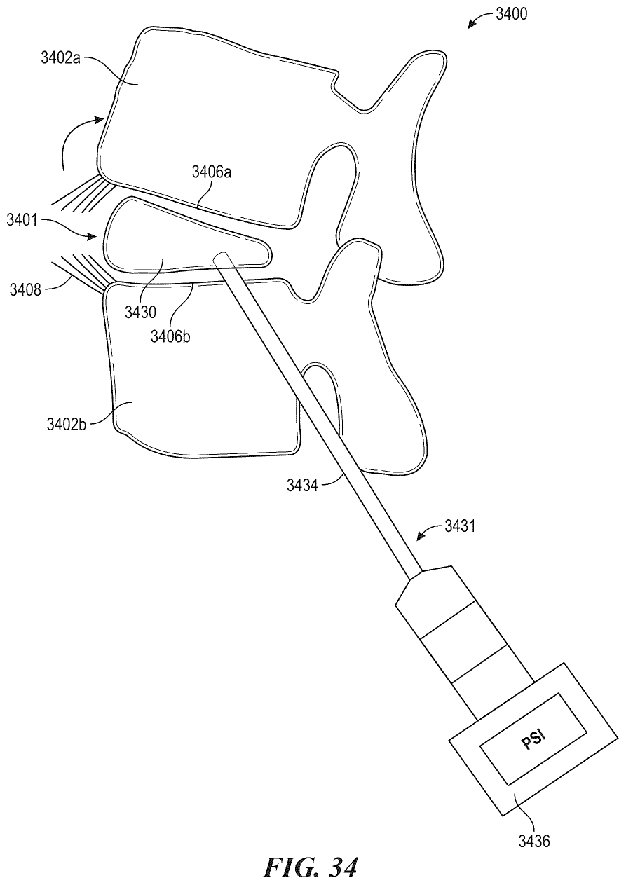

FIG. 34 is a side (e.g., lateral) view of a balloon device 3431 deployed and expanded within a disc space 3401 of a spine 3400 of a patient in accordance with embodiments of the present technology. In the illustrated embodiment, the balloon device 3431 includes a balloon 3430 operably coupled to a pressure sensing assembly 3436. In some embodiments, an inflation shaft 3434 (e.g., the outer balloon shaft 134 of FIGS. 1F and 1H) fluidly connects the balloon 3430 to the pressure sensing assembly 3436. The inflation shaft 3434 and balloon 3430 can be inserted through an introducer/trocar (not shown). The pressure sensing assembly 3436 can sense a pressure and/or volume within the balloon 3430 and provide feedback to an operator (e.g., surgeon) based on the sensed pressure and/or volume. For example, the pressure sensing assembly 3436 can use the pressure measurements to determine a degree of disruption of a disc in the disc space 3401, such as a degree of disruption of a ligamentous ring 3408 of the disc (e.g., including an anterior longitudinal ligament (ALL), a posterior longitudinal ligament (PLL), and/or a disc annular ligament). For example, the pressure in the balloon 3430 can suddenly decrease when the ligamentous ring 3408 is broken and the balloon 3430 suddenly increases in volume when no longer constrained by the ligamentous ring 3408. The pressure in the balloon 3430 can further provide an indication of the sufficiency/quality of a previous discectomy step carried out on the disc. Similarly, the pressure sensing assembly 3436 can detect a sudden decrease in pressure in the balloon 3403 to determine if the balloon 3430 has malfunctioned (e.g., ruptured). In some embodiments, pressure and/or volume can be measured by turns of a syringe handle of the pressure sensing assembly 3436, including a zero button when the balloon 3430 is just above zero atmospheres and/or zero volume when dead space in the system is taken up.

Additionally, the pressure sensing assembly 3436 can continuously monitor the pressure of the balloon 3430 during expansion of the balloon 3430 to determine a degree of degeneration of the disc, a degree of calcification of the disc, and/or the like. The pressure sensing assembly 3436 can further use the measured final expansion pressure, when the balloon 3430 is fully inflated, to predict how much force a subsequently implanted intervertebral device and/or posterior fixation assembly is likely to experience, which can be used to select an optimal fill material for the intervertebral device, an optimal volume of fill material to be injected into the intervertebral device, an optimal posterior fixation assembly, and/or the like. That is, for example, the pressure sensing assembly 3436 can determine the volume of the balloon 3430 to determine an optimal fill volume for the intervertebral device. The measured expansion force can also be used to predict or model whether endplates 3406a-b of vertebrae 3402a-b, respectively, adjacent the disc space 3401 might fracture or whether there will be subsidence. Additionally, the measured expansion force can help determine the volume and pressure of fill that enables the intervertebral device to be optimally tensioned or achieve a desirable shape for posture restoration.

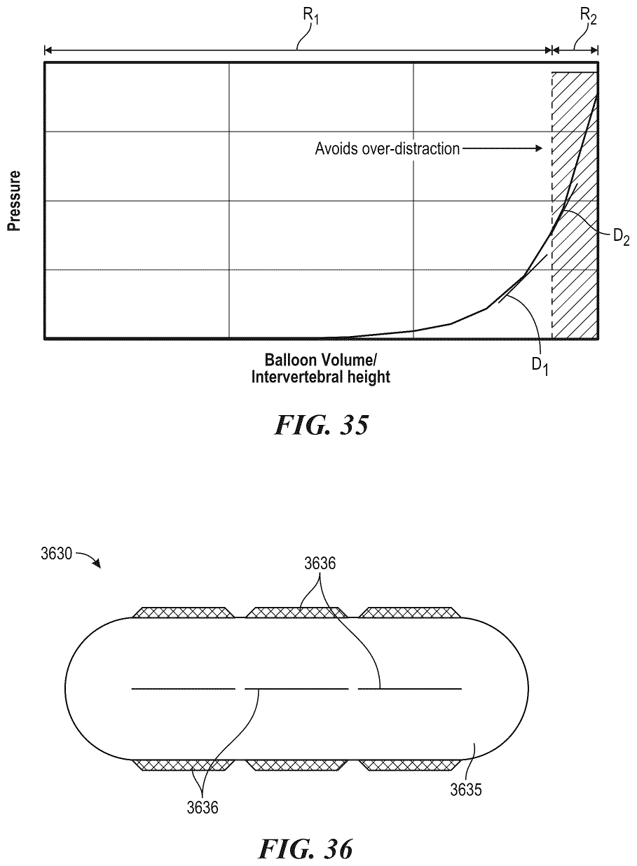

In further aspects of the present technology, the pressure sensing assembly 3436 can sense a pressure and/or volume within the balloon 3430 to provide feedback to inhibit or even prevent over distraction of the disc space 3401. FIG. 35, for example, is a graph illustrating a representative pressure-volume curve sensed by the pressure sensing assembly 3436 during expansion of the balloon 3430 in accordance with embodiments of the present technology. Referring to FIGS. 34 and 35, in some embodiments the volume of the balloon 3430 sensed by the pressure sensing assembly 3436 can directly correspond to an intervertebral height between the vertebrae 3402a-b, such as when the balloon 3430 is constrained to expand to a preselected shape. As the volume of the balloon 3430 increases, the pressure of the balloon 3430 can increase at a greater rate as the vertebrae become further distracted. In some embodiments, the pressure sensing assembly 3436 can measure/calculate a derivate of the pressure-volume curve. There can be a first region R₁ of the pressure-volume curve in which the intervertebral space 3401 is not over distracted as indicated by, for example, a derivate D₁ of the pressure-volume curve indicating that the pressure is increasing at a rate less than a predetermined threshold rate. Likewise, there can be a second region R₂ of the pressure-volume curve in which the intervertebral space 3401 beings to become or is over distracted as indicated by, for example, a derivate D₂ of the pressure-volume curve indicating that the pressure is increasing at a rate greater than the predetermined threshold rate. Accordingly, the pressure sensing assembly 3436 can stop inflation of the balloon 3430, indicate a warning (e.g., an audible or visual alarm, warning, etc.), and/or the like when the derivate of the pressure-volume curve exceeds the predetermined threshold rate to avoid over-distraction of the disc space 3401.

In some embodiments, pressure, volume, and/or height data measured with the pressure sensing assembly 3436 can be used to guide filling of an intervertebral device subsequently implanted within the disc space 3401. Specifically, the pressure, volume, and/or height data can be used to configure a desired (e.g., corresponding) pressure, volume, and/or height of the intervertebral device. For example, measuring the amount of resistance and/or force required to fill the intervertebral device can help correlate filling of the intervertebral device to a target pressure measured/determined via the pressure sensing assembly 3436. Similarly, modeling the packing configuration and/or packing density of a fill material used to fill the intervertebral device can help determine how much of the fill material to inject into the intervertebral device to achieve a target volume and/or height measured/determined via the pressure sensing assembly 3436.

FIG. 36 is a side view of a balloon 3630 of a balloon device in accordance with embodiments of the present technology. In the illustrated embodiment, the balloon 3630 includes a wall 3635 and a plurality of cutting (e.g., scoring) blades 3636 coupled to and extending outward from the wall 3635. The cutting blades 3636 can engage, cut, and/or disrupt disc material when the balloon 3630 is expanded in a disc space. The cutting blades 3636 can be positioned evenly about the wall 3635, or can be differentially distributed along certain portions off the wall 3635.

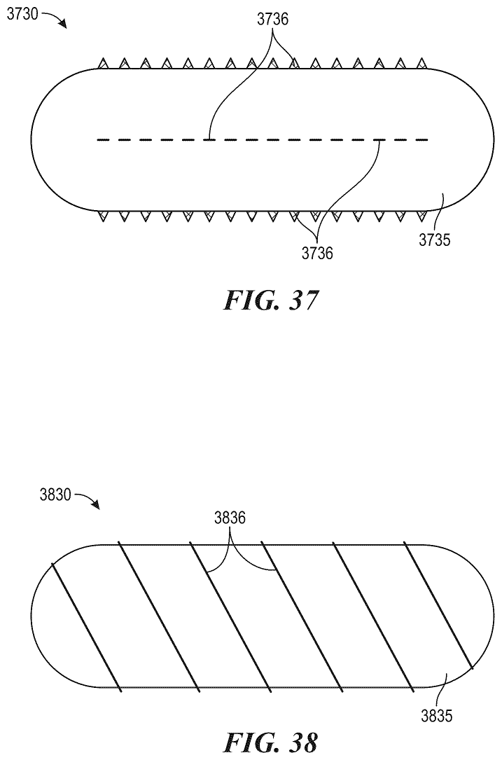

FIG. 37 is a side view of a balloon 3730 of a balloon device in accordance with embodiments of the present technology. In the illustrated embodiment, the balloon 3730 includes a wall 3735 and a plurality of cutting (e.g., scoring) teeth 3736 coupled to and extending outward from the wall 3735. The cutting teeth 3736 can engage, cut, and/or disrupt disc material when the balloon 3730 is expanded in a disc space. The cutting teeth 3736 can be positioned evenly about the wall 3735, or can be differentially distributed along certain portions off the wall 3735.

FIG. 38 is a side view of a balloon 3830 of a balloon device in accordance with embodiments of the present technology. In the illustrated embodiment, the balloon 3830 includes a wall 3835 and a helical scoring element 3836 coupled to and extending outward from the wall 3835. The helical scoring element 3836 can engage, cut, and/or disrupt disc material when the balloon 3830 is expanded in a disc space. In some embodiments, the helical scoring element 3836 can be formed from nitinol, steel, and/or another suitable material. While one helical scoring element 3836 is shown in FIG. 38, the balloon 3830 can include multiple of the helical scoring elements 3836 extending thereabout.

Referring to FIGS. 36-38, a balloon in accordance with embodiments of the present technology can include a combination of cutting elements, including one or more of the cutting blades 3636, the cutting teeth 3736, the helical scoring element 3836, and/or other cutting elements.

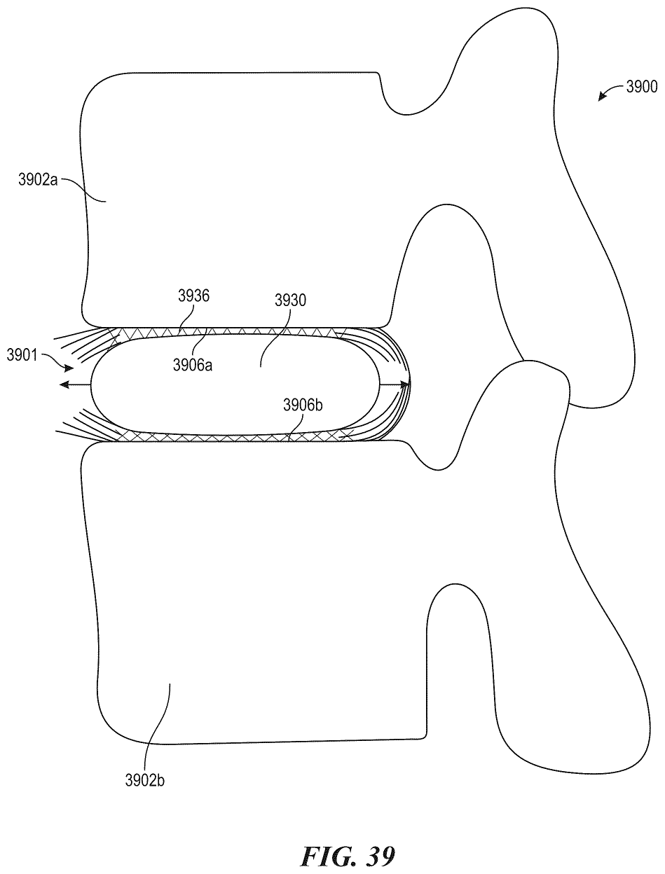

FIG. 39 is a side (e.g., lateral) view of a balloon 3930 of a balloon device deployed and expanded within a disc space 3901 of a spine 3900 of a patient in accordance with embodiments of the present technology. In the illustrated embodiment, the balloon 3930 includes a cover 3936 extending at least partially therearound. The cover 3936 can be formed from a stent-like (e.g., metal, mesh) material and can provide a layer of protection between the balloon 3930 and endplates 3906a-b of vertebrae 3902a-b, respectively, adjacent the disc space 3901. Accordingly, when the balloon 3930 is expanded in the disc space 3901, the cover 3936 can inhibit or even prevent rupture of the balloon 3930 by the endplates 3906a-b, by an introducer trocar, and/or other components within the disc space 3901. In some embodiments, the cover 3936 comprises the body of an intervertebral device to be implanted within the disc space 3901.

### V. SELECTED EMBODIMENTS OF INTERVERTEBRAL DEVICES, AND ASSOCIATED SYSTEMS AND METHODS

FIGS. 40A-48B illustrate embodiments of intervertebral devices that can be inserted through a trocar and deployed within a disc space, such as described in detail above with reference to FIGS. 1I-1K and blocks 286 and 287 of the method 280 of FIG. 2 and FIGS. 3L and 3M and blocks 589 and 590 of the method 580 of FIG. 5. Accordingly, the embodiments described with reference to FIGS. 40A-48B can be utilized in the workflow of the spinal surgical procedure described in detail with reference to FIGS. 1A-2, FIGS. 3A-5, and/or elsewhere herein.

FIG. 40A is as perspective side view of an intervertebral device 4040 in accordance with embodiments of the present technology. The intervertebral device 4040 can be a braid, mesh, and/or knit of strands or filaments 4042 that terminate and/or are joined together at a proximal hub 4041 and a distal hub 4043. The filaments 4042 can define a plurality of openings or pores 4047 therebetween. The filaments 4042 can comprise stainless steel, cobalt-chrome, titanium, nitinol, tungsten, composite materials, other metal materials, and/or the like. In the illustrated embodiment, the filaments 4042 are generally identical to one another and are woven/braided together in an over-under pattern. In other embodiments, some or all of the filaments 4042 can have different cross-sectional dimensions, cross-sectional thicknesses, cross-sectional shapes, etc., and/or the filaments 4042 can be woven together in a different pattern. FIG. 40B, for example, illustrates various patterns in which the filaments 4042 of the intervertebral device 4040 of FIG. 40A can be braided together in accordance with embodiments of the present technology. Referring to FIGS. 40A and 40B, in some aspects of the present technology different ones of the illustrated braid patterns can result in the intervertebral device 4040 having a different overall compliance and/or different sections of the intervertebral device 4040 having different compliances. Varying the compliance of the intervertebral device 4004 can vary the shape of the intervertebral device 4040 when expanded. In some embodiments, one or more of the filaments 4042 of the intervertebral device 4040 can be axial reinforcement filaments. For example, FIG. 40C illustrates various patterns in which the filaments 4042 of the intervertebral device 4040 of FIG. 40A can be woven together and/or can include axial reinforcement filaments in accordance with embodiments of the present technology. In some aspects of the present technology, the axial reinforcement filaments can reduce the compliance of the intervertebral device 4040.

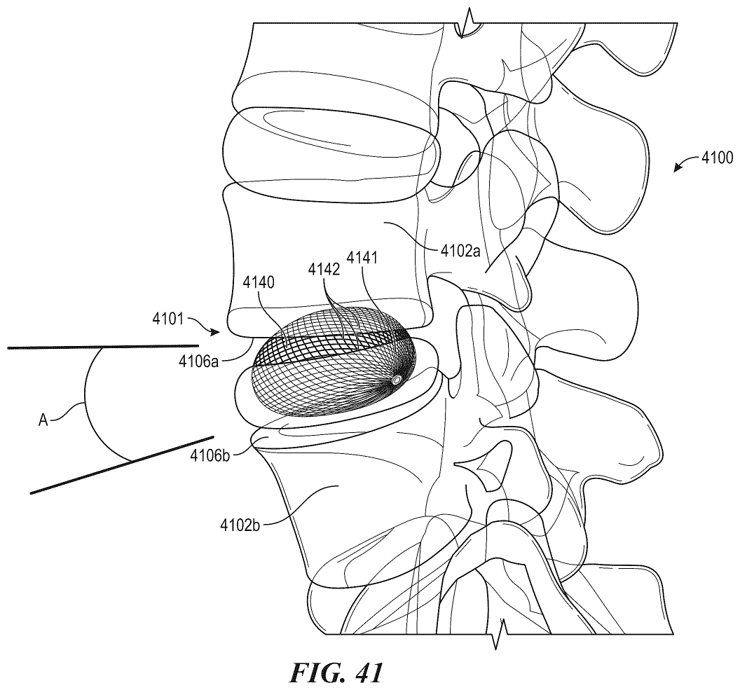

FIG. 41 is side (e.g., lateral) view of an intervertebral device 4140 deployed and expanded within a disc space 4101 of a spine 4100 of a patient in accordance with embodiments of the present technology. In the illustrated embodiment, the intervertebral device 4140 comprises a braid of filaments 4142. The filaments 4142 can be interlocked or otherwise braided together differently at a proximal portion 4141 such that intervertebral device 4140 differentially expands when, for example, expanded by a balloon or fill material inserted therein. For example, the proximal portion 4141 of the intervertebral device 4140 can expand less than the rest of the intervertebral device 4140 such that the intervertebral device 4140 conforms to the endplates 4106a-b of vertebrae 4102a-b, respectively, adjacent the disc space 4101. That is, the proximal portion 4141 can be braided in a manner that restrains expansion of the proximal portion 4141 relative to the rest of the intervertebral device 4140. In some aspects of the present technology, such differential expansion of the intervertebral device 4140 can restore a natural lordotic angle A (e.g., of about) 20° between the vertebrae 4102a-b.

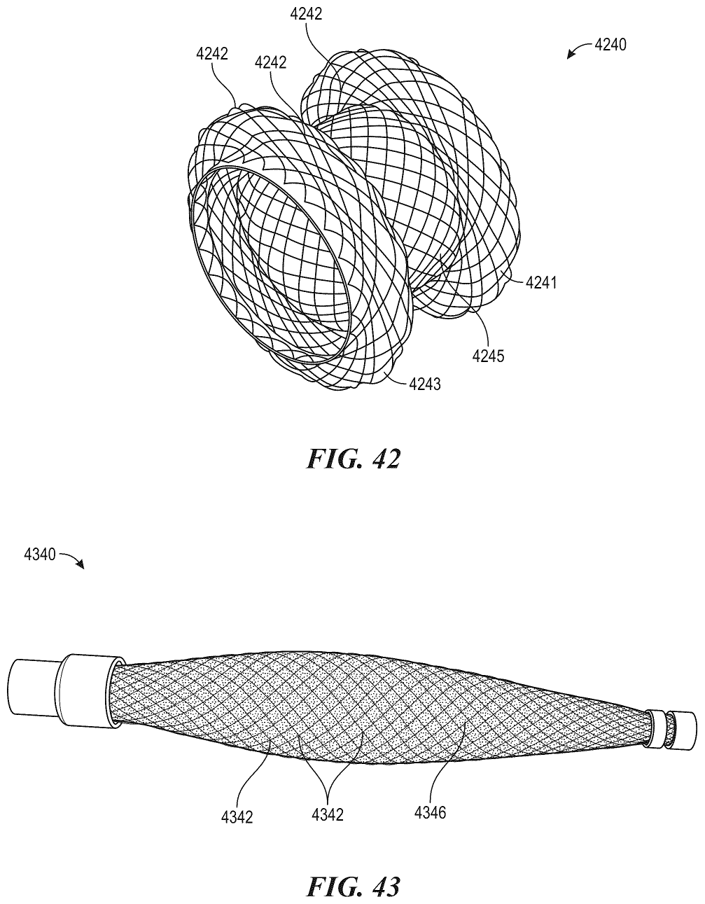

FIG. 42 is a perspective view of an intervertebral device 4240 in accordance with embodiments of the present technology. In the illustrated embodiment, the intervertebral device 4240 comprises a braid of filaments 4242 that overlap, have a different braid density, and/or have different diameters such that the intervertebral device 4240 differentially expands. In some embodiments, when expanded, the intervertebral device 4240 can have a proximal portion 4241 and distal portion 4243 that are expanded more than a middle portion 4245 (e.g., such that the intervertebral device 4240 has a dumbbell shape). In other embodiments, the overlap and/or different diameters of the filaments 4242 can cause the intervertebral device 4240 to have different shapes when expanded. For example, the middle portion 4245 and the distal portion 4243 can be more radially expanded than the proximal portion 4241 (e.g., to induce a natural lordotic angle of a spine).

FIG. 43 is as perspective side view of an intervertebral device 4340 in accordance with embodiments of the present technology. The intervertebral device 4340 can include some features that are at least generally similar in structure and function, or identical in structure and function, to the corresponding features of the intervertebral device 4040 described in detail above with reference to FIG. 40A. For example, the intervertebral device 4340 includes the braid of filaments 4042. In the illustrated embodiment, the intervertebral device 4340 further includes a barrier layer 4346 over the filaments 4042. The barrier layer 4346 can be a coating or other layer of material positioned radially inside and/or radially outside the filaments 4042. The barrier layer 4346 can substantially cover and close the pores 4047 (FIG. 40A) between the filaments 4042. Accordingly, in some aspects of the present technology the barrier layer 4346 can help contain a fill material within the intervertebral device 4340 after the intervertebral device 4340 is filled with the fill material. That is, the barrier layer 4346 can inhibit or even prevent the fill material from egressing through the pores 4047. In some embodiments, the barrier layer 4346 is dissolvable such that the barrier layer 4346 can dissolve after the intervertebral device 4340 is implanted within a patient.

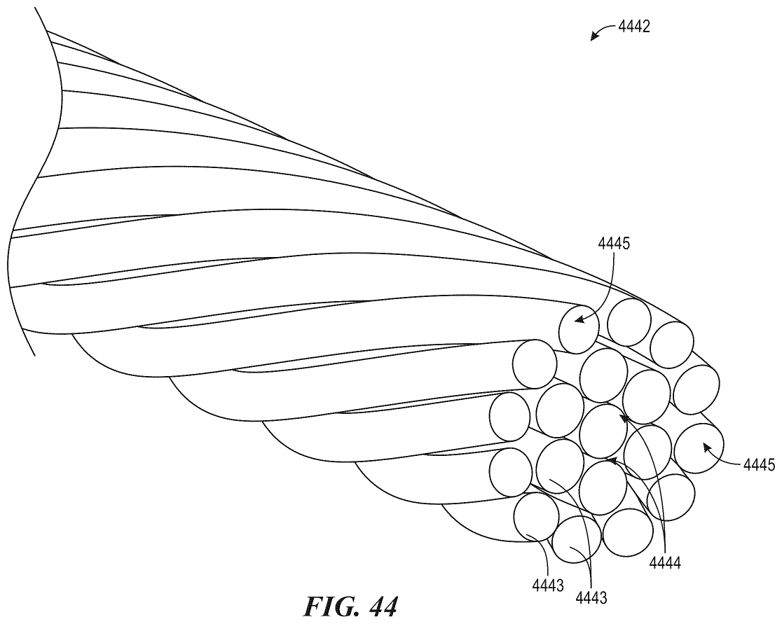

FIG. 44 is a perspective of a filament 4442 of an intervertebral device in accordance with embodiments of the present technology. In the illustrated embodiment, the filament 4442 is a cable comprising multiple wires/filars 4443 wound together. For example, the filament 4442 can include a first group 4444 of the filars 4443 that extend generally linearly/longitudinally, and a second group 4445 of the filars 4443 that extend generally helically about the first group 4444. The filars 4443 can comprise stainless steel, cobalt-chrome, titanium, nitinol, tungsten, composite materials, other metal materials, and/or the like. In some embodiments, the filars 4443 are identical and the filament 4442 comprises 19 of the filars 4443 (e.g., with seven in the first group 4444 and twelve in the second group 4445). In other embodiments, the filament 4442 can have a different number of the filars 4443 and/or the filars 4443 can be arranged differently. Referring additionally to, for example, FIG. 40A, the intervertebral device 4040 can comprise many of the filaments 4442 (e.g., in place of the filaments 4042) woven together, such as between about 200-400 of the filaments 4442, between about 250-300 of the filaments 4442, between about 280-290 of the filaments 4442, about 288 of the filaments 4442, etc. In some aspects of the present technology, forming each of the filaments 4442 from multiple ones of the filars 4443 can reduce a stiffness of the filaments 4442 and/or provide greater strength compared to, for example, a filament consisting of a single wire of comparable dimension. This can, for example, enable the intervertebral device to be inserted through a smaller trocar. In additional aspects of the present technology, forming each of the filaments 4442 from multiple ones of the filars 4443 can increase a surface area of the intervertebral device to provide for osteointegration and/or improve the conformance of the intervertebral device.

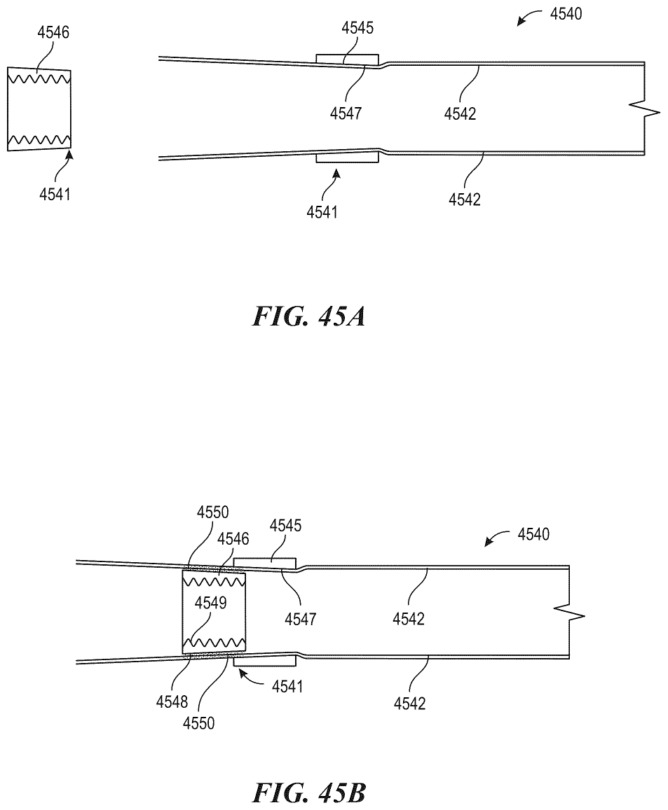

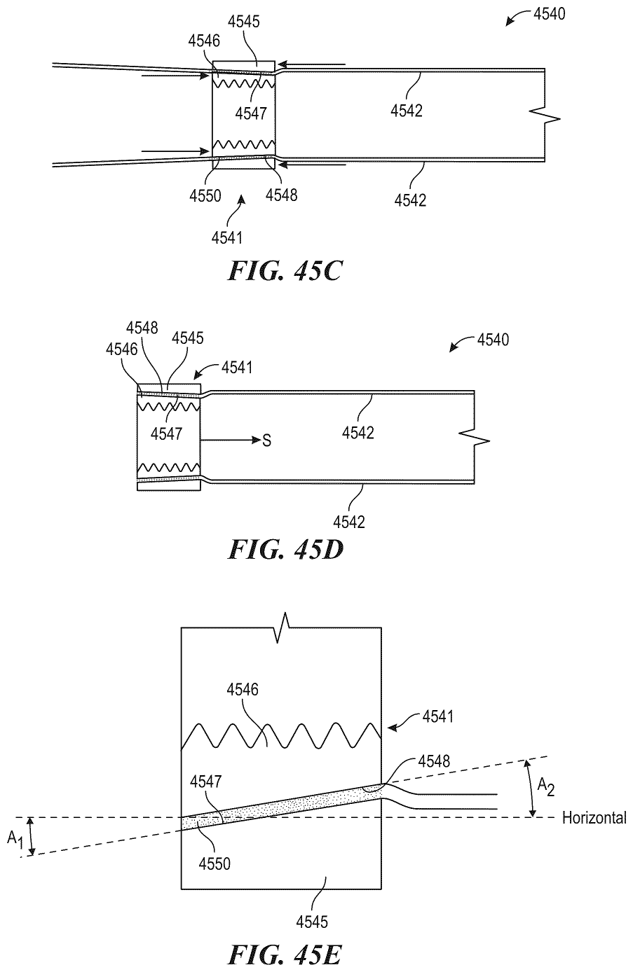

FIGS. 45A-45D are side views of different steps of a method for securing multiple filaments 4542 of an intervertebral device 4540 together to a hub 4541 in accordance with embodiments of the present technology. FIG. 45E is an enlarged view of a portion of FIG. 45D in accordance with embodiments of the present technology. Referring to FIGS. 45A-45D, the hub 4541 can be a proximal hub secured to proximal end portions of the filaments 4542 (e.g., the proximal hub 4041 shown in FIG. 40A) or a distal hub secured to distal end portions of the filaments 4542 (e.g., the distal hub 4043 shown in FIG. 40A). In the illustrated embodiment, the hub 4541 comprises an outer member 4545 and an inner member 4546. The inner and outer members 4545, 4546 can have a ring-like and/or annular shape.

Referring to FIG. 45A, in a first step the outer member 4545 can be positioned over (e.g., threaded over) the filaments 4542 at a desired position along the intervertebral device 4540. For example, the filaments 4542 can be threaded through a lumen defined by an inner surface 4547 of the outer member 4545. Referring to FIG. 45B, in a second step the inner member 4546 can be positioned inside the filaments 4542 and moved toward the outer member 4545. For example, the filaments 4542 can be threaded over an outer surface 4548 of the inner member 4546. In some embodiments, an inner surface 4549 of the inner member 4546 can be threaded to, for example, facilitate tensioning of the filaments 4542, closure of the intervertebral device 4540 after a fill material is deposited therein, etc., as described in detail herein. In the illustrated embodiment, a brazing paste 4550 is applied to the outer surface 4548 of the inner member 4546, to the filaments 4542, and/or to the inner surface 4547 of the outer member 4545.

Referring to FIG. 45C, in a third step the inner member 4546 can be positioned at least partially (e.g., fully) within the outer member 4545 such that (i) the outer surface 4548 of the inner member 4546 faces the inner surface 4547 of the outer member 4545 and (ii) the inner member 4546 and the outer member 4545 sandwich the filaments 4542 therebetween. Heat can then be applied to flow the brazing paste 4550 and secure the inner member 4546 to the outer member 4545. In some embodiments, the brazing paste 4550 can substantially fill any gaps between the filaments 4542 to create a strong connection of the filaments 4542 between the inner member 4546 and the outer member 4545. In some aspects of the present technology, the brazing process uses a heat selected not to melt the inner member 4546 or the outer member 4545. In other embodiments, the inner member 4546 can alternatively or additionally be secured to the outer member 4545 via welding (e.g., melting and subsequent cooling of a portion of the outer member 4545 to the inner member 4546), soldering, adhesives (e.g., epoxy), crimping, swaging, and/or the like.

Referring to FIG. 45D, in a fourth step the filaments 4542 can be cut to terminate (e.g., distally or proximally) at the hub 4541. Referring to FIGS. 45D and 45E, in some embodiments the inner surface 4547 of the outer member 4545 can be sloped/tapered/angled at a first angle A₁ relative to horizontal and the outer surface 4548 of the inner member 4546 can be angled at a second angle A₂ relative to horizontal. The first angle A₁ can substantially match the second angle A₂ such that inner surface 4547 and the outer surface 4548 cooperate to form a wedge shape for securing the filaments 4542 therebetween. For example, the first angle A₁ can be equal and opposite to the second angle A₂ such that outer surface 4548 and the inner surface 4547 extend parallel to one another. In some embodiments, the angle A₁ and/or the angle A₂ are less than 25 degrees, less than 20 degrees, less than 15 degrees, less than 10 degrees, between about 1-5 degrees, between about 1-3 degrees, between about 1-2 degrees, etc.

In some aspects of the present technology, the tapered inner surface 4547 and the tapered outer surface 4548 provide a mechanical advantage that inhibits the filaments 4542 from being drawn out of the hub 4541. For example, tension forces on the filaments 4542 during expansion, filling, tensioning, loading, etc., of the intervertebral device 4540 can act to draw the inner member 4546 in the direction of arrow S, which pulls the wedge shape of the inner member 4546 toward/against the wedge shape of the outer member 4545 to provide frictional, compressive, constrictive, interference, and/or similar forces that act to inhibit the filaments 4542 from moving out from between the inner member 4546 and the outer member 4545. Such forces can be in addition to the resistive forces provided by the brazed, welded, and/or soldered connection between the inner member 4546 and the outer member 4545. Additionally, the brazing paste 4550 (and/or an epoxy, melted and re-solidified material from welding, etc.) can provide a bulk that also acts to inhibit the filaments 4542 from moving out from between the inner member 4546 and the outer member 4545. Specifically, such a bulk of material would need to be significantly compressed to be pulled out from between the inner member 4546 and the outer member 4545—thereby providing a significant termination force that acts to inhibit the filaments 4542 from moving out from between the inner member 4546 and the outer member 4545. In some aspects of the present technology, a termination force provided by the hub 4541 (e.g., a force required to pull the filaments 4542 out from between the inner member 4546 and the outer member 4545) can be as great as a force of the filaments 4542 themselves. That is, for example, the filaments 4542 can break when excessive forces are applied before the filaments 4542 move out from between the inner member 4546 and the outer member 4545.

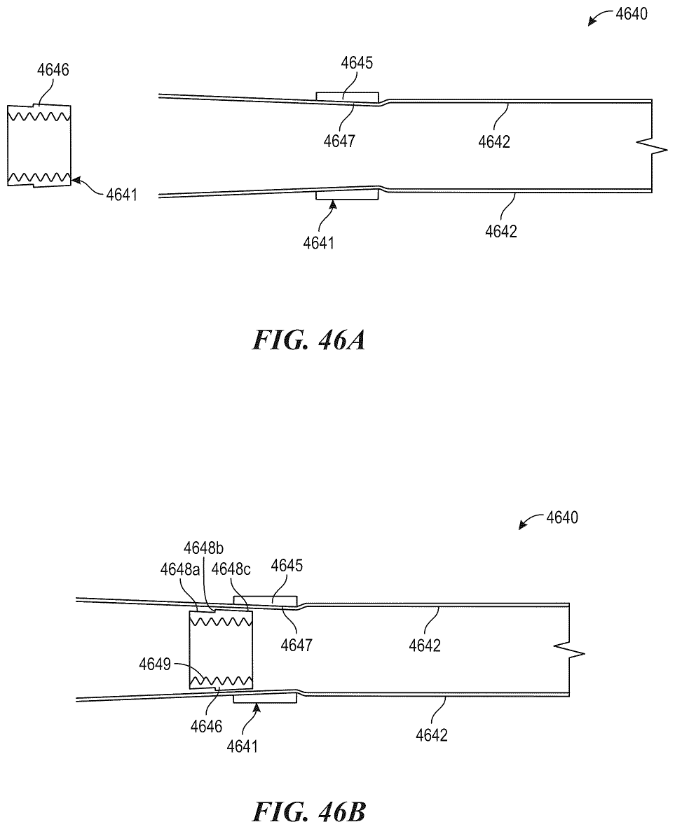

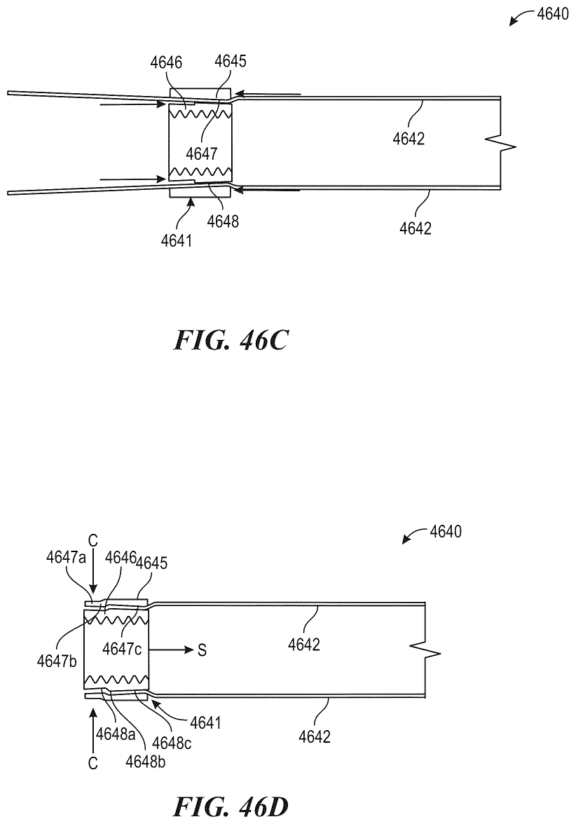

FIGS. 46A-46D are side views of different steps of a method for securing multiple filaments 4642 of an intervertebral device 4640 together to a hub 4641 in accordance with additional embodiments of the present technology. Referring to FIGS. 46A-46D, the hub 4641 can be a proximal hub secured to proximal end portions of the filaments 4642 (e.g., the proximal hub 4041 shown in FIG. 40A) or a distal hub secured to distal end portions of the filaments 4642 (e.g., the distal hub 4043 shown in FIG. 40A). The intervertebral device 4640 can include some features that are at least generally similar in structure and function, or identical in structure and function, to the corresponding features of the intervertebral device 4540 described in detail above with reference to FIGS. 45A-45D. In the illustrated embodiment, for example, the hub 4641 comprises an outer member 4645 and an inner member 4646. The inner and outer members 4645, 4646 can have a ring-like and/or annular shape.

Referring to FIG. 46A, in a first step the outer member 4645 can be positioned over (e.g., threaded over) the filaments 4642 at a desired position along the intervertebral device 4640. For example, the filaments 4642 can be threaded through a lumen defined by an inner surface 4647 of the outer member 4645. Referring to FIG. 46B, in a second step the inner member 4646 can be positioned inside the filaments 4642 and moved toward the outer member 4645. For example, the filaments 4642 can be threaded over an outer surface 4648 (including an individually identified first surface portion 4648a, stepped surface portion 4648b, and second surface portion 4648c) of the inner member 4646. In some embodiments, an inner surface 4649 of the inner member 4646 can be threaded to, for example, facilitate tensioning of the filaments 4642, closure of the intervertebral device 4640 after a fill material is deposited therein, etc., as described in detail herein. The second surface portion 4650c can have a greater maximum diameter than the first surface portion 4650a.

Referring to FIG. 46C, in a third step the inner member 4646 can be positioned at least partially (e.g., fully) within the outer member 4645 such that (i) the outer surface 4648 of the inner member 4646 faces the inner surface 4647 of the outer member 4645 and (ii) the inner member 4646 and the outer member 4645 sandwich the filaments 4642 therebetween. Referring to FIG. 46D, in a fourth step the outer member 4645 can be crimped by, for example, applying a radial inward force in the direction of arrows C such that the inner surface 4647 of the outer member 4645 at least partially conforms to the stepped shape of the outer surface 4648 of the inner member 4646. More particularly, after crimping, the inner surface 4647 of the outer member 4645 can comprise a first surface portion 4647a, a stepped surface portion 4647b, and a second surface portion 4647c similar in shape (e.g., matching) to the first surface portion 4648a, the stepped surface portion 4648b, and the second surface portion 4648c, respectively, of the inner member 4646.

In some aspects of the present technology, the stepped surface portion 4647b of the outer member 4645 and the stepped surface portion 4648b of the inner member 4646 cooperate to provide a mechanical advantage that inhibits the filaments 4642 from being drawn out of the hub 4641. For example, tension forces on the filaments 4642 during expansion, filling, tensioning, loading, etc., of the intervertebral device 4640 can act to draw the inner member 4646 in the direction of arrow S, which pulls the stepped surface portion 4648b of the inner member 4646 toward the stepped surface portion 4647b of the outer member 4645 to provide frictional, compressive, constrictive, interference, and/or similar forces that act to inhibit the filaments 4642 from moving out from between the inner member 4646 and the outer member 4645. In some embodiments, the first and second surface portions 4647a, c of the outer member 4645 and the first and second surface portions 4648a, c of the inner member 4646 are tapered/angled as described in detail above with reference to FIG. 45D to further provide a mechanical advantage that inhibits the filaments 4642 from being drawn out of the hub 4641. In some embodiments, the inner member 4646 can further be connected to the outer member 4645 via brazing, welding, soldering, and/or the like. As further shown in FIG. 46D, the filaments 4642 can be cut to terminate (e.g., distally or proximally) at the hub 4641.

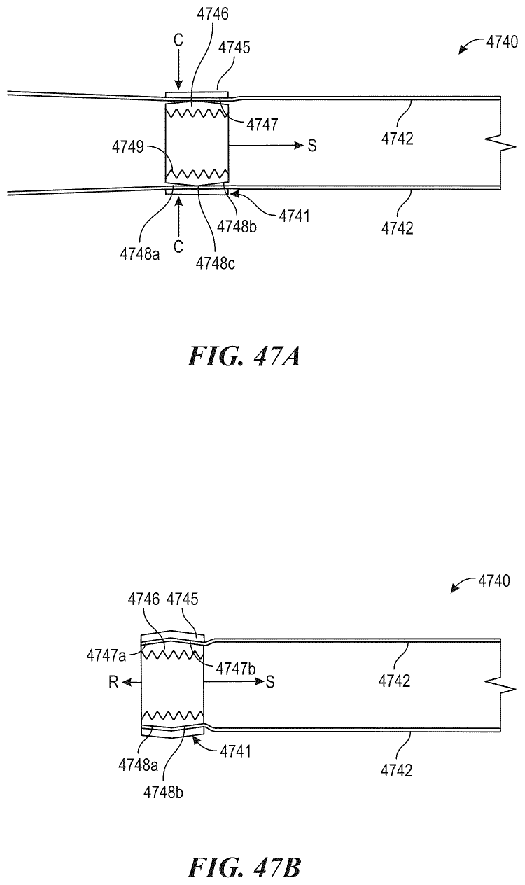

FIGS. 47A and 47B are side views of different steps of a method for securing multiple filaments 4742 of an intervertebral device 4740 together to a hub 4741 in accordance with embodiments of the present technology. Referring to FIGS. 47A and 47B, the hub 4741 can be a proximal hub secured to proximal end portions of the filaments 4742 (e.g., the proximal hub 4041 shown in FIG. 40A) or a distal hub secured to distal end portions of the filaments 4742 (e.g., the distal hub 4043 shown in FIG. 40A). The intervertebral device 4740 can include some features that are at least generally similar in structure and function, or identical in structure and function, to the corresponding features of the intervertebral device 4540 and/or the intervertebral device 4640 described in detail above with reference to FIGS. 45A-46D. In the illustrated embodiment, for example, the hub 4741 comprises an outer member 4745 and an inner member 4746. The inner and outer members 4745, 4746 can have a ring-like and/or annular shape.

Referring to FIG. 47A, in one or more first steps (i) the outer member 4745 can be positioned over (e.g., threaded over) the filaments 4742 at a desired position along the intervertebral device 4740 and (ii) the inner member 4746 can be positioned inside the filaments 4742 and at least partially (e.g., fully) within the outer member 4745 such that the outer surface 4748 of the inner member 4746 faces the inner surface 4747 of the outer member 4745, and such that the inner member 4746 and the outer member 4745 sandwich the filaments 4742 therebetween. For example, the filaments 4742 can be threaded (i) through a lumen defined by an inner surface 4747 of the outer member 4745 and (ii) over an outer surface 4748 (including an individually identified first surface portion 4748a and second surface portion 4748b) of the inner member 4746. In some embodiments, an inner surface 4749 of the inner member 4746 can be threaded to, for example, facilitate tensioning of the filaments 4742, closure of the intervertebral device 4740 after a fill material is deposited therein, etc., as described in detail herein. In the illustrated embodiment, the first surface portion 4748a is sloped/tapered such that a diameter of the inner member 4746 increases there along in the direction of arrow S, and the second surface portion 4748b is sloped/tapered such that the diameter of the inner member 4746 decreases there along in the direction of the arrow S. Accordingly, the tapered first and second surface portions 4748a-b can meet at a peak 4748c at which the inner member 4746 has a maximum diameter.

Referring to FIG. 47B, in a second step the outer member 4745 can be crimped by, for example, applying a radial inward force in the direction of arrows C (FIG. 47A) such that the inner surface 4747 of the outer member 4745 at least partially conforms to the tapered shape of the outer surface 4748 of the inner member 4746. More particularly, after crimping, the inner surface 4747 of the outer member 4745 can comprise a first surface portion 4747a and a second surface portion 4747b similar in shape (e.g., matching) to the first surface portion 4748a and the second surface portion 4748b, respectively, of the inner member 4746.

In some aspects of the present technology, the tapered first and second surface portions 4747a-b of the outer member 4745 cooperate with the tapered first and second surface portions 4748a-b, respectively, of the inner member 4746 to provide a mechanical advantage that inhibits the filaments 4742 from being drawn out of the hub 4741. For example, tension forces on the filaments 4742 during expansion, filling, tensioning, loading, etc., of the intervertebral device 4740 can act to draw the inner member 4746 in the direction of the arrow S, which pulls the second surface portion 4748b of the inner member 4746 toward the second surface portion 4747b of the outer member 4745 to provide frictional, compressive, constrictive, interference, and/or similar forces that act to inhibit the filaments 4742 from moving out from between the inner member 4746 and the outer member 4745. Similarly, forces on the filaments 4742 can act to draw the inner member 4746 in the direction of arrow R opposite to the direction of arrow S, which pulls the first surface portion 4748a of the inner member 4746 toward the second surface portion 4747a of the outer member 4745 to provide frictional, compressive, constrictive, interference, and/or similar forces that act to inhibit the filaments 4742 from moving out from between the inner member 4746 and the outer member 4745. In some embodiments, the inner member 4746 can further be connected to the outer member 4745 via brazing, welding, soldering, and/or the like. As further shown in FIG. 47B, the filaments 4742 can be cut to terminate (e.g., distally or proximally) at the hub 4741.

In other embodiments, a hub of an intervertebral device can include different combinations of crimping, welding, brazing, soldiering, tapering, wedging, and/or the like to provide a mechanical advantage that works to resist forces pulling on filaments of the intervertebral device. For example, in some embodiments a hub can comprise a spelter socket or spelter lock in which filaments of the intervertebral device terminate in a wedge-shaped (e.g., conical) opening of the socket. A resin or other fill material can be flowed around the filaments in the wedge-shaped opening. Accordingly, when a load is applied to the filaments in a direction out of the socket, friction between the resin, the filaments, and the wall of the socket surrounding the opening can act to inhibit or even prevent the filaments from being pulled out of the socket.

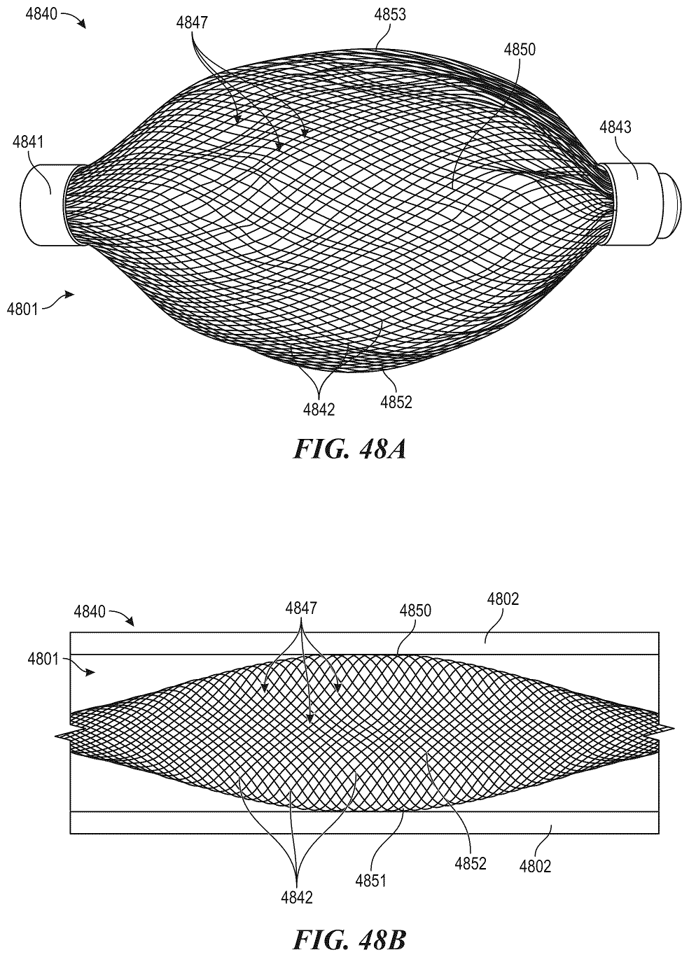

FIGS. 48A and 48B are a perspective top view and a perspective side view, respectively, of an intervertebral device 4840 in accordance with embodiments of the present technology. Referring to FIGS. 48A and 48B, in the illustrated embodiment the intervertebral device 4840 is expanded between a pair of clear plates 4802 (FIG. 48B; e.g., planes) that simulate the adjacent vertebrae (e.g., vertebral endplates) of a disc space 4801. The intervertebral device 4840 can be a braid, mesh, and/or knit of strands or filaments 4842 (e.g., wires) that terminate and/or are joined together at a proximal hub 4841 (FIG. 48A) and a distal hub 4843 (FIG. 48A). The filaments 4842 can define a plurality of openings or pores 4847 therebetween.

In some embodiments, when the intervertebral device 4840 is expanded within the confined disc space 4801, the intervertebral device 4840 assumes a flattened or pancake-like shape having an upper portion 4850 adjacent to and/or contacting an upper one of the plates 4802, a lower portion 4851 (FIG. 48B) opposite the upper portion 4850 and adjacent to and/or contacting a lower one of the plates 4802, a first side portion 4852 extending between the upper portion 4850 and the lower portion 4851, and a second side portion 4853 (FIG. 48A) opposite the first side portion 4852 and extending between the upper portion 4850 and the lower portion 4851. The first and second side portions 4852, 4853 can together form an equatorial band of the intervertebral device 4840. In the illustrated embodiment, the filaments 4842 expand more at the upper portion 4850 and the lower portion 4851 than at the first side portion 4852 and the second side portion 4853 such that the pores 4847 are generally larger at the upper portion 4850 and the lower portion 4851 than at the first side portion 4852 and the second side portion 4853. That is, the filaments 4842 extending across/through/around the upper portion 4850 and the lower portion 4851 expand greater distances while the filaments 4842 extending across the first side portion 4852 and the second side portion 4853 (e.g., equatorially) tend to collapse and, in some instances, form a solid wall (e.g., with none of the pores 4847 and/or very small ones of the pores 4847 formed at the first and second side portions 4852, 4853).

In some aspects of the present technology, the configuration of the vertebral device 4840 to have smaller ones (or none) of the pores 4847 positioned along the first side portion 4852 and the second side portion 4853 can provide several advantages. For example, this can inhibit a fill material and/or graft material inserted into the intervertebral device 4840 from escaping through the first and second side portions 4852, 4853. At the upper and lower portions 4850, 4851, where the pores 4847 are larger, the intervertebral device 4840 is bounded by the plates 4802 (e.g., vertebral endplates) which inhibit the fill material and/or the graft material from escaping from the intervertebral device 4840. Additionally, the larger pores 4847 at the first and second side portions 4850, 4851 can allow for greater bone growth from the vertebral endplates into the intervertebral device 4840 for fusion.

In some aspects of the present technology, the filaments 4842 at the first and second side portions 4852, 4853 can collapse together to increase the hoop strength of the intervertebral device 4840. More specifically, in the illustrated pancake-like shape of the intervertebral device 4840, the filaments 4842 along the first and second side portions 4852, 4853 lengthen more and therefore see more load, and the collapsed-together configuration of the filaments 4842 at the first and second side portions 4852, 4853 (e.g., at the equatorial band) can have increased strength to bear the load. In some embodiments, each of the filaments 4842 can extend through (e.g., cross through) the first side portion 4852 and/or the second side portion 4853 to maximize a number of the filaments 4842 in the first side portion 4852 and/or the second side portion 4853 that collapse to increase the strength of the intervertebral device 4840. For example, the filaments 4842 can each traverse a spiral or helix like shape along the intervertebral device 4840 between the proximal hub 4841 and the distal hub 4843 such that each of the filaments 4842 extends through the first side portion 4852 and/or the second side portion 4853. A count of the filaments 4842 (e.g., a wire count), a braid angle of the filaments 4842, a size of the filaments 4842, a configuration of the filaments 4842, and/or the like, can be selected/controlled such that each of the filaments 4842 extends through the through the first side portion 4852 and/or the second side portion 4853. In contrast, for example, if any of the filaments 4842 do not extend through the first side portion 4852 and/or the second side portion 4853, such filaments will likely be subjected to less load and/or may be in slack and therefore not contributing materially to the overall strength of the intervertebral device 4840 when the intervertebral device 4840 is expanded within the disc space 4801.

### VI. SELECTED EMBODIMENTS OF FILL MATERIALS FOR INTERVERTEBRAL DEVICES, AND ASSOCIATED SYSTEMS AND METHODS

FIGS. 49-60B illustrate embodiments of fill materials that can be used to fill an intervertebral device, such as described in detail above with reference to FIGS. 1L-1Q and block 288 of the method 280 of FIG. 2 and FIG. 3L and blocks 589 and 590 of the method 580 of FIG. 5. Accordingly, the embodiments described with reference to FIGS. 49-60B can be utilized in the workflow of the spinal surgical procedure described in detail with reference to FIGS. 1A-2, FIGS. 3A-5, and/or elsewhere herein.

FIG. 49 is an enlarged perspective view of a fill material 4960 in accordance with embodiments of the present technology. In the illustrated embodiment, the fill material 4960 comprises a plurality of particles 4962 that, when filled within an intervertebral device, can form a gabion structure. The particles 4962 can be formed from trabecular bone tissue, titanium, metal, silica, metal, biomaterial, sand, demineralized bone, and/or the like. The particles 4962 can have a three-dimensional lattice structure with an irregular (e.g., roughened) outer surface 4963. The irregular outer surfaces 4963 can provide a high-friction interface and/or interlock interface between the particles 4962 when filled with the intervertebral device—for example, providing a “Velcro-like” coupling between the particles 4962 to enhance a gabion effect.

FIG. 50 is an enlarged side view of a fill material 5060 in accordance with embodiments of the present technology. In the illustrated embodiment, the fill material 5060 comprises a plurality of beads 5062 coupled to/along a string 5064. The beads 5062 can be formed from trabecular bone tissue, titanium, metal, silica, metal, biomaterial, sand, demineralized bone, and/or the like. In some aspects of the present technology, the fill material 5060 can be deployed through an introducer shaft (e.g., the balloon shaft 152 of FIGS. 1L-1M) into an intervertebral device and, if necessary, removed from the intervertebral device by pulling the string 5064 proximally through the introducer shaft. That is, the fill material 5060 can be reversibly/removably deployed into the intervertebral device.

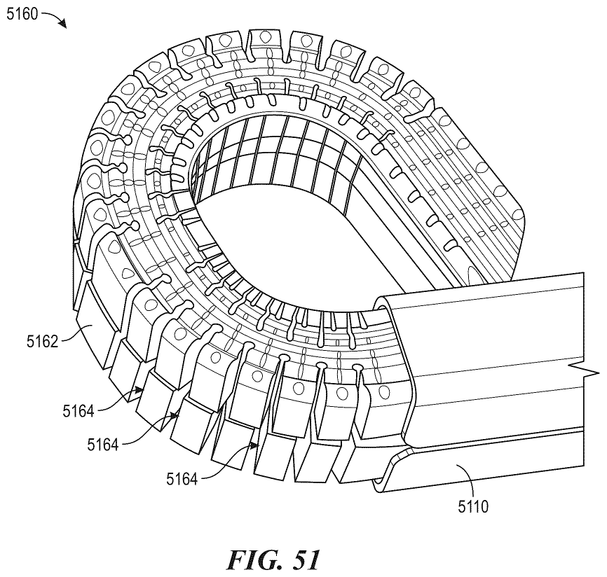

FIG. 51 is an enlarged perspective view of a fill material 5160 extending from an introducer 5110 in accordance with embodiments of the present technology. In the illustrated embodiment, the fill material 5160 comprises a material body 5162 having a plurality of grooves or channels 5164 extending therethrough. The channels 5164 can be laser cut into the material body 5162 and can allow the material body 5162 to assume a coiled shape when deployed within an intervertebral device. The material body 5162 can comprise polyetheretherketone (PEEK), hydroxyapatite (HA), and/or other materials described herein. In some aspects of the present technology, the fill material 5160 can be deployed through the introducer 5110 into the intervertebral device and, if necessary, removed from the intervertebral device by pulling the material body 5162 proximally through the introducer 5110. That is, the fill material 5160 can be reversibly/removably deployed into the intervertebral device.

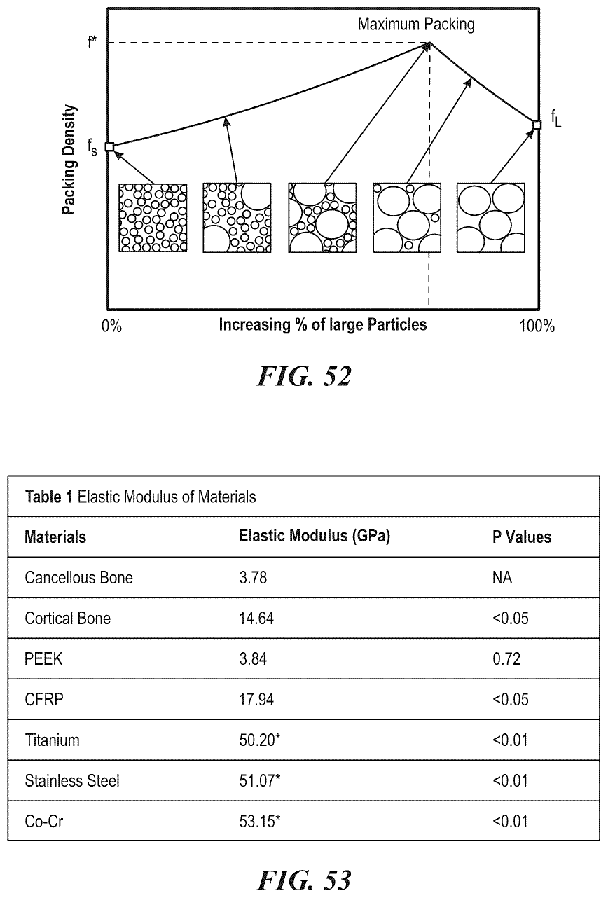

In some embodiments, a fill material in accordance with the present technology can include a plurality of particles having different sizes. The variable size of the particles can help the particles interlock when filled within an intervertebral device to increase the strength of the fill material and to inhibit or even prevent subsidence of the intervertebral device. In some embodiments, such a fill material can include large particles and small particles. FIG. 52, for example, is a graph illustrating a packing density (y-axis) versus a percentage of large fill particles relative to small fill particles (x-axis) in accordance with embodiments of the present technology. As shown, there is a percentage (˜80%) of large fill particles relative to small fill particles at which the packing density is maximized. In some embodiments, a fill material in accordance with the present technology can include a ratio of large and small fill particles selected to maximize the packing density to, for example, inhibit or even prevent subsidence of the intervertebral device.

In some embodiments, a fill material in accordance with the present technology can include particles having different moduli of elasticity. By varying the moduli of elasticity of the particles the fill material can be configured to have a desired overall modulus of elasticity selected to, for example, be similar to that of vertebral bone. FIG. 53, for example, is a table of the different moduli of elasticity of various materials that can be used for the particles of a fill material in accordance with embodiments of the present technology.

In some embodiments, a fill material in accordance with the present technology can comprise (i) a plurality of particles configured to interlock together and (ii) and an infill material configured to surround and infill the particles. For example, the particles can be configured to interlock together in a gabion-like structure, and the infill material can infill the gabion-like structure. Accordingly, the fill material can comprise different types of fill materials used together such that the overall fill material is not homogenous. The particles can have different sizes and/or moduli of elasticity, and can comprise different types of particles (e.g., a heterogenous mixture of particles). The infill material can be a cement, bone graft, polymer, or similar material, and can be amorphous or liquid when injected between the particles. In some aspects of the present technology, there are different characteristics imparted by different types and combinations of fill material. For example, smaller fill particles can pack together more densely and provide greater load bearing performance. Larger particles can impart greater porosity and negative space for the infill material to be injected. A fill material including particles of different sizes can allow for the control of such different characteristics. In some embodiments, the infill material is adhesive to allow for greater and/or faster interlocking between the particles, which can help maintain the intended shape/volume/height of the intervertebral device. In some embodiments, the infill material can help the fill material resist tensile forces, whereas the particles (e.g., arranged in a gabion structure) primarily withstand compressive forces. In some embodiments, the infill material contains materials that promote bone growth and fusion.

The gabion characteristics and behavior of a fill material can be influenced by the geometry of the individual particles of the fill material. In particular, macro features (e.g., shape, size, geometry) of the fill material can be more important to the gabion structure than micro features (e.g., surface texture, friction) of the fill material when considering the relatively large forces the fill material is subjected to when deployed within an intervertebral device. For example, micro features can sustain less outward/radial force due to smaller interface surfaces, therefore small forces will cause breakage of micro features, leading to gabion reconfiguration. On the gross scale, this can lead to more consistent outward radial force until final settling of the fill material. In contrast, macro features can be configured to require larger feature breakage forces, leading to a higher threshold for applied force to cause gabion reconfiguration. On the gross scale, this can lead to higher resistance to initial settling, or faster interlock of the fill material as load is applied. Macro features can reduce the radial component vector and keep the ratio of axial component vector to axial applied force high. In some aspects of the present technology, this can advantageously reduce the forces on the braid of the intervertebral device, allowing for smaller profile (e.g., thinner wire, thinner diameter) intervertebral devices.

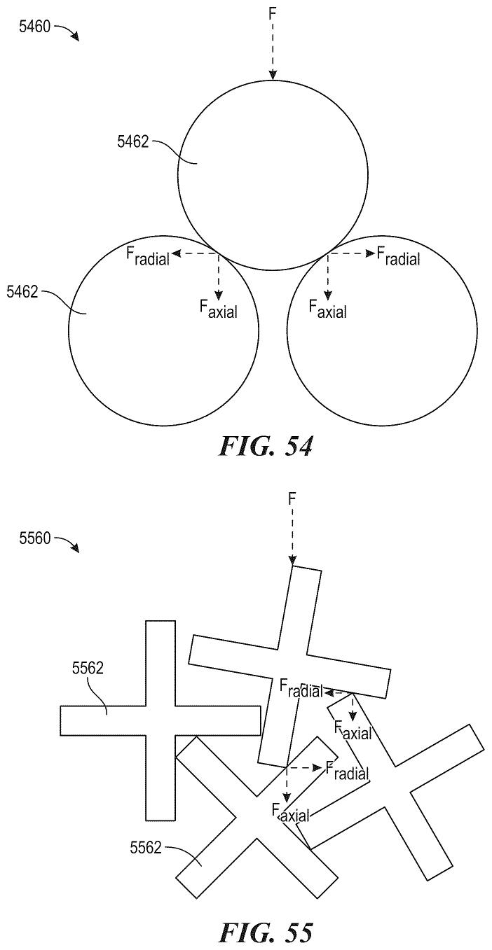

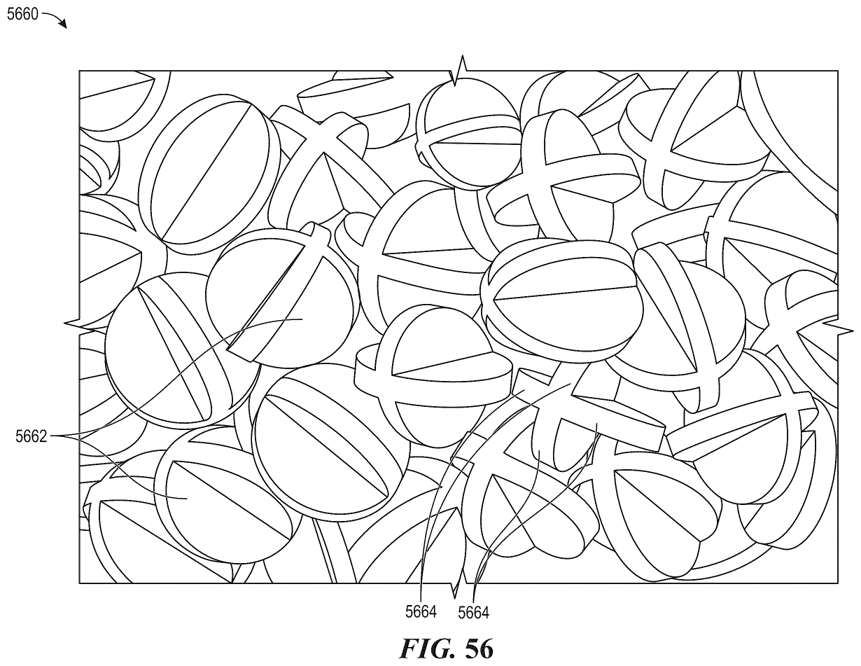

More particularly, for example, FIG. 54 is an enlarged side view of a fill material 5460 in accordance with embodiments of the present technology. In the illustrated embodiment, the fill material 5460 comprises a plurality of particles 5462 having a generally circular cross-sectional shape. When an axial force F is applied to the fill material 5460 (e.g., when an intervertebral device filled with the fill material 5460 is loaded), the interfaces between the particles 5462 can transmit the force F through the fill material 5460 with a relatively large radial vector force component F_{radial} and correspondingly small axial vector force component Fₐₓᵢₐₗ due to the circular shapes of the particles 5462. In contrast, FIG. 55 is an enlarged side view of a fill material 5560 in accordance with embodiments of the present technology. In the illustrated embodiment, the fill material 5560 comprises a plurality of particles 5562 having a generally cross-like or plus-like cross-sectional shape. When an axial force F is applied to the fill material 5560 (e.g., when an intervertebral device filled with the fill material 5560 is loaded), the interfaces between the particles 5562 can transmit the force F through the fill material 5460 with a relatively large axial vector force component Fₐₓᵢₐₗ and correspondingly small radial vector force component F_{radial} due to the generally cross-like shapes of the particles 5562. In some aspects of the present technology, this can reduce radial forces on a braid of the intervertebral device and reduce subsidence of the fill material 5560. The result is that more of the overall axial force F is transmitted via/carried by a gabion structure of the fill material 5560 rather than the intervertebral device.

FIGS. 56-60B are views of different fill materials configured in accordance with embodiments of the present technology and configured to form a gabion structure with high axial force transmission relative to radial force transmission. FIG. 56, for example, is a perspective view of a fill material 5660 comprising a plurality of particles 5662 in accordance with embodiments of the present technology. In the illustrated embodiment, the particles 5662 each have a generally spheroidal shape comprising a plurality of (e.g., four) fins 5664. That is, the particles 5662 can each comprise a unitary body comprising a pair of elliptical cylinders extending orthogonal to and intersecting one another.

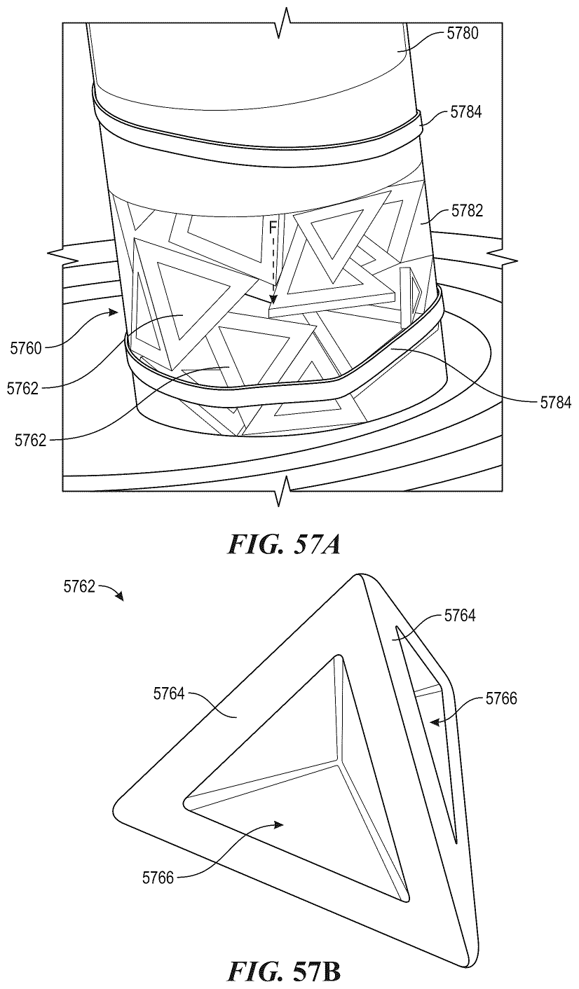

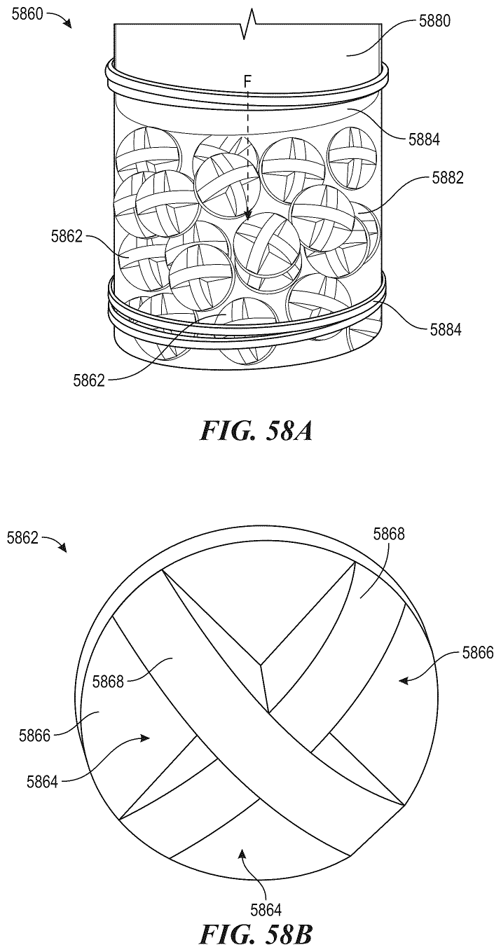

FIG. 57A is a perspective view of a fill material 5760 comprising a plurality of particles 5762 subject to an axial force F via a loading machine 5780 in accordance with embodiments of the present technology. FIG. 57B is a perspective view of one of the particles 5762 of the fill material 5760 in accordance with embodiments of the present technology. Referring to FIG. 57B, the particles 5762 can each have a pyramidal shape comprising triangular faces 5764 (e.g., four triangular faces). Some or all (e.g., all) of the triangular faces 5764 can have a pyramidal-shaped cutout 5766 formed therein. Referring to FIG. 57A, the shape of the particles 5762 can cause the fill material 5760 to form a strong gabion structure in which a substantial portion of the axial force F (e.g., 90% or more) is transmitted axially rather than radially. For example, in the illustrated embodiment the fill material 5760 is retained radially only by a plastic sheet 5782 secured with rubber bands 5784 when the axial force F is high (e.g., greater than 100 pounds per inch)—showing that most of the axial force F is transmitted axially rather than radially by the fill material 5760.

FIG. 58A is a perspective view of a fill material 5860 comprising a plurality of particles 5862 subject to the axial force F via the loading machine 5780 in accordance with embodiments of the present technology. FIG. 58B is a perspective view of one of the particles 5862 of the fill material 5860 in accordance with embodiments of the present technology. Referring to FIG. 58B, the particles 5862 can each have a generally spherical shape including a plurality of (e.g., eight) wedge-shaped cutouts 5864. That is, the particles 5862 can each include a unitary body comprising a generally cylindrical middle portion 5866 defining opposing sides, and a pair of partially-cylindrical fins 5868 extending from each side and intersecting orthogonal to one another. Referring to FIG. 58A, the shape of the particles 5862 can cause the fill material 5860 to form a strong gabion structure in which a substantial portion of the axial force F (e.g., 90% or more) is transmitted axially rather than radially. For example, in the illustrated embodiment the fill material 5860 is retained radially only by the plastic sheet 5782 secured with the rubber bands 5784 when the axial force F is high (e.g., greater than 100 pounds per inch)—showing that most of the axial force F is transmitted axially rather than radially by the fill material 5860.

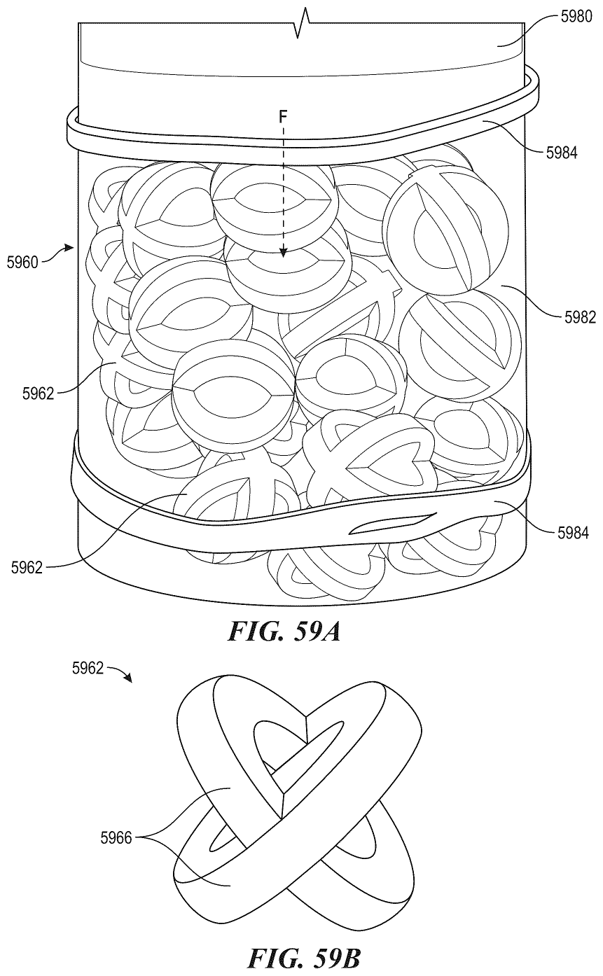

FIG. 59A is a perspective view of a fill material 5960 comprising a plurality of particles 5962 subject to the axial force F via the loading machine 5780 in accordance with embodiments of the present technology. FIG. 59B is a perspective view of one of the particles 5962 of the fill material 5960 in accordance with embodiments of the present technology. Referring to FIG. 59B, the particles 5962 can each have a generally spherical shape formed by a unitary body comprising a pair of circular rings 5964 extending orthogonal to and intersecting one another. Referring to FIG. 59A, the shape of the particles 5962 can cause the fill material 5960 to form a strong gabion structure in which a substantial portion of the axial force F (e.g., 90% or more) is transmitted axially rather than radially. For example, in the illustrated embodiment the fill material 5960 is retained radially only by the plastic sheet 5782 secured with the rubber bands 5784 when the axial force F is high (e.g., greater than 100 pounds per inch)—showing that most of the axial force F is transmitted axially rather than radially by the fill material 5960.

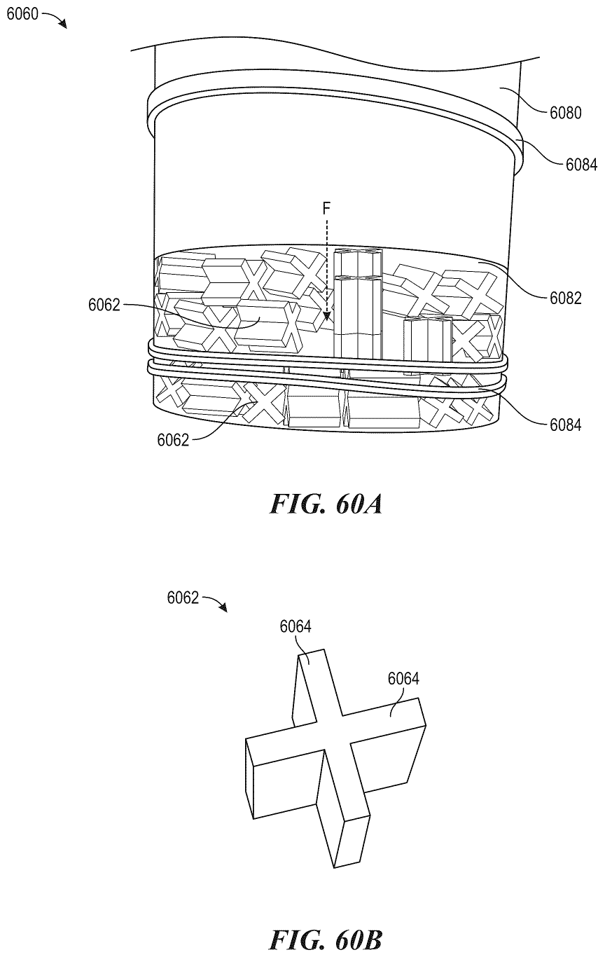

FIG. 60A is a perspective view of a fill material 6060 comprising a plurality of particles 6062 subject to the axial force F via the loading machine 5780 in accordance with embodiments of the present technology. FIG. 60B is a perspective view of one of the particles 6062 of the fill material 6060 in accordance with embodiments of the present technology. Referring to FIG. 60B, the particles 6062 can each comprise a unitary body comprising a pair of rectangular prisms 6064 extending orthogonal to and intersecting one another. Referring to FIG. 60A, the shape of the particles 6062 can cause the fill material 6060 to form a strong gabion structure in which a substantial portion of the axial force F (e.g., 90% or more) is transmitted axially rather than radially. For example, in the illustrated embodiment the fill material 6060 is retained radially only by the plastic sheet 5782 secured with the rubber bands 5784 when the axial force F is high (e.g., greater than 100 pounds per inch)—showing that most of the axial force F is transmitted axially rather than radially by the fill material 6060.

### VII. SELECTED EMBODIMENTS OF FILLING DEVICES FOR FILLING INTERVERTEBRAL DEVICES WITH A FILL MATERIAL, AND ASSOCIATED SYSTEMS AND METHODS

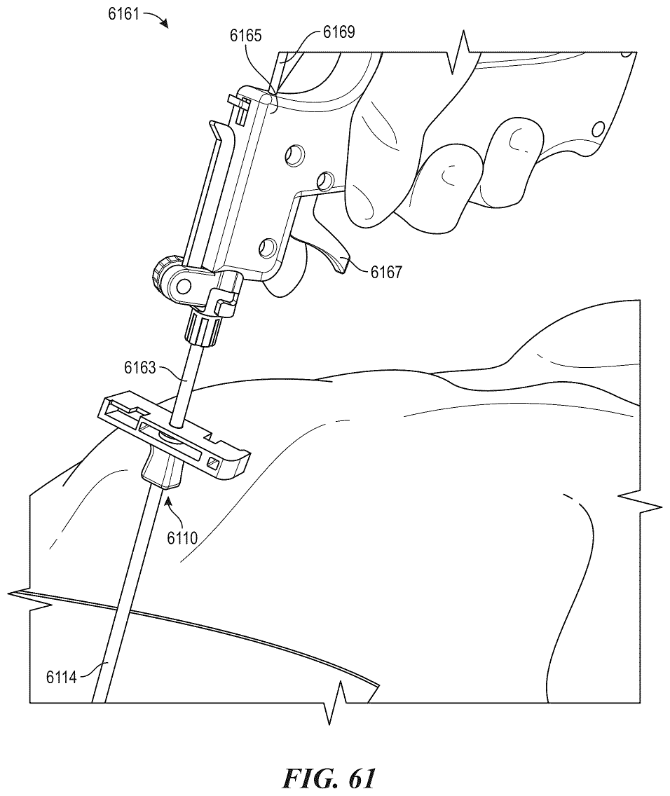

FIGS. 61-64 illustrate filling devices for filling, or aiding in filling, intervertebral devices with a fill material, such as described in detail above with reference to FIGS. 1L-1Q and block 288 of the method 280 of FIG. 2 and FIG. 3L and blocks 589 and 590 of the method 580 of FIG. 5. Accordingly, the embodiments described with reference to FIGS. 61-64 can be utilized in the workflow of the spinal surgical procedure described in detail with reference to FIGS. 1A-2, FIGS. 3A-5, and/or elsewhere herein.

FIG. 61 is a perspective side view of a proximal portion of a filling device 6161 inserted through an introducer 6110 in accordance with embodiments of the present technology. In the illustrated embodiment, the filling device 6161 includes an elongate member 6163 coupled to a handle 6165. The handle 6165 further includes an actuator 6167 operably coupled to a plunger 6169 configured to move through a lumen of the elongate member 6163. In the illustrated embodiment, the actuator 6167 is a trigger. The elongate member 6163 can contain a fill material in the lumen, and can be inserted through a cannula 6114 of the introducer 6110 positioned to access a disc space including an intervertebral device deployed therein. The actuator 6167 can be actuated (e.g., squeezed) to drive the plunger 6169 distally through the lumen of the elongate member 6163 to eject the fill material out of the elongate member 6163 into the intervertebral device. In some aspects of the present technology, the actuator 6167 provides a mechanical advantage that facilitates injection of the fill material into the intervertebral device from the elongate member 6163. In other embodiments, the actuator 6167 can be a rotatable member or other member configured to provide a mechanical advantage for moving the plunger 6169 through the elongate member 6163.

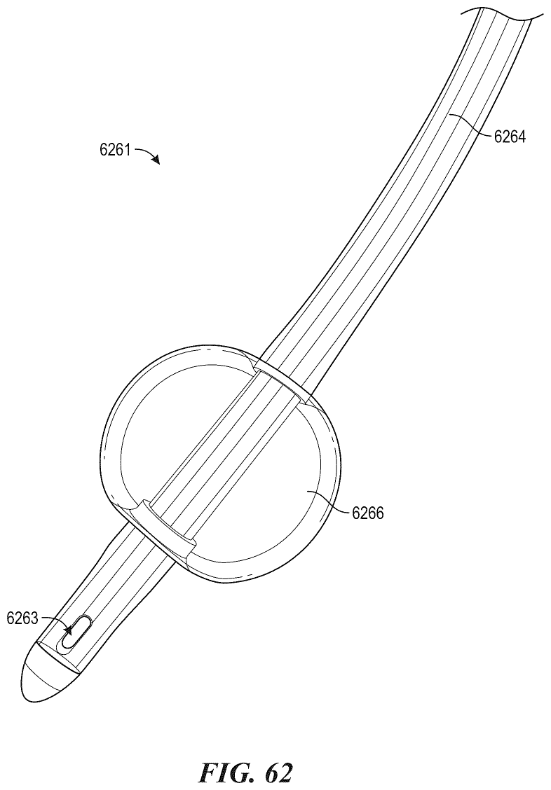

FIG. 62 is a perspective side view of a distal portion of a filling device 6261 in accordance with embodiments of the present technology. In the illustrated embodiment, the filling device 6261 comprises an elongate member 6264 having a balloon 6266 coupled thereto. The balloon 6266 can have a donut or toroidal shape about the elongate member 6264. The elongate member 6264 defines a fill lumen (e.g., a primary lumen) and includes one or more injection ports 6263 at a distal end portion thereof. The elongate member 6264 can further define one or more inflation lumens (e.g., secondary lumens) that are fluidly coupled to the balloon 6266 for inflating the balloon 6266. The filling device 6261 can be inserted through an introducer such that the balloon 6266 and the injection ports 6263 are positioned within an intervertebral device. A fill material can be injected through the fill lumen for egress out of the injection ports 6263 into the intervertebral device. The balloon 6266 can be inflated before and/or during filling of the intervertebral device to at least partially expand the intervertebral device and thereby reduce a filling resistance of the intervertebral device and/or to influence a shape of the intervertebral device.

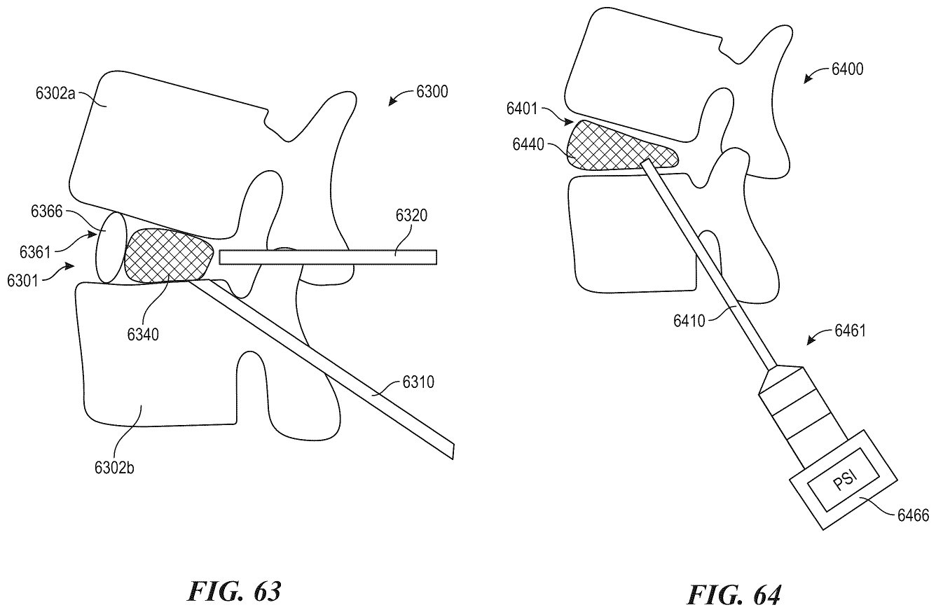

FIG. 63 is a side (e.g., lateral) view of a distal portion of a filling device 6361 and an intervertebral device 6340 deployed and expanded within a disc space 6301 of a spine 6300 of a patient in accordance with embodiments of the present technology. In the illustrated embodiment, the filling device 6361 comprises a balloon 6366 positioned outside the intervertebral device 6340. The intervertebral device 6340 can be filled with a fill material through a first introducer 6310. The balloon 6346 can be deployed through the first introducer 6310 (e.g., in parallel to the intervertebral device 6340), or can be deployed through a second introducer 6320 (e.g., a trocar) positioned to access the disc space 6301 separately from the first introducer 6310 (e.g., via a transforaminal approach). The balloon 6366 can be inflated before and/or during filling of the intervertebral device 6340 to hold the disc space 6301 open (e.g., by pushing vertebrae 6302a-b adjacent the disc space 6301 away from one another) and thereby reduce a filling resistance of the intervertebral device 6340. In some embodiments, the balloon 6346 contacts the intervertebral device 6340 to expand the intervertebral device 6340.

Similarly, in some embodiments the balloon 6346 can be positioned on a first side of the disc space 6301 to lift the first side while the intervertebral device 6340 is deployed on a second side of the disc space 6301. The balloon 6346 can therefore work in tandem with the intervertebral device 6340 as the intervertebral device 6340 is expanded and filled with a fill material.

FIG. 64 is a side (e.g., lateral) view of a filling device 6461 and an intervertebral device 6440 deployed and expanded within a disc space 6401 of a spine 6400 of a patient in accordance with embodiments of the present technology. In the illustrated embodiment, the filling device 6451 includes a pressure sensing assembly 6466 coupled to an introducer 6410. Fill material can be injected through the introducer 6410 while the pressure sensing assembly 6466 measures/detects a pressure within the intervertebral device 6440 and/or a volume of the fill material injected into the intervertebral device 6440 and provide feedback to an operator (e.g., surgeon) based on the sensed pressure and/or volume. For example, the pressure sensing assembly 6466 can determine when (i) a desired/optimal volume of the fill material has been injected into the intervertebral device 6440, (ii) the intervertebral device 6440 has reached an optimal internal pressure, and/or (iii) the intervertebral device 6440 has reached an optimal braid tension. In some embodiments, the desired volume can be determined from a pressure/volume previously determined by a balloon used to expand the intervertebral device 6440 (e.g., via the balloon device 3431 described in detail with reference to FIG. 34).

In further aspects of the present technology, the pressure sensing assembly 6466 can sense a pressure and/or volume within the intervertebral device 6440 to provide feedback to inhibit or even prevent over filling of the intervertebral device 6440 and/or over tensioning of the braid of the intervertebral device 6440. For example, in an analogous or identical manner as described in detail above with reference to FIG. 35, the pressure sensing assembly 6466 can determine a pressure-volume (and/or like) curve during filling of the intervertebral device 6440 with the fill material. As the volume of fill material increases, the pressure within the intervertebral device 6440 can increase. In some embodiments, the pressure sensing assembly 6466 can measure/calculate a derivate of the pressure-volume curve. There can be a first region of the pressure-volume curve in which the intervertebral device 6440 is not over filled and/or the braid is not over tensioned as indicated by, for example, a derivate of the pressure-volume curve indicating that the pressure is increasing at a rate less than a predetermined threshold rate. Likewise, there can be a second region of the pressure-volume curve in which the intervertebral device 6440 beings to become or is over filled and/or the braid beings to become or is over tensioned as indicated by, for example, the derivate of the pressure-volume curve indicating that the pressure is increasing at a rate greater than the predetermined threshold rate. Accordingly, the pressure sensing assembly 6466 can stop filling of the intervertebral device 6440, indicate a warning (e.g., an audible or visual alarm, warning, etc.), and/or the like when the derivate of the pressure-volume curve exceeds the predetermined threshold rate to avoid over filling of the intervertebral device 6440 and/or over tensioning of the braid of the intervertebral device 6440.

In some embodiments, the pressure sensing assembly 6466 can be coupled to a balloon that is expanded within the intervertebral device 6440. The fill material can be injected into the balloon. Such a balloon can be fully or partially dissolvable such that the balloon remains after a fill material is injected therein before subsequently dissolving. In such embodiments, the balloon can be inflated to establish a desired tension on the braid of the intervertebral device 6440. The pressure sensing assembly 6466 can sense a pressure of the balloon that indicates that the desired braid tension has been achieved.

### VIII. SELECTED EMBODIMENTS OF DEVICES FOR CLOSING, TENSIONING, AND/OR DETACHING INTERVERTEBRAL DEVICES, AND ASSOCIATED SYSTEMS AND METHODS

FIGS. 65-70D illustrate embodiments of methods of closing, tensioning, and/or detaching intervertebral devices, such as described in detail above with reference to FIG. 1R and blocks 289-291 of the method 280 of FIG. 2 and FIG. 3L and blocks 589 and 590 of the method 580 of FIG. 5. Accordingly, the embodiments described with reference to FIGS. 65-70D can be utilized in the workflow of the spinal surgical procedure described in detail with reference to FIGS. 1A-2, FIGS. 3A-5, and/or elsewhere herein.

FIG. 65 is a side view of a closure mechanism 6546 in accordance with embodiments of the present technology. In the illustrated embodiment, the closure mechanism 6546 is a screw comprising a threaded head portion 6542 and a body portion 6544. Referring to FIGS. 1R and 65, the closure mechanism 6546 can be inserted into the opening 145 in the proximal portion 141 of the intervertebral device 140 and rotated such that the threaded head portion 6542 mates with corresponding threads along the proximal portion 141 surrounding the opening 145. The body portion 6544 can extend into the intervertebral device 140 and displace some of the fill material 160 therein to increase a total volume of material within the intervertebral device 140. In some aspects of the present technology, increasing the volume within the intervertebral device 140 in this manner can increase a tension of the filaments 142 to, for example, better pack the fill material 160 within the intervertebral device 140 and inhibit or even prevent subsidence of the intervertebral device 140 after implantation in the disc space 101. In some embodiments, the closure mechanism 6546 can be rotated (e.g., torqued) to a specified torque with a torque limiter to set the filaments 142 at a specified tension.

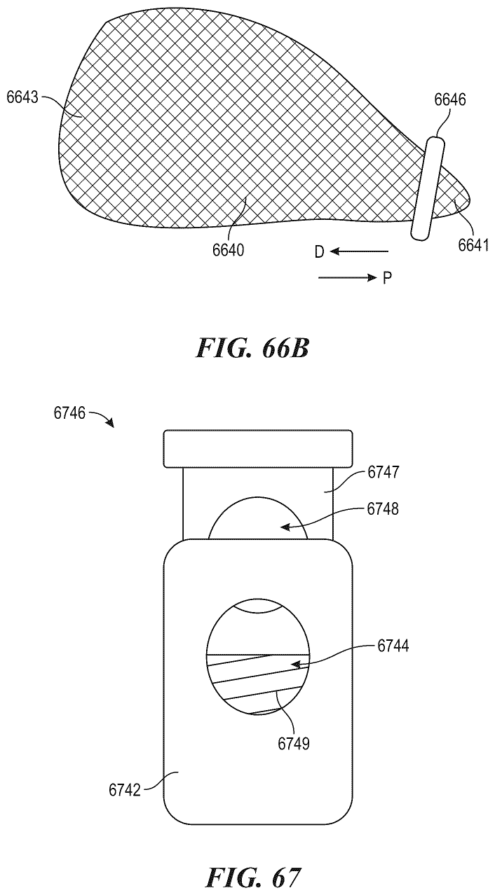

FIG. 66A is a front view of a tensioning and/or closure mechanism 6646 (“mechanism 6646”) in accordance with embodiments of the present technology. In the illustrated embodiment, the mechanism 6646 includes a body 6647 defining an opening 6648. FIG. 66B is a side view of the mechanism 6646 installed on/deployed over an intervertebral device 6640 in accordance with embodiments of the present technology. The intervertebral device 6640 comprises a braid of filaments including a proximal portion 6641 and a distal portion 6643. Referring to FIGS. 66A and 66B, the mechanism 6646 can be positioned over a portion of the intervertebral device 6640 such that, for example, a portion of the proximal portion 6641 extends through the opening 6648 and the mechanism 6646 cinches the intervertebral device 6640. The mechanism 6646 can be slid distally as indicated by arrow D (e.g., by a cinching/tensioning shaft inserted through an introducer) to further cinch the intervertebral device 6640 (e.g., akin to a cinching collar). Alternatively or additionally, the proximal portion 6641 of the intervertebral device 6640 can be pulled proximally as indicated by arrow P (e.g., by a deployment shaft coupled thereto) to further cinch the intervertebral device 6640. Cinching the intervertebral device 6640 can increase a tension of the braided filaments. In some embodiments, the mechanism 6646 is configured to close the intervertebral device 6640 (e.g., to inhibit or even prevent a fill material from egressing therefrom) while, in other embodiments, the mechanism 6646 can be applied over the intervertebral device 6640 after a separate closure mechanism has been attached thereto.

In some embodiments, rather than having a stationary opening 6648, the mechanism 6646 can be similar to a cord lock. FIG. 67, for example, is a side view of a tensioning and/or closure mechanism 6746 (“mechanism 6746”) in accordance with additional embodiments of the present technology. In the illustrated embodiment, the mechanism 6746 includes a barrel portion 6742 defining a first opening 6744, a plunger portion 6747 movably positioned within the barrel portion 6742 and defining a second opening 6748, and a spring 6749 operably coupling the barrel portion 6742 to the plunger portion 6747. The spring 6749 biases the second opening 6748 away from the first opening 6744, and the plunger portion 6747 can be depressed to substantially align the first opening 6744 and the second opening 6748. Referring to FIGS. 66A and 67, the mechanism 6746 can be installed over the proximal portion 6641 of the intervertebral device 6640 such that the proximal portion 6641 extends through the first and second openings 6744, 6748. The mechanism 6746 can then be slid distally as indicated by the arrow D to cinch the intervertebral device 6640 and/or the proximal portion 6641 of the intervertebral device 6640 can be pulled proximally as indicated by arrow P to cinch the intervertebral device 6640. The spring 6749 can bias the plunger portion 6747 away from the barrel portion 6742 such that the mechanism 6746 is locked in position relative to the intervertebral device 6640.

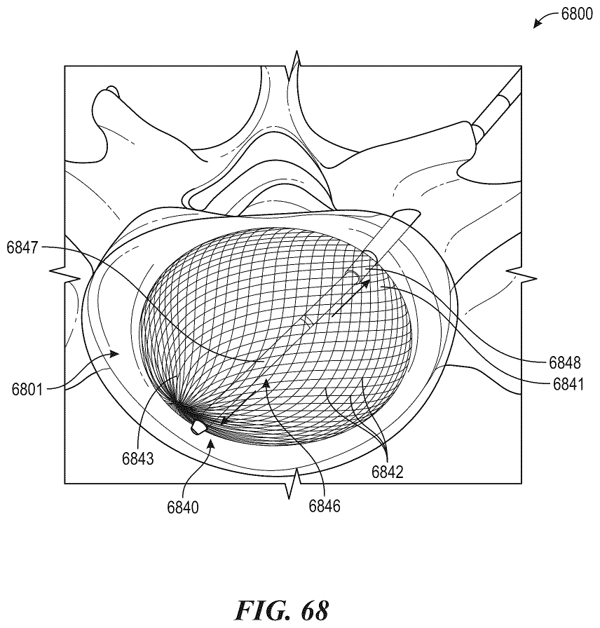

FIG. 68 is a top (e.g., axial) view of an intervertebral device 6840 deployed within a disc space 6801 of a spine 6800 and including a tensioning mechanism 6846 in accordance with embodiments of the present technology. In the illustrated embodiment, the intervertebral device 6840 comprises a braid of filaments 6842 including a proximal portion 6841 and a distal portion 6843. The tensioning mechanism 6846 includes an inner shaft 6847 coupled to the distal portion 6843 and an outer shaft 6848 coupled to the proximal portion 6841. The inner shaft 6847 can be moved (e.g., proximally or distally) relative to the outer shaft 6848 and/or the outer shaft 6848 can be moved (e.g., proximally or distally) relative to the inner shaft 6847 to longitudinally lengthen/shorten the intervertebral device 6840 to affect a tension of the braid of filaments 6842. The inner shaft 6847 and the outer shaft 6848 can remain implanted within the intervertebral device 6840 after deployment to maintain the tension of the filaments 6842.

In some embodiments, a tensioning mechanism in accordance with embodiments of the present technology can include one or more circumferential bands that can be pulled tightly together and locked under a locking a set screw (e.g., the closure mechanism 6546 described in detail with reference to FIG. 65) or clamp mechanism (e.g., the mechanism 6646 and and/or the mechanism 6746 described in detail with reference to FIGS. 66A-67) to tension (e.g., cinch) an intervertebral device. Further, such a tensioning mechanism can include an internal gear that can be actuated to cinch the bands to tension the intervertebral device.

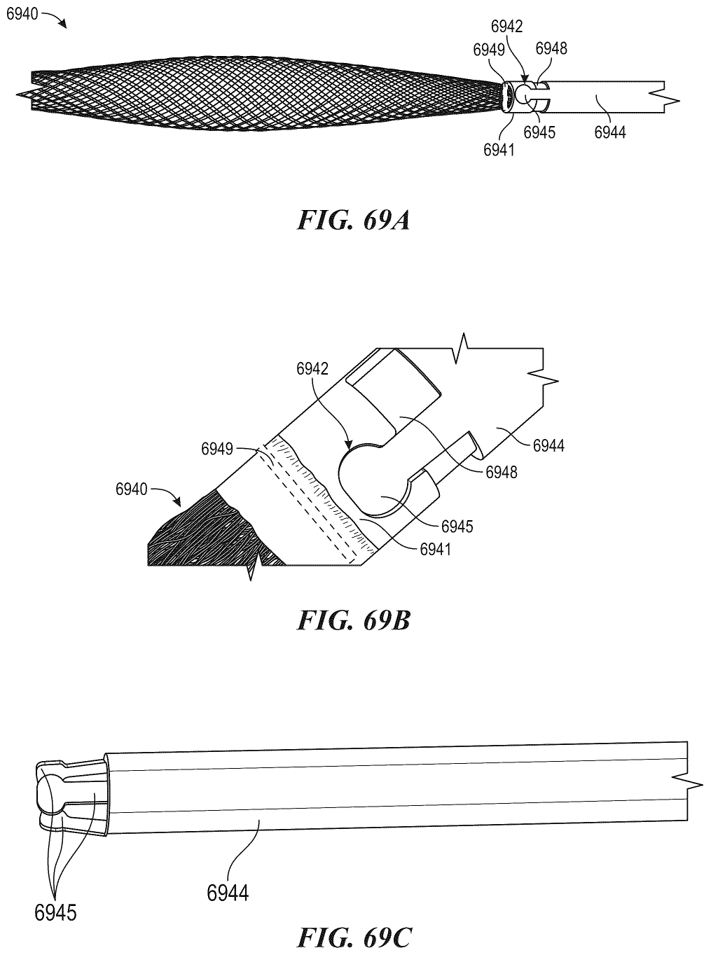

FIG. 69A is a side view of an intervertebral device 6940 coupled to a deployment shaft 6944 in accordance with embodiments of the present technology. FIGS. 69B and 69C are an enlarged view of a coupling between the intervertebral device 6940 and the deployment shaft 6944, and a side view of the deployment shaft 6944, respectively, in accordance with embodiments of the present technology. Referring to FIGS. 69A and 69B, the intervertebral device 6940 includes a proximal hub 6941 comprising a plurality of grooves 6942 configured to receive corresponding tabs 6945 of the deployment shaft 6944. The engagement between the locking grooves 6942 and the tabs 6954 can maintain an operable connection between the deployment shaft 6944 such that the intervertebral device 6940 can be translated (e.g., proximally and/or distally) and/or rotated via corresponding translation/rotation of the deployment shaft 6944.

In the illustrated embodiment, a locking shaft 6948 is inserted through the deployment shaft 6944 to extend at least partially past the tabs 6945. Referring to FIG. 69C, the tabs 6945 are biased radially inward such that they flex radially inward in the absence of external forces. In some embodiments, the tabs 6945 are formed from nitinol, spring steel, and/or the like. Referring to FIGS. 69A-69C, the locking shaft 6948 contacts the tabs 6945 and flexes the tabs 6945 radially outward into the corresponding grooves 6942 when inserted there past. Accordingly, pulling the locking shaft 6948 proximally past the tabs 6945 allows the tabs 6945 to flex radially inward out of the grooves 6942 to decouple the deployment shaft 6944 from the intervertebral device 6940. In this manner, the intervertebral device 6940 can be detached from the deployment shaft 6944 after delivery to a disc space.

In some embodiments, a fill material can be inserted through the locking shaft 6948 for injection into the intervertebral device 6940. The intervertebral device 6940 can include a valve 6949 (shown schematically) at the proximal hub 6941. In some embodiments, the locking shaft 6948 extends through the valve 6949 to open the valve 6949 such that the fill material can be injected into the intervertebral device 6940. Pulling the locking shaft 6948 proximally through the valve 6949 (e.g., during detachment of the intervertebral device 6940 from the deployment shaft 6944) can close the valve 6949—or allow the valve 6949 to close—such that the valve 6949 inhibits or even prevents egress of the fill material past the valve 6949 out of the intervertebral device 6940. In other embodiments, the locking shaft 6948 need not extend through the valve 6949, and the pressure of the fill material can be used to open the valve 6949 to allow for injection of the fill material into the intervertebral device 6940. In such embodiments, the valve 6949 can passively close after injection of the fill material.

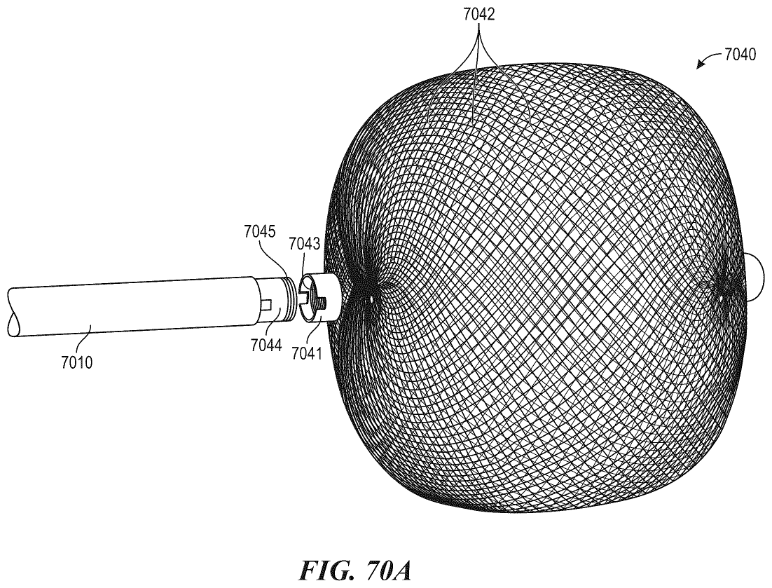

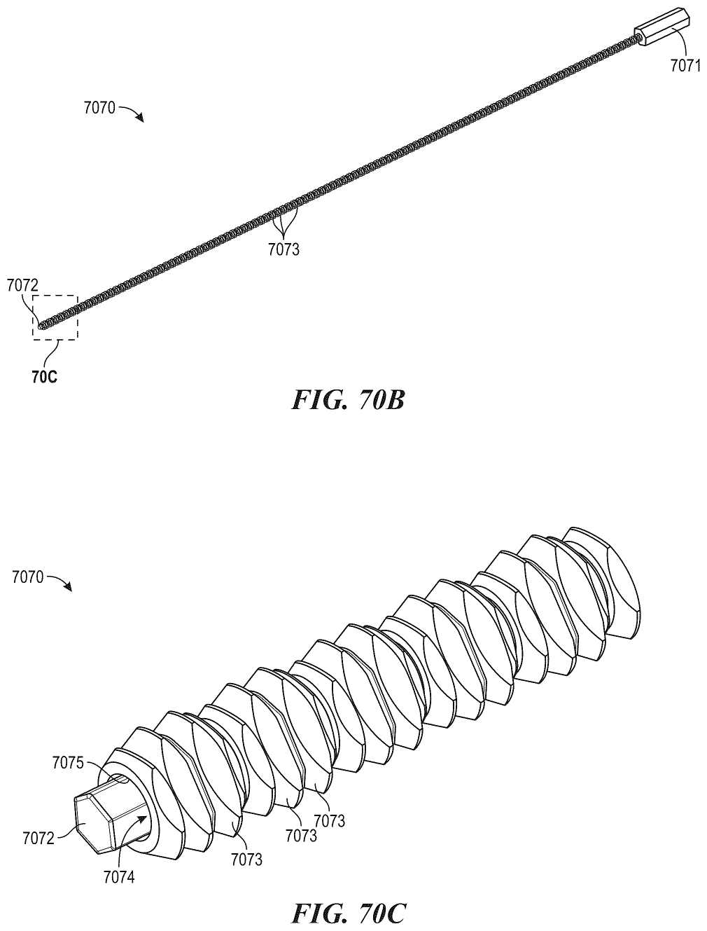

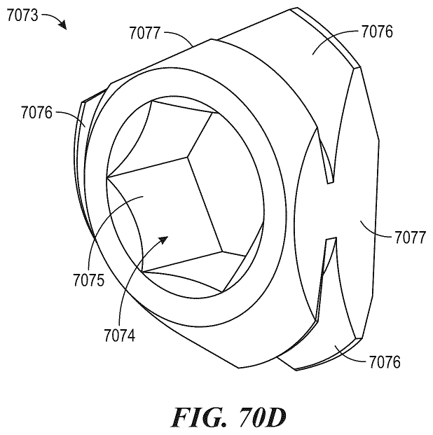

FIG. 70A is a perspective view of an intervertebral device 7040 and a deployment shaft 7044 in accordance with embodiments of the present technology. The intervertebral device 7040 is shown detached from the deployment shaft 7044 in FIG. 70A for clarity. In the illustrated embodiment, the intervertebral device 7040 includes a proximal hub 7041 secured to a mesh or braid of woven filaments 7042 and comprising an at least partially threaded inner surface 7043. The proximal hub 7041 can include some features generally similar or identical in structure and/or function to any of the hubs 4541, 4641, and/or 4741 described in detail above with reference to FIGS. 45A-47B. The deployment shaft 7044 can include a distal portion with an at least partially threaded outer surface 7045. The deployment shaft 7044 can be secured to the proximal hub 7041 by screwing the threaded outer surface 7045 into the threaded inner surface 7043 of the proximal hub 7041 of the intervertebral device 7040. When so connected, the intervertebral device 7040 can be translated (e.g., proximally and/or distally) and/or rotated via corresponding translation/rotation of the deployment shaft 7044. For example, the deployment shaft 7044 can be used to advance the intervertebral device 7040 through an access trocar 7010.

FIG. 70B is a perspective view of a fill cartridge 7070, such as for use in filling the intervertebral device 7040 of FIG. 70A with a fill material in accordance with embodiments of the present technology. FIG. 70C is an enlarged view of a portion of the fill cartridge 7070 of FIG. 70B in accordance with embodiments of the present technology. Referring to FIGS. 70B and 70C, the fill cartridge 7070 can include a proximal hub 7071 coupled to an elongated shaft 7072, and a plurality of fill members 7073 slidably positioned along the elongated shaft 7072. FIG. 70D is a perspective view of one of the fill members 7073 in accordance with embodiments of the present technology. Referring to FIGS. 70C and 70D, each of the fill members 7073 can define a lumen 7074 configured to slide lengthwise over the elongated shaft 7072. In some embodiments, the lumens 7074 of the fill members 7073 and the elongated shaft 7072 can be shaped to inhibit or even prevent rotation of the fill members 7073 about the elongated shaft 7072. In the illustrated embodiment, for example, the lumen 7074 of each of the fill members 7073 is defined by an inner surface 7075 having a polygonal (e.g., hexagonal) shape, and the elongated shaft 7072 has a corresponding polygonal shape such that the fill members 7073 are indexed along the elongated shaft 7072 to rotate with the elongated shaft 7072 but not independently from the elongated shaft 7072. In other embodiments, the lumens 7074 of the fill members 7073 and the elongated shaft 7072 can have other shapes (e.g., irregular) selected to inhibit rotation of the fill members 7073 relative to the elongated shaft 7072.

Referring to FIG. 70D, the fill members 7073 can comprise polyetheretherketone (PEEK), hydroxyapatite (HA), titanium, and/or other rigid material described herein. In some embodiments, the fill members 7073 can have an outer surface comprising screw features 7076 separated by flat portions or cutouts 7077.

Referring to FIGS. 70A-70D, the proximal hub 7071 of the fill cartridge 7070 can be configured to be coupled to a filling device (e.g., a filling gun) and/or other actuation source, and the elongated shaft 7072 and the fill members 7073 thereon are configured to be inserted through the deployment shaft 7044 (FIG. 70A) and/or another access pathway to the proximal hub 7041 of the intervertebral device 7040 (FIG. 70A). The filling device can be actuated to (i) rotate the elongated shaft 7072 and (ii) drive the fill members 7073 distally along the elongated shaft 7072. Such movement can drive the fill members 7073 through the proximal hub 7041 and into the intervertebral device 7040. More particularly, the fill members 7073 can be rotated (e.g., screwed) through the proximal hub 7041 with the screw features 7076 of the fill members 7073 engaging the threaded inner surface 7043 of the proximal hub 7041. In some aspects of the present technology, rotation of the fill members 7073 through the proximal hub 7041 can cause the proximal hub 7041 to rotate to tension the filaments 7042. As the fill members 7073 fill the intervertebral device 7040 and the filaments 7042 are tensioned, the fill members 7073 can form a gabion-like structure within the intervertebral device 7040. In some aspects of the present technology, the fill members 7073 can be shaped such that the fill members 7073 interlock generally vertically (e.g., in a direction between adjacent vertebrae) to provide rigid vertical support and strength. In contrast, for example, spherical fill members may divert substantial forces radially via their non-vertical engagement. Likewise, the cutouts 7077 of the fill members 7073 can help maximize the surface area of the resultant (e.g., gabion-like) structure to provide for osteointegration. In some embodiments, a finally-inserted one of the fill members 7073 remains within the proximal hub 7041 to cap or close the intervertebral device 7040.

### IX. SELECTED EMBODIMENTS OF INTERVERTEBRAL DEVICES, SYSTEMS, AND METHODS FOR CORPECTOMY AND/OR OTHER SPINAL SURGICAL PROCEDURES

Although many of the embodiments described above are described in the context of spinal fusion procedures, the devices, systems, and methods described herein can be used in various other spinal surgical procedures, such as corpectomy procedures and/or the like. A corpectomy procedure involves removing some (e.g., the vertebral body) or all of a vertebra and the adjacent discs. A corpectomy procedure in accordance with embodiments can similarly include accessing the vertebra through a minimally-invasive access port (e.g., a trocar), removing the vertebra and the adjacent discs via instruments inserted through the minimally-invasive access port, inserting an interbody device through the minimally-invasive access port into the spinal space where the vertebra and adjacent discs previously were, expanding the interbody device within the spinal space via instruments inserted through minimally-invasive access port, and filling the interbody device via instruments inserted through minimally-invasive access port.

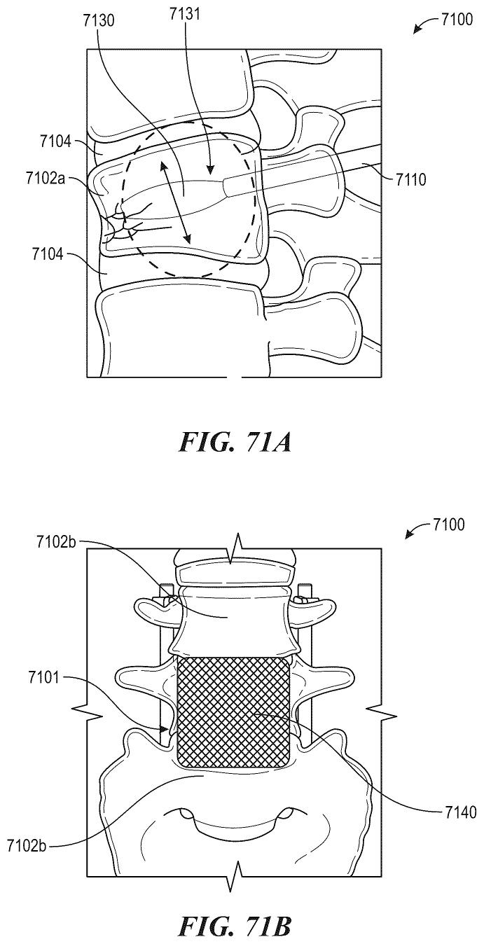

More specifically, for example, FIG. 71A is a side (e.g., lateral) view of a spine 7100 during a corpectomy procedure in accordance with embodiments of the present technology. The spine 7100 includes a vertebra 7102a and discs 7104 adjacent the vertebra 7102a to be removed during the corpectomy procedure. In the illustrated embodiment, an introducer 7110 is positioned to access the vertebra 7102a, and a balloon device 7131 including a balloon 7130 is inserted through the introducer 7110. The balloon 7130 can be expanded within the vertebra 7102a to mechanically disrupt (e.g., break apart) the vertebra 7102a and/or the adjacent discs 7104. In some embodiments, other mechanical components (e.g., disc-shavers, bone-shavers, cutting instruments) can be inserted though the introducer 7110 to facilitate removal of the vertebra 7102a and the adjacent discs 7104.

FIG. 71B is a side view (e.g., an anteriorly-facing view) of the spine 7100 after removal of the vertebra 7102a and the discs 7104 to form a spinal space 7101 in accordance with embodiments of the present technology. In the illustrated embodiment, an interbody device 7140 has been deployed in the spinal space 7101. The interbody device 7140 can comprise a mesh or braid of filaments that is filled with a fill material, as described in detail above. The interbody device 7140 can contact, conform to, and provide support between a pair of remaining vertebra 7102b and/or other spinal structures. In some aspects of the present technology, the interbody device 7140 can be deployed through the same introducer 7110 (FIG. 71A) such that the entire corpectomy procedure is carried out through an open surgery, minimally-invasive or percutaneous access port.

### X. SELECTED EMBODIMENTS OF DEVICES, SYSTEMS, AND METHODS FOR MEASURING SPINAL ANGLES

FIGS. 72A-77 illustrate embodiments of systems and methods for correcting and/or measuring lordosis, kyphosis, scoliosis and/or other curvatures of a spine of a patient, such as described in detail above with reference to FIGS. 3G-3L and blocks 586-589 of the method 580 of FIG. 5. Accordingly, the embodiments described with reference to FIGS. 72A-77 can be utilized in the workflow of the spinal surgical procedures described in detail with reference to FIGS. 1A-2, 3A-5, and/or elsewhere herein.

FIG. 72A is a side view (e.g., a lateral view) of a portion of a spinal fixation system 7210 attached to a spine 7200 of a patient including an upper (e.g., first) vertebra 7202a and a lower (e.g., second) vertebra 7202b in accordance with embodiments of the present technology. In general, the spinal fixation system 7210 is configured to allow (i) for the relative movement of the vertebrae 7202 to establish a desired angle of the spine 7200, (ii) for measurement/determination of the angle, and (iii) for the subsequent fixation of the vertebrae 7202 at the desired angle with the intervertebral device 7240 therebetween. In the illustrated embodiment, a first fixation member 7272a is secured to/within the upper vertebra 7202a and a second fixation member 7272b is secured to/within the lower vertebra 7202b. The first and second fixation members 7272a-b (collectively “fixation members 7272”) can be pedicle screws, cortical screws, anchors, rivets, wires, bands, interspinous clamps, interlaminar clamps, plates, dowels, cement, friction devices, adhesive, epoxy, and/or the like. For example, in the illustrated embodiment the fixation members 7272 are pedicle screws each including (i) a threaded screw body 7273 (including an individually identified first screw body 7273a and a second screw body 7273b) having a head 7274 (including an individually identified first head 7274a and a second head 7274b) and configured to be screwed into and secured within the corresponding ones of the vertebrae 7202 and (ii) a polyaxial head or tulip 7275 (including an individually identified first tulip 7275a and a second tulip 7275b) coupled to the head 7274. The tulips 7275 can rotate relative to the heads 7274 or can be fixed in orientation relative to the heads 7274. In some embodiments, the fixation members 7272 can include some features generally similar or identical in structure and/or function to the fixation member 1572 described in detail with reference to FIGS. 15A and 15B.

In the illustrated embodiment, a first tower member 7284a (e.g., tower, tube, rigid member, positioning tube, and/or the like) is releasably secured to the first head 7274a of the first fixation member 7272a, and a second tower member 7284b is releasably secured to the second head 7274b of the second fixation member 7272b. The tower members 7284 can provide an access channel for accessing the heads 7274 of the fixation members 7272. In some embodiments, one or more positioning ties 7278 (e.g., locking devices; including an individually identified first positioning tie 7278a and a second positioning tie 7278b) can be secured between the tower members 7284. In some embodiments, the positioning ties 7278 include one or more joints 7279 (each including an individually identified first joint 7279a and a second joint 7279b) that may be fixed or movable. The positioning ties 7278 can be integrated with the tower members 7284 and/or can be separate therefrom and releasably coupled thereto. The positioning ties 7278 and the tower members 7284 can together define a positioning system or assembly 7220. In the illustrated embodiment, an intervertebral device 7240 is implanted in a disc space 7207 between the vertebrae 7202, such as any of the expandable, fillable, tensionable, etc., intervertebral devices described in detail herein. The intervertebral device 7240 may replace a diseased disc that has been at least partially removed. In other embodiments, the intervertebral device 7240 can comprise a balloon that is inflatable to distract the disc space 7207, as described in detail herein.

FIG. 72B is an identical side view (e.g., a lateral view) of the portion of the spinal fixation system 7210 attached to the spine 7200 of the patient of FIG. 72A illustrating various distances, angles, and/or points of rotation that can be manipulated to drive other target distances, angles, and/or points of rotation in accordance with embodiments of the present technology. For clarity, the reference numerals of the various components of the spinal fixation system 7210 are omitted in FIG. 72A.

Referring to FIGS. 72A and 72B together, (i) a first distance d₁ can be defined as the intervertebral distance along the anterior side of the disc space 7207, (ii) a second distance d₂ can be defined as the distance between two anatomical landmarks along the posterior side of the disc space 7207, such as a distance between the heads 7274 of the fixation members 7272 or a distance between the tulips 7275 of the fixation members 7272, (iii) a third distance d₃ can be defined as a distance between the tower members 7284 along the first positioning tie 7278a, and (iv) a fourth distance d₄ can be defined as a distance between the tower members 7284 along the second positioning tie 7278b. Likewise, the system 7210 can define one or more points of rotation, such as: (i) a first point of rotation R₁ of the first tulip 7275a about the first head 7274a, (ii) a second point of rotation R₂ of the second tulip 7275b about the second head 7274b, (iii) a third point of rotation R₃ at the first joint 7279a of the first positioning tie 7278a, (iv) a fourth point of rotation R₄ at the second joint 7279b of the first positioning tie 7278a, (v) a fifth point of rotation R₅ at the first joint 7279a of the second positioning tie 7278b, and (vi) a sixth point of rotation R₆ at the second joint 7279b of the second positioning tie 7278b. The points of rotation R₁₋₆ can be constrained to move partially around one axis, two axes, and/or three axes, or can be entirely movable around one axis, two axes, and/or three axes. In some embodiments, the spinal fixation system 7210 can have more or fewer of the distances d₁₋₄ and/or the points of rotation R₁₋₆. For example, one or both of the tulips 7275 can be fixed to the heads 7274 such that the spinal fixation system 7210 does not include the first and second points of rotation R₁₋₂, the spinal fixation system 7210 can include only one of the positioning ties 7278 (e.g., the first positioning tie 7278a) such that the spinal fixation system 7210 does not include the fourth distance d₄ and the fifth and sixth points of rotation R₅₋₆, and so on.

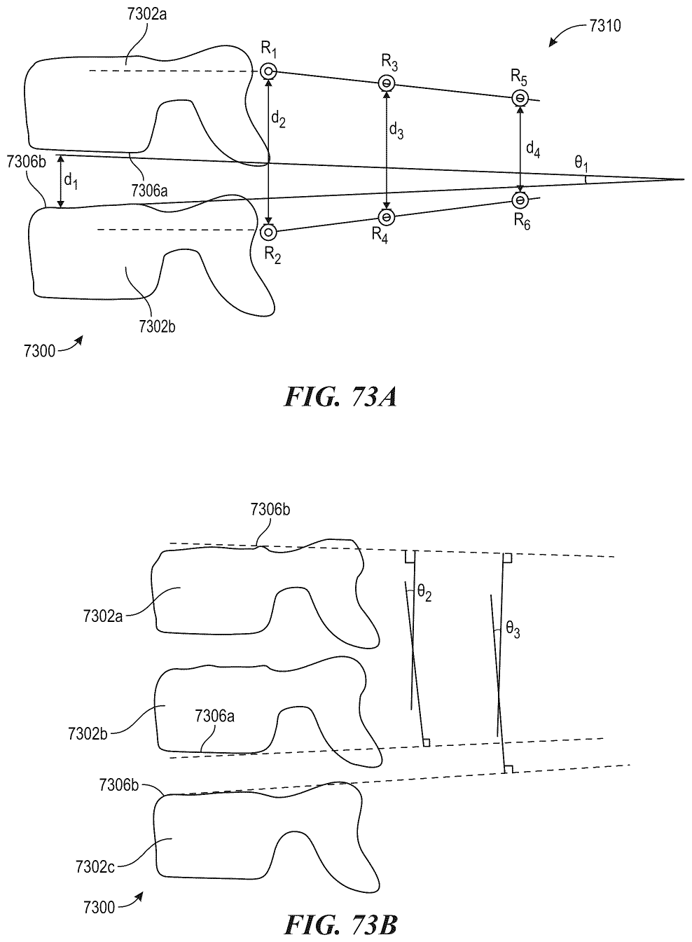

In some embodiments, a lordotic, kyphotic, and/or other spinal angle is desired between the two adjoining vertebrae 7202, across two non-consecutive vertebrae, or along a section of one or multiple vertebrae. FIG. 73A, for example, is a partially schematic side view (e.g., a lateral view) of the portion of the spinal fixation system 7210 attached to the spine 7200 in accordance with embodiments of the present technology. In the illustrated embodiment, a lordotic angle θ₁ is defined between the upper vertebra 7202a and the lower vertebra 7202b. More specifically, the lordotic angle θ₁ can be defined between a lower (e.g., inferior) surface or endplate 7306a of the upper (e.g., superior) vertebra 7202a and an upper (e.g., superior) surface or endplate 7306b of the lower (e.g., inferior) vertebra 7202b. FIG. 73B is a side view (e.g., a lateral view) of a portion of the spine 7200 further illustrating an additional lower vertebra 7202c below the lower vertebra 7202b in accordance with embodiments of the present technology. In the illustrated embodiment, a lordotic angle θ₂ can alternatively or additionally be defined between a projection along/onto the sagittal plane of an upper (e.g., superior) surface or endplate 7306b of the upper vertebra 7202a and the projection along the sagittal plane of a lower (e.g., inferior) surface or endplate 7306a of the adjacent lower vertebra 7202b. A lordotic angle θ₃ can alternatively or additionally be defined between the projection along the sagittal plane of the upper surface 7306b of the upper vertebra 7202a and the projection along the sagittal plane of an upper (e.g., superior) surface or endplate 7306b of the lower vertebra 7202c. In other embodiments, the lordotic angle can be defined in other manners. In some embodiments, the lordotic angle is determined preoperatively before a spinal surgical procedure, such as via measurements of a preoperative scan of the spine 7200 (e.g., a computed tomography (CT) scan, a magnetic resonance imaging (MRI) scan, and/or the like).

Referring to FIGS. 72A-73B together, some of the various distances d₁₋₄ and/or the points of rotation R₁₋₆ of the spinal fixation system 7210 can be manipulated to affect other ones of the distances d₁₋₄ and/or the points of rotation R₁₋₆ to, for example, change a lordotic angle (e.g., any or all of the lordotic angles θ₁₋₃) and/or another spinal angle (e.g., kyphotic, scoliotic) of the spine 7200. More specifically, some or all of the various distances d₁₋₄ and/or the points of rotation R₁₋₆ can be actively manipulated to achieve a spinal angle correction, some or all of the various distances d₁₋₄ and/or the points of rotation R₁₋₆ can be fixed (e.g., unable to change in distance and/or orientation) during manipulation of the spinal fixation system 7210, and/or some or all of the various distances d₁₋₄ and/or the points of rotation R₁₋₆ can be free to move and driven by the active manipulation of the spinal fixation system 7210. For example, the lordotic angles θ₁₋₃ can be adjusted by adjusting the first distance d₁ relative to second distance d₂, adjusting the second distance d₂ relative to the first distance d₁, and/or adjusting both of the first distance d₁ and the second distance d₂ in relation to each other. Accordingly, a general goal of the spinal fixation system 7210 can be defined as changing the relative sizes of the first and/or second distances d₁₋₂ to change the lordotic angle of the spine 7200. While focus is drawn herein to adjusting the lordotic angle of the spine 7200, one of ordinary skill in the art will understand that the spinal fixation system 7210 can be manipulated similarly in different planes (e.g., coronal, sagittal, etc.) to achieve correction of other spinal angles (e.g., kyphotic, scoliotic) of the spine 7200. For example, for coronal adjustment of the spine 7200 one side of the spinal fixation system 7210 can be locked while the other side is adjusted for distraction and/or angle change of the spine 7200.

As a first example, the spinal fixation system 7210 can be configured in accordance with the embodiments described in detail with reference to FIGS. 3G-3K. In such embodiment, the spinal fixation system 7210 can include only one of the positioning ties 7278 (e.g., the first positioning tie 7278a) such that the spinal fixation system 7210 does not include the fourth distance d₄ and the fifth and sixth points of rotation R₅₋₆. The first positioning tie 7278a can be a rigid clamp or locking device (e.g., the locking device 378 of FIGS. 3G-3K) such that third and fourth points of rotation R₃₋₄ are fixed (e.g., omitted). Further, the second and third distances d₂₋₃ can be fixed. Accordingly, during a spinal surgical procedure, the intervertebral device 7240 can be expanded to affect/manipulate the first distance d₁. Manipulation of the first distance d₁ via the intervertebral device 7240 causes a corresponding change in the first and second points of rotation R₁₋₂ via the rotation of the heads 7274 of the fixation members 7272 within the tulips 7275 as the screw bodies 7273 move with the vertebrae 7202. A change in the lordotic angle can be determined by measuring a change in rotation of the heads 7274 within the tulips 7275 mechanically, optically, electronically, and/or the like. The actual lordotic angle can be determined by mapping the change in angle to the original lordotic angle (e.g., pre-manipulation of the spinal fixation system 7210) as, for example, determined preoperatively via measurements of a preoperative scan of the spine 7200. The spinal fixation system 7210 can be manipulated similarly while additionally including the second positioning tie 7288b—for example, with the fifth and sixth points of rotation R₅₋₆ fixed and the fourth distance d₄ fixed.

As a second example, the third and/or fourth distances d₃₋₄ can be manipulated to change the first and/or second distances d₁₋₂ to change the lordotic angle. For example, the third distance d₃ and/or the fourth distance da can be decreased to correspondingly increase the first and/or second distances d₁₋₂.

As a third example, the first, third, and fifth points of rotation R_{1, 3, 5} are along a first rigid structure comprising the first tulip 7275a and the first tower member 7284a, and the second, fourth, and sixth points of rotation R_{2, 4, 6} are along a second rigid structure comprising the second tulip 7275b and the second tower member 7284b. The two rigid structures can be constrained to be within the same plane, or can be in different spatial planes. One or both of the positioning ties 7278 can be actuatable to adjust the third distance d₃ and/or the fourth distance d₄ between the first and second rigid structures. A third positioning tie (not shown) can extend between the first and second rigid structures (e.g., the tulips 7275) at the first and second points of rotation R₁ and R₂ and can be actuatable (e.g., by a user) to control the distance d₂. The first positioning tie 7278a, the second positioning tie 7278b, and/or the third positioning tie can comprise a linear motion mechanism for changing the second through fourth distances d₂₋₄, such as rack and pinion gears, worm drives, toggle arms, threaded rods, rods with clamps, and/or the like. While keeping each of the points of rotation R₁₋₆ free to rotate, the user can set the distances d₃ and da via the first and second positioning ties 7278a-b, respectively, to change (e.g., establish) the second distance d₂. In establishing the second distance d₂ relative to the first distance d₁, the user can configure the lordotic angle (e.g., any or all of the lordotic angles θ₁₋₃) to adjust lordosis and/or to configure another spinal angle.

As a fourth example, the first distance d₁ can initially be fixed, and the second distance d₂, the third distance d₃, and/or the fourth distance da can be mobilized to change the lordotic angle (e.g., any or all of the lordotic angles θ₁₋₃). As a fifth example, the second distance d₂ can initially be fixed, and the first distance d₁, the third distance d₃, and/or the fourth distance d₄ can be mobilized to change the lordotic angle (e.g., any or all of the lordotic angles θ₁₋₃). As a sixth example, the third distance d₃ can initially be fixed, and the first distance d₁, the second distance d₂, and/or the fourth distance da can be mobilized to change the lordotic angle (e.g., any or all of the lordotic angles θ₁₋₃). As a seventh example, the fourth distance da can initially be fixed, and the first distance d₁, the second distance d₂, and/or the third distance d₃ can be mobilized to change the lordotic angle (e.g., any or all of the lordotic angles θ₁₋₃).

In some embodiments, the various structures/components of the spinal fixation system 7210 can be coupled to, attached to, and/or integrated into retraction, compression, and/or distraction apparatuses as a means for user control some or all of the various distances d₁₋₄ and/or the points of rotation R₁₋₆.

In some embodiments, the various structures/components of the spinal fixation system 7210 can be coupled to, attached to, and/or integrated into spinal implants and their associated instrumentation. For example, in the illustrated embodiment the first and second points of rotation R₁₋₂ comprise the polyaxial heads of pedicle screws (e.g., the heads 7274 rotatable within the tulips 7275). In some embodiments, a rigid structure that contains the first, third, and fifth points of rotation R_{1, 3, 5} comprises part of a tulip (e.g., the first tulip 7275a) attached to a pedicle screw (e.g., the first fixation member 7272a). In some embodiments, a rigid structure that contains the second, fourth, and sixth points of rotation R_{2, 4, 6} comprises part of a tulip (e.g., the second tulip 7275b) attached to a pedicle screw (e.g., the second fixation member 7272b). In some embodiments, a rigid structure that contains the first, third, and fifth points of rotation R_{1, 3, 5} comprises part of a percutaneous tower (e.g., the first tower member 7284a) used for the implantation of a minimally-invasive pedicle screw (e.g., the first fixation member 7272a). In some embodiments, a rigid structure that contains the second, fourth, and sixth points of rotation R_{2, 4, 6} comprises part of a percutaneous tower (e.g., the second tower member 7284a) used for the implantation of a minimally-invasive pedicle screw (e.g., the second fixation member 7272b).

In some embodiments, the desired distances d₁₋₄ and/or the points of rotation R₁₋₆ are achieved while some or all the distances d₁₋₄ and/or the points of rotation R₁₋₆ are free to move. When the desired configuration is reached, some or all of the distances d₁₋₄ and/or the points of rotation R₁₋₆ can be locked to maintain the configuration. In some embodiments, once the desired configuration is achieved and optionally locked, some or all the various structures/components of the spinal fixation system 7210 are removed from the patient—for example, the tower members 7284. In some embodiments, once the desired configuration is achieved and optionally locked, none of the various structures/components of the spinal fixation system 7210 are removed from the patient and remain as an implanted device.

In some embodiments, the second distance d₂ can be fixed while still allowing for rotation of the tulips 7275 about the first and second points of rotation R₁₋₂ (e.g., while still allowing for polyaxiality of the tulips 7275 about the heads 7274) to, for example, allow for manipulation of the tower members 7284 and the positioning ties 7278 to define the lordotic angle. For example, a notched spinal rod can be inserted between the fixation members 7272 to fix the second distance d₂ between the fixation members 7272, while still allowing for rotation of the tulips 7275 about the first and second points of rotation R₁₋₂. In other embodiments, the second distance d₂ can be fixed by inserting a wedged component that adopts geometry of the void in between two fixation members 7272 to fix the second distance d₂.

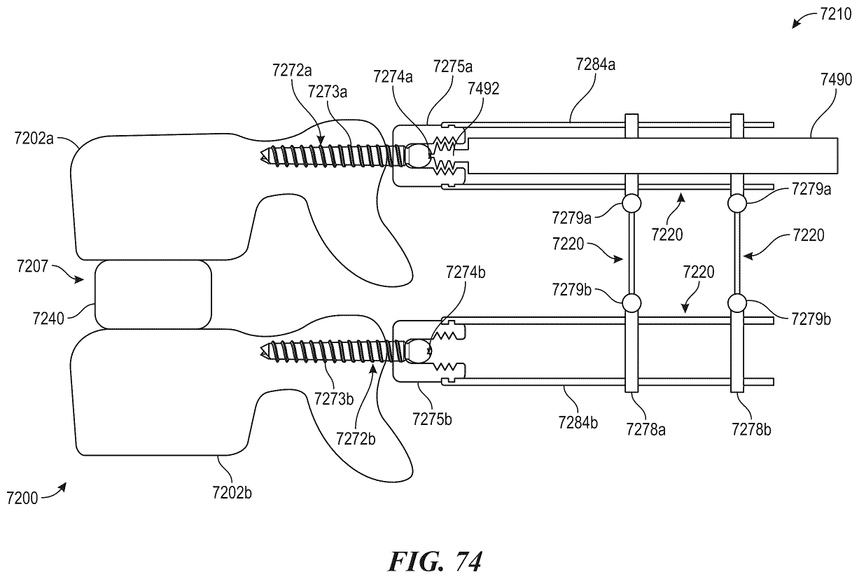

In other embodiments it can be desirable to fix rotation of the tulips 7275 about the first and second points of rotation R₁₋₂, while still allowing for manipulation of second distance d₂. FIG. 74, for example, is an identical side view (e.g., a lateral view) of the portion of the spinal fixation system 7210 attached to the spine 7200 of the patient of FIG. 72A illustrating an additional driver 7490 inserted through the first tower member 7284a and engaging the first fixation member 7272a in accordance with embodiments of the present technology. The driver 7490 can have a keyed distal tip portion 7492 configured to engage the first head 7274a (e.g., a screw thread thereof) and the first tulip 7275a (e.g., threads thereof) to inhibit or even prevent rotation therebetween (e.g., about the first point of rotation R₁; FIG. 72B). In some embodiments, the driver 7490 can also be used to manipulate the third and fourth distances d₃ and d₄ (FIG. 72B). In some embodiments, a separate driver can similarly be inserted through the second tower member 7284b to engage the second screw head 7274b and the second tulip 7275b.

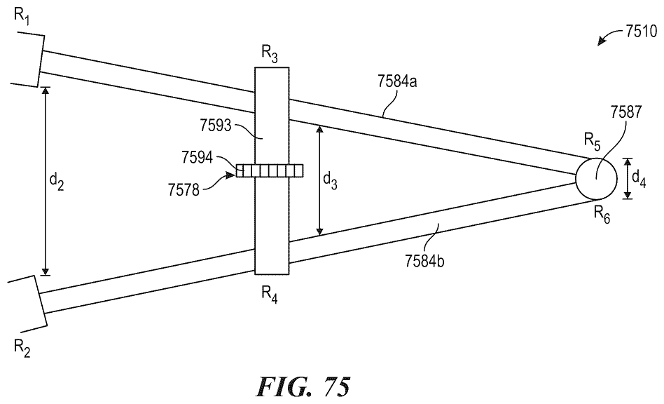

FIG. 75 is a side view of a posterior spinal fixation instrument/system 7510 in accordance with additional embodiments of the present technology. The spinal fixation system 7510 include some features generally similar or identical in structure and/or function to the spinal fixation system 7210 described in detail above with reference to FIGS. 72A-74 and/or elsewhere herein. In the illustrated embodiment, the spinal fixation system 7510 comprises a first rigid structure 7584a, a second rigid structure 7584b, a rotatable/pivotable articulation 7587 joining a proximal portion of the first rigid structure 7584a to a proximal portion of the second rigid structure 7584b, and a positioning tie 7578 extending between and coupling the first and second rigid structures 7584a-b distal to the articulation 7587. In some embodiments, the positioning tie 7578 comprises a threaded rod 7593 coupled to an actuator 7594 (e.g., a screw wheel).

The spinal fixation system 7510 can define/comprise the same distances d₂₋₄ and/or the points of rotation R₁₋₆ as described in detail above with reference to FIGS. 72A-74. In the illustrated embodiment, the fourth distance da is fixed within the articulation 7587 and the fifth and sixth points of rotation R₅₋₆ comprise the same point of rotation. The second distance d₂ can be controlled/changed by actuating the actuator 7594 to move the threaded rod 7593 to change (e.g., increase, decrease) the third distance d₃.

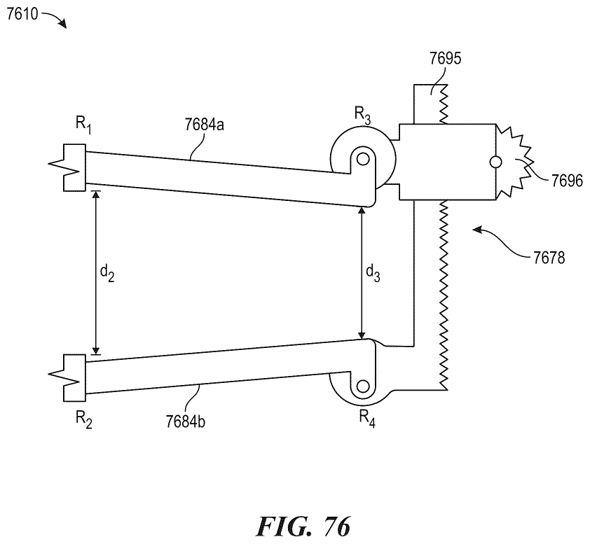

FIG. 76 is a side view of a posterior spinal fixation instrument/system 7610 in accordance with additional embodiments of the present technology. The spinal fixation system 7610 include some features generally similar or identical in structure and/or function to the spinal fixation system 7210 described in detail above with reference to FIGS. 72A-74, the spinal fixation system 7510 described in detail above with reference to FIG. 75, and/or elsewhere herein. In the illustrated embodiment, the spinal fixation system 7610 comprises a first rigid structure 7684a, a second rigid structure 7684b, a positioning tie 7678 coupled to the first and second rigid structures 7684a-b and comprising a rack 7695 and a pinion 7696 mechanism. More particularly, a proximal portion of the first rigid structure 7684a can be rotatably or fixedly coupled to the pinion 7696, and a proximal portion of the second rigid structure 7684b can be rotatably or fixedly coupled to the rack 7695.

The spinal fixation system 7610 can define/comprise the same second and third distances d₂₋₃ and/or the points of rotation R₁₋₄ as described in detail above with reference to FIGS. 72A-75. In some embodiments, the second distance d₂ is controlled/changed by actuating the rack 7695 and the pinion 7696 mechanism while the third and fourth points of rotation R₃₋₄ are locked. In other embodiments, the second distance d₂ is controlled/changed by locking the third distance d₃ using the rack 7695 and the pinion 7696 mechanism and then changing the third and/or fourth points of rotation R₃₋₄.

In some embodiments, a spinal fixation system in accordance with embodiments of the present technology can include one or more mechanisms for measuring/capturing/determining a change in lordotic and/or spinal angles (e.g., kyphotic angle, scoliotic angle). Such mechanisms can verify and validate effects of the spinal fixation system and provide real time or near real time quantifiable data to a user (e.g., surgeon) of the spinal fixation system. Such mechanisms can include, for example, mechanical, electrical encoding, and/or optical encoding mechanisms. Referring to FIGS. 72A and 72B, such mechanisms can be implemented in/on any or all of the fixation members 7272, the tower members 7284, and/or the positioning ties 7278, and/or such mechanisms can be embedded in the intervertebral device 7240.

In some embodiments, a mechanical mechanism to measure changes in lordotic and/or other spinal angles can measure the changes in angle via a ratcheting mechanism that captures incremental changes in lordotic angle as d₁ and d₂ are defined via manipulation of spinal fixation system 7210. In other embodiments, a mechanical mechanism can utilize a dial indicator that captures changes in lordotic angle via use of a plunger that captures changes in any or all of the distances d₁₋₄, which is then translated to angle measurements.

In some embodiments, an electrical encoding mechanism to measure changes in lordotic and/or other spinal angles can utilize a small circuit with strain gauges that can be used to capture changes in lordotic angle and/or other spinal angles as the tower members 7284, the positioning ties 7278, the fixation members 7272, and/or the intervertebral device 7240 are manipulated. In other embodiments, electrical encoding feedback can be gathered by implementing a software code that plots pressure-volume and/or pressure-height curves during manipulation (e.g., expansion) of the intervertebral device 7240 to generate the first distance d₁ against a calibration curve to relate an anterior edge distance (e.g., the first distance d₁) and/or a posterior edge distance (e.g., the second distance d₂) with the lordotic angle (e.g., the lordotic angle θ₁). Data captured to generate such curves can be achieved by use of pressure sensors and/or or electrical sensors that alert the user of real time or near real time pressure during manipulation of the intervertebral device 7240 to define d₁. In other embodiments, electrical encoding feedback can be gathered by implementing a balloon with known pressure-volume and/or pressure-height curves, and tracking an inflation volume of the balloon within the intervertebral device 7240, which can be used to correlate anterior expansion to changes in the lordotic angle.

In some embodiments, an optical encoding mechanism to measure changes in lordotic and/or other spinal angles can utilize fiducial markers that are placed on the tower members 7284 to calculate changes in lordotic angle and/or other spinal angles via image and processing techniques. In other embodiments, optical encoding feedback can be gathered by implementing a light sensor that casts a shadow on a dial that is attached to the fixation members 7272 to capture changes in lordotic angle and/or other spinal angles.

As described above, the actual lordotic angle can be determined by mapping the change in angle determined by a mechanical, electrical encoding, optical encoding, and/or other measurement mechanism to the original lordotic angle as, for example, determined preoperatively via measurements of a preoperative scan of the spine 7200.

In some embodiments, one or more sensors can be attached to the vertebral body (not necessarily via a pedicle screw) to measure the lordotic angle and/or a change in height between vertebrae. The sensors can each comprise (i) a pin (e.g., a caspar pin) positioned on the spinous process of a vertebrae or anywhere else on the bony anatomy and (ii) a sensing system attached to, embedded in, integrated in, and/or otherwise coupled to the pin. The sensing system can comprise an inertial motion sensor, a gyroscope, a tilt meter, an electromagnetic marker/fiducial, an infrared marker/fiducial, and/or the like. For example, a sensor (e.g., the pin thereof) can be coupled to each of the pair of vertebrae adjacent a disc space. The sensors can each comprise an inertial measurement sensor configured to measure motion, and the relative motions of the two sensors can be used to derive changes to height and lordotic angle. As another example, the sensors can comprise infrared and/or electromagnetic fiducials than can be read/tracked by a navigation system to detect changes to height and lordotic angle.

As yet another example, one or more of the sensors can comprise an emitting array and one or more of the sensors can comprise a receiving array. The emitting array can be configured to emit sound, light, electromagnetic waves, and/or other signals, and the receiving array can receive/detect the emitted sound, light, electromagnetic waves, and/or other signals. One of the sensors with an emitting array can be coupled to one of a pair of vertebrae adjacent a disc space, and one of the sensors with a receiving array can be coupled to the other of the pair of vertebrae adjacent the disc space. The sensors can be coupled to a processor configured to utilize time-of-flight, relative distance, changes in capacitance, triangulation, and/or other processing techniques to determine relative positions of the emitting and receiving arrays to determine changes in intervertebral height and angle. Multiple emitting arrays or a single emitting array can be used. Likewise, multiple receiving arrays or a single receiving array can be used.

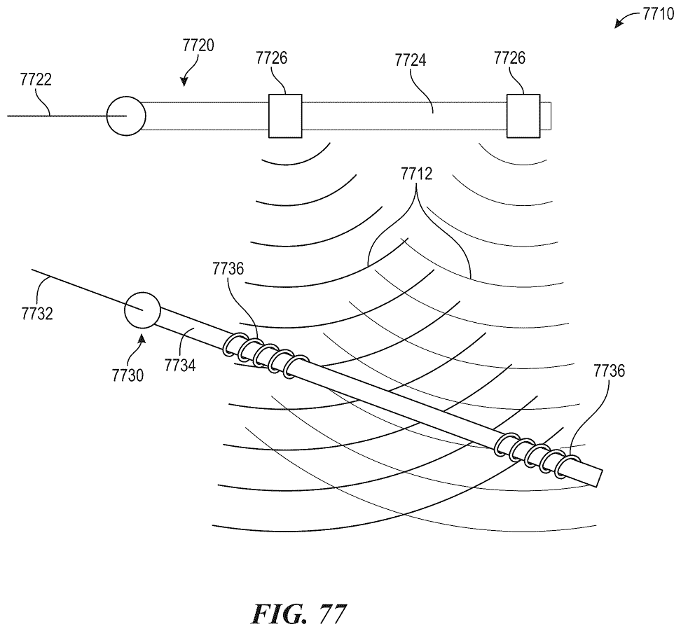

FIG. 77, for example, is a side view (e.g., a lateral view) of a portion of a spinal position sensing system 7710 configured to be attached to a spine of a patient in accordance with embodiments of the present technology. In the illustrated embodiment, the spinal positions sensing system 7710 includes a first sensor 7720 and a second sensor 7730. The first sensor 7720 can include (i) a pin 7722 configured to be secured to a vertebra or other rigid structure of the patient and (ii) a transmitting array 7724 coupled to the pin 7722 and having one or more transmitting elements 7726. The second sensor 7730 can include (i) a pin 7732 configured to be secured to a vertebra or other rigid structure of the patient and (ii) a receiving array 7734 coupled to the pin 7732 and having one or more receiving elements 7736. The transmitting elements 7726 of the transmitting array 7724 can generate signals 7712 (e.g., light, sound, electromagnetic, and/or other waves). Accordingly, the transmitting elements 7726 can comprise light-emitting diodes (LEDs), speakers, an electromagnetic wave generator, and/or the like. The receiving elements 7736 of the receiving array 7734 can be positioned to receive the signals 7712 and, for example, convert the signals to electric signals. Accordingly, the receiving elements 7736 can comprise photovoltaic cells, microphones, and/or the like. The spinal position sensing system 7710 can further include a processor coupled to the first and second sensors 7720, 7730 and configured to utilize time-of-flight, relative distance, changes in capacitance, triangulation, and/or other processing techniques to process data from the transmitting and receiving elements 7726, 7736 to determine relative positions of the first and second sensors 7720, 7730 to determine changes in intervertebral height and angle. The transmitting elements 7726 can be configured to transmit the same type of signals (e.g., with a common frequency, amplitude, mode, etc.) or can transmit different types of signals. The receiving elements 7736 can be tuned to receive signals from one or more of the transmitting elements 7726.

### XIII. SELECTED EMBODIMENTS OF ROBOTIC INTEGRATION

The systems and devices described in detail herein are suitable for integration within a robotic system. That is, some or all of the various components can be coupled to a robot configured to move, translate, rotate, torque, deploy, etc., the component. More specifically, such a robot can provide a planned trajectory for the various components. As one example, referring to FIGS. 1A-1R, the trocar 110 can be coupled to robot configured to insert the trocar 110 along a planned trajectory into the disc space 101, the discectomy device 120 can be coupled to the same or a different robot configured to insert the discectomy device 120 through the trocar 110 and actuate the discectomy device 120 within the disc space 101 to remove/disrupt the diseased disc 104a, the inner balloon shaft 132 can be coupled to the same or a different robot configured to insert the inner balloon shaft 132 and the first balloon 130 through the trocar 110 and/or to inflate the first balloon 130, the one or more balloon shafts 152 can be coupled to the same or a different robot configured to insert the one or more balloon shafts 152 and the second balloon 150 through the trocar 110 and/or to inflate the second balloon 150, and so on.

### XIV. SELECTED EMBODIMENTS OF INTERVERTEBRAL DEVICES, SYSTEMS, AND METHODS FOR USE DURING OPEN AND/OR PARTIALLY OPEN SURGICAL PROCEDURES

Although many of the embodiments described above are described in the context of minimally invasive spinal surgical procedures, many of the devices, systems, and methods described herein can be used in various other spinal surgical procedures, such open or at least partially open spinal surgical procedures. For example, during an open spinal surgical procedure, the muscles and soft tissue around a portion of a spine of a patient can be moved to at least partially expose the portion of the spine of the patient. A surgeon can then access the spine to, for example, remove a diseased disc and fix a posterior fixation assembly to the spine. In some embodiments, a trocar in accordance with embodiments of the present technology can be used to deploy a balloon (e.g., for lordosis and/or distraction) and/or an intervertebral device into a disc space of the removed disc as described in detail above. The trocar can be coupled to the posterior fixation assembly during the procedure via a connector guide member. In some embodiments, the connector guide member is configured to be secured to a tower member of the posterior fixation assembly, and the tower member can provide for polyaxiality or at least one degree of freedom to move the trocar relative to the portion of the spine and align the trocar with the disc space. In some embodiments, the connector guide member is configured to be secured to a spanning member (e.g., a rod) of the posterior fixation assembly, and the connector guide member can provide for polyaxiality or at least one degree of freedom to move the trocar relative to the portion of the spine and align the trocar with the disc space.

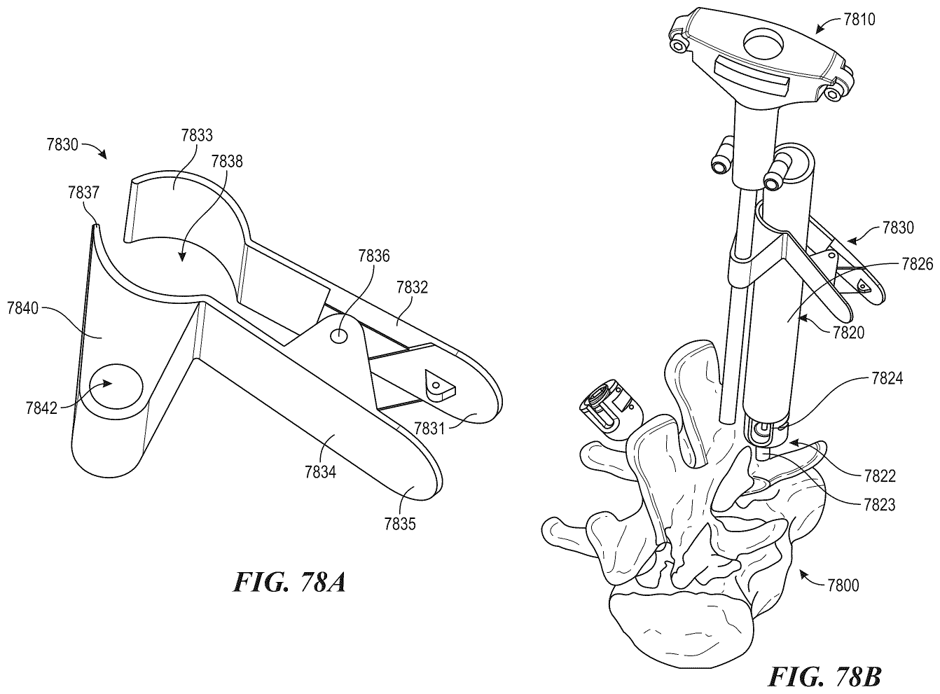

FIGS. 78A-81B illustrate various connector guide members in accordance with the present technology that are configured to secure a trocar to a tower member of a posterior fixation assembly. In some aspects of the present technology, tower members can have several universal sizes (e.g., diameters) and a connector guide member can be adjustably secured to tower members of different sizes. In contrast, there are many fixation members having different shapes and arrangements that are commonly used during spinal surgical procedures (e.g., produced by different manufacturers), and to which tower members may be secured to. Accordingly, by securing the connector guide member to a tower member rather than a fixation member of a posterior fixation assembly, the connector guide member can more easily be used with a wide variety of posterior fixation assemblies.

FIG. 78A is an isometric view of a connector guide member 7830 in accordance with embodiments of the present technology. FIG. 78B is an isometric view of the connector guide member 7830 of FIG. 78A coupling a trocar 7810 to a spinal fixation system 7820 (e.g., a posterior fixation assembly) attached to a portion of a spine 7800 of a patient in accordance with embodiments of the present technology. Referring to FIG. 78A, the connector guide member 7830 can be a spring clip having a first arm 7832 pivotably coupled to a second arm 7834 at a pivot joint 7836. The first arm 7832 can include a first end portion 7831 and a second end portion 7833, and the second arm 7834 can include a first end portion 7835 and a second end portion 7837. The second end portion 7833 of the first arm 7832 and the second end portion 7837 of the second arm 7834 can together define an opening or lumen 7838. In some embodiments, a biasing member (e.g., a torsion spring) is positioned between the first and second arms 7832, 7834 to bias (i) the first end portion 7831 of the first arm 7832 away from the first end portion 7835 of the second arm 7834 and (ii) the second end portion 7833 of the first arm 7832 toward the second end portion 7837 of the second arm 7834 (e.g., thereby decreasing a cross-sectional dimension of the lumen 7838). In some embodiments, the second arm 7834 includes a coupling portion 7840 defining a through-hole 7842 (e.g., an opening, a lumen).

Referring to FIG. 78B, the spinal fixation system 7820 can include a fixation member 7822, such as a pedicle screw, having a screw body 7823 secured to the spine 7800 (e.g., a vertebra thereof) and a polyaxial head or tulip 7824 rotatably coupled to the screw body 7823. In the illustrated embodiment, the spinal fixation system 7820 further includes a tower member 7826 releasably coupled to the tulip 7824. Referring to FIGS. 78A and 78B, the connector guide member 7830 can be releasably coupled to the tower member 7826 by, for example, (i) moving (e.g., squeezing by a user) the first end portion 7831 of the first arm 7832 toward the first end portion 7835 of the second arm 7834 to move the second end portion 7833 of the first arm 7832 away from the second end portion 7837 of the second arm 7834 against the biasing force of the biasing member, (ii) positioning the tower member 7826 within the lumen 7838, and (iii) releasing the first and second arms 7832, 7834 such that the biasing member biases the first and second arms 7832, 7834 to clamp the tower member 7826 within the lumen 7838.

A trocar 7810 can be inserted through the through-hole 7842 such that the trocar 7810 is fixed to move with the tower member 7826. That is, the connector guide member 7830 provides a rigid coupling of the trocar 7810 to the tower member 7826. The tower member 7826 can be moved (e.g., pivoted relative to the tulip 7824) to adjust the position of the trocar 7810 relative to the spine 7800. Accordingly, the tower member 7826 can provide for polyaxiality or at least one degree of freedom to move the trocar 7810 relative to the spine 7800 and align the trocar 7810 with a disc space for routing one or more balloons, intervertebral devices, and/or the like through the trocar 7810 to treat the disc space. The connector guide member 7830 can be released from the tower member 7826 by (i) moving (e.g., squeezing by a user) the first end portion 7831 of the first arm 7832 toward the first end portion 7835 of the second arm 7834 to move the second end portion 7833 of the first arm 7832 away from the second end portion 7837 of the second arm 7834 and (ii) removing the tower member 7826 from within the lumen 7838.

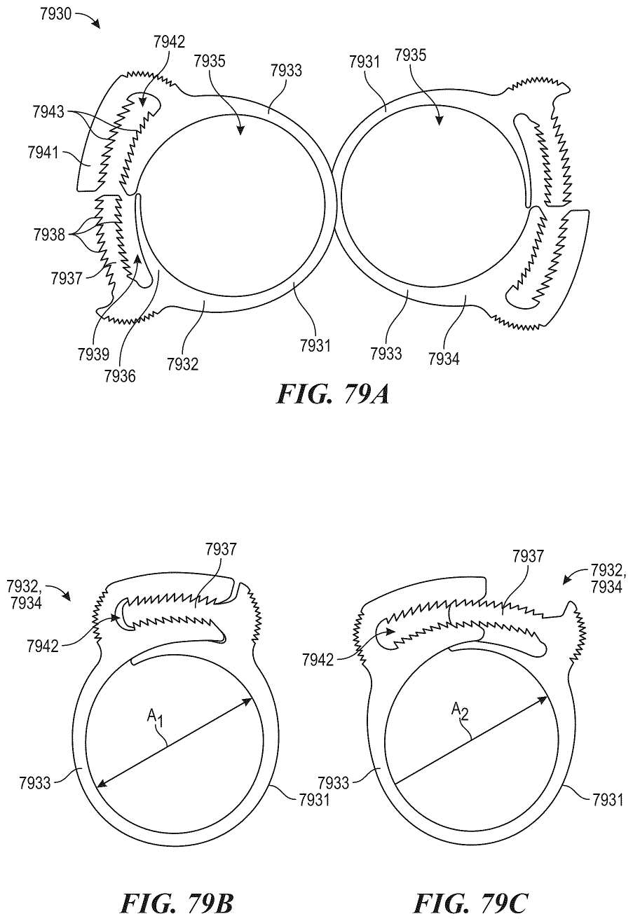

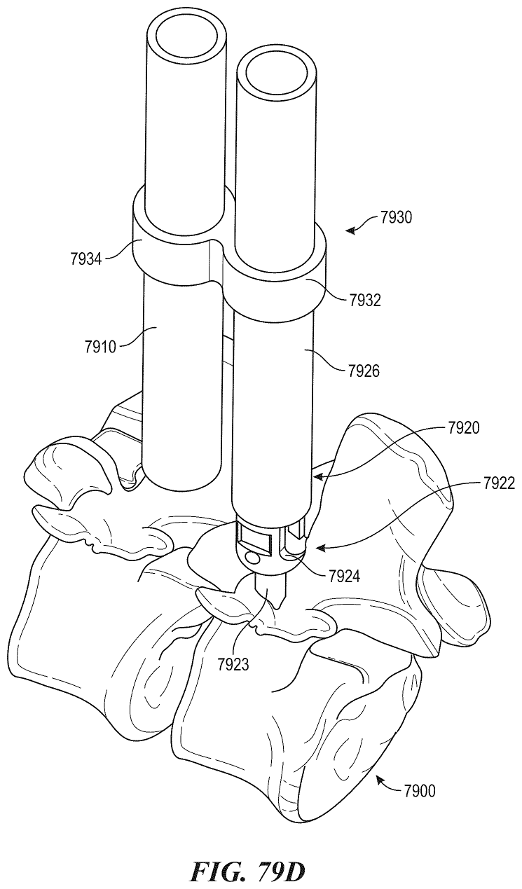

FIG. 79A is a top view of a connector guide member 7930 in accordance with embodiments of the present technology. The connector guide member 7930 can be a hose clamp having a first clamp member 7932 fixed to a second clamp member 7934 member. In some embodiments, the first and second clamp members 7932, 7934 can be identical (e.g., including the same components and/or sizes). For example, in the illustrated embodiment the first and second clamp members 7932, 7934 each include a first arm 7931 and a second arm 7933 together defining a lumen 7935 (e.g., a generally circular lumen). The first and second arms 7931, 7933 can be flexible and formed of, for example, plastic. The first arms 7931 can include a first clamp portion 7936 adjacent to the lumen 7935 and a first locking portion 7937 positioned radially outside the first clamp portion 7936. The first locking portion 7937 can include/define a plurality of teeth 7938 along a length thereof. The first clamp portion 7936 and the first locking portion 7937 can define a first channel 7939 therebetween. The second arms 7931 can similarly include a second clamp portion 7940 adjacent the lumen 7935 and a second locking portion 7941 positioned radially outside the second clamp portion 7940. The second clamp portion 7940 and the second locking portion 7941 can define a second channel 7942 therebetween. The second clamp portion 7940 and the second locking portion 7941 can include/define a plurality of teeth 7943 along a length thereof adjacent the second channel 7942.

The first arm 7931 of each of the first and second clamp members 7932, 7934 can be secured to the second arm 7933 by inserting the first locking portion 7937 into the second channel 7942. The teeth 7938 of the first locking portion 7937 can engage the teeth 7943 along the second channel 7942 to inhibit the first arm 7931 from moving away from the second arm 7933. More specifically, FIGS. 79B and 79C are side views of one of the first and second clamp members 7932, 7934 in a fully engaged position and a partially engaged position, respectively, in accordance with embodiments of the present technology. Referring to FIGS. 79A and 79B, the first locking portion 7937 of the first arm 7931 is fully inserted into the second channel 7942 of the second arm 7933 with the teeth 7938, 7943 engaging one another. In this position, (i) the engagement of the teeth 7938, 7943 inhibits the first locking portion 7937 from being withdrawn from the second channel 7942 (e.g., fixing the first arm 7931 to the second arm 7933) and (ii) the lumen 7935 can have a first (e.g., minimum) cross-sectional dimension A₁ (e.g., diameter, area, etc.). Referring to FIGS. 79A and 79C, the first locking portion 7937 of the first arm 7931 is partially inserted into the second channel 7942 of the second arm 7933 with the teeth 7938, 7943 engaging one another (e.g., three of the teeth 7938, 7943 engaging one another). In this position, (i) the engagement of the teeth 7938, 7943 inhibits the first locking portion 7937 from being withdrawn from the second channel 7942 (e.g., fixing the first arm 7931 to the second arm 7933) and (ii) the lumen 7935 can have a second cross-sectional dimension A₂ (e.g., diameter, area, etc.) greater than the first cross-sectional dimension A₁. By adjusting how far the locking portion 7937 is inserted into the second channel 7942, the connector guide member 7930 can be sized to engage and couple to tower members of different dimension (e.g., diameter). Referring to FIGS. 79A-79C, the first arm 7931 can be decoupled from the second arm 7933 by flexing the first arm 7931 and/or the second arm 7933 laterally (e.g., in a direction into and/or out of the page) such that the first locking portion 7937 slides out of the second channel 7942.

FIG. 79D is an isometric view of the connector guide member 7930 of FIGS. 79A-79C coupling a trocar 7910 to a spinal fixation system 7920 (e.g., a posterior fixation assembly) attached to a portion of a spine 7900 of a patient in accordance with embodiments of the present technology. In the illustrated embodiment, the spinal fixation system 7920 includes a fixation member 7922, such as a pedicle screw, having a screw body 7923 secured to the spine 7900 (e.g., a vertebra thereof) and a polyaxial head or tulip 7924 rotatably coupled to the screw body 7923. In the illustrated embodiment, the spinal fixation system 7920 further includes a tower member 7926 releasably coupled to the tulip 7924. Referring to FIGS. 79A-79D, the connector guide member 7930 can be releasably coupled to the tower member 7926 by, for example, (i) flexing the first arm 7931 and the second arm 7933 of the first clamp member 7932 away from one another and positioning the tower member 7926 within the lumen 7935 (and/or sliding the first clamp member 7932 over the tower member 7926) and then (ii) pressing the first locking portion 7937 into the second channel 7942 until the first clamp member 7932 is clamped to the tower member 7926. The lumen 7935 can sized by inserting more of less of the first locking portion 7937 into the second channel 7942 to match the size of the tower member 7926 and securely clamp the first clamp member 7932 to the tower member 7926. Likewise, the connector guide member 7930 can be releasably coupled to the trocar 7910 by, for example, (i) flexing the first arm 7931 and the second arm 7933 of the second clamp member 7934 away from one another and positioning the trocar 7910 within the lumen 7935 and then (ii) pressing the first locking portion 7937 into the second channel 7942 until the second clamp member 7934 is clamped to the trocar 7910. The lumen 7935 can sized by inserting more of less of the first locking portion 7937 into the second channel 7942 to match the size of the trocar 7910 and securely clamp the second clamp member 7932 to the trocar 7910.

In some aspects of the present technology, the connector guide member 7930 provides a rigid coupling of the trocar 7910 to the tower member 7926. The tower member 7926 can be moved (e.g., pivoted relative to the tulip 7924) to adjust the position of the trocar 7910 relative to the spine 7900. That is, the tower member 7926 can provide for polyaxiality or at least one degree of freedom to move the trocar 7910 relative to the spine 7900 and align the trocar 7910 with a disc space of the spine 7900 for routing one or more balloons, intervertebral devices, and/or the like through the trocar 7910 to treat the disc space. The connector guide member 7930 can be released from the tower member 7926 and the trocar 7910 by flexing the first arm 7931 and/or the second arm 7933 of the first and second clamp members 7932, 7934 laterally (e.g., in a direction into and/or out of the page in FIGS. 79A-79C) such that the first locking portion 7937 slides out of the second channel 7942.

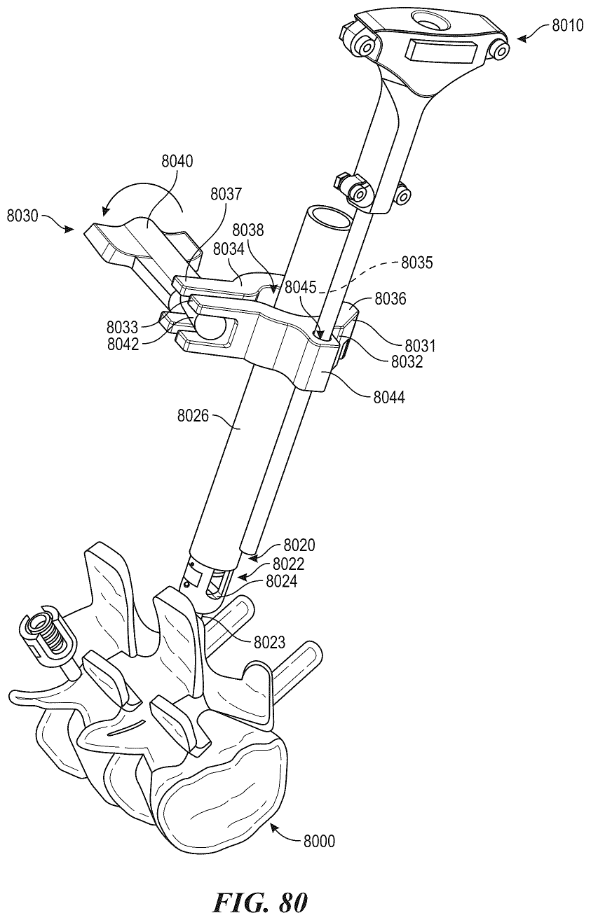

FIG. 80 is an isometric view of a connector guide member 8030 coupling a trocar 8010 to a spinal fixation system 8020 (e.g., a posterior fixation assembly) attached to a portion of a spine 8000 of a patient in accordance with embodiments of the present technology. In the illustrated embodiment, the spinal fixation system 8020 includes a fixation member 8022, such as a pedicle screw, having a screw body 8023 secured to the spine 8000 (e.g., a vertebra thereof) and a polyaxial head or tulip 8024 rotatably coupled to the screw body 8023. In the illustrated embodiment, the spinal fixation system 8020 further includes a tower member 8026 releasably coupled to the tulip 8024.

The connector guide member 8030 can be a pipe clamp having (i) a first arm 8032 having a first end portion 8031 and a second end portion 8033 and (ii) a second arm 8034 having a first end portion 8035 (obscured in FIG. 80) and a second end portion 8037. The first end portion 8031 of the first arm 8032 can be pivotably coupled to the first end portion 8035 of the second arm 8034 at a pivot joint 8036. The first and second arms 8032, 8034 can together define an opening or lumen 8038 configured to receive the tower member 8026. The connector guide member 8030 can further include an actuator 8040 coupled to a threaded rod 8042. The threaded rod 8042 can be fixedly coupled to the second end portion 8033 of the first arm 8032 and movably (e.g., threadably) coupled to the second end portion 8037 of the second arm 8034. Accordingly, rotation of the actuator 8040 in a first direction can rotate the threaded rod 8042 to move the second end portion 8037 of the second arm 8034 toward the second end portion 8033 of the first arm 8032 to decrease a cross-sectional dimension (e.g., diameter, area) of the lumen 8038, and rotation of the actuator 8040 in a second direction opposite the first direction can rotate the threaded rod 8042 to move the second end portion 8037 of the second arm 8034 away from the second end portion 8033 of the first arm 8032 to increase the cross-sectional dimension of the lumen 8038. In some embodiments, the first arm 8032 includes a coupling portion 8044 defining a through-hole 8045 (e.g., an opening, a lumen).

The connector guide member 8030 can be releasably coupled to the tower member 8026 by, for example, (i) positioning the tower member 8026 within the lumen 8038 of the connector guide member 8030 (e.g., by sliding the connector guide member 8030 over the tower member 8026) and (ii) rotating the actuator 8040 in the first direction to move the first and second arms 8032, 8034 toward one another to decrease the cross-sectional dimension of the lumen 8038 and to clamp the tower member 8026 therebetween. The trocar 8010 can be inserted through the through-hole 8045 such that the trocar 8010 is fixed to move with the tower member 8026. That is, the connector guide member 8030 can provide a rigid coupling of the trocar 8010 to the tower member 8026. The tower member 8026 can be moved (e.g., pivoted relative to the tulip 8024) to adjust the position of the trocar 8010 relative to the spine 8000. Accordingly, the tower member 8026 can provide for polyaxiality or at least one degree of freedom to move the trocar 8010 relative to the spine 8000 and align the trocar 8010 with a disc space of the spine 8000 for routing one or more balloons, intervertebral devices, and/or the like through the trocar 8010 to the disc space. The connector guide member 8030 can be released from the tower member 8026 by (i) rotating the actuator 8040 in the second direction to move the first and second arms 8032, 8034 away from one another to increase the cross-sectional dimension of the lumen 8038 and to loosen the coupling to the tower member 8026 and (ii) removing the tower member 8026 from the lumen 8038 of the connector guide member 8030 (e.g., by sliding the connector guide member 8030 off of the tower member 8026).

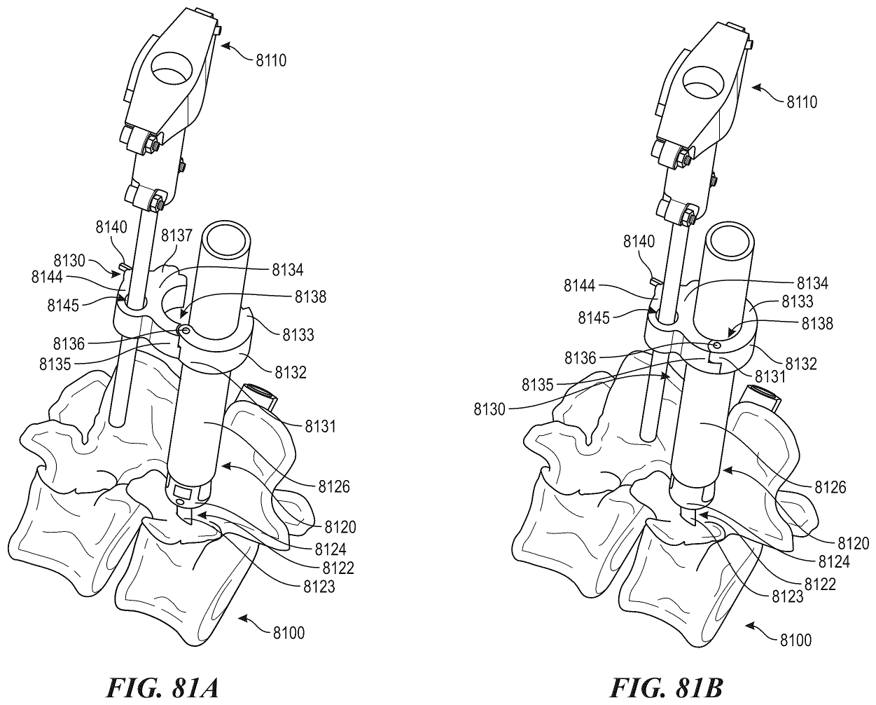

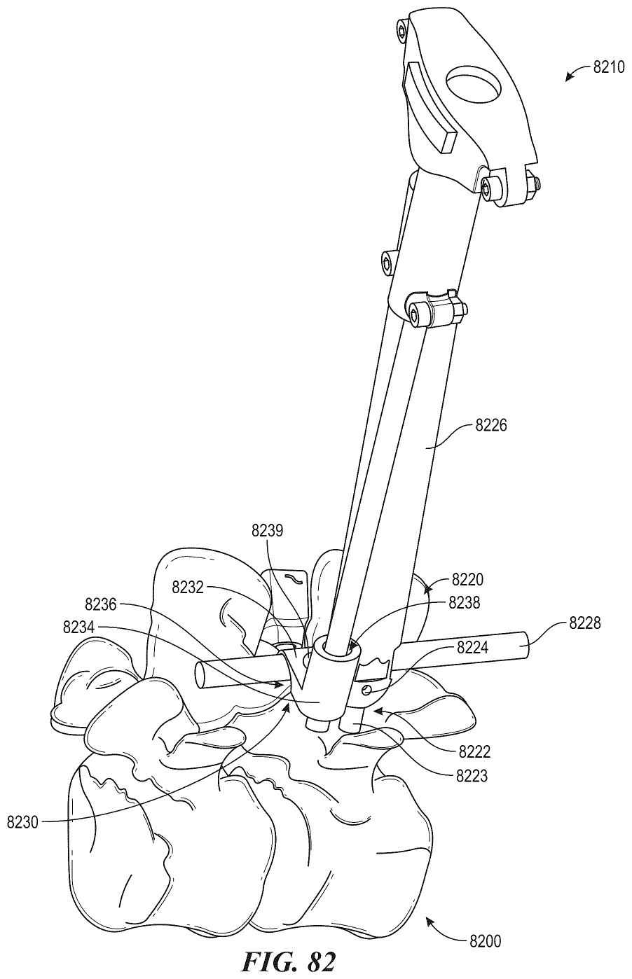

FIGS. 81A and 81B are isometric views of a connector guide member 8130 coupling a trocar 8110 to a spinal fixation system 8120 (e.g., a posterior fixation assembly) attached to a portion of a spine 8100 of a patient in accordance with embodiments of the present technology. The connector guide member 8130 is in a first (e.g., released) position in FIG. 81A and a second (e.g., clamped) position in FIG. 81B. Referring to FIGS. 81A and 81B, the spinal fixation system 8120 can include a fixation member 8122, such as a pedicle screw, having a screw body 8123 secured to the spine 8100 (e.g., a vertebra thereof) and a polyaxial head or tulip 8124 rotatably coupled to the screw body 8123. In the illustrated embodiment, the spinal fixation system 8120 further includes a tower member 8126 releasably coupled to the tulip 8124.

Referring to FIGS. 81A and 81B, the connector guide member 8130 can be a ratchet clamp having (i) a first arm 8132 having a first end portion 8131 and a second end portion 8133 and (ii) a second arm 8134 having a first end portion 8135 and a second end portion 8137 (obscured in FIG. 81B). The first end portion 8131 of the first arm 8132 can be pivotably coupled to the first end portion 8135 of the second arm 8134 at a pivot joint 8136. The first and second arms 8132, 8134 can together define an opening or lumen 8138 configured to receive the tower member 8126. In some embodiments, the second end portion 8133 of the first arm 8132 can include teeth (not shown) configured to mate with corresponding teeth on the second end portion 8137 of the second arm 8134 (e.g., in a ratchet arrangement). The mating of the teeth can secure the second end portion 8133 of the first arm 8132 to the second end portion 8137 of the second arm 8134 and allow for adjustability of a cross-sectional dimension (e.g., diameter, area) of the lumen 8138. The connector guide member 8130 can further include an actuator 8140 configured to be actuated to release the teeth from one another such that the connector guide member 8130 can be moved from the second position shown in FIG. 81B to the first position shown in FIG. 81A. In some embodiments, the second arm 8134 includes a coupling portion 8144 defining a through-hole 8145 (e.g., an opening, a lumen).

The connector guide member 8130 can be releasably coupled to the tower member 8126 by, for example, (i) positioning the tower member 8126 within the lumen 8138 of the connector guide member 8130 with the connector guide member 8130 in the first position as shown in FIG. 81A and then (ii) moving the first and second arms 8132, 8134 toward one another (e.g., closing the connector guide member 8130 and moving the connector guide member 8130 to the second position shown in FIG. 81B) to decrease the cross-sectional dimension of the lumen 8138 and to clamp the tower member 8126 therebetween. As the connector guide member 8130 closes, the teeth on the second end portions 8133, 8137 of the first and second arms 8132, 8134 engage one another to inhibit movement of the connector guide member 8130 back to the first position shown in FIG. 81A. The trocar 8110 can be inserted through the through-hole 8145 such that the trocar 8110 is fixed to move with the tower member 8126. That is, the connector guide member 8130 can provide a rigid coupling of the trocar 8110 to the tower member 8126. The tower member 8126 can be moved (e.g., pivoted relative to the tulip 8124) to adjust the position of the trocar 8110 relative to the spine 8100. Accordingly, the tower member 8126 can provide for polyaxiality or at least one degree of freedom to move the trocar 8110 relative to the spine 8100 and align the trocar 8110 with a disc space of the spine 8100 for routing one or more balloons, intervertebral devices, and/or the like through the trocar 8110. The connector guide member 8130 can be released from the tower member 8126 by actuating the actuator 8140 to disengage the teeth on the second end portions 8133, 8137 of the first and second arms 8132, 8134, thereby permitting the connector guide member 8130 to move to the first position shown in FIG. 81A.

FIGS. 82-84C illustrate various connector guide members in accordance with the present technology that are configured to secure a trocar to a spanning member (e.g., a rod) of a posterior fixation assembly. In some aspects of the present technology, spanning members can have several universal sizes (e.g., diameters) and a connector guide member can be adjustably secured to spanning members of different sizes. In contrast, there are many fixation members having different shapes and arrangements that are commonly used during spinal surgical procedures (e.g., produced by different manufacturers), and to which spanning members are secured to. Accordingly, by securing the connector guide member to a spanning member rather than a fixation member of a posterior fixation assembly, the connector guide member can more easily be used with a wide variety of posterior fixation assemblies.

FIG. 82 is an isometric view of a connector guide member 8230 coupling a trocar 8210 to a spinal fixation system 8220 (e.g., a posterior fixation assembly) attached to a portion of a spine 8200 of a patient in accordance with embodiments of the present technology. In the illustrated embodiment, the spinal fixation system 8220 includes a fixation member 8222 and a spanning member 8228 coupled to the fixation member 8222. The fixation member 8222 can be a pedicle screw having a screw body 8223 secured to the spine 8200 (e.g., a vertebra thereof) and a polyaxial head or tulip 8224 rotatably coupled to the screw body 8223. The spanning member 8228 can comprise a rod, wire, band, plate, clamp, and/or the like, and can be coupled to the tulip 8224. In the illustrated embodiment, the spinal fixation system 8220 further includes a tower member 8226 releasably coupled to the tulip 8224.

In the illustrated embodiment, the connector guide member 8230 includes a rod coupling portion 8232 and a trocar coupling portion 8234. The rod coupling portion 8232 includes/defines a channel 8236 configured to receive a portion of the spanning member 8228. The trocar coupling portion 8234 can include/define a through-hole 8238 configured to receive the trocar 8210 therethrough. The channel 8236 can have a closed shape (e.g., circular) such that the connector guide member 8230 can be slid onto and off of the spanning member 8228, or can have an open shape (e.g., semicircular) such that the connector guide member 8230 can be positioned over the spanning member 8228. The channel 8236 can be sized to fit a variety of standard sizes of the spanning member 8228, such as a diameter of 4.5 millimeters, 5.0 millimeters, 5.5 millimeters, 6.0 millimeters, and/or the like. In other embodiments, the rod coupling portion 8232 can have two or more movable arms and/or other locking features that can be locked together to clamp the connector guide member 8230 to the spanning member 8228 as, for example, described in detail above with reference to the connector guide members of FIGS. 78A-81B.

In the illustrated embodiment, the connector guide member 8230 can further include a set screw 8239. The set screw 8239 can be loosened (e.g., unlocked) to permit axial (e.g., sliding) movement of the connector guide member 8230 along the spanning member 8228 and/or circumferential (e.g., rotational) movement of the connector guide member 8230 about the spanning member 8228. Conversely, the set screw 8239 can be tightened (e.g., locked) to inhibit or even prevent axial (e.g., sliding) movement of the connector guide member 8230 along the spanning member 8228 and/or circumferential (e.g., rotational) movement of the connector guide member 8230 about the spanning member 8228. With the set screw 8239 in loosened, the connector guide member 8230 can be moved to adjust the position of the trocar 8210 relative to the spine 8200. That is, the connector guide member 8230 provides for at least two degrees of freedom (e.g., axial and circumferential relative to the spanning member 8228) to move the trocar 8210 relative to the spine 8200 and align the trocar 8210 with a disc space of the spine 8200 for routing one or more balloons, intervertebral devices, and/or the like through the trocar 8210 to the disc space. The set screw 8239 can be tightened to lock the connector guide member 8230 and the trocar 8210 in a desired position and/or orientation relative to the spine 8200 and the fixation member 8222.

In some embodiments, during a spinal surgical procedure, the connector guide member 8230 can be positioned along the spinal fixation system 8220 to facilitate lordosis and distraction of a disc space of the spine 8200 when a balloon or other device is inserted the trocar 8210 and expanded within the disc space. For example, the set screw 8239 can be tightened to lock the connector guide member 8230 in axial position along the spanning member 8228 adjacent to the fixation member 8222. Accordingly, the set screw 8239 can inhibit or even prevent the tulip 8224 from sliding (e.g., axially) along the spanning member 8228 during expansion of the balloon or other device. This can constrain the fixation member 8222 to pivot rather than move laterally to induce lordosis rather than parallel distraction as, for example, described in detail above with reference to FIGS. 3G-3I. In other embodiments, the set screw 8239 need not be locked during expansion of the balloon or other device and a clamp device can be secured to the tower member 8226 to constrain the fixation member 8222 to induce lordosis of the spine 8200 during distraction of the disc space.

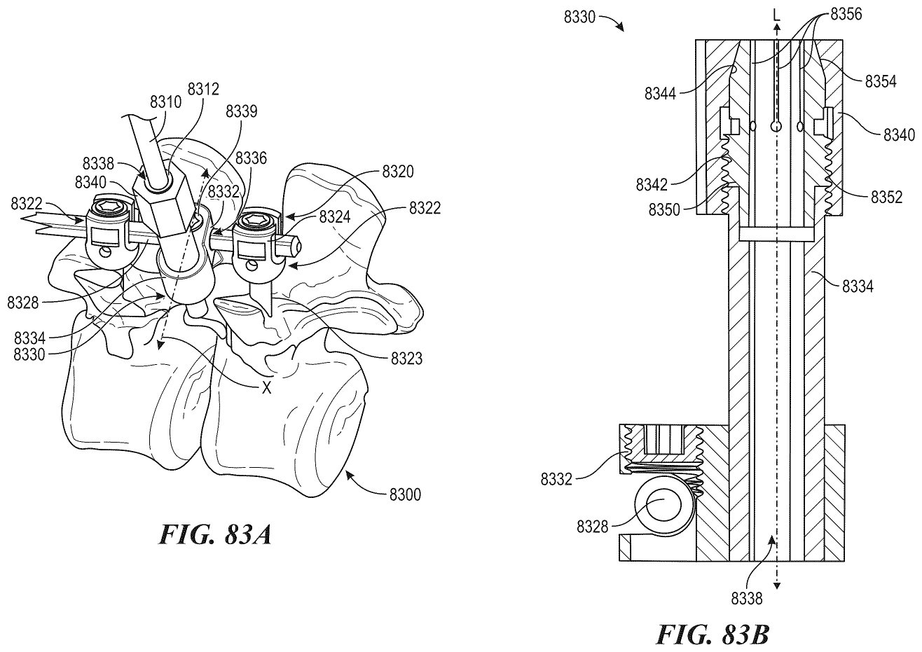

FIG. 83A is an isometric view of a connector guide member 8330 coupling a trocar 8310 to a spinal fixation system 8320 (e.g., a posterior fixation assembly) attached to a portion of a spine 8300 of a patient in accordance with embodiments of the present technology. In the illustrated embodiment, the spinal fixation system 8320 includes multiple fixation members 8322 and a spanning member 8328 coupled to the fixation members 8322. The fixation members 8322 can be pedicle screws each having a screw body 8323 secured to the spine 8300 (e.g., a vertebra thereof) and a polyaxial head or tulip 8324 rotatably coupled to the screw body 8323. The spanning member 8328 can comprise a rod, wire, band, plate, clamp, and/or the like, and can be coupled to the tulips 8324.

The connector guide member 8330 can include some features that are at least generally similar in structure and function, or identical in structure and function, to the corresponding features of the connector guide member 8230 of FIG. 82. For example, in the illustrated embodiment the connector guide member 8330 includes (i) a rod coupling portion 8332 including/defining a channel 8336 configured to receive a portion of the spanning member 8328, (ii) a trocar coupling portion 8334 including/defining a through-hole 8338 configured to receive the trocar 8310, and (iii) a set screw 8339 configured to be tightened to clamp/fix/secure the connector guide member 8330 to the spanning member 8328.

In the illustrated embodiment, the connector guide member 8330 further includes an actuator 8340 configured to be actuated (e.g., rotated) to secure the trocar 8310 within the through-hole and to fix an insertion depth of the trocar 8310 within the through-hole 8338. FIG. 83B is a cross-sectional side view of the connector guide member 8330 in accordance with embodiments of the present technology. In the illustrated embodiment, the trocar coupling portion 8334 includes a wall 8350 defining the through-hole 8338 (which extends along a longitudinal axis L) and having a threaded outer surface portion 8352 and an angled outer surface portion 8354. The wall 8350 can further define one or more slots 8356 extending longitudinally along an upper portion thereof. The actuator 8340 can be positioned around the wall 8350 and can have a threaded inner surface portion 8342 and an angled inner surface portion 8344. The threaded outer surface portion 8352 of the wall 8350 can threadably engage the threaded inner surface portion 8342 of the actuator 8340, and the angled outer surface portion 8354 of the wall 8350 can abut/engage the angled inner surface portion 8344 of the actuator 8340.

In operation, the actuator 8340 can be rotated about the wall 8350 in a first direction to drive the actuator 8340 downward along the longitudinal axis L via the engagement of the threaded outer surface portion 8352 and the threaded inner surface portion 8342. As the actuator 8340 moves in this direction, the angled inner surface portion 8344 of the actuator 8340 can press against the angled outer surface portion 8354 of the wall 8350, thereby deflecting/driving the wall 8350 radially inward along the slots 8356 in a collet-like arrangement. This deflection of the wall 8350 along the slots 8356 reduces the diameter of the through-hole 8338 and causes the wall 8350 to clasp/grip the trocar 8310 (FIG. 83A) therein-fixing the position of the trocar 8310 within the through-hole 8338 along the longitudinal axis L. The actuator 8340 can be rotated in a second direction opposite to the first direction to permit the wall 8350 to deflect radially outward along the slots 8356 to release the trocar 8310 and again allow for translation of the trocar 8310 through the through-hole 8338.

Referring to FIG. 83A, in some embodiments the trocar 8310 can include an indicator 8312, such as a laser marking. The indicator 8312 can indicate to a user a depth at which the trocar 8310 should be inserted through the through-hole 8338 before tightening the actuator 8340 to clamp the connector guide member 8330 to the trocar 8310.

Referring to FIG. 83A, when coupled to the spanning member 8328, the connector guide member 8330 allows for (i) axial movement of the trocar 8310 along the spanning member 8328, (ii) rotational movement of the trocar 8310 about the spanning member 8328, (iii) longitudinal movement of the trocar 8310 through the through-hole 8338, and (iv) rotational movement of the trocar 8310 within the through-hole 8338. In some embodiments, the connector guide member 8330 can further include a joint between the rod coupling portion 8332 and the trocar coupling portion 8334 to allow for rotation of the trocar 8310 about an axis X extending generally orthogonal to the longitudinal axis L (FIG. 83B) and the spanning member 8328.

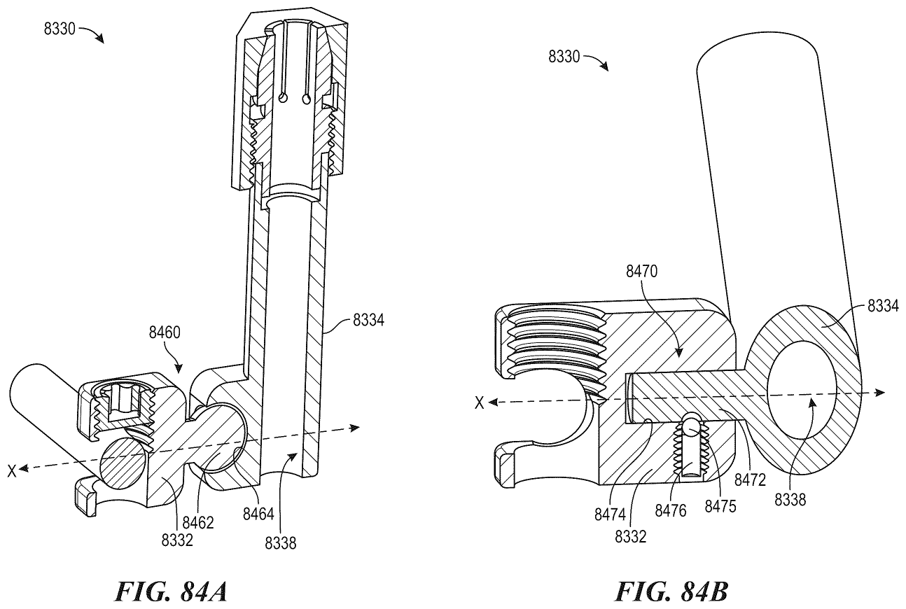

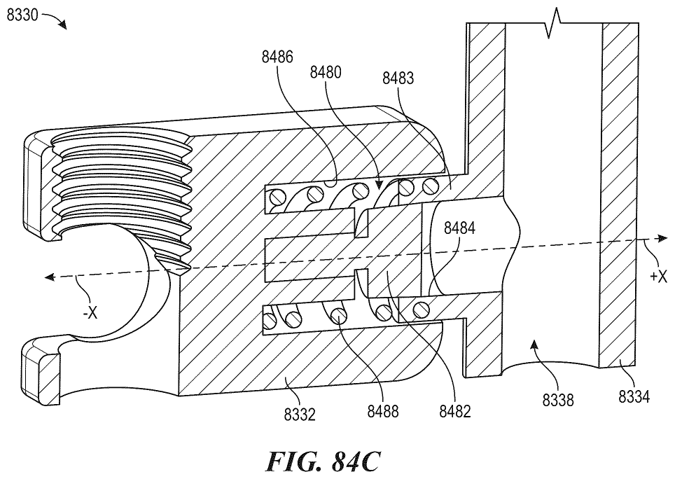

FIGS. 84A-84C, for example, are cross-sectional isometric views of the connector guide member 8330 in accordance additional embodiments of the present technology. Referring to FIG. 84A, the connector guide member 8330 includes a ball joint 8460 coupling the rod coupling portion 8332 to the trocar coupling portion 8334 to allow for rotation of the trocar coupling portion 8334 (and the trocar 8310 secured within the through-hole 8338 as shown in FIG. 83A) about the axis X. More specifically, the ball joint 8460 can include a ball 8462 coupled to (e.g., fixed to) the rod coupling portion 8332 and a socket 8464 formed in the trocar coupling portion 8334 and configured to rotatably receive the ball 8462. The ball joint 8460 can permit full circumferential rotation (e.g., 360 degrees rotation) of the trocar coupling portion 8334 about the axis X relative to the rod coupling portion 8332. In some embodiments, the connector guide member 8330 can further include a set screw or other locking mechanism (not shown) configured to selectively lock a rotational position of the trocar coupling portion 8334 relative to the rod coupling portion 8332.

Referring to FIG. 84B, the connector guide member 8330 can include a ball and detent joint 8470 coupling the rod coupling portion 8332 to the trocar coupling portion 8334 to allow for rotation of the trocar coupling portion 8334 (and the trocar 8310 secured within the through-hole 8338 as shown in FIG. 83A) about the axis X. More specifically, the ball and detent joint 8470 can include a shaft 8472 coupled to (e.g., fixed to) the trocar coupling portion 8334 and a socket 8474 (e.g., a channel) formed in the rod coupling portion 8332 and configured to rotatably receive the shaft 8472. The ball and detent joint 8470 can further include a ball 8475 positioned on a spring-loaded shaft 8476, and the shaft 8472 can include one or more detents 8478 circumferentially arranged thereabout. As the shaft 8472 is rotated, the spring-loaded shaft 8476 can drive the ball 8475 into a corresponding one of the detents 8478 to selectively lock a rotational position of the trocar coupling portion 8334 relative to the rod coupling portion 8332. A number and/or positioning of the detents 8478 can be selected to provide rotational locking at desired angles about the axis X. The shaft 8472 can be rotated from a locked position in which the ball 8475 is positioned within a corresponding one of the detents 8478 by applying a rotational force to the trocar coupling portion 8334 that overcomes the spring force of the spring-loaded shaft 8476 and thereby moves the ball 8475 out of the corresponding ones of the detents 8478.

Referring to FIG. 84C, the connector guide member 8330 can include a cone lock joint 8480 coupling the rod coupling portion 8332 to the trocar coupling portion 8334 to allow for rotation of the trocar coupling portion 8334 (and the trocar 8310 secured within the through-hole 8338 as shown in FIG. 83A) about the axis X. More specially, the cone lock joint 8480 can include a locking member 8482 coupled to (e.g., fixed to) the rod coupling portion 8332 and a socket 8484 formed in the trocar coupling portion 8334 and configured to receive the locking member 8482. The rod coupling portion 8332 can further include a channel 8486 positioned around the locking member 8482. The trocar coupling portion 8334 can include a hollow shaft 8483 defining the socket 8484 and configured to move at least partially through the channel 8486. The locking member 8482 can have a conical or wedge-like shape and the socket 8484 can have a corresponding conical or wedge-like shape. A biasing member 8488 (e.g., a compression spring) can be positioned within the channel 8486 between the shaft 8483 and the rod coupling portion 8332.

In operation, the biasing member 8488 can bias the shaft 8483 and the trocar coupling portion 8334 along the axis X in a +X direction (e.g., away from the rod coupling portion 8332) to a first (e.g., locked) position shown in FIG. 84C. In the first position, the locking member 8482 engages the shaft 8483 within the socket 8484 to inhibit or even prevent rotation of the trocar coupling portion 8334 about the axis X. That is, for example, the conical or wedge-like shape of the locking member 8482 can provide a friction or interference fit within the socket 8484 that inhibits or even prevents rotation of the trocar coupling portion 8334. To rotate the trocar coupling portion 8334 about the axis X, the trocar coupling portion 8334 can be pushed to a second (e.g., unlocked) position along the axis X in a −X direction (e.g., toward the rod coupling portion 8332) against the biasing force of the biasing member 8488. This movement moves the shaft 8483 over the locking member 8482 such that the locking member 8482 provides no or less of a frictional or interference force within the socket 8484—permitting the trocar coupling portion 8334 to be freely rotated to a desired angle. Release of the trocar coupling portion 8334 will cause the biasing member 8488 to again bias the shaft 8483 in the +X direction to the locked first position. Accordingly, in some aspects of the present technology the cone lock joint 8480 permits permit full circumferential rotation (e.g., 360 degrees rotation) of the trocar coupling portion 8334 about the axis X relative to the rod coupling portion 8332 and automatic locking at any selected angle.

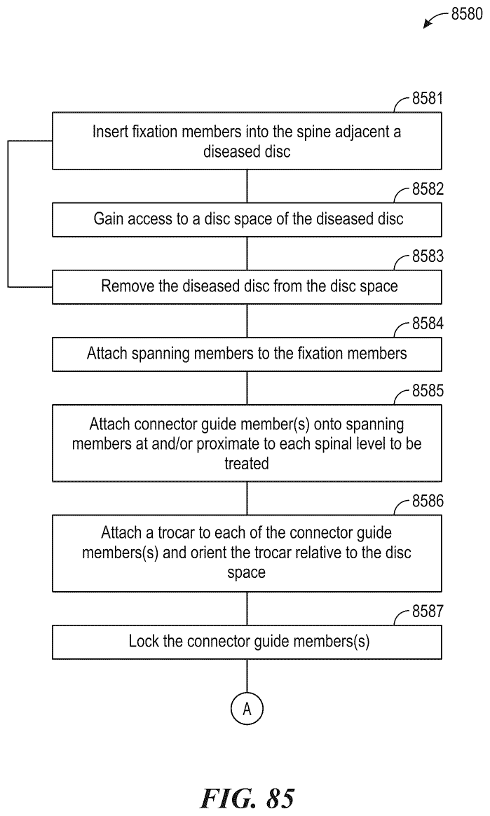

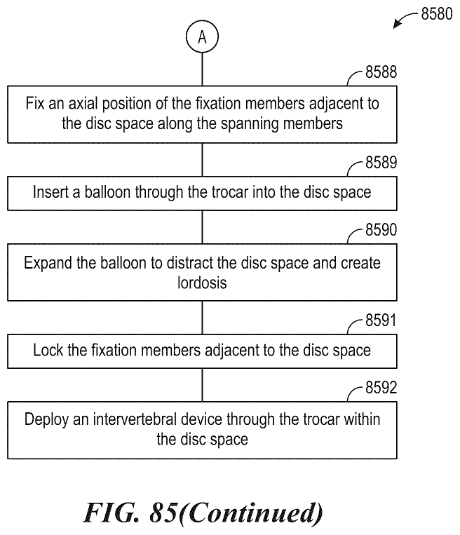

FIG. 85 is a flow diagram of a process or method 8580 for performing a spinal surgical procedure (e.g., a spinal fusion procedure) on a spine of a patient in which a connector guide member is used to fix a trocar to a spanning member (e.g., a rod) of a posterior fixation system in accordance with embodiments of the present technology. The connector guide member can be, for example, of the type described in detail with reference to FIGS. 82-84C. Likewise, many of the blocks of the method 8580 can include features similar or identical to other methods described in detail herein and utilize any of the various devices described in detail herein.

At block 8581, the method 8580 can include inserting fixation members, such as pedicle screws, into the spine adjacent to a diseased disc. At block 8582, the method 8580 can include gaining access to a disc space of the diseased disc. For example, a surgeon may perform a laminectomy and/or a facetectomy in a partially-open or fully-open procedure. At block 8583, the method 8580 can include removing the diseased disc from the disc space, such as by incising into the diseased disc and performing an open discectomy. If the spinal surgical procedure is a multilevel procedure, after block 8583, the method 8580 can return to carry out blocks 8581-8583 for each spinal level to be treated (e.g., for each diseased disc to be treated).

At block 8584, after the disc at each spinal level to be treated is removed, the method 8580 can include attaching spanning members (e.g., rods) to the fixation members. For example, two spanning members can be bilaterally affixed to the fixation members with set screws of the fixation members not fully tightened. At block 8585, the method 8580 can include attaching connector guides onto the spanning members at and/or proximate to each spinal level to be treated. For example, for a single-level procedure, only one connector guide may be secured to the spanning member adjacent the single level to be treated. For a two-level procedure, two connector guides may be secured to the spanning member adjacent the two levels to be treated, and so on. The connector guides can be coupled to the spanning members as described in detail above, for example, with reference to FIGS. 82-84C.

At block 8586, the method 8580 can include attaching a trocar to each of the connector guides and orienting the trocar relative to the disc space to be treated. For example, the angle, axial position, depth, etc., of the trocar relative to the disc space can be adjusted using the connector guide member as described in detail above, for example, with reference to FIGS. 82-84C. In some embodiments, a single trocar is used for the spinal surgical procedure and, accordingly, can be inserted serially into each of the connector guide members to orient the connector guide member and the trocar at a desired position relative to the disc space. In other embodiments, an individual trocar can be connected to each connector guide member.

At block 8587, after orienting the trocar and the connector guide member for a given spinal level, the connector guide member and/or the trocar can be locked in position and orientation relative to the disc space as described in detail above, for example, with reference to FIGS. 82-84C. For example, referring to FIGS. 83A-84C, (i) the set screw 8339 can be tightened to lock the connector guide member 8330 (and the coupled trocar 8310) to the spanning member 8328 at a desired rotational and axial position, (ii) the actuator 8340 can be rotated to lock the trocar 8310 to the connector guide member 8330 at a desired depth, and (iii) the ball joint 8460, the ball and detent joint 8470, the cone lock joint 8480, and/or another coupling can be locked to fix the connector guide member 8330 (and the coupled trocar 8310) at a desired angle about the axis X.

Referring again to FIG. 85, at block 8588 the method 8580 can include locking an axial position of the fixation members adjacent to the disc space being treated along the spanning members. For example, tower members can be coupled to the fixation members and clamped together as described in detail above with reference to FIGS. 3G-3J. Alternatively or additionally, one or more clips, clamps, limiters, and/or the like can be fixed to the spanning members adjacent the fixation members to inhibit or even prevent the fixation members from sliding (e.g., axially) along the spanning members during distraction of the disc space.

At block 8589, the method 8580 can include inserting a balloon through the trocar into disc space. At block 8590, the method 8580 can include expanding the balloon to distract the disc space and create lordosis of the spine. Volume, pressure, etc., can be read of the balloon and/or the amount of created lordosis can be measured in real-time or near real-time. At block 8591, the method 8580 can include locking the fixation members adjacent to the disc space being treated by, for example, fully tightening set screws of the fixation members to maintain the created lordosis of the spine. At block 8592, the method can include (e.g., after removing the balloon) deploying an intervertebral device through the trocar including, for example, expanding the intervertebral device, filling the intervertebral device, tensioning the intervertebral device, locking the intervertebral device, etc.

After block 8592, the method 8580 can return to block 8586 to attach a trocar to another one of the connector guides for treating the disc space at another spinal level if the spinal surgical procedure is a multilevel procedure. Blocks 8586-8592 can repeated for each spinal level. Once all the spinal levels are treated, the connector guides and/or other surgical tools may be removed from the patient.

In some aspects of the present technology, the method 8580 requires that the spanning members be inserted through the fixation members early in the spinal surgical procedure (e.g., before blocks 8585-8592). Some surgeons may be used to inserting spanning members toward the end of a spinal surgical procedure. Accordingly, in some embodiments, a connector guide member in accordance with the present technology can be integrated with a short, temporary “spanning member” that allows the method 8580 to proceed similarly but allows for the permanent spanning members to be implanted at or near the end of the spinal surgical procedure.

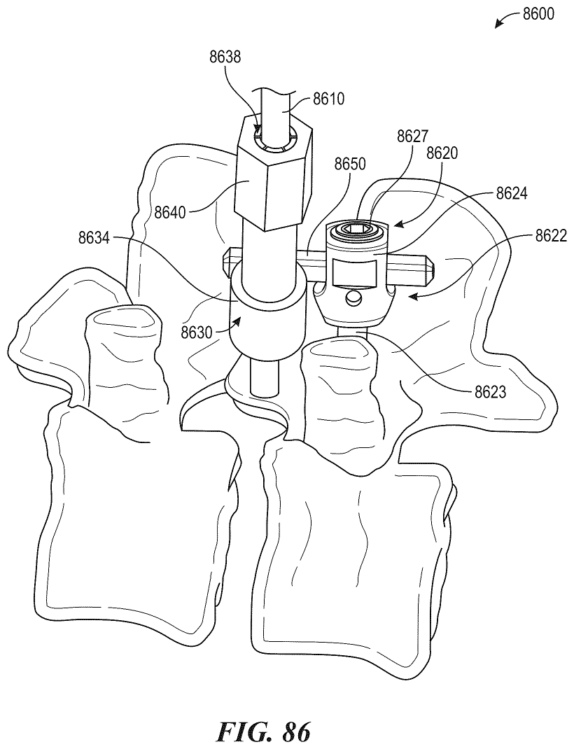

FIG. 86, for example, is an isometric view of a connector guide member 8630 coupling a trocar 8610 to a spinal fixation system 8620 (e.g., a posterior fixation assembly) attached to a portion of a spine 8600 of a patient in accordance with embodiments of the present technology. In the illustrated embodiment, the spinal fixation system 8620 includes a fixation member 8622, such as a pedicle screw having a screw body 8623 secured to the spine 8600 (e.g., a vertebra thereof) and a polyaxial head or tulip 8624 rotatably coupled to the screw body 8623.

The connector guide member 8630 can include some features that are at least generally similar in structure and function, or identical in structure and function, to the corresponding features of the connector guide member 8330 of FIGS. 83A-83B. For example, in the illustrated embodiment the connector guide member 8630 includes a trocar coupling portion 8634 including/defining a through-hole 8638 configured to receive the trocar 8610 and an actuator 8640 configured to be actuated (e.g., rotated) to fix a position and/or orientation of the trocar 8610 within the through-hole 8638.

In the illustrated embodiment, the connector guide member 8630 further includes a rod portion 8650 coupled to and extending laterally away from the trocar coupling portion 8634. The rod portion 8650 can be configured (e.g., sized, shaped) to extend through and be coupled to the tulip 8624 of the fixation member 8622. For example, the rod portion 8650 can have a diameter corresponding to a standard spinal surgical rod, such as a diameter of 4.5 millimeters, 5.0 millimeters, 5.5 millimeters, 6.0 millimeters, and/or the like. The rod portion 8650 can rotate and translate within the tulip 8624 to adjust an axial and/or rotational position of the connector guide member 8630 (and the trocar 8610 coupled thereto) relative to the fixation member 8622 and a disc space of the spine 8600. In some embodiments, the fixation member 8622 includes a set screw 8627 that can be tightened to lock the position and orientation of the connector guide member 8630 (and the trocar 8610 coupled thereto) relative to the fixation member 8622 and the disc space of the spine 8600. Moreover, the tulip 8624 can be pivoted relative to the screw body 8623 to correspondingly pivot the connector guide member 8630 (and the trocar 8610 coupled thereto) to provide for additional degrees of freedom in aligning the trocar 8610 with the disc space of the spine 8600. In some aspects of the present technology, the connector guide member 8630 provides a similar coupling as the connector guide member 8330 of FIGS. 83A-84C but can be connected directly to the fixation member 8622 via the rod portion 8650 rather than to a static, standard spinal surgical rod. Moreover, the rod portion 8650 can be low profile such that the rod portion 8650 does not interfere with the disc space.

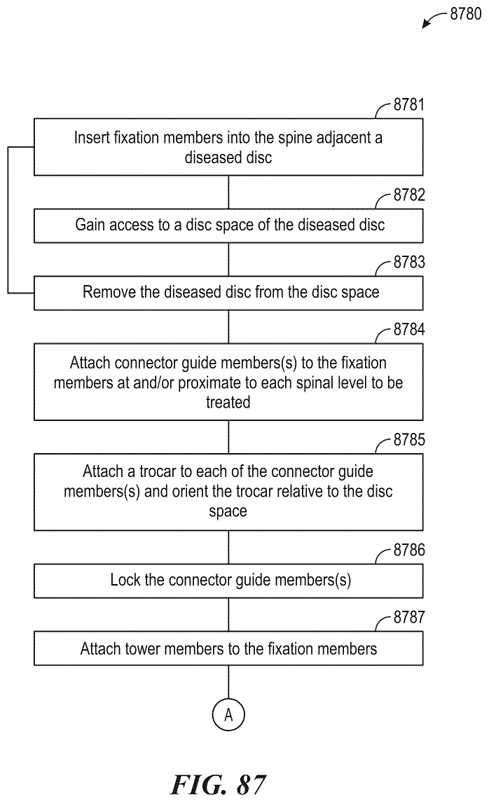

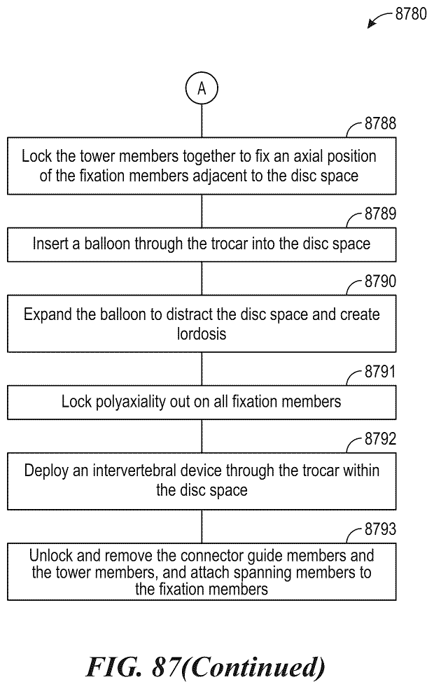

FIGS. 87-89 are flow diagrams of processes or methods 8780, 8880, and 8980, respectively, for performing a spinal surgical procedure (e.g., a spinal fusion procedure) on a spine of a patient in which the connector guide member 8630 of FIG. 86 is used to fix a trocar to a fixation member of a posterior fixation system in accordance with embodiments of the present technology. Many of the blocks of the methods 8780, 8880, and 8980 can include features similar or identical to one another and/or the other methods described in detail herein and can utilize any of the various devices described in detail herein.

Referring to FIGS. 87-89, each of the methods 8780, 8880, and 8980 can have in common blocks 8781-8785. At block 8781, the method 8780 can include inserting fixation members, such as pedicle screws, into the spine adjacent to a diseased disc. At block 8782, the method 8780 can include gaining access to a disc space of the diseased disc. For example, a surgeon may perform a laminectomy and/or a facetectomy in a partially-open or fully-open procedure. At block 8783, the method 8780 can include removing the diseased disc from the disc space, such as by incising into the diseased disc and performing an open discectomy. If the spinal surgical procedure is a multilevel procedure, after block 8783, the method 8780 can return to carry out blocks 8781-8783 for each spinal level to be treated (e.g., for each diseased disc to be treated).

At block 8784, after the disc at each spinal level to be treated is removed, the method 8780 can include attaching one or more of connector guide members 8630 of FIG. 86 to a corresponding one of the fixation members at and/or proximate to each spinal level to be treated. For example, for a single-level procedure, only one of the connector guide members 8630 may be secured to a fixation member adjacent the single level to be treated. For a two-level procedure, two of the connector guide members 8630 may be secured to fixation members adjacent the two levels to be treated, and so on. The connector guide members 8630 can be coupled to the fixation members as described in detail above, for example, with reference to FIG. 86.

At block 8785, the method 8780 can include attaching a trocar to each of the connector guide members 8630 and orienting the trocar relative to the disc space to be treated. For example, the angle, axial position, depth, etc., of the trocar relative to the disc space can be adjusted using the connector guide member 8630 as described in detail above, for example, with reference to FIG. 86. In some embodiments, a single trocar is used for the spinal surgical procedure and, accordingly, can be inserted serially into each of the connector guide members 8630 to orient the connector guide member 8630 and the trocar at a desired position relative to the disc space. In other embodiments, an individual trocar can be connected to each of the connector guide members 8630.

At block 8786, after orienting the trocar and the connector guide members 8630, the method 8680 can include locking the connector guide members 8630 and/or the trocar in position and orientation relative to the disc space. For example, referring to FIG. 86, (i) the set screw 8627 of the fixation member 8622 can be tightened to lock the connector guide member 8630 (and the coupled trocar 8610) at a desired rotational and axial position relative to the fixation member 8672 and (ii) the actuator 8640 can be rotated to lock the trocar 8610 to the connector guide member 8630 at a desired depth.

Referring to FIG. 87, at block 8787 the method 8700 can include attaching tower members to the fixation members, such as to polyaxial tulip heads of the fixation members. At block 8788, the method 8700 can include locking the towers together to fix an axial position of the fixation members adjacent to the disc space. For example, the tower members can be clamped together as described in detail above with reference to FIGS. 3G-3J. In some embodiments, tower members are attached to all the fixation members.

At block 8789, the method 8780 can include inserting a balloon through the trocar into disc space (e.g., for each disc space). At block 8790, the method 8780 can include expanding the balloon to distract the disc space and create lordosis of the spine. Volume, pressure, etc., can be read of the balloon and/or the amount of created lordosis can be measured in real-time or near real-time.

At block 8791, the method 8780 can include locking polyaxiality on all the fixation members using, for example, threaded pistons. For example, the threaded pistons can engage corresponding tulips of the fixation members to inhibit or even prevent the tulips from pivoting relative to a screw body of the fixation members. In this position, the clamped tower members can maintain (e.g., hold) the lordosis and distraction of the disc space even when the balloon is deflated.

At block 8792, the method 8780 can include (e.g., after removing the balloon) deploying an intervertebral device through the trocar including, for example, expanding the intervertebral device, filling the intervertebral device, tensioning the intervertebral device, locking the intervertebral device, etc. At block 8793, the method 8780 can include unlocking and removing the connector guide members 8630 and the tower members, and attaching spanning members to the fixation members.

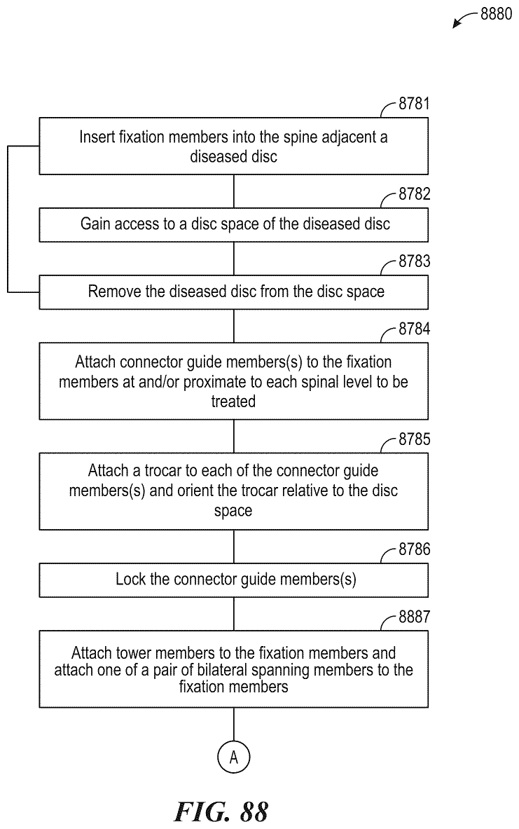

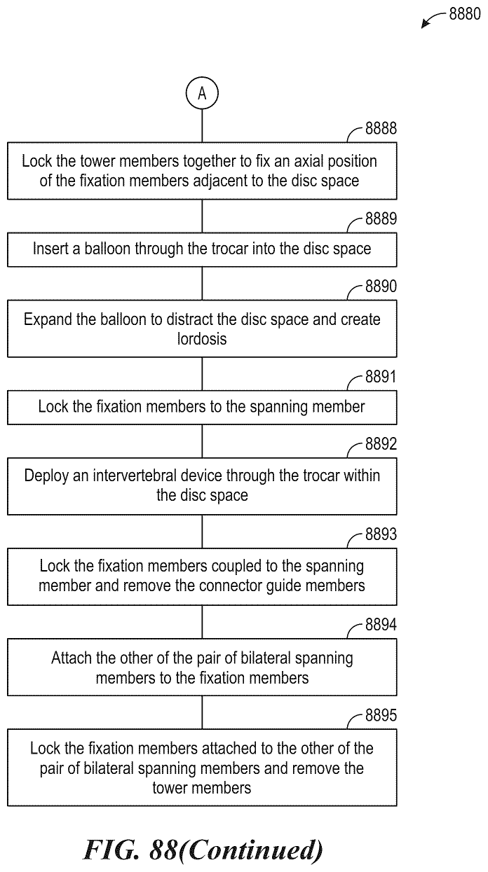

Referring to FIG. 88, after locking the connector guide members 8630 at block 8786, at block 8887 the method 8880 can include attaching one of a pair of bilateral spanning members to corresponding ones of the fixation members and attaching tower members to the fixation members. For example, a spanning member can be attached to the fixation members on the contralateral side of the patient with set screws of the fixation members not fully tightened. The connector guide members 8630 can be attached to the fixation members on the ipsilateral side of the patient (e.g., at block 8784).

At block 8888, the method 8800 can include locking the towers together to fix an axial position of the fixation members adjacent to the disc space. For example, the tower members can be clamped together as described in detail above with reference to FIGS. 3G-3J. At block 8889, the method 8880 can include inserting a balloon through the trocar into disc space (e.g., for each disc space). At block 8890, the method 8880 can include expanding the balloon to distract the disc space and create lordosis of the spine. Volume, pressure, etc., can be read of the balloon and/or the amount of created lordosis can be measured in real-time or near real-time.

At block 8891, the method 8880 can include locking the fixation members to the spanning member by, for example, tightening a set screw of each of the fixation members coupled to the spanning member. In this position, the clamped tower members (e.g., on the ipsilateral side of the patient) and the fixed spanning member (e.g., on the contralateral side of the patient) can maintain (e.g., hold) the lordosis and distraction of the disc space even when the balloon is deflated.

At block 8892, the method 8880 can include (e.g., after removing the balloon) deploying an intervertebral device through the trocar including, for example, expanding the intervertebral device, filling the intervertebral device, tensioning the intervertebral device, locking the intervertebral device, etc.

At block 8893, the method 8880 can include locking the fixation members coupled to the spanning member (e.g., those at the contralateral side of the patient) and removing the connector guide members. Locking the fixation members can include tightening a set screw thereof.

At block 8894, the method 8880 can attaching the other of the pair of bilateral spanning members to the fixation members (e.g., those on the ipsilateral side of the patient, by threading the spanning members through tulip heads of the fixation members). At block 8895, the method 8880 can include (i) locking the fixation members to the other of the pair of bilateral spanning members by, for example, tightening a set screw of the fixation members, and (ii) removing the tower members.

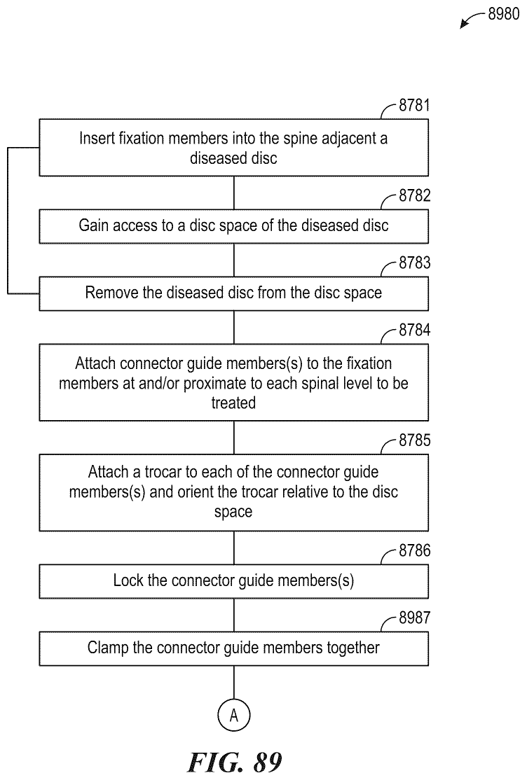

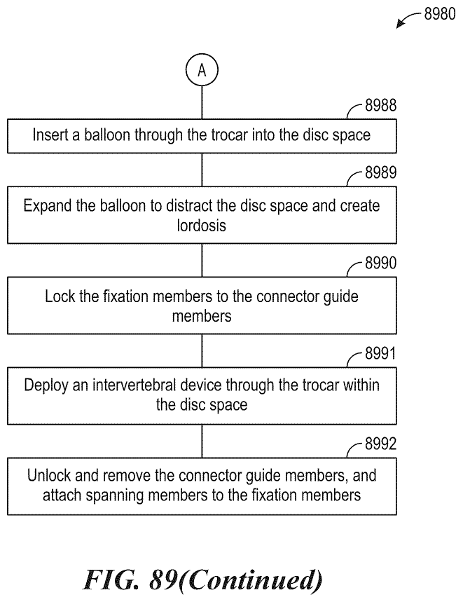

Referring to FIG. 89, after locking the connector guide members 8630 at block 8786, at block 8987 the method 8980 can include clamping the connector guide members 8230 together. Referring to FIG. 86, in some embodiments a clamp can be attached to the vertically-extending trocar coupling portions 8634 and/or another portion of the connector guide members 8230 to inhibit or even prevent movement therebetween. For a bilateral spinal surgical procedure, the connector guide members 8230 can be attached bilaterally and clamped together. For a unilateral procedure, the connector guide members 8230 can be attached to first ones of a bilateral set of fixation members (e.g., the fixation members extending along the ipsilateral side of the patient), and a spanning member can be coupled to second ones of the bilateral set of fixation members (e.g., the fixation members extending along the contralateral side of the patient). One or more clips, limiters, etc., can be fixed to the spanning member adjacent corresponding ones of the fixation members coupled thereto to inhibit axial movement thereof during balloon expansion to induce lordosis.

At block 8988, the method 8900 can include inserting a balloon through the trocar into disc space (e.g., for each disc space). At block 8989, the method 8980 can include expanding the balloon to distract the disc space and create lordosis of the spine. Volume, pressure, etc., can be read of the balloon and/or the amount of created lordosis can be measured in real-time or near real-time.

At block 8990, the method 8980 can include locking the fixation members to the connector guide members 8230 by, for example, tightening set screws thereof. In this position, the clamped connector guide members 8230 can retain the lordosis and distraction of the disc space even when the balloon is deflated (e.g., like a pin distractor or screw-based distractor). For a unilateral procedure in which the spanning member is attached to the seconds ones of the bilateral set of fixation members (e.g., as described in detail with reference to block 8987), the fixation members coupled to the spanning member can additionally or alternatively be tightened to hold open the disc space and maintain lordosis and distraction.

At block 8991, the method 8980 can include (e.g., after removing the balloon) deploying an intervertebral device through the trocar including, for example, expanding the intervertebral device, filling the intervertebral device, tensioning the intervertebral device, locking the intervertebral device, etc.

At block 8992, the method 8980 can include unlocking and removing the connector guide members 8230 from the fixation members and attaching spanning members to the fixation members. In some embodiments, the connector guide members 8230 along one side of the patient can be removed from the fixation members first, and a first spanning member coupled to the fixation members, before the connector guide members 8230 are removed from the other side of the patient and a second spanning member is coupled to the remaining fixation members.

### XV. SELECTED EMBODIMENTS OF CORRIDORS FOR ACCESSING THE L4/L5 DISC SPACE AND/OR THE L5/S1 DISC SPACE

In many typical spinal surgical procedures, the L4/L5 disc space and the L5/S1 disc space cannot be accessed via a lateral approach. In particular, the iliac crest can block lateral access to the L4/L5 disc space using retractors and typical spinal instrumentation, and the iliac crest and the sacrum can block lateral access to the L5/S1 disc space using retractors and typical spinal instrumentation. Accordingly, these disc spaces are frequently accessed via an anterior approach that requires a vascular surgeon and poses increased risk to a patient. In some aspects of the present technology, the small and minimally invasive profile of the devices and systems of the preset technology described herein can allow direct lateral access to the L4/L5 and L5/S1 disc spaces via a lateral approach.

For example, FIGS. 90A-90D are different views of a spinal surgical procedure (e.g., a spinal surgical method) on a spine 9000 of a patient 9001 (shown as partially transparent for clarity) in accordance with additional embodiments of the present technology. More specifically, FIG. 90A is a side view (e.g., a lateral view) of a portion of the spine 9000 illustrating a navigation and trajectory planning step of the spinal surgical procedure in accordance with embodiments of the present technology. Referring to FIG. 90A, the spine 9000 includes a plurality of vertebrae 9002. More specifically, the vertebrae 9002 can include an L1 lumbar vertebra 9002a, an L2 lumbar vertebra 9002b, an L3 lumbar vertebra 9002c, an L4 lumbar vertebra 9002d, an L5 lumbar vertebra 9002e (obscured in FIG. 90A), and an S1 sacral vertebra 9002f (obscured in FIG. 90A). The vertebrae 9002 can be separated by corresponding discs 9004 (e.g., intervertebral discs; including individually identified first through fifth discs 9004a-e with the fourth and fifth discs 9004d-e obscured in FIG. 90A). A surgical navigation system 9090, such as the StealthStation Surgical Navigation System sold by Medtronic PLC, can be used to determine lateral trajectories 9092 to some or all of the discs 9004, such as first through fifth lateral trajectories 9092a-e to the first through fifth discs 9004a-c, respectively. In some aspects of the present technology, the fourth lateral trajectory 9092d to the fourth disc 9004d between the L4 lumbar vertebra 9002d and the L5 lumbar vertebra 9002e extends through an iliac crest IC of the patient 9001, and the fifth lateral trajectory 9092e to the fifth disc 8404e between the L5 lumbar vertebra 9002e and the S1 sacral vertebra 9002f extends through the iliac crest IC and a sacrum S of the spine 9000.

FIG. 90B is a side view (e.g., a lateral view) of a portion of the spine 9000 illustrating an access step of the spinal surgical procedure in accordance with embodiments of the present technology. One or more introducers 9008 can be inserted along one or more of the lateral trajectories 9092. For example, in the illustrated embodiment first through fifth introducers 9008a-e are inserted at least partially along the first through fifth lateral trajectories 9092a-e (FIG. 90A), respectively. The introducers 9008 can provide surgical access at least part way to discs 9004.

FIG. 90C is a side view (e.g., a lateral view) and an enlarged front view (e.g., anterior view) of a portion of the spine 9000 illustrating a first intervertebral device deployment step of the spinal surgical procedure in accordance with embodiments of the present technology. In the illustrated embodiment, a trocar 9010 can be inserted through the fourth introducer 9008d and used to access a disc space between the L4 lumbar vertebra 9002d and the L5 lumbar vertebra 9002c. A balloon can be deployed through the trocar 9010 to create lordosis and/or to distract the disc space, and/or a first intervertebral device 9040a can be deployed in the disc space through the trocar 9010 as described in detail herein. In some aspects of the present technology, the trocar 9010 can extend directly through the iliac crest IC of the patient 9001 due the small profile of the trocar 9010. The iliac crest IC can provide a rigid support for the trocar 9010 during operation, and the lateral path through the iliac crest IC reduces exposure to blood vessels, nerves, etc., as compared to, for example, an anterior approach.

FIG. 90D is a side view (e.g., a lateral view) and an enlarged front view (e.g., anterior view) of a portion of the spine 9000 illustrating a second intervertebral device deployment step of the spinal surgical procedure in accordance with embodiments of the present technology. In the illustrated embodiment, the same or a different trocar 9010 can be inserted through the fifth introducer 9008e and used to access a disc space between the L5 lumbar vertebra 9002e and the S1 sacral vertebra 9002f. A balloon can be deployed through the trocar 9010 to create lordosis and/or to distract the disc space, and/or a second intervertebral device 9040b can be deployed in the disc space through the trocar 9010 as described in detail herein. In some aspects of the present technology, the trocar 9010 can extend directly through the iliac crest IC the patient 9001 and the sacrum S of the spine 9000 due the small profile of the trocar 9010. The iliac crest IC and the sacrum S can provide a rigid support for the trocar 9010 during operation, and the lateral path through the iliac crest IC and the sacrum S reduces exposure to blood vessels, nerves, etc., as compared to, for example, an anterior approach. In some embodiments, the same or a different trocar 9010 can similarly be inserted through one or more of the first through third introducers 9008a-c and used to create lordosis, deploy intervertebral devices, etc., in the corresponding disc spaces accessed thereby.

### XVI. ADDITIONAL EXAMPLES

The following examples are illustrative of several embodiments of the present technology:

1. A method of treating a spine of a patient, the method comprising:
inserting a trocar to proximate a diseased disc within a disc space of the spine along a lateral, transpedicular, transfacet, or transforaminal access path;
inserting a discectomy device through the trocar;
disrupting at least a portion of the diseased disc with the discectomy device;
inserting an intervertebral device through the trocar into the disc space;
expanding the intervertebral device within the disc space; and filling the intervertebral device with a fill material.

2. The method of example 1 wherein the intervertebral device comprises a braid of filaments.

3. The method of example 2 wherein the method further comprises tensioning the braid of filaments after expanding the intervertebral device within the disc space.

4. The method of any one of examples 1-3 wherein the method further comprises:
inserting a balloon through the trocar into the disc space; and
expanding the balloon within the disc space to distract the disc space.

5. The method of any one of examples 1˜4 wherein the trocar is a first trocar, wherein the method further comprises inserting a second trocar through the first trocar, and wherein the second trocar has a curved distal portion configured to extend from the first trocar.

6. The method of example 5 wherein inserting the discectomy device includes inserting the discectomy device through the second trocar.

7. The method of example 5 or example 6 wherein the method further comprises:
inserting a balloon through the second trocar into the disc space; and
expanding the balloon within the disc space to distract the disc space.

8. The method of any one of examples 1-7 wherein inserting the intervertebral device includes inserting the intervertebral device through the second trocar.

9. The method of any one of examples 1-8, further comprising disrupting at least a portion of a ligamentous structure positioned around the diseased disc.

10. The method of any one of examples 1-9 wherein the disc space is an L4/L5 disc space of the spine, and wherein inserting the trocar to proximate the diseased disc within the L4/L5 disc space comprises inserting the trocar along a lateral approach that extends through an iliac crest of the patient.

11. The method of any one of examples 1-9 wherein the disc space is an L5/S1 disc space of the spine, and wherein inserting the trocar to proximate the diseased disc within the L5/S1 disc space comprises inserting the trocar along a lateral approach that extends through an iliac crest of the patient and a sacrum of the spine of the patient.

12. A system for treating a spine of a patient, comprising:
a trocar configured to be inserted to proximate a diseased disc within a disc space of the spine along a lateral, transpedicular, transfacet, or transforaminal access path;
a discectomy device configured to be inserted through the trocar and actuated to disrupt at least a portion of the diseased disc;
an intervertebral device configured to be inserted through the trocar and expanded within the disc space; and
a fill material configured to be inserted through the trocar and into the expanded intervertebral device.

13. The system of example 12 wherein the intervertebral device comprises a braid of filaments.

14. The system of example 13, further comprising a tensioning device configured to be inserted through the trocar and actuated to tension the braid of filaments within the disc space.

15. The system of any one of examples 12-14, further comprising a balloon configured to be inserted through the trocar and expanded within the disc space to distract the disc space.

16. The system of any one of examples 12-15 wherein the trocar is a first trocar, and further comprising a second trocar configured to be inserted through the first trocar, wherein the second trocar has a curved distal portion configured to extend from the first trocar.

17. The system of example 16 wherein the discectomy device is configured to be inserted through the second trocar.

18. The system of example 16 or example 17 wherein the intervertebral device is configured to be inserted through the second trocar.

19. A method of treating a spine of a patient, the method comprising:
attaching a first posterior fixation member to a first vertebra of the spine;
attaching a second posterior fixation member to a second vertebra of the spine adjacent the first vertebra, wherein a disc space is positioned between the first vertebra and the second vertebra;
inserting a trocar into the disc space;
inserting a balloon through the trocar into the disc space;
locking a position and orientation of a first portion of the first posterior fixation member to a first portion of the second posterior fixation member;
expanding the balloon within the disc space to distract the disc space and create lordosis between the first vertebra and the second vertebra;
locking a position and orientation of a second portion of the first posterior fixation member relative to a position and orientation of a second portion of the second posterior fixation member to maintain the created lordosis; and deploying an intervertebral device within the disc space.

20. The method of example 19 wherein locking the position and orientation of the second portion of the first posterior fixation member relative to the position and orientation of the second portion of the second posterior fixation member comprises tightening a first set screw of the first posterior fixation member and tightening a second set screw of the second posterior fixation member.

21. The method of example 19 or example 20 wherein the method further comprises filling the intervertebral device with a fill material.

22. The method of any one of examples 19-21 wherein inserting the trocar into the disc space comprises inserting the trocar via a lateral approach.

23 The method of any one of examples 19-22 wherein the method further comprises:
attaching a first tower member to the first posterior fixation member; and
attaching a second tower member to the second posterior fixation member.

24. The method of example 23 wherein locking the position and orientation of the first portion of the first posterior fixation member to the first portion of the second posterior fixation member comprises clamping the first tower member to the second tower member.

25. The method of any one of examples 19-24 wherein locking the position and orientation of the first portion of the first posterior fixation member to the first portion of the second posterior fixation member comprises attaching a limiter to a spanning member coupling the first portion of the first posterior fixation member to the first portion of the second posterior fixation member.

26. The method of any one of examples 19-25 wherein the first portion of the first posterior fixation member comprises a first tulip, wherein the first portion of the second posterior fixation member comprises a second tulip, wherein the second portion of the first posterior fixation member comprises a first screw body, and wherein the second portion of the second posterior fixation member comprises a second screw body.

27. The method of any one of examples 19-26 wherein the first vertebra is an L4 vertebra of the spine, wherein the second vertebra is an L5 vertebra of the spine, wherein the disc space is an L4/L5 disc space of the spine, and wherein inserting the trocar into the disc space comprises inserting the trocar along a lateral approach that extends through an iliac crest of the patient.

28. The method of any one of examples 19-26 wherein the first vertebra is an L5 vertebra of the spine, wherein the second vertebra is an S1 vertebra of the spine, wherein the disc space is an L5/S1 disc space of the spine, and wherein inserting the trocar into the disc space comprises inserting the trocar along a lateral approach that extends through an iliac crest of the patient and a sacrum of the spine of the patient.

29 The method of any one of examples 19-28 wherein the method further comprises measuring a lordotic angle of the spine in real time or near real time while expanding the balloon within the disc space to create the lordosis between the first vertebra and the second vertebra.

30. A system for treating a spine of a patient, comprising:
a trocar configured to be inserted to proximate a diseased disc within a disc space of the spine along a lateral, transpedicular, transfacet, or transforaminal access path;
a balloon configured to be inserted through the trocar and expanded within the disc space to distract the disc space;
a locking device configured to be coupled to a posterior fixation assembly secured to the spine to lock a position and orientation of (a) a portion of a first posterior fixation member secured to a first vertebra adjacent the disc space relative to (b) a portion of a second posterior fixation member secured to a second vertebra adjacent the disc space, thereby causing expansion of the balloon to create lordosis between the first vertebra and the second vertebra; and
an intervertebral device configured to be inserted through the trocar and expanded within the disc space.

31. The system of example 30, further comprising a fill material configured to be inserted through the trocar and into the expanded intervertebral device.

32. The system of example 30 or example 31 further comprising a spinal angle measuring device configured to measure the created lordosis in real time or near real time.

33. The system of any one of examples 30-32 wherein the locking device comprises a clamp configured to fixedly couple a first tower member attached to the first posterior fixation member to a second tower member attached to the second posterior fixation member.

34 The system of any one of examples 30-33 wherein the locking device comprises a limiter configured to be secured to a spanning member coupling the portion of the first posterior fixation member to the portion of the second posterior fixation member.

35 The system of any one of examples 30-34 wherein the portion of the first posterior fixation member comprises a tulip of the first posterior fixation member coupled to a screw body of the first posterior fixation member, and wherein the portion of the second posterior fixation member comprises a tulip of the second posterior fixation member coupled to a screw body of the second posterior fixation member.

36. A method of treating a spine of a human patient, the method comprising:
inserting a trocar to proximate a diseased disc within an L4/L5 disc space between an LA vertebra and an L5 vertebra of the spine along a lateral access path that extends through an iliac crest of the patient;
inserting an intervertebral device through the trocar into the disc space; and
expanding the intervertebral device within the disc space.

37. The method of example 36 wherein the method further comprises filling the intervertebral device with a fill material.

38. The method of example 36 or example 37 wherein the method further comprises:
inserting a balloon through the trocar into the disc space; and
expanding the balloon within the disc space to distract the disc space and create lordosis between the L4 vertebra and the L5 vertebra.

39. A method of treating a spine of a human patient, the method comprising:
inserting a trocar to proximate a diseased disc within an L5/S1 disc space between an L5 vertebra and an S1 vertebra of the spine along a lateral access path that extends through an iliac crest of the patient and a sacrum of the spine of the patient;
inserting an intervertebral device through the trocar into the disc space; and expanding the intervertebral device within the disc space.

40. The method of example 39 wherein the method further comprises filling the intervertebral device with a fill material.

41. The method of example 39 or example 40 wherein the method further comprises:
inserting a balloon through the trocar into the disc space; and expanding the balloon within the disc space to distract the disc space and create lordosis between the L5 vertebra and the S1 vertebra.

### XVII. CONCLUSION

All numeric values are herein assumed to be modified by the term about whether or not explicitly indicated. The term about, in the context of numeric values, generally refers to a range of numbers that one of skill in the art would consider equivalent to the recited value (e.g., having the same function and/or result). For example, the term about can refer to the stated value plus or minus ten percent. For example, the use of the term about 100 can refer to a range of from 90 to 110, inclusive. In instances in which the context requires otherwise and/or relative terminology is used in reference to something that does not include, or is not related to, a numerical value, the terms are given their ordinary meaning to one skilled in the art.

The above detailed description of embodiments of the present technology are not intended to be exhaustive or to limit the technology to the precise forms disclosed above. Although specific embodiments of, and examples for, the technology are described above for illustrative purposes, various equivalent modifications are possible within the scope of the technology, as those skilled in the relevant art will recognize. For example, although steps may be presented in a given order, in other embodiments, the steps may be performed in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

As used herein, the phrase and/or as in A and/or B refers to A alone, B alone, and A and B. Additionally, the term comprising is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A method of treating a spine of a patient, the method comprising:
inserting a trocar to proximate a diseased disc within a disc space of the spine along a lateral, transpedicular, transfacet, or transforaminal access path;
inserting a discectomy device through the trocar;
disrupting at least a portion of the diseased disc with the discectomy device;
inserting an intervertebral device through the trocar into the disc space;
expanding the intervertebral device within the disc space; and
filling the intervertebral device with a fill material.

2. The method of claim 1 wherein the intervertebral device comprises a braid of filaments.

3. The method of claim 2 wherein the method further comprises tensioning the braid of filaments after expanding the intervertebral device within the disc space.

4. The method of claim 1 wherein the method further comprises:
inserting a balloon through the trocar into the disc space; and
expanding the balloon within the disc space to distract the disc space.

5. The method of claim 1 wherein the trocar is a first trocar, wherein the method further comprises inserting a second trocar through the first trocar, and wherein the second trocar has a curved distal portion configured to extend from the first trocar.

6. The method of claim 5 wherein inserting the discectomy device includes inserting the discectomy device through the second trocar.

7. The method of claim 5 wherein the method further comprises:
inserting a balloon through the second trocar into the disc space; and
expanding the balloon within the disc space to distract the disc space.

8. The method of claim 5 wherein inserting the intervertebral device includes inserting the intervertebral device through the second trocar.

9. The method of claim 1, further comprising disrupting at least a portion of a ligamentous structure positioned around the diseased disc.

10. The method of claim 1 wherein the disc space is an L4/L5 disc space of the spine, and wherein inserting the trocar to proximate the diseased disc within the L4/L5 disc space comprises inserting the trocar along a lateral approach that extends through an iliac crest of the patient.

11. The method of claim 1 wherein the disc space is an L5/S1 disc space of the spine, and wherein inserting the trocar to proximate the diseased disc within the L5/S1 disc space comprises inserting the trocar along a lateral approach that extends through an iliac crest of the patient and a sacrum of the spine of the patient.

12. A system for treating a spine of a patient, comprising:
a trocar configured to be inserted to proximate a diseased disc within a disc space of the spine along a lateral, transpedicular, transfacet, or transforaminal access path;
a discectomy device configured to be inserted through the trocar and actuated to disrupt at least a portion of the diseased disc;
an intervertebral device configured to be inserted through the trocar and expanded within the disc space; and
a fill material configured to be inserted through the trocar and into the expanded intervertebral device.

13. The system of claim 12 wherein the intervertebral device comprises a braid of filaments.

14. The system of claim 13, further comprising a tensioning device configured to be inserted through the trocar and actuated to tension the braid of filaments within the disc space.

15. The system of claim 12, further comprising a balloon configured to be inserted through the trocar and expanded within the disc space to distract the disc space.

16. The system of claim 12 wherein the trocar is a first trocar, and further comprising a second trocar configured to be inserted through the first trocar, wherein the second trocar has a curved distal portion configured to extend from the first trocar.

17. The system of claim 16 wherein the discectomy device is configured to be inserted through the second trocar.

18. The system of claim 16 wherein the intervertebral device is configured to be inserted through the second trocar.

19. A method of treating a spine of a patient, the method comprising:
attaching a first posterior fixation member to a first vertebra of the spine;
attaching a second posterior fixation member to a second vertebra of the spine adjacent the first vertebra, wherein a disc space is positioned between the first vertebra and the second vertebra;
inserting a trocar into the disc space;
inserting a balloon through the trocar into the disc space;
locking a position and orientation of a first portion of the first posterior fixation member to a first portion of the second posterior fixation member;
expanding the balloon within the disc space to distract the disc space and create lordosis between the first vertebra and the second vertebra;
locking a position and orientation of a second portion of the first posterior fixation member relative to a position and orientation of a second portion of the second posterior fixation member to maintain the created lordosis; and
deploying an intervertebral device within the disc space.

20. The method of claim 19 wherein locking the position and orientation of the second portion of the first posterior fixation member relative to the position and orientation of the second portion of the second posterior fixation member comprises tightening a first set screw of the first posterior fixation member and tightening a second set screw of the second posterior fixation member.

21. The method of claim 19 wherein the method further comprises filling the intervertebral device with a fill material.

22. The method of claim 19 wherein inserting the trocar into the disc space comprises inserting the trocar via a lateral approach.

23. The method of claim 19 wherein the method further comprises:
attaching a first tower member to the first posterior fixation member; and
attaching a second tower member to the second posterior fixation member.

24. The method of claim 23 wherein locking the position and orientation of the first portion of the first posterior fixation member to the first portion of the second posterior fixation member comprises clamping the first tower member to the second tower member.

25. The method of claim 19 wherein locking the position and orientation of the first portion of the first posterior fixation member to the first portion of the second posterior fixation member comprises attaching a limiter to a spanning member coupling the first portion of the first posterior fixation member to the first portion of the second posterior fixation member.

26. The method of claim 19 wherein the first portion of the first posterior fixation member comprises a first tulip, wherein the first portion of the second posterior fixation member comprises a second tulip, wherein the second portion of the first posterior fixation member comprises a first screw body, and wherein the second portion of the second posterior fixation member comprises a second screw body.

27. The method of claim 19 wherein the first vertebra is an L4 vertebra of the spine, wherein the second vertebra is an L5 vertebra of the spine, wherein the disc space is an L4/L5 disc space of the spine, and wherein inserting the trocar into the disc space comprises inserting the trocar along a lateral approach that extends through an iliac crest of the patient.

28. The method of claim 19 wherein the first vertebra is an L5 vertebra of the spine, wherein the second vertebra is an S1 vertebra of the spine, wherein the disc space is an L5/S1 disc space of the spine, and wherein inserting the trocar into the disc space comprises inserting the trocar along a lateral approach that extends through an iliac crest of the patient and a sacrum of the spine of the patient.

29. The method of claim 19 wherein the method further comprises measuring a lordotic angle of the spine in real time or near real time while expanding the balloon within the disc space to create the lordosis between the first vertebra and the second vertebra.

30. A system for treating a spine of a patient, comprising:
a trocar configured to be inserted to proximate a diseased disc within a disc space of the spine along a lateral, transpedicular, transfacet, or transforaminal access path;
a balloon configured to be inserted through the trocar and expanded within the disc space to distract the disc space;
a locking device configured to be coupled to a posterior fixation assembly secured to the spine to lock a position and orientation of (a) a portion of a first posterior fixation member secured to a first vertebra adjacent the disc space relative to (b) a portion of a second posterior fixation member secured to a second vertebra adjacent the disc space, thereby causing expansion of the balloon to create lordosis between the first vertebra and the second vertebra; and
an intervertebral device configured to be inserted through the trocar and expanded within the disc space.

31. The system of claim 30, further comprising a fill material configured to be inserted through the trocar and into the expanded intervertebral device.

32. The system of claim 30 further comprising a spinal angle measuring device configured to measure the created lordosis in real time or near real time.

33. The system of claim 30 wherein the locking device comprises a clamp configured to fixedly couple a first tower member attached to the first posterior fixation member to a second tower member attached to the second posterior fixation member.

34. The system of claim 30 wherein the locking device comprises a limiter configured to be secured to a spanning member coupling the portion of the first posterior fixation member to the portion of the second posterior fixation member.

35. The system of claim 30 wherein the portion of the first posterior fixation member comprises a tulip of the first posterior fixation member coupled to a screw body of the first posterior fixation member, and wherein the portion of the second posterior fixation member comprises a tulip of the second posterior fixation member coupled to a screw body of the second posterior fixation member.

36. A method of treating a spine of a human patient, the method comprising:
inserting a trocar to proximate a diseased disc within an L4/L5 disc space between an L4 vertebra and an L5 vertebra of the spine along a lateral access path that extends through an iliac crest of the patient;
inserting an intervertebral device through the trocar into the disc space; and
expanding the intervertebral device within the disc space.

37. The method of claim 36 wherein the method further comprises filling the intervertebral device with a fill material.

38. The method of claim 36 wherein the method further comprises:
inserting a balloon through the trocar into the disc space; and
expanding the balloon within the disc space to distract the disc space and create lordosis between the L4 vertebra and the L5 vertebra.

39. A method of treating a spine of a human patient, the method comprising:
inserting a trocar to proximate a diseased disc within an L5/S1 disc space between an L5 vertebra and an S1 vertebra of the spine along a lateral access path that extends through an iliac crest of the patient and a sacrum of the spine of the patient;
inserting an intervertebral device through the trocar into the disc space; and
expanding the intervertebral device within the disc space.

40. The method of claim 39 wherein the method further comprises filling the intervertebral device with a fill material.

41. The method of claim 39 wherein the method further comprises:
inserting a balloon through the trocar into the disc space; and
expanding the balloon within the disc space to distract the disc space and create lordosis between the L5 vertebra and the S1 vertebra.
